(19) United States
(12) Patent Application Publication
(10) Pub. No.: US 2011/0053997 A1
(43) Pub. Date: Mar. 3, 2011

(54) Salts and Crystal Forms

(76) Inventors: Alexander Beliaev, Mindelo (PT); David Alexander Learmonth, Alfena (PT); Melanie J. Roe, Lafayette, IN (US); Petinka Vlahova, West Lafayette, IN (US); Eric Hagen, Lafayette, IN (US); Valeriya Smolenskaya, Lafayette, IN (US); Donglai Yang, Annandale, NJ (US)

(21) Appl. No.: 12/746,239

(22) PCT Filed: Dec. 5, 2008
(86) PCT No.: PCT/PT08/00052

§ 371 Date: Oct. 4, 2010

Related U.S. Application Data
(60) Provisional application No. 60/992,398, filed on Dec. 5, 2007.

(51) Int. Cl. A61K 31/4178; C07D 405/04; A61P 9/00

(52) U.S. Cl. 514/397; 548/311.4

(57) ABSTRACT

The present invention relates to novel salts of the compound (R)-5-(2-Aminoethyl)-1-(6,8-difluorochroman-3-yl)-1,3-dihydroimidazole-2-thione, polymorphs of the salts and methods of their preparation.

This invention relates to salts of (R)-5-(2-Aminoethyl)-1-(6,8-difluorochroman-3-yl)-1,3-dihydroimidazole-2-thione, polymorphs of the salts and methods of their preparation.

(R)-5-(2-Aminoethyl)-1-(6,8-difluorochroman-3-yl)-1,3-dihythoimidazole-2-thione hydrochloride (the compound of formula I, below) is a potent, non-toxic and peripherally selective inhibitor of DβM, which can be used for treatment of certain cardiovascular disorders. It is disclosed in WO2004/033447, along with processes for its preparation.

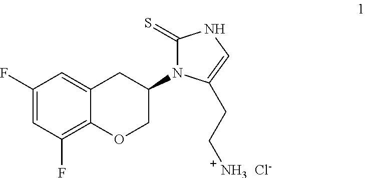

The process disclosed in WO2004/033447 for preparing compound 1 (see example 16) results in the amorphous form of compound 1. The process of example 16 is described in WO2004/033447 on page 5, lines 16 to 21 and in Scheme 2 on page 7. Prior to formation of compound 1, a mixture of intermediates is formed (compounds V and VI in scheme 2). The mixture of intermediates is subjected to a high concentration of HCl in ethyl acetate. Under these conditions, the primary product of the reaction is compound I, which precipitates as it forms as the amorphous form.

WO2007/139413 discloses polymorphic forms of compound 1.

The compounds disclosed in WO2004/033447 may exhibit advantageous properties. The polymorphs disclosed in WO2007/139413 may also exhibit advantageous properties. For example, the products may be advantageous in terms of their ease of production, for example easier filterability or drying. The products may be easy to store. The products may have increased processability. The products may be produced in high yield and/or high purity. The products may be advantageous in terms of their physical characteristics, such as solubility, melting point, hardness, density, hygroscopicity, stability, compatibility with excipients when formulated as a pharmaceutical. Furthermore, the products may have physiological advantages, for example they may exhibit high bioavailability.

We have now found certain new and advantageous salts of (R)-5-(2-Aminoethyl)-1-(6,8-difluorochroman-3-yl)-1,3-dihydroimidazole-2-thione and new and advantageous polymorphs thereof.

Accordingly, the present invention provides salts of (R)-5-(2-Aminoethyl)-1-(6,8-difluorochroman-3-yl)-1,3-dihydroimidazole-2-thione, other than the hydrochloride salt, and crystalline polymorphs of the salts. (R)-5-(2-Aminoethyl)-1-(6,8-difluorochroman-3-yl)-1,3-dihydroimidazole-2-thione has the following structure and is hereinafter referred to as compound 2.

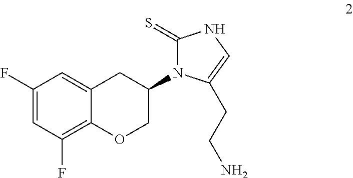

The present invention provides salts of (R)-5-(2-Aminoethyl)-1-(6,8-difluorochroman-3-yl)-1,3-dihydroimidazole-2-thione other than the hydrochloride salt. In particular, the present invention provides the following acid addition salts of compound 2: L-tartaric, malonic, toluenesulfonic, camphorsulfonic, fumaric, acetic, adipic, glutaric, glycolic, L-malic, citric, gentisic, maleic, hydrobromide, succinic, phosphoric and sulfuric. Each of the salts was found to exist in at least one crystalline polymorphic form and the present invention provides the characterisation of each of the forms.

Unless otherwise stated, all peak positions expressed in units of °2θ are subject to a margin of ±0.2 °2θ.

In the following description of the present invention, the polymorphic forms are described as having an XRPD pattern with peaks at the positions listed in the respective Tables. It is to be understood that, in one embodiment, the polymorphic form has an XRPD pattern with peaks at the °2θ positions listed±0.2 °2θ with any intensity (% (I/Io)) value; or in another embodiment, an XRPD pattern with peaks at the °2θ positions listed±0.1 °2θ. It is to be noted that the intensity values are included for information only and the definition of each of the peaks is not to be construed as being limited to particular intensity values.

According to one aspect of the present invention, there is provided the L-tartaric acid salt of (R)-5-(2-Aminoethyl)-1-(6,8-difluorochroman-3-yl)-1,3-dihydroimidazole-2-thione, i.e. (R)-5-(2-Aminoethyl)-1-(6,8-difluorochroman-3-yl)-1,3-dihydroimidazole-2-thione L-tartrate.

In an embodiment, there is provided (R)-5-(2-Aminoethyl)-1-(6,8-difluorochroman-3-yl)-1,3-dihydroimidazole-2-thione L-tartrate in amorphous form.

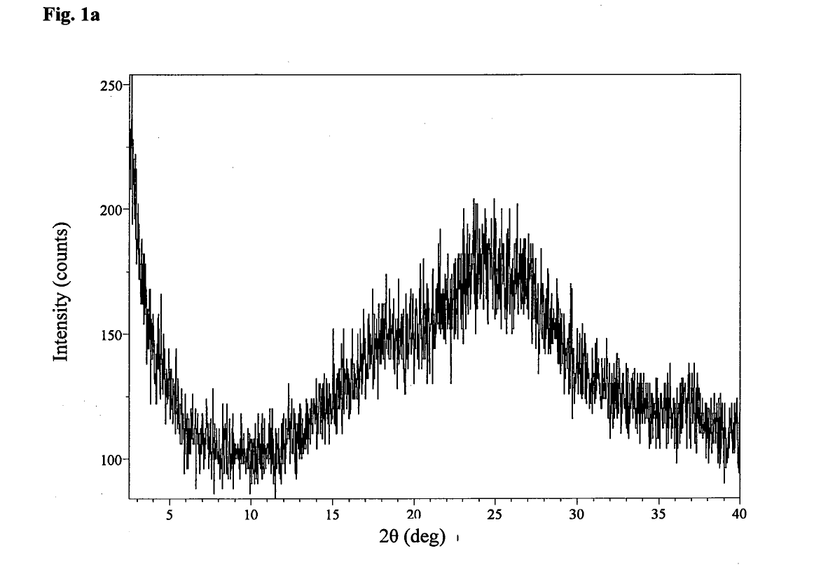

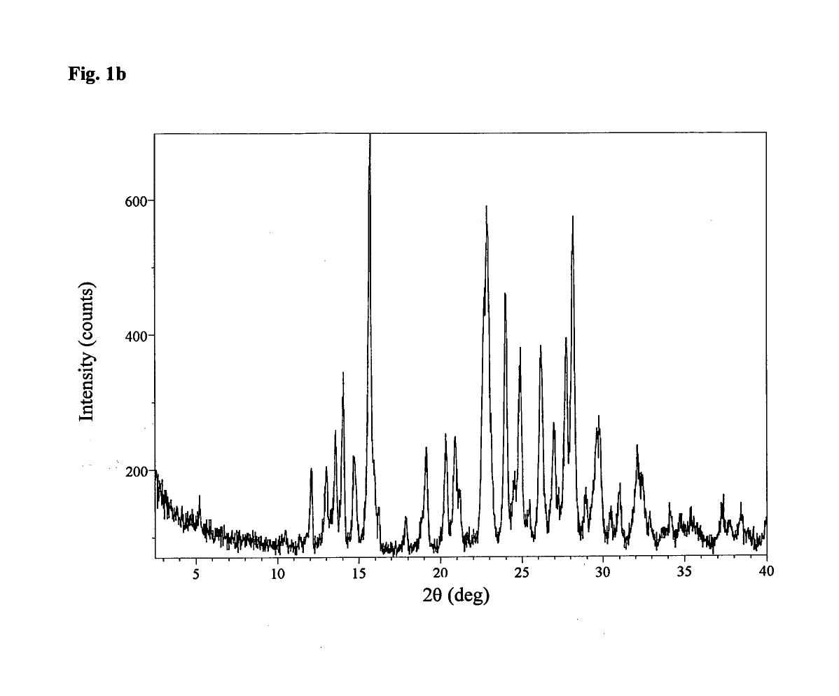

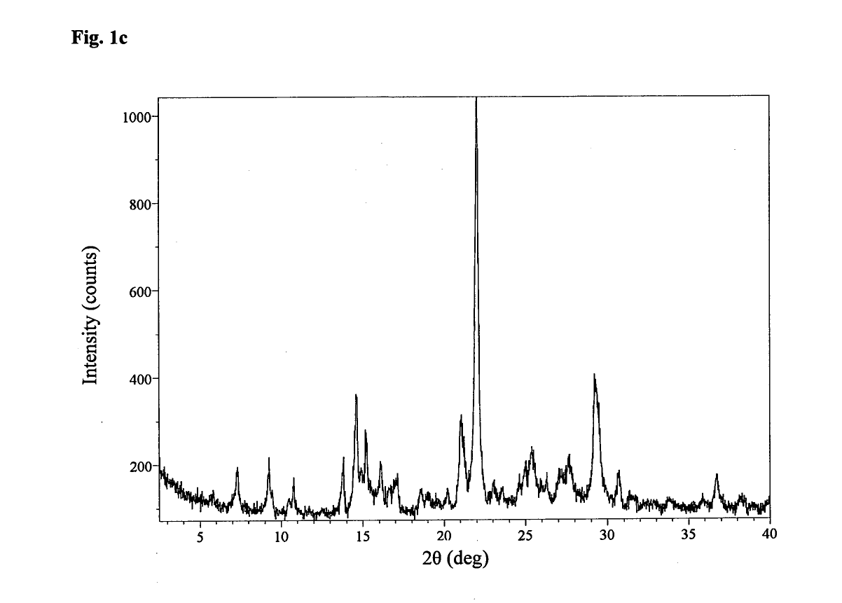

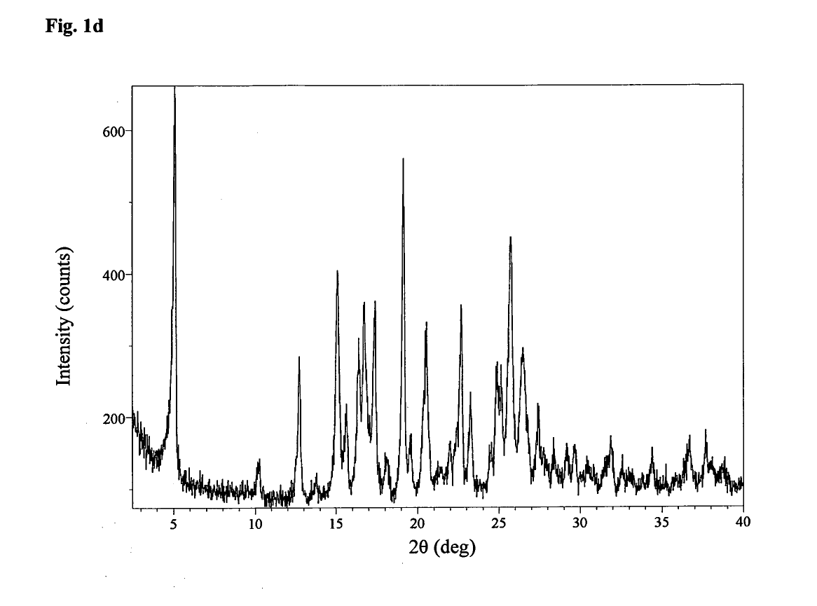

In an embodiment, the amorphous form of (R)-5-(2-Aminoethyl)-1-(6,8-difluorochroman-3-yl)-1,3-dihydroimidazole-2-thione L-tartrate has an XRPD as shown in FIG. 1a.

In another embodiment, there is provided crystalline Form A of (R)-5-(2-Aminoethyl)-1-(6,8-difluorochroman-3-yl)-1,3-dihydroimidazole-2-thione L-tartrate.

Form A may be characterised as having an XRPD pattern with peaks at 4.7, 6.0, 10.5, 11.5 and 14.0 °2θ±0.2 °2θ. The XRPD pattern may have further peaks at 16.4, 17.6 and 19.1 °2θ±0.2 °2θ. Form A may be characterised as having an absence of XRPD peaks between 6.5 and 10.0 °2θ.

In an embodiment, Form A has an XRPD pattern with peaks at the positions listed in Table 1 below.

TABLE 1

| ° 2θ | d space (Å) | Intensity % (I/Io) |
|---|---|---|
| 4.7 ± 0.1 | 18.842 ± 0.410 | 54 |
| 6.0 ± 0.1 | 14.780 ± 0.251 | 27 |
| 10.5 ± 0.1 | 8.417 ± 0.081 | 45 |
| 11.5 ± 0.1 | 7.715 ± 0.068 | 79 |
| 14.0 ± 0.1 | 6.317 ± 0.045 | 34 |
| 16.4 ± 0.1 | 5.389 ± 0.033 | 35 |
| 17.6 ± 0.1 | 5.034 ± 0.029 | 100 |
| 19.1 ± 0.1 | 4.649 ± 0.024 | 69 |

In another embodiment, Form A has an XRPD pattern with peaks at the positions listed in Table 2 below.

TABLE 2

| | | Intensity |
|---|---|---|
| ° 2θ | d space (Å) | % (I/Io) |
| 4.7 ± 0.1 | 18.842 ± 0.410 | 54 |
| 6.0 ± 0.1 | 14.780 ± 0.251 | 27 |
| 10.5 ± 0.1 | 8.417 ± 0.081 | 45 |
| 11.5 ± 0.1 | 7.715 ± 0.068 | 79 |
| 14.0 ± 0.1 | 6.317 ± 0.045 | 34 |
| 14.4 ± 0.1 | 6.160 ± 0.043 | 34 |
| 14.8 ± 0.1 | 5.998 ± 0.041 | 62 |
| 16.4 ± 0.1 | 5.389 ± 0.033 | 35 |
| 17.1 ± 0.1 | 5.173 ± 0.030 | 66 |
| 17.6 ± 0.1 | 5.034 ± 0.029 | 100 |
| 19.1 ± 0.1 | 4.649 ± 0.024 | 69 |

In yet another embodiment, Form A has an XRPD pattern with peaks at the positions listed in Table 3 below.

TABLE 3

| ° 2θ | d space (Å) | Intensity(%) |
|---|---|---|
| 4.7 ± 0.1 | 18.842 ± 0.410 | 54 |
| 6.0 ± 0.1 | 14.780 ± 0.251 | 27 |
| 10.5 ± 0.1 | 8.417 ± 0.081 | 45 |
| 11.5 ± 0.1 | 7.715 ± 0.068 | 79 |
| 11.9 ± 0.1 | 7.425 ± 0.063 | 26 |
| 12.6 ± 0.1 | 7.003 ± 0.056 | 15 |
| 13.2 ± 0.1 | 6.718 ± 0.051 | 13 |
| 14.0 ± 0.1 | 6.317 ± 0.045 | 34 |
| 14.4 ± 0.1 | 6.160 ± 0.043 | 34 |
| 14.8 ± 0.1 | 5.998 ± 0.041 | 62 |
| 15.2 ± 0.1 | 5.844 ± 0.039 | 50 |
| 16.4 ± 0.1 | 5.389 ± 0.033 | 35 |
| 17.1 ± 0.1 | 5.173 ± 0.030 | 66 |
| 17.6 ± 0.1 | 5.034 ± 0.029 | 100 |
| 18.1 ± 0.1 | 4.901 ± 0.027 | 30 |
| 19.1 ± 0.1 | 4.649 ± 0.024 | 69 |
| 19.8 ± 0.1 | 4.482 ± 0.023 | 54 |
| 20.0 ± 0.1 | 4.442 ± 0.022 | 49 |
| 20.9 ± 0.1 | 4.259 ± 0.020 | 36 |
| 21.2 ± 0.1 | 4.193 ± 0.020 | 61 |
| 21.9 ± 0.1 | 4.057 ± 0.018 | 31 |
| 22.8 ± 0.1 | 3.894 ± 0.017 | 38 |
| 24.1 ± 0.1 | 3.693 ± 0.015 | 77 |
| 24.8 ± 0.1 | 3.592 ± 0.014 | 51 |
| 25.7 ± 0.1 | 3.468 ± 0.013 | 27 |
| 26.5 ± 0.1 | 3.360 ± 0.012 | 33 |
| 27.1 ± 0.1 | 3.290 ± 0.012 | 28 |
| 28.2 ± 0.1 | 3.160 ± 0.011 | 38 |
| 28.8 ± 0.1 | 3.099 ± 0.011 | 28 |
| 29.6 ± 0.1 | 3.013 ± 0.010 | 38 |

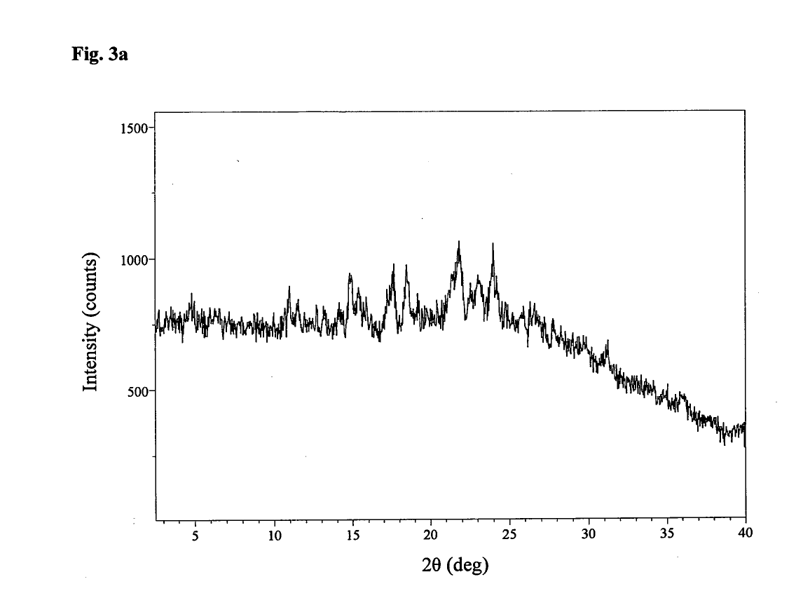

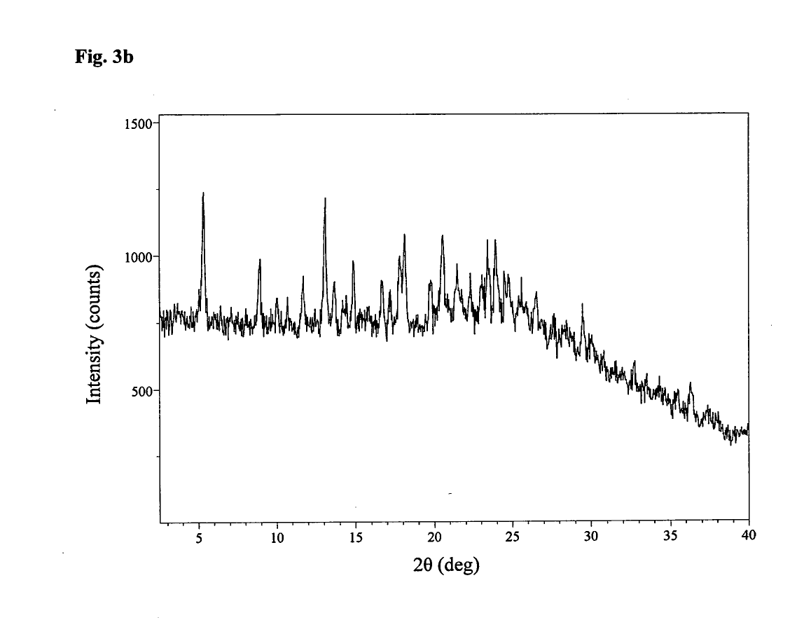

In an embodiment, Form A of (R)-5-(2-Aminoethyl)-1-(6,8-difluorochroman-3-yl)-1,3-dihydroimidazole-2-thione L-tartrate has the XRPD pattern as shown in FIG. 3a.

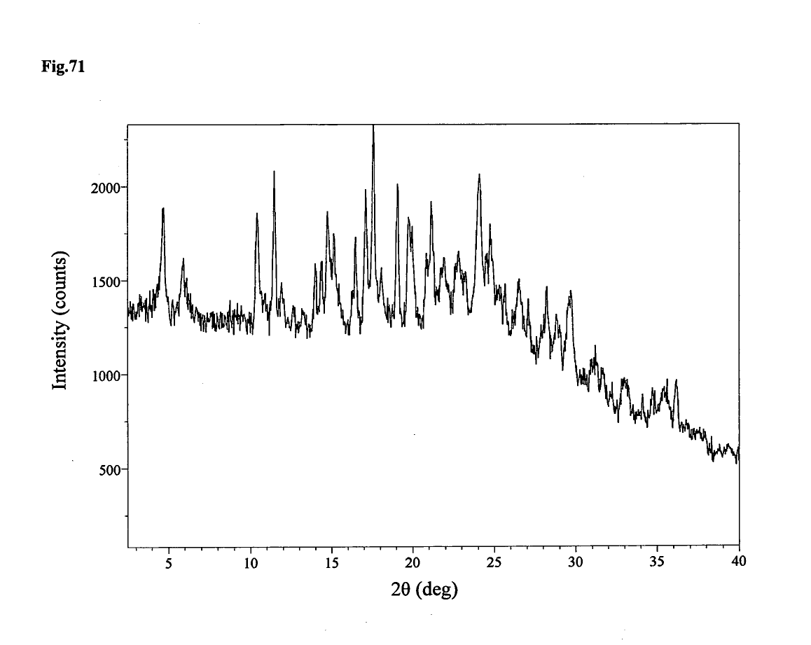

In an embodiment, Form A of (R)-5-(2-Aminoethyl)-1-(6,8-difluorochroman-3-yl)-1,3-dihydroimidazole-2-thione L-tartrate has the XRPD pattern as shown in FIG. 71.

In another embodiment, there is provided crystalline Form B of (R)-5-(2-Aminoethyl)-1-(6,8-difluorochroman-3-yl)-1,3-dihydroimidazole-2-thione L-tartrate.

Form B may be characterised as having an XRPD pattern with peaks at 5.4, 9.0 and 13.7 °2θ±0.2 °2θ. The XRPD pattern may have further peaks at 16.7 and 20.6 °2θ±0.2 °2θ. The XRPD pattern may have still further peaks at 11.7, 13.1 and 14.9 °2θ±0.2°θ.

In an embodiment, Form B has an XRPD pattern with peaks at the positions listed in Table 4 below.

TABLE 4

| ° 2θ | d space (Å) | Intensity % (I/Io) |
|---|---|---|
| 5.4 ± 0.1 | 16.519 ± 0.314 | 100 |
| 9.0 ± 0.1 | 9.881 ± 0.111 | 57 |
| 13.7 ± 0.1 | 6.468 ± 0.047 | 40 |
| 16.7 ± 0.1 | 5.312 ± 0.032 | 41 |
| 20.6 ± 0.1 | 4.320 ± 0.021 | 71 |

In another embodiment, Form B has an XRPD pattern with peaks at the positions listed in Table 5 below.

TABLE 5

| ° 2θ | d space (Å) | Intensity % (I/Io) |
|---|---|---|
| 5.4 ± 0.1 | 16.519 ± 0.314 | 100 |
| 9.0 ± 0.1 | 9.881 ± 0.111 | 57 |
| 11.7 ± 0.1 | 7.557 ± 0.065 | 42 |
| 13.1 ± 0.1 | 6.764 ± 0.052 | 94 |
| 13.7 ± 0.1 | 6.468 ± 0.047 | 40 |
| 14.9 ± 0.1 | 5.950 ± 0.040 | 54 |
| 16.7 ± 0.1 | 5.312 ± 0.032 | 41 |
| 17.8 ± 0.1 | 4.983 ± 0.028 | 58 |
| 18.1 ± 0.1 | 4.893 ± 0.027 | 75 |
| 19.8 ± 0.1 | 4.482 ± 0.023 | 39 |
| 20.6 ± 0.1 | 4.320 ± 0.021 | 71 |

In yet another embodiment, Form B has an XRPD pattern with peaks at the positions listed in Table 6 below.

TABLE 6

| ° 2θ | d space (Å) | Intensity % (I/Io) |
|---|---|---|
| 5.4 ± 0.1 | 16.519 ± 0.314 | 100 |
| 9.0 ± 0.1 | 9.881 ± 0.111 | 57 |
| 11.7 ± 0.1 | 7.557 ± 0.065 | 42 |
| 13.1 ± 0.1 | 6.764 ± 0.052 | 94 |
| 13.7 ± 0.1 | 6.468 ± 0.047 | 40 |
| 14.9 ± 0.1 | 5.950 ± 0.040 | 54 |
| 16.7 ± 0.1 | 5.312 ± 0.032 | 41 |
| 17.2 ± 0.1 | 5.147 ± 0.030 | 34 |
| 17.8 ± 0.1 | 4.983 ± 0.028 | 58 |
| 18.1 ± 0.1 | 4.893 ± 0.027 | 75 |
| 19.8 ± 0.1 | 4.482 ± 0.023 | 39 |
| 20.6 ± 0.1 | 4.320 ± 0.021 | 71 |
| 21.5 ± 0.1 | 4.135 ± 0.019 | 49 |
| 22.3 ± 0.1 | 3.981 ± 0.018 | 39 |
| 23.1 ± 0.1 | 3.854 ± 0.017 | 43 |
| 23.4 ± 0.1 | 3.800 ± 0.016 | 62 |
| 24.0 ± 0.1 | 3.716 ± 0.015 | 69 |
| 24.5 ± 0.1 | 3.631 ± 0.015 | 45 |
| 26.6 ± 0.1 | 3.356 ± 0.012 | 40 |
| 29.5 ± 0.1 | 3.031 ± 0.010 | 44 |

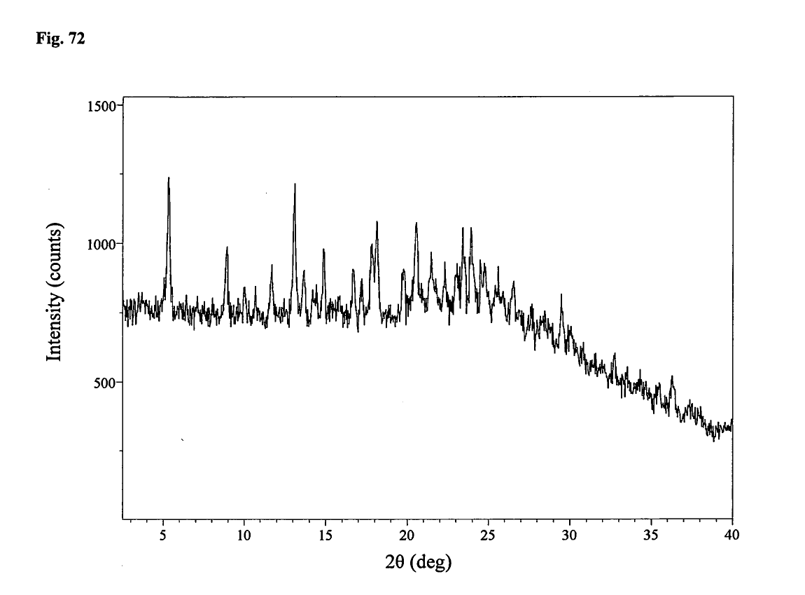

In an embodiment, Form B of (R)-5-(2-Aminoethyl)-1-(6,8-difluorochroman-3-yl)-1,3-dihydroimidazole-2-thione L-tartrate has the XRPD pattern as shown in FIG. 3b. In an embodiment, Form B of (R)-5-(2-Aminoethyl)-1-(6,8-difluorochroman-3-yl)-1,3-dihydroimidazole-2-thione L-tartrate has the XRPD pattern as shown in FIG. 72.

In another embodiment, Form B is characterised as being in the form of a solvate of tetrahydrofuran (THF). The number of moles of tetrahydrofuran per mole of Form B may range from 0.4 to 0.9. Typically, the number of moles ranges from 0.5 to 0.8. In an embodiment, there is 0.7 mole of THF per 1 mole of Form B.

According to another aspect of the present invention, there is provided the malonic acid salt of (R)-5-(2-Aminoethyl)-1-(6,8-difluorochroman-3-yl)-1,3-dihydroimidazole-2-thione, i.e. (R)-5-(2-Aminoethyl)-1-(6,8-difluorochroman-3-yl)-1,3-dihydroimidazole-2-thione malonate.

In an embodiment, there is provided crystalline Form A of (R)-5-(2-Aminoethyl)-1-(6,8-difluorochroman-3-yl)-1,3-dihydroimidazole-2-thione malonate.

Form A may be characterised as having an XRPD pattern with peaks at 5.2, 12.1, 13.0, 13.6, 14.1 and 14.8 °2θ±0.2 °2θ. The XRPD pattern may have a further peak at 15.7 °2θ±0.2 °2θ. The XRPD pattern may have still further peaks at 19.2 and 20.4 °2θ±0.2°θ.

In an embodiment, Form A has an XRPD pattern with peaks at the positions listed in Table 7 below.

TABLE 7

| ° 2θ | d space (Å) | Intensity % (I/Io) |
|---|---|---|
| 5.2 ± 0.1 | 16.897 ± 0.329 | 15 |
| 12.1 ± 0.1 | 7.297 ± 0.060 | 32 |
| 13.0 ± 0.1 | 6.795 ± 0.052 | 28 |
| 13.6 ± 0.1 | 6.511 ± 0.048 | 44 |
| 14.1 ± 0.1 | 6.290 ± 0.045 | 58 |
| 14.8 ± 0.1 | 5.998 ± 0.041 | 28 |
| 15.7 ± 0.1 | 5.645 ± 0.036 | 100 |
| 19.2 ± 0.1 | 4.628 ± 0.024 | 27 |
| 20.4 ± 0.1 | 4.364 ± 0.021 | 30 |

In another embodiment, Form B has an XRPD pattern with peaks at the positions listed in Table 8 below.

TABLE 8

| ° 2θ | d space (Å) | Intensity % (I/Io) |
|---|---|---|
| 5.2 ± 0.1 | 16.897 ± 0.329 | 15 |
| 10.5 ± 0.1 | 8.441 ± 0.081 | 4 |
| 11.5 ± 0.1 | 7.695 ± 0.067 | 4 |
| 12.1 ± 0.1 | 7.297 ± 0.060 | 32 |
| 13.0 ± 0.1 | 6.795 ± 0.052 | 28 |
| 13.6 ± 0.1 | 6.511 ± 0.048 | 44 |
| 14.1 ± 0.1 | 6.290 ± 0.045 | 58 |
| 14.8 ± 0.1 | 5.998 ± 0.041 | 28 |
| 15.7 ± 0.1 | 5.645 ± 0.036 | 100 |
| 16.2 ± 0.1 | 5.478 ± 0.034 | 12 |
| 17.9 ± 0.1 | 4.958 ± 0.028 | 9 |
| 19.2 ± 0.1 | 4.628 ± 0.024 | 27 |
| 20.4 ± 0.1 | 4.364 ± 0.021 | 30 |
| 20.9 ± 0.1 | 4.246 ± 0.020 | 26 |
| 21.2 ± 0.1 | 4.193 ± 0.020 | 15 |
| 22.7 ± 0.1 | 3.919 ± 0.017 | 40 |
| 22.9 ± 0.1 | 3.879 ± 0.017 | 70 |
| 24.0 ± 0.1 | 3.702 ± 0.015 | 54 |
| 24.6 ± 0.1 | 3.626 ± 0.015 | 14 |
| 24.9 ± 0.1 | 3.570 ± 0.014 | 44 |
| 25.4 ± 0.1 | 3.500 ± 0.014 | 7 |
| 26.2 ± 0.1 | 3.398 ± 0.013 | 34 |
| 27.0 ± 0.1 | 3.298 ± 0.012 | 23 |
| 27.8 ± 0.1 | 3.210 ± 0.011 | 43 |
| 28.2 ± 0.1 | 3.163 ± 0.011 | 66 |
| 29.0 ± 0.1 | 3.083 ± 0.010 | 9 |
| 29.9 ± 0.1 | 2.992 ± 0.010 | 22 |

In an embodiment, Form A of (R)-5-(2-Aminoethyl)-1-(6,8-difluorochroman-3-yl)-1,3-dihydroimidazole-2-thione malonate has the XRPD pattern as shown in FIG. 1b.

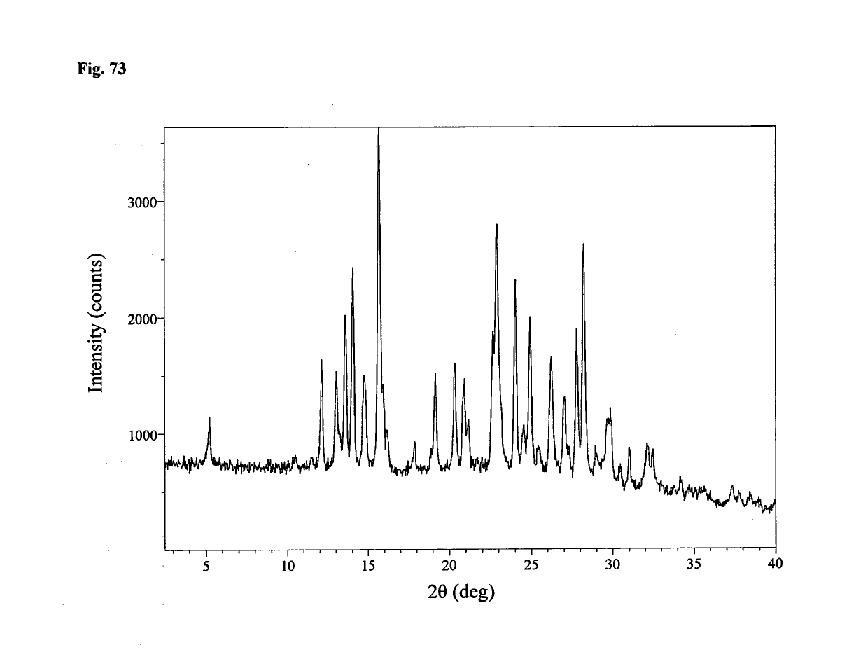

In an embodiment, Form A of (R)-5-(2-Aminoethyl)-1-(6,8-difluorochroman-3-yl)-1,3-dihydroimidazole-2-thione malonate has the XRPD pattern as shown in FIG. 73.

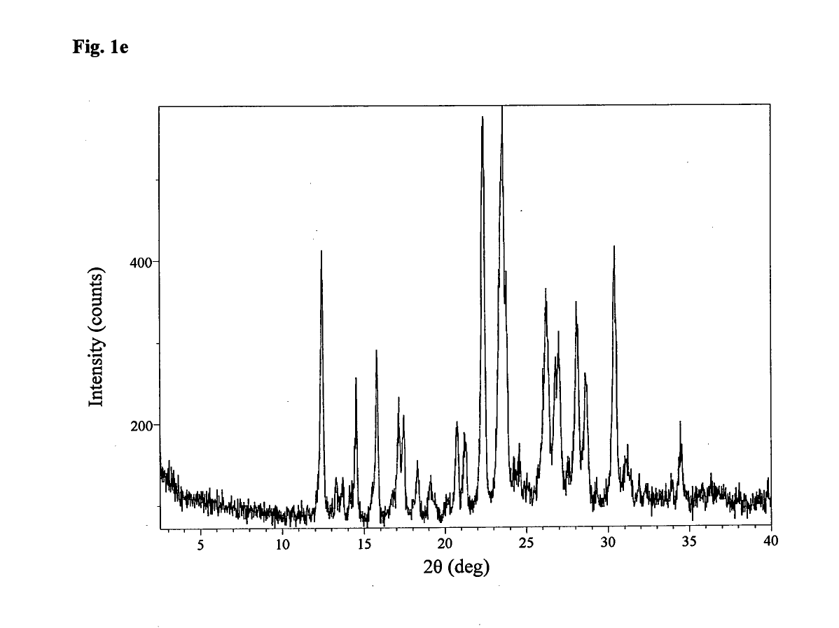

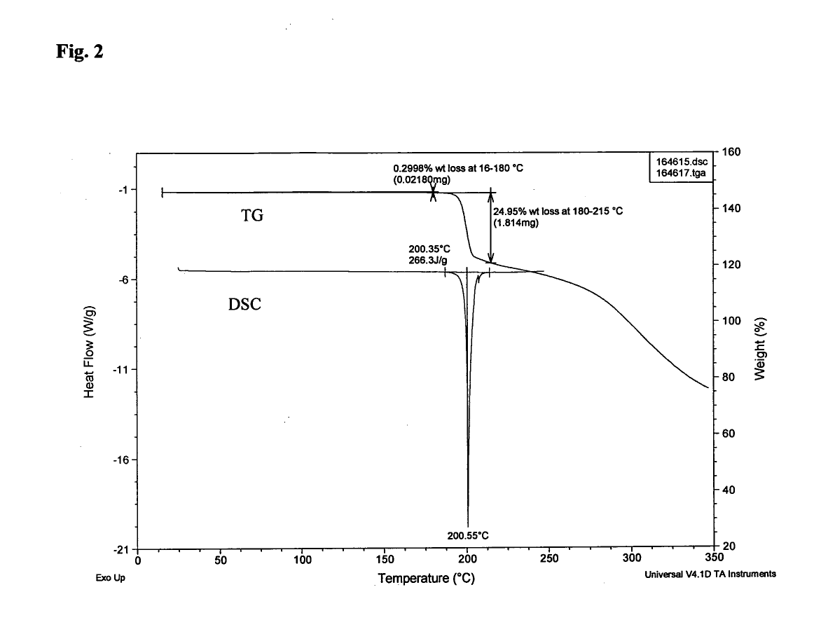

Form A of (R)-5-(2-Aminoethyl)-1-(6,8-difluorochroman-3-yl)-1,3-dihydroimidazole-2-thione malonate may also be characterised as having the DSC thermogram as shown in FIG. 2.

According to another aspect of the present invention, there is provided the camphorsulfonic acid salt of (R)-5-(2-Aminoethyl)-1-(6,8-difluorochroman-3-yl)-1,3-dihydroimidazole-2-thione, i.e. (R)-5-(2-Aminoethyl)-1-(6,8-difluorochroman-3-yl)-1,3-dihydroimidazole-2-thione camphorsulfonate or camsylate.

In an embodiment, there is provided crystalline Form A of (R)-5-(2-Aminoethyl)-1-(6,8-difluorochroman-3-yl)-1,3-dihydroimidazole-2-thione camsylate.

Form A may be characterised as having an XRPD pattern with a peak at 5.0 °2θ±0.2 °2θ. The XRPD pattern may have further peaks at 10.2 and 12.7 °2θ±0.2 °2θ. The XRPD pattern may have yet further peaks at 15.1, 15.6, 16.4, 16.7 and 17.4 °2θ±0.2 °2θ.

In an embodiment, Form A has an XRPD pattern with peaks at the positions listed in Table 9 below.

TABLE 9

| ° 2θ | d space (Å) | Intensity % (I/Io) |
|---|---|---|
| 5.0 ± 0.1 | 17.499 ± 0.353 | 100 |
| 10.2 ± 0.1 | 8.639 ± 0.085 | 10 |
| 12.7 ± 0.1 | 6.954 ± 0.055 | 25 |
| 15.1 ± 0.1 | 5.879 ± 0.039 | 69 |
| 15.6 ± 0.1 | 5.677 ± 0.036 | 27 |
| 16.4 ± 0.1 | 5.418 ± 0.033 | 31 |
| 16.7 ± 0.1 | 5.312 ± 0.032 | 34 |
| 17.4 ± 0.1 | 5.111 ± 0.029 | 35 |
| 19.1 ± 0.1 | 4.642 ± 0.024 | 42 |
| 20.5 ± 0.1 | 4.326 ± 0.021 | 23 |
| 25.7 ± 0.1 | 3.464 ± 0.013 | 40 |

In another embodiment, Form A has an XRPD pattern with peaks at the positions listed in Table 10 below.

TABLE 10

| ° 2θ | d space (Å) | Intensity % (I/Io) |
|---|---|---|
| 5.0 ± 0.1 | 17.499 ± 0.353 | 100 |
| 8.5 ± 0.1 | 10.366 ± 0.123 | 6 |
| 10.2 ± 0.1 | 8.639 ± 0.085 | 10 |
| 12.7 ± 0.1 | 6.954 ± 0.055 | 25 |
| 13.8 ± 0.1 | 6.440 ± 0.047 | 5 |
| 15.1 ± 0.1 | 5.879 ± 0.039 | 69 |
| 15.6 ± 0.1 | 5.677 ± 0.036 | 27 |
| 16.4 ± 0.1 | 5.418 ± 0.033 | 31 |
| 16.7 ± 0.1 | 5.312 ± 0.032 | 34 |
| 17.4 ± 0.1 | 5.111 ± 0.029 | 35 |
| 18.1 ± 0.1 | 4.901 ± 0.027 | 6 |
| 19.1 ± 0.1 | 4.642 ± 0.024 | 42 |
| 19.5 ± 0.1 | 4.543 ± 0.023 | 9 |
| 20.5 ± 0.1 | 4.326 ± 0.021 | 23 |
| 22.0 ± 0.1 | 4.046 ± 0.018 | 7 |
| 22.4 ± 0.1 | 3.971 ± 0.018 | 7 |
| 22.7 ± 0.1 | 3.924 ± 0.017 | 12 |
| 23.3 ± 0.1 | 3.824 ± 0.016 | 11 |
| 24.5 ± 0.1 | 3.635 ± 0.015 | 5 |
| 24.9 ± 0.1 | 3.575 ± 0.014 | 24 |
| 25.1 ± 0.1 | 3.545 ± 0.014 | 23 |
| 25.7 ± 0.1 | 3.464 ± 0.013 | 40 |
| 26.5 ± 0.1 | 3.367 ± 0.013 | 15 |
| 27.4 ± 0.1 | 3.252 ± 0.012 | 8 |
| 28.4 ± 0.1 | 3.144 ± 0.011 | 6 |
| 29.2 ± 0.1 | 3.062 ± 0.010 | 6 |
| 29.6 ± 0.1 | 3.013 ± 0.010 | 5 |

In an embodiment, Form A of (R)-5-(2-Aminoethyl)-1-(6,8-difluorochroman-3-yl)-1,3-dihydroimidazole-2-thione camsylate has the XRPD pattern as shown in FIG. 1d.

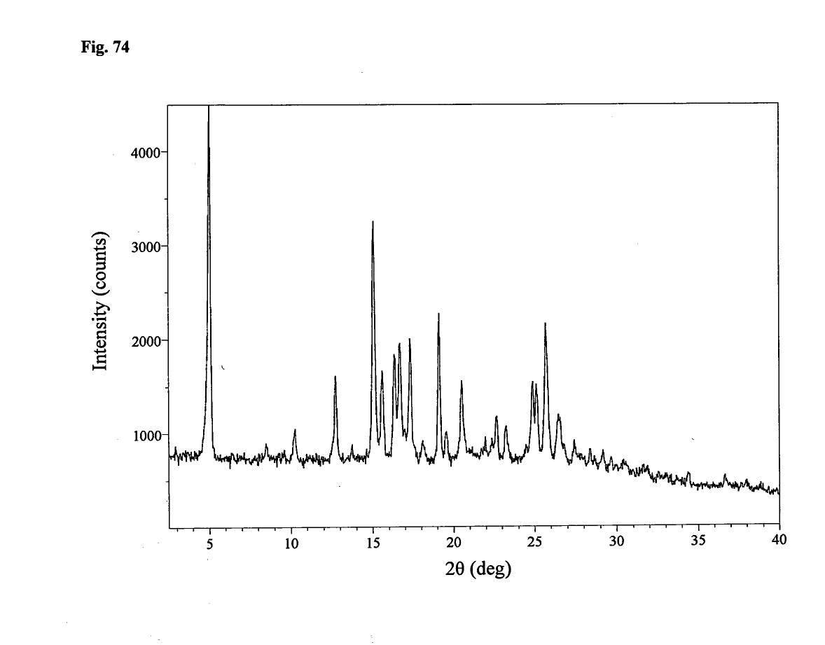

In an embodiment, Form A of (R)-5-(2-Aminoethyl)-1-(6,8-difluorochroman-3-yl)-1,3-dihydroimidazole-2-thione camsylate has the XRPD pattern as shown in FIG. 74.

According to another aspect of the present invention, there is provided the fumaric acid salt of (R)-5-(2-Aminoethyl)-1-(6,8-difluorochroman-3-yl)-1,3-dihydroimidazole-2-thione, i.e. (R)-5-(2-Aminoethyl)-1-(6,8-difluorochroman-3-yl)-1,3-dihydroimidazole-2-thione fumarate.

In an embodiment, there is provided crystalline Form A of (R)-5-(2-Aminoethyl)-1-(6,8-difluorochroman-3-yl)-1,3-dihydroimidazole-2-thione fumarate.

Form A may be characterised as having an XRPD pattern with peaks at 12.5 and 14.6 °2θ±0.2 °2θ. The XRPD pattern may have further peaks at 13.3 and 13.7 °2θ±0.2 °2θ. The XRPD pattern may have yet further peaks at 15.8, 17.5, 22.5 and 23.6 °2θ±0.2 °2θ.

In an embodiment, Form A has an XRPD pattern with peaks at the positions listed in Table 11 below.

TABLE 11

| | | Intensity % |
|---|---|---|
| ° 2θ | d space (Å) | (I/Io) |
| 12.5 ± 0.1 | 7.070 ± 0.057 | 100 |
| 13.3 ± 0.1 | 6.642 ± 0.050 | 15 |
| 13.7 ± 0.1 | 6.454 ± 0.047 | 15 |
| 14.6 ± 0.1 | 6.084 ± 0.042 | 41 |
| 15.8 ± 0.1 | 5.602 ± 0.035 | 44 |
| 17.2 ± 0.1 | 5.164 ± 0.030 | 24 |
| 17.5 ± 0.1 | 5.068 ± 0.029 | 28 |
| 18.3 ± 0.1 | 4.838 ± 0.026 | 17 |
| 20.8 ± 0.1 | 4.271 ± 0.020 | 23 |
| 21.3 ± 0.1 | 4.170 ± 0.019 | 15 |
| 22.5 ± 0.1 | 3.955 ± 0.017 | 77 |
| 23.6 ± 0.1 | 3.767 ± 0.016 | 59 |

In another embodiment, Form A has an XRPD pattern with peaks at the positions listed in Table 12 below.

TABLE 12

| ° 2θ | d space (Å) | Intensity % (I/Io) |
|---|---|---|
| 12.5 ± 0.1 | 7.070 ± 0.057 | 100 |
| 13.3 ± 0.1 | 6.642 ± 0.050 | 15 |
| 13.7 ± 0.1 | 6.454 ± 0.047 | 15 |
| 14.6 ± 0.1 | 6.084 ± 0.042 | 41 |
| 15.8 ± 0.1 | 5.602 ± 0.035 | 44 |
| 17.2 ± 0.1 | 5.164 ± 0.030 | 24 |
| 17.5 ± 0.1 | 5.068 ± 0.029 | 28 |
| 18.3 ± 0.1 | 4.838 ± 0.026 | 17 |
| 19.2 ± 0.1 | 4.620 ± 0.024 | 7 |
| 20.3 ± 0.1 | 4.383 ± 0.022 | 6 |
| 20.8 ± 0.1 | 4.271 ± 0.020 | 23 |
| 21.3 ± 0.1 | 4.170 ± 0.019 | 15 |
| 22.5 ± 0.1 | 3.955 ± 0.017 | 77 |
| 23.6 ± 0.1 | 3.767 ± 0.016 | 59 |
| 24.6 ± 0.1 | 3.617 ± 0.015 | 11 |
| 26.3 ± 0.1 | 3.390 ± 0.013 | 28 |
| 26.8 ± 0.1 | 3.327 ± 0.012 | 23 |
| 27.1 ± 0.1 | 3.294 ± 0.012 | 24 |
| 27.6 ± 0.1 | 3.234 ± 0.012 | 8 |
| 28.2 ± 0.1 | 3.160 ± 0.011 | 16 |
| 28.8 ± 0.1 | 3.099 ± 0.011 | 15 |

In an embodiment, Form A of (R)-5-(2-Aminoethyl)-1-(6,8-difluorochroman-3-yl)-1,3-dihydroimidazole-2-thione fumarate has the XRPD pattern as shown in FIG. 1e.

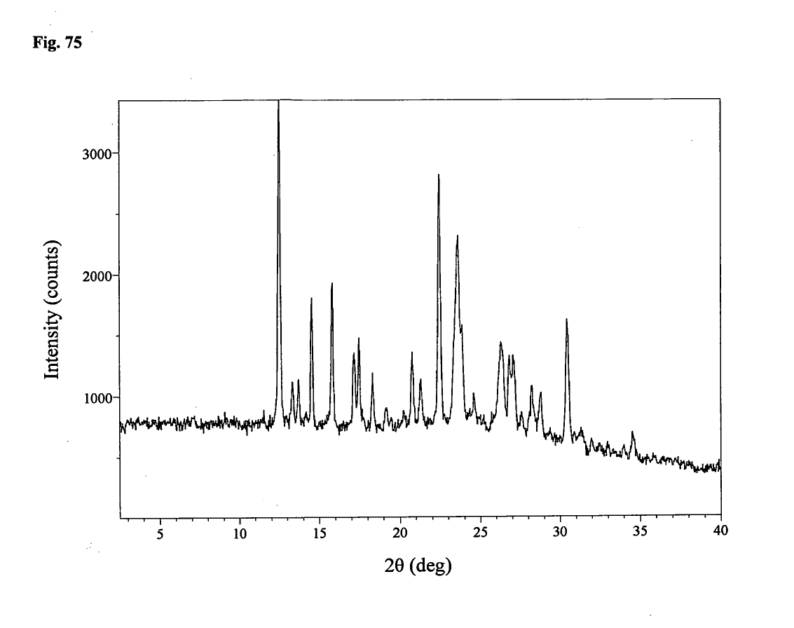

In an embodiment, Form A of (R)-5-(2-Aminoethyl)-1-(6,8-difluorochroman-3-yl)-1,3-dihydroimidazole-2-thione fumarate has the XRPD pattern as shown in FIG. 75.

According to another aspect of the present invention, there is provided the toluenesulfonic acid salt of (R)-5-(2-Aminoethyl)-1-(6,8-difluorochroman-3-yl)-1,3-dihydroimidazole-2-thione, i.e. (R)-5-(2-Aminoethyl)-1-(6,8-difluorochroman-3-yl)-1,3-dihydroimidazole-2-thione tosylate.

In an embodiment, there is provided crystalline Form A of (R)-5-(2-Aminoethyl)-1-(6,8-difluorochroman-3-yl)-1,3-dihydroimidazole-2-thione tosylate.

Form A may be characterised as having an XRPD pattern with peaks at 7.3, 9.2 and 14.6 °2θ±0.2 °2θ. The XRPD pattern may have further peaks at 10.8, 13.8 and 14.9 °2θ±0.2 °2θ.

The XRPD pattern may have still further peaks at 16.1, 22.0 and 25.0 °2θ±0.2°θ.

In an embodiment, Form A has an XRPD pattern with peaks at the positions listed in Table 13 below.

TABLE 13

| °2θ | d space (Å) | Intensity % (I/Io) |
|---|---|---|
| 7.3 ± 0.1 | 12.110 ± 0.168 | 39 |
| 9.2 ± 0.1 | 9.561 ± 0.104 | 31 |
| 14.6 ± 0.1 | 6.059 ± 0.042 | 81 |

In another embodiment, Form A has an XRPD pattern with peaks at the positions listed in Table 14 below.

TABLE 14

| °2θ | d space (Å) | Intensity % (I/Io) |
|---|---|---|
| 7.3 ± 0.1 | 12.110 ± 0.168 | 39 |
| 8.1 ± 0.1 | 10.862 ± 0.135 | 11 |
| 9.2 ± 0.1 | 9.561 ± 0.104 | 31 |
| 10.8 ± 0.1 | 8.207 ± 0.077 | 21 |
| 12.5 ± 0.1 | 7.104 ± 0.057 | 10 |
| 13.2 ± 0.1 | 6.687 ± 0.051 | 11 |
| 13.8 ± 0.1 | 6.426 ± 0.047 | 50 |
| 14.6 ± 0.1 | 6.059 ± 0.042 | 81 |
| 14.9 ± 0.1 | 5.938 ± 0.040 | 87 |
| 16.1 ± 0.1 | 5.498 ± 0.034 | 88 |
| 16.7 ± 0.1 | 5.321 ± 0.032 | 21 |
| 17.1 ± 0.1 | 5.192 ± 0.030 | 15 |
| 18.6 ± 0.1 | 4.783 ± 0.026 | 14 |
| 18.9 ± 0.1 | 4.686 ± 0.025 | 11 |
| 20.2 ± 0.1 | 4.390 ± 0.022 | 23 |
| 21.3 ± 0.1 | 4.175 ± 0.019 | 37 |
| 22.0 ± 0.1 | 4.035 ± 0.018 | 100 |
| 25.0 ± 0.1 | 3.558 ± 0.014 | 94 |
| 25.4 ± 0.1 | 3.500 ± 0.014 | 60 |
| 26.0 ± 0.1 | 3.421 ± 0.013 | 21 |
| 27.0 ± 0.1 | 3.305 ± 0.012 | 25 |
| 27.7 ± 0.1 | 3.224 ± 0.011 | 38 |
| 28.6 ± 0.1 | 3.121 ± 0.011 | 16 |
| 29.4 ± 0.1 | 3.037 ± 0.010 | 36 |

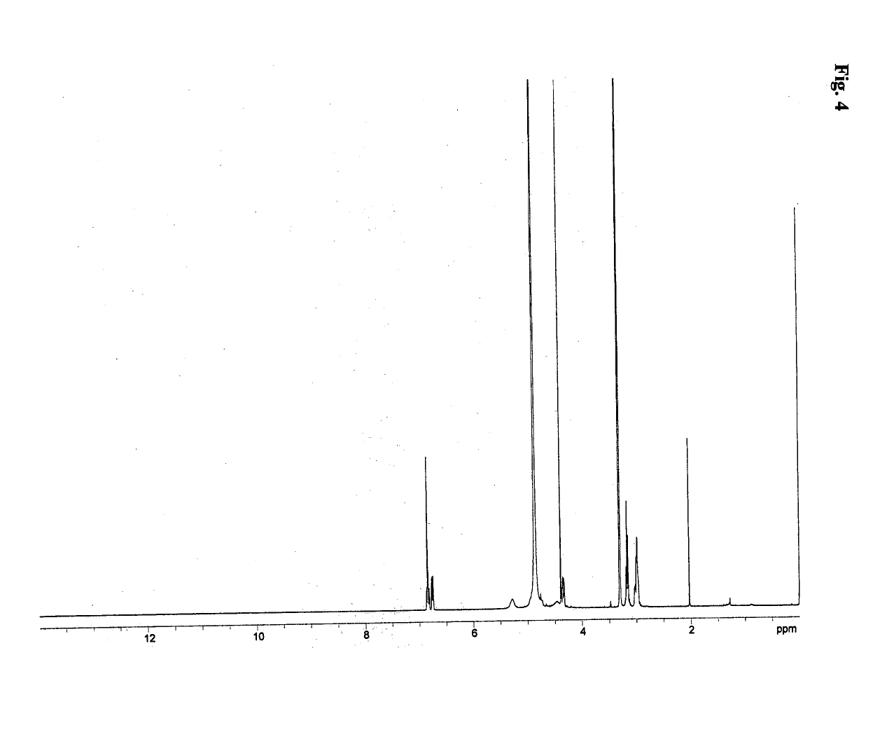

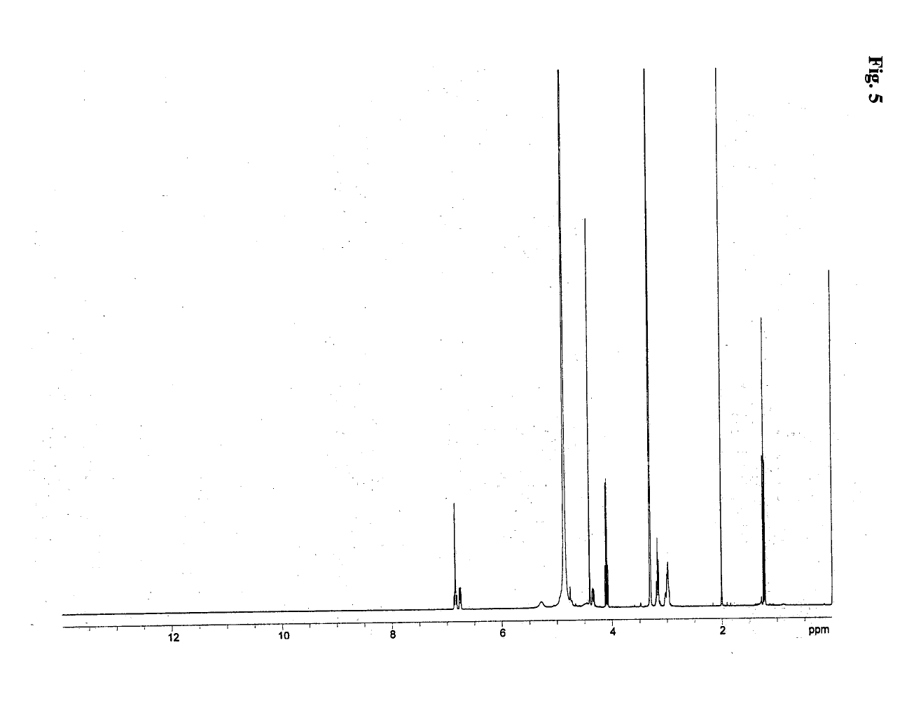

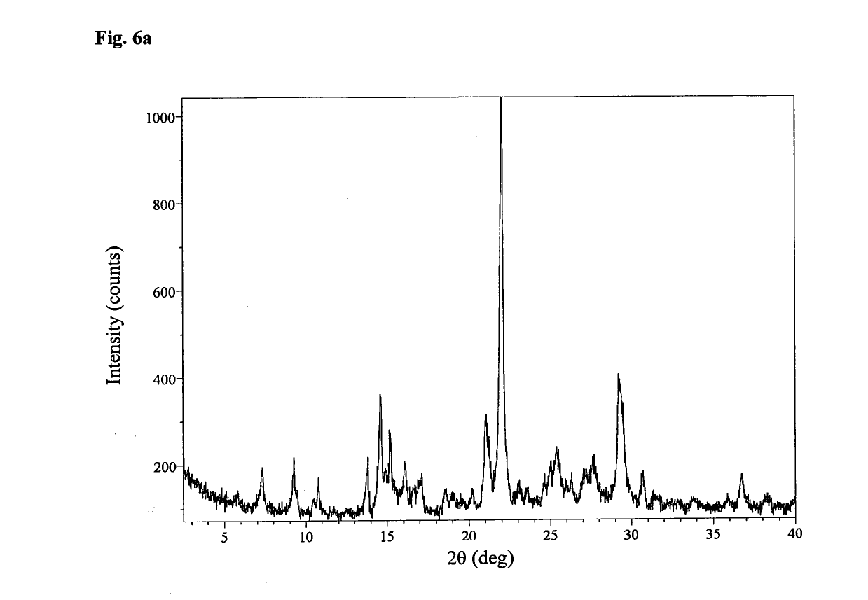

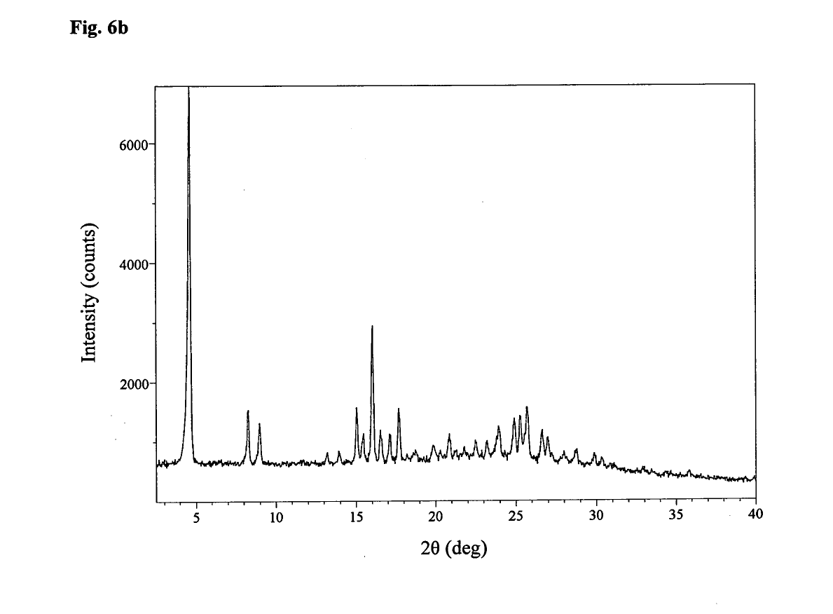

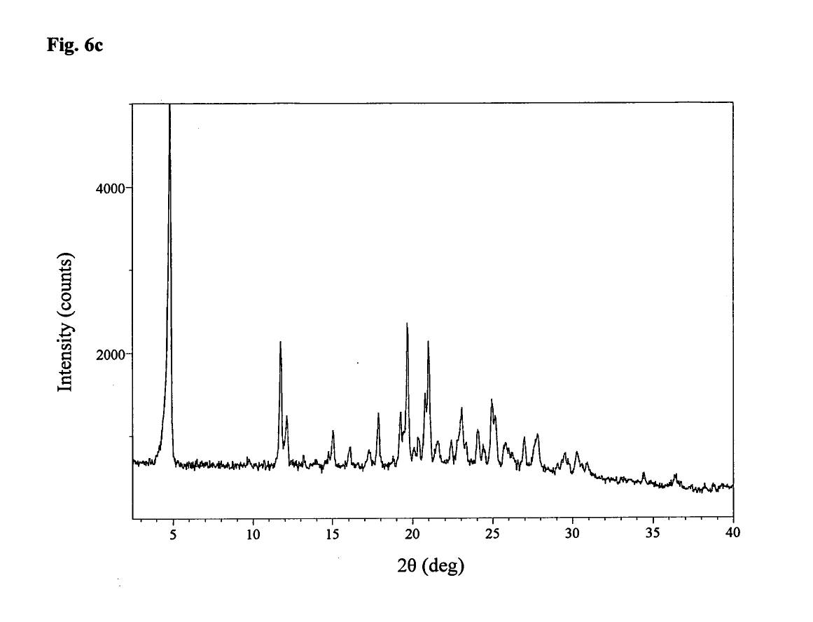

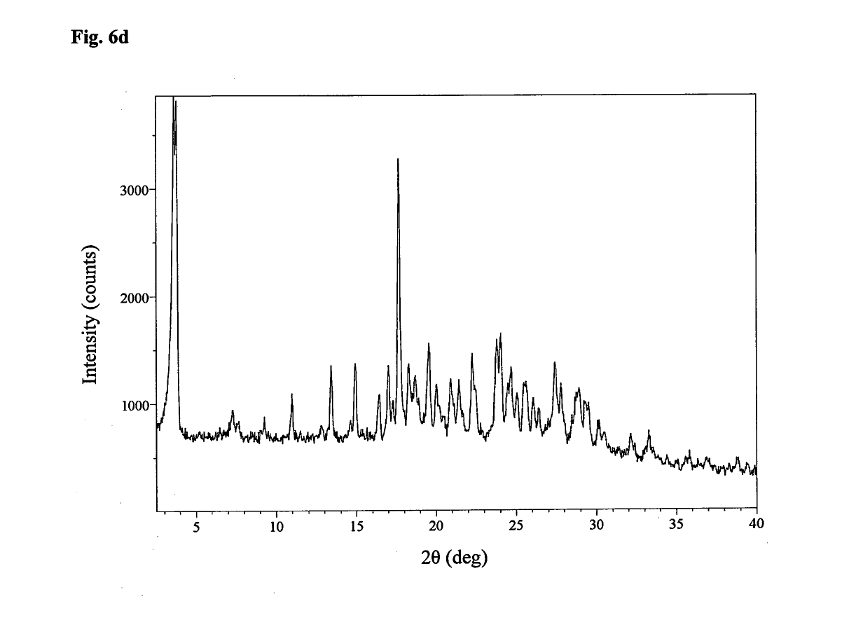

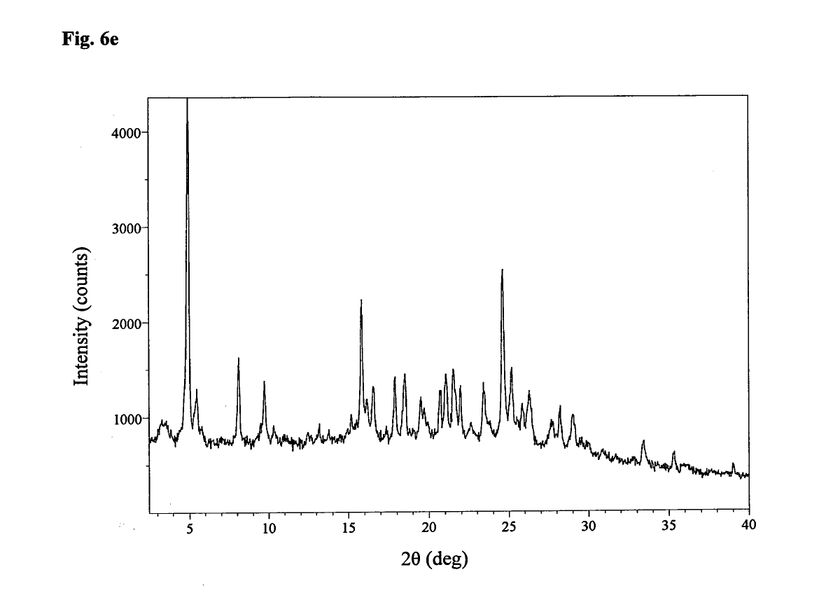

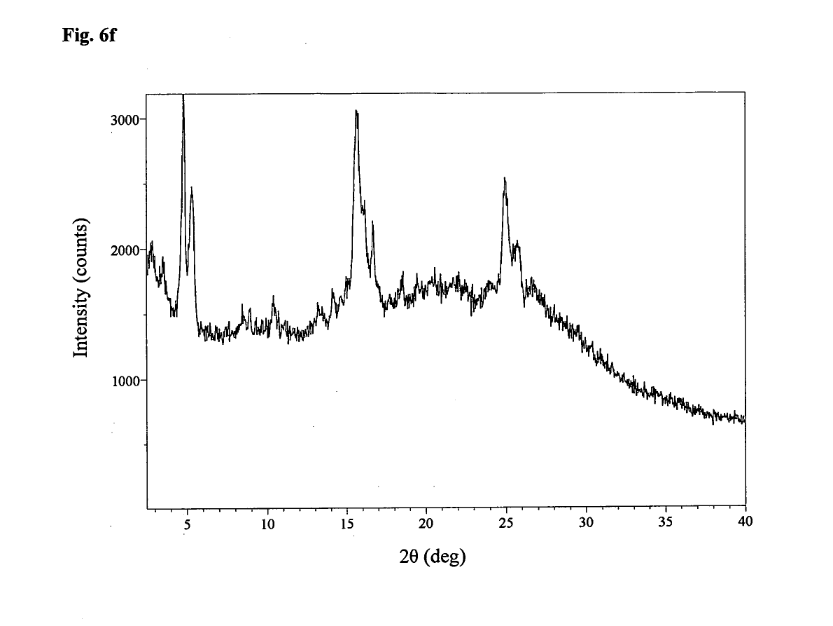

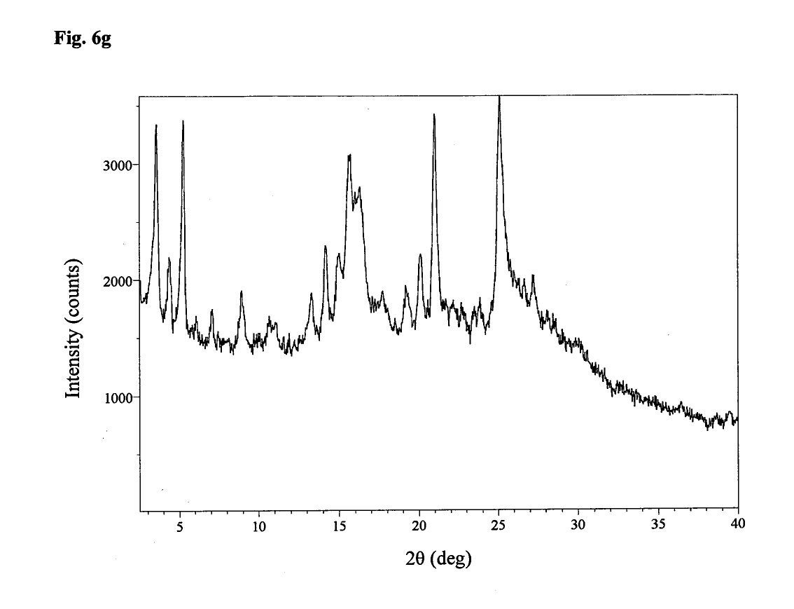

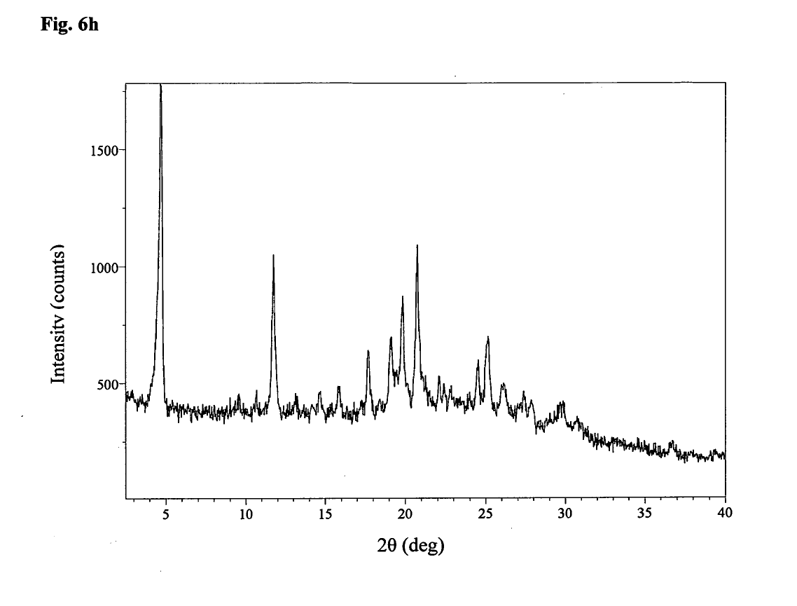

In an embodiment, Form A of (R)-5-(2-Aminoethyl)-1-(6,8-difluorochroman-3-yl)-1,3-dihydroimidazole-2-thione tosylate has the XRPD pattern as shown in FIG. 6a.

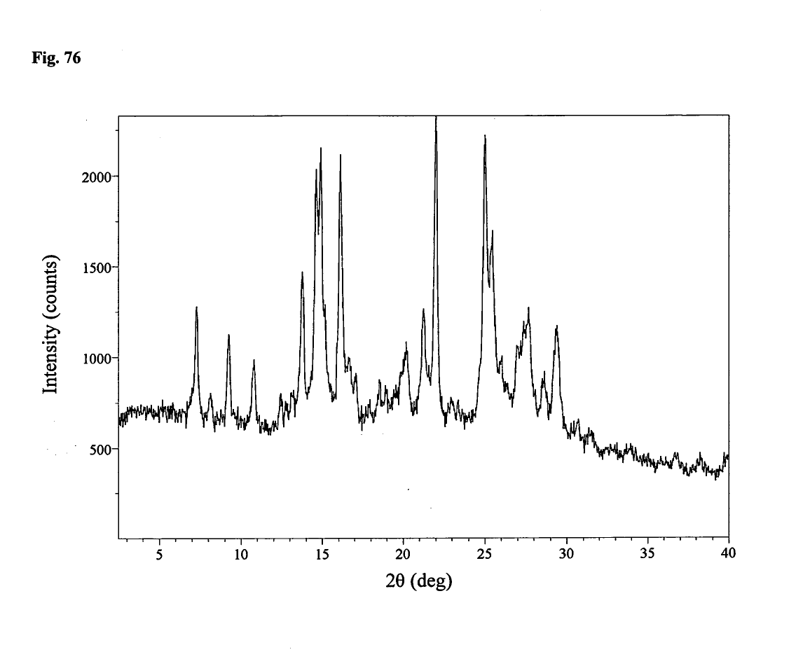

In an embodiment, Form A of (R)-5-(2-Aminoethyl)-1-(6,8-difluorochroman-3-yl)-1,3-dihydroimidazole-2-thione tosylate has the XRPD pattern as shown in FIG. 76.

In another embodiment, there is provided crystalline Form B of (R)-5-(2-Aminoethyl)-1-(6,8-difluorochroman-3-yl)-1,3-dihydroimidazole-2-thione tosylate.

Form B may be characterised as having an XRPD pattern with peaks at 4.6, 8.3, 9.0 and 15.0 °2θ±0.2 °2θ. The XRPD pattern may have further peaks at 16.0 and 17.7 °2θ±0.2 °2θ.

In an embodiment, Form B has an XRPD pattern with peaks at the positions listed in Table 15 below.

TABLE 15

| °2θ | d space (Å) | Intensity % (I/Io) |
|---|---|---|
| 4.6 ± 0.1 | 19.086 ± 0.421 | 100 |
| 8.3 ± 0.1 | 10.666 ± 0.130 | 15 |
| 9.0 ± 0.1 | 9.848 ± 0.111 | 11 |
| 15.0 ± 0.1 | 5.891 ± 0.039 | 15 |
| 16.0 ± 0.1 | 5.529 ± 0.034 | 37 |
| 17.7 ± 0.1 | 5.008 ± 0.028 | 15 |

In another embodiment, Form B has an XRPD pattern with peaks at the positions listed in Table 16 below.

TABLE 16

| °2θ | d space (Å) | Intensity % (I/Io) |
|---|---|---|
| 4.6 ± 0.1 | 19.086 ± 0.421 | 100 |
| 8.3 ± 0.1 | 10.666 ± 0.130 | 15 |
| 9.0 ± 0.1 | 9.848 ± 0.111 | 11 |
| 13.2 ± 0.1 | 6.702 ± 0.051 | 3 |
| 14.0 ± 0.1 | 6.344 ± 0.046 | 3 |
| 15.0 ± 0.1 | 5.891 ± 0.039 | 15 |
| 15.5 ± 0.1 | 5.732 ± 0.037 | 8 |
| 16.0 ± 0.1 | 5.529 ± 0.034 | 37 |
| 16.5 ± 0.1 | 5.360 ± 0.032 | 9 |
| 17.1 ± 0.1 | 5.173 ± 0.030 | 8 |
| 17.7 ± 0.1 | 5.008 ± 0.028 | 15 |
| 18.8 ± 0.1 | 4.730 ± 0.025 | 3 |
| 19.9 ± 0.1 | 4.468 ± 0.022 | 4 |
| 20.9 ± 0.1 | 4.252 ± 0.020 | 6 |
| 21.8 ± 0.1 | 4.079 ± 0.019 | 4 |
| 22.5 ± 0.1 | 3.950 ± 0.017 | 5 |
| 23.2 ± 0.1 | 3.834 ± 0.016 | 5 |
| 24.0 ± 0.1 | 3.716 ± 0.015 | 9 |
| 24.9 ± 0.1 | 3.575 ± 0.014 | 12 |
| 25.3 ± 0.1 | 3.524 ± 0.014 | 13 |
| 25.7 ± 0.1 | 3.468 ± 0.013 | 15 |
| 26.6 ± 0.1 | 3.349 ± 0.012 | 9 |
| 27.0 ± 0.1 | 3.305 ± 0.012 | 7 |
| 28.0 ± 0.1 | 3.187 ± 0.011 | 4 |
| 28.8 ± 0.1 | 3.102 ± 0.011 | 5 |
| 29.9 ± 0.1 | 2.992 ± 0.010 | 4 |

In an embodiment, Form B of (R)-5-(2-Aminoethyl)-1-(6,8-difluorochroman-3-yl)-1,3-dihydroimidazole-2-thione tosylate has the XRPD pattern as shown in FIG. 6b.

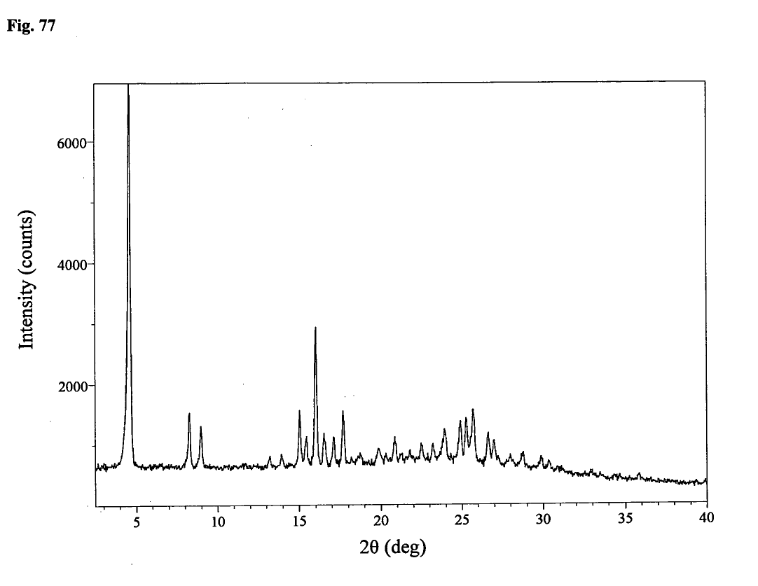

In an embodiment, Form B of (R)-5-(2-Aminoethyl)-1-(6,8-difluorochroman-3-yl)-1,3-dihydroimidazole-2-thione tosylate has the XRPD pattern as shown in FIG. 77.

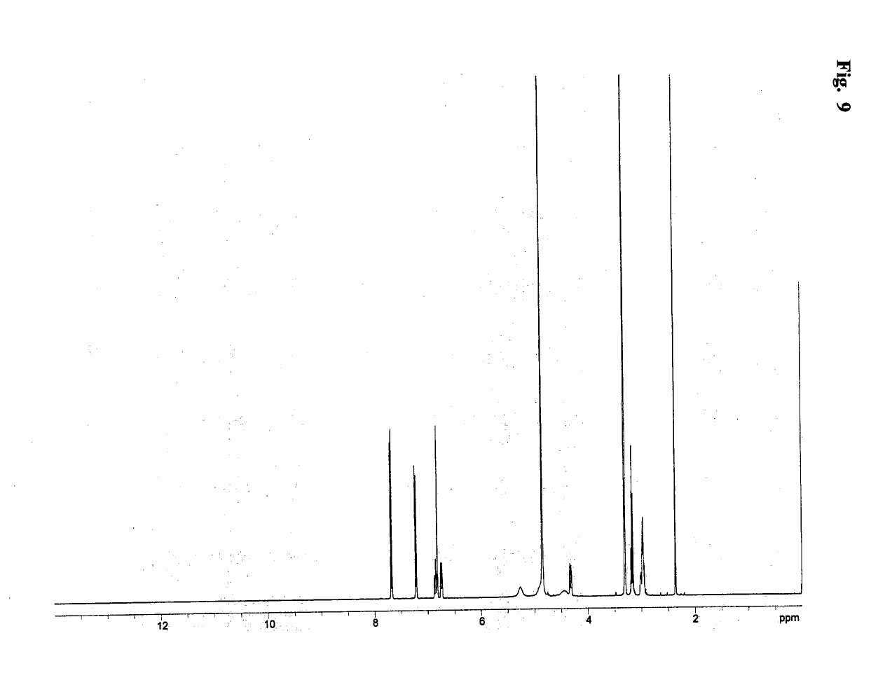

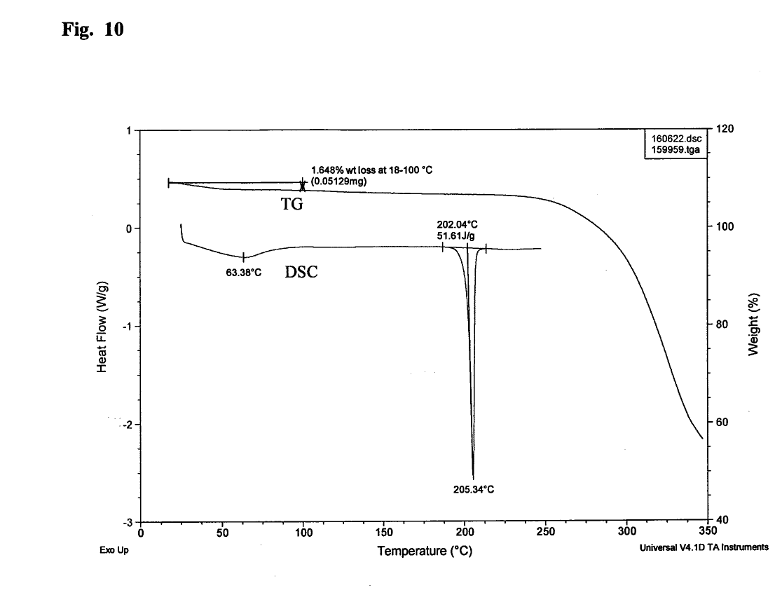

Form B of (R)-5-(2-Aminoethyl)-1-(6,8-difluorochroman-3-yl)-1,3-dihydroimidazole-2-thione tosylate may also be characterised as having the DSC thermogram as shown in FIG. 10.

In another embodiment, there is provided crystalline Form C of (R)-5-(2-Aminoethyl)-1-(6,8-difluorochroman-3-yl)-1,3-dihydroimidazole-2-thione tosylate. Form C may be characterised as having an XRPD pattern with peaks at 11.8 and 12.1 °2θ±0.2 °2θ. The XRPD pattern may have a further peak at 4.8°2θ±0.2 °2θ. The XRPD pattern may have yet further peaks at 17.9, 19.2, 19.7 and 21.0 °2θ±0.2°θ.

In an embodiment, Form C has an XRPD pattern with peaks at the positions listed in Table 17 below.

TABLE 17

| | | Intensity % |
|---|---|---|
| °2θ | d space (Å) | (I/Io) |
| 11.8 ± 0.1 | 7.519 ± 0.064 | 65 |
| 12.1 ± 0.1 | 7.297 ± 0.060 | 23 |

In another embodiment, Form C has an XRPD pattern with peaks at the positions listed in Table 18 below.

TABLE 18

| °2θ | d space (Å) | Intensity % (I/Io) |
|---|---|---|
| 4.8 ± 0.1 | 18.372 ± 0.390 | 100 |
| 11.8 ± 0.1 | 7.519 ± 0.064 | 65 |
| 12.1 ± 0.1 | 7.297 ± 0.060 | 23 |
| 17.9 ± 0.1 | 4.966 ± 0.028 | 28 |
| 19.2 ± 0.1 | 4.620 ± 0.024 | 25 |
| 19.7 ± 0.1 | 4.509 ± 0.023 | 69 |
| 21.0 ± 0.1 | 4.222 ± 0.020 | 51 |

In yet another embodiment, Form C has an XRPD pattern with peaks at the positions listed in Table 19 below.

TABLE 19

| | | Intensity |
|---|---|---|
| °2θ | d space (Å) | % (I/Io) |
| 4.8 ± 0.1 | 18.372 ± 0.390 | 100 |
| 11.8 ± 0.1 | 7.519 ± 0.064 | 65 |
| 12.1 ± 0.1 | 7.297 ± 0.060 | 23 |
| 13.2 ± 0.1 | 6.718 ± 0.051 | 5 |
| 14.0 ± 0.1 | 6.330 ± 0.045 | 4 |
| 14.8 ± 0.1 | 5.998 ± 0.041 | 6 |
| 15.1 ± 0.1 | 5.879 ± 0.039 | 13 |
| 16.1 ± 0.1 | 5.498 ± 0.034 | 10 |
| 17.3 ± 0.1 | 5.129 ± 0.030 | 7 |
| 17.9 ± 0.1 | 4.966 ± 0.028 | 28 |
| 19.2 ± 0.1 | 4.620 ± 0.024 | 25 |
| 19.7 ± 0.1 | 4.509 ± 0.023 | 69 |
| 20.4 ± 0.1 | 4.358 ± 0.021 | 11 |
| 20.8 ± 0.1 | 4.277 ± 0.020 | 27 |
| 21.0 ± 0.1 | 4.222 ± 0.020 | 51 |
| 21.6 ± 0.1 | 4.118 ± 0.019 | 11 |
| 22.4 ± 0.1 | 3.966 ± 0.018 | 10 |
| 23.0 ± 0.1 | 3.859 ± 0.017 | 17 |
| 24.1 ± 0.1 | 3.693 ± 0.015 | 18 |
| 24.9 ± 0.1 | 3.575 ± 0.014 | 27 |
| 25.2 ± 0.1 | 3.541 ± 0.014 | 24 |
| 25.8 ± 0.1 | 3.456 ± 0.013 | 11 |
| 26.3 ± 0.1 | 3.394 ± 0.013 | 6 |
| 27.0 ± 0.1 | 3.308 ± 0.012 | 9 |
| 27.6 ± 0.1 | 3.231 ± 0.012 | 14 |
| 29.5 ± 0.1 | 3.031 ± 0.010 | 10 |

In an embodiment, Form C of (R)-5-(2-Aminoethyl)-1-(6,8-difluorochroman-3-yl)-1,3-dihydroimidazole-2-thione tosylate has the XRPD pattern as shown in FIG. 6c.

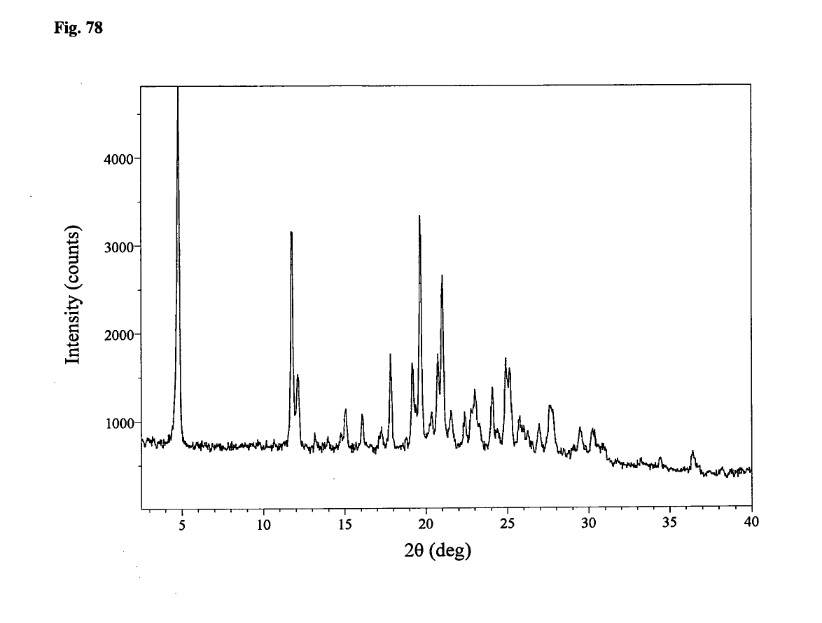

In an embodiment, Form C of (R)-5-(2-Aminoethyl)-1-(6,8-difluorochroman-3-yl)-1,3-dihydroimidazole-2-thione tosylate has the XRPD pattern as shown in FIG. 78.

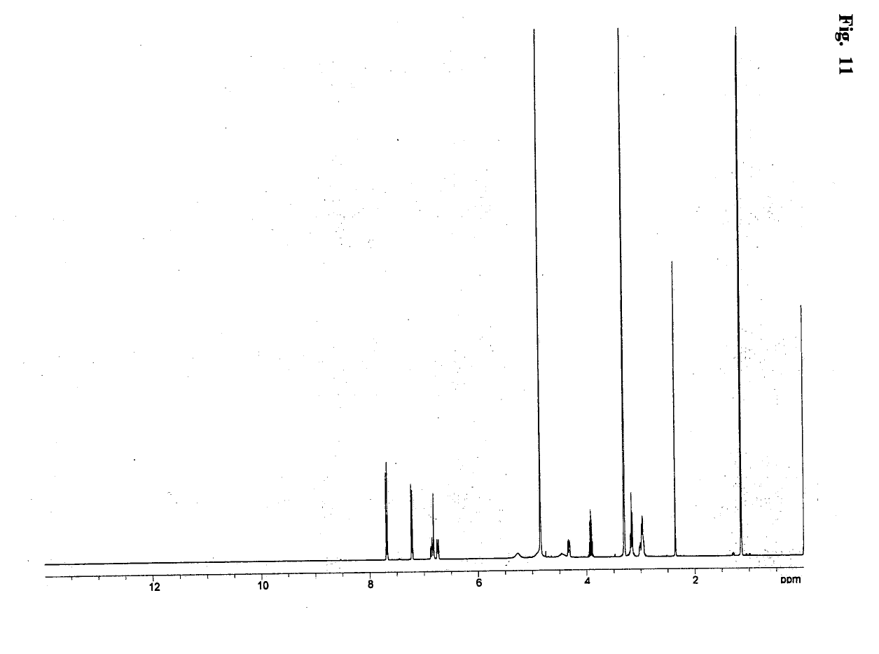

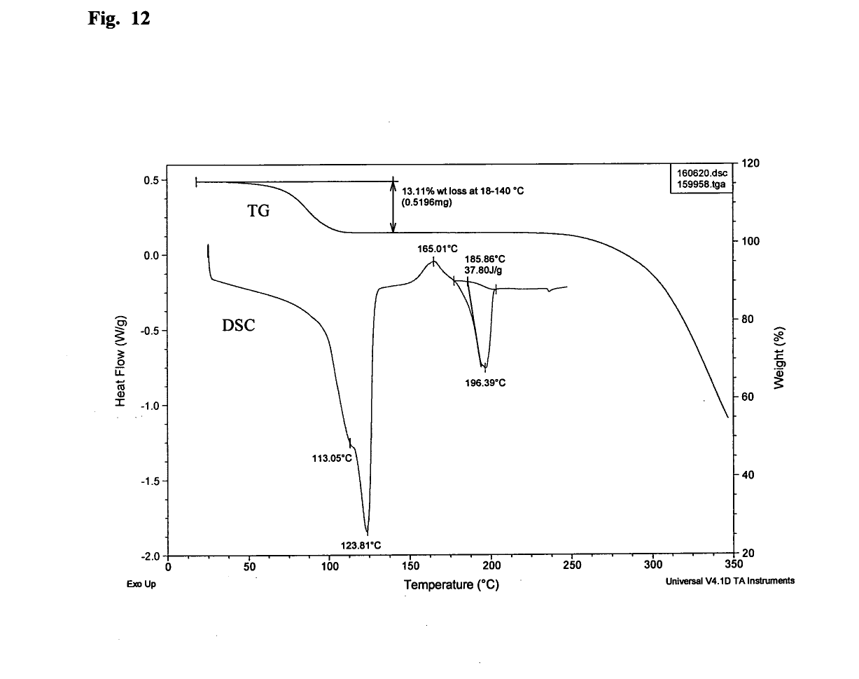

Form C of (R)-5-(2-Aminoethyl)-1-(6,8-difluorochroman-3-yl)-1,3-dihydroimidazole-2-thione tosylate may be characterised as having the DSC thermogram as shown in FIG. 12.

In another embodiment, Form C of the tosylate salt is characterised as being in the form of a solvate of isopropanol. The number of moles of isopropanol per mole of Form C may range from 0.5 to 2.0. Typically, the number of moles ranges from 0.8 to 1.5, more typically from 1 to 1.5. In an embodiment, there is 0.91 mole of isopropanol per 1 mole of Form C.

In another embodiment, there is provided crystalline Form E of (R)-5-(2-Aminoethyl)-1-(6,8-difluorochroman-3-yl)-1,3-dihydroimidazole-2-thione tosylate.

Form E may be characterised as having an XRPD pattern with a peak at 9.7 °2θ±0.2 °2θ. The XRPD pattern may have a further peak at 24.6 °2θ±0.2 °2θ. The XRPD pattern may have yet further peaks at 4.9 and 8.1 °2θ±0.2 °2θ. The XRPD pattern may have a still further peak at 15.8 °2θ±0.2°θ. The XRPD pattern may have yet a further peak at 17.9 °2θ±0.2°θ.

In an embodiment, Form E has an XRPD pattern with peaks at the positions listed in Table 20 below.

TABLE 20

| | | Intensity |
|---|---|---|
| °2θ | d space (Å) | % (I/Io) |
| 9.7 ± 0.1 | 9.073 ± 0.094 | 18 |
| 24.6 ± 0.1 | 3.613 ± 0.014 | 54 |

In another embodiment, Form E has an XRPD pattern with peaks at the positions listed in Table 21 below.

TABLE 21

| | | Intensity |
|---|---|---|
| °2θ | d space (Å) | % (I/Io) |
| 4.9 ± 0.1 | 17.916 ± 0.371 | 100 |
| 8.1 ± 0.1 | 10.935 ± 0.137 | 22 |
| 9.7 ± 0.1 | 9.073 ± 0.094 | 18 |
| 15.8 ± 0.1 | 5.593 ± 0.035 | 67 |
| 24.6 ± 0.1 | 3.613 ± 0.014 | 54 |

In yet another embodiment, Form E has an XRPD pattern with peaks at the positions listed in Table 22 below.

TABLE 22

| | | Intensity |
|---|---|---|
| °2θ | d space (Å) | % (I/Io) |
| 3.4 ± 0.1 | 25.927 ± 0.784 | 4 |
| 4.9 ± 0.1 | 17.916 ± 0.371 | 100 |
| 5.5 ± 0.1 | 16.107 ± 0.299 | 11 |
| 8.1 ± 0.1 | 10.935 ± 0.137 | 22 |
| 9.7 ± 0.1 | 9.073 ± 0.094 | 18 |
| 13.2 ± 0.1 | 6.719 ± 0.051 | 6 |
| 13.8 ± 0.1 | 6.433 ± 0.047 | 6 |
| 15.2 ± 0.1 | 5.834 ± 0.038 | 12 |
| 15.8 ± 0.1 | 5.593 ± 0.035 | 67 |
| 16.2 ± 0.1 | 5.486 ± 0.034 | 16 |
| 16.5 ± 0.1 | 5.361 ± 0.032 | 18 |
| 17.4 ± 0.1 | 5.106 ± 0.029 | 5 |
| 17.9 ± 0.1 | 4.949 ± 0.028 | 25 |
| 18.5 ± 0.1 | 4.802 ± 0.026 | 22 |
| 19.5 ± 0.1 | 4.549 ± 0.023 | 15 |
| 19.7 ± 0.1 | 4.501 ± 0.023 | 14 |
| 20.7 ± 0.1 | 4.285 ± 0.021 | 21 |
| 21.1 ± 0.1 | 4.216 ± 0.020 | 27 |
| 21.5 ± 0.1 | 4.129 ± 0.019 | 31 |
| 22.0 ± 0.1 | 4.045 ± 0.018 | 17 |
| 22.6 ± 0.1 | 3.935 ± 0.017 | 5 |
| 23.4 ± 0.1 | 3.797 ± 0.016 | 21 |
| 23.8 ± 0.1 | 3.732 ± 0.015 | 11 |
| 24.6 ± 0.1 | 3.613 ± 0.014 | 54 |
| 25.2 ± 0.1 | 3.540 ± 0.014 | 24 |
| 25.8 ± 0.1 | 3.447 ± 0.013 | 17 |
| 26.3 ± 0.1 | 3.384 ± 0.013 | 26 |
| 27.8 ± 0.1 | 3.215 ± 0.011 | 13 |
| 28.2 ± 0.1 | 3.164 ± 0.011 | 14 |
| 29.0 ± 0.1 | 3.076 ± 0.010 | 13 |

In an embodiment, Form E of (R)-5-(2-Aminoethyl)-1-(6,8-difluorochroman-3-yl)-1,3-dihydroimidazole-2-thione tosylate has the XRPD pattern as shown in FIG. 6e.

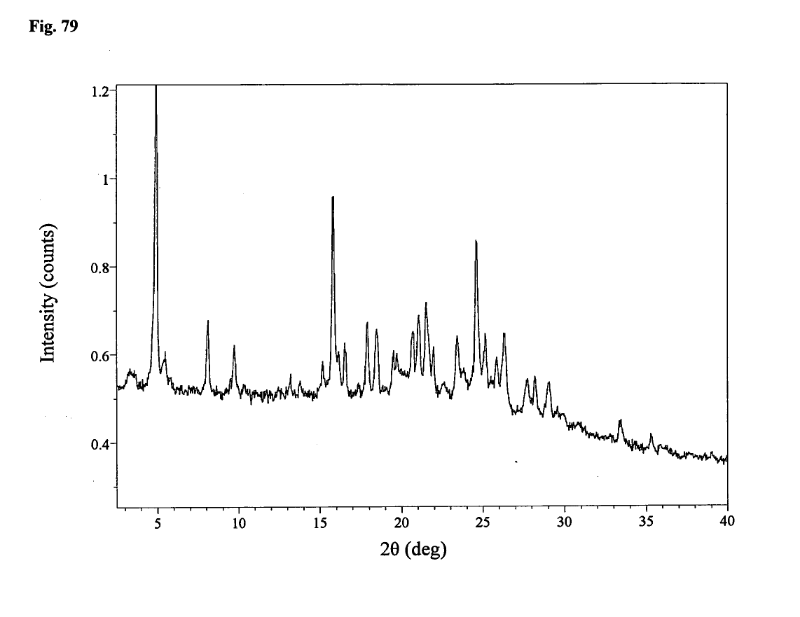

In an embodiment, Form E of (R)-5-(2-Aminoethyl)-1-(6,8-difluorochroman-3-yl)-1,3-dihydroimidazole-2-thione tosylate has the XRPD pattern as shown in FIG. 79.

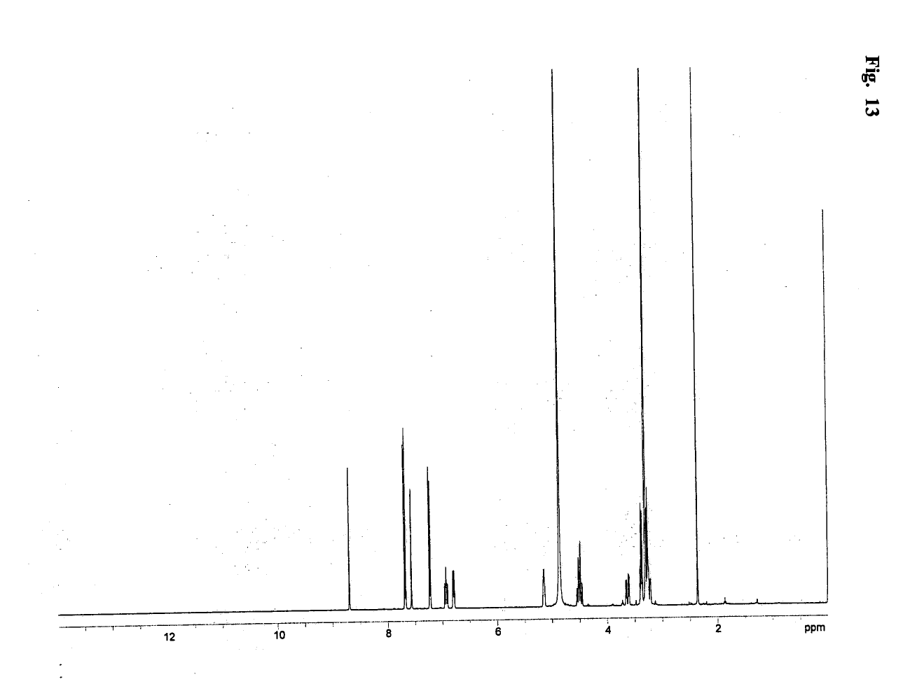

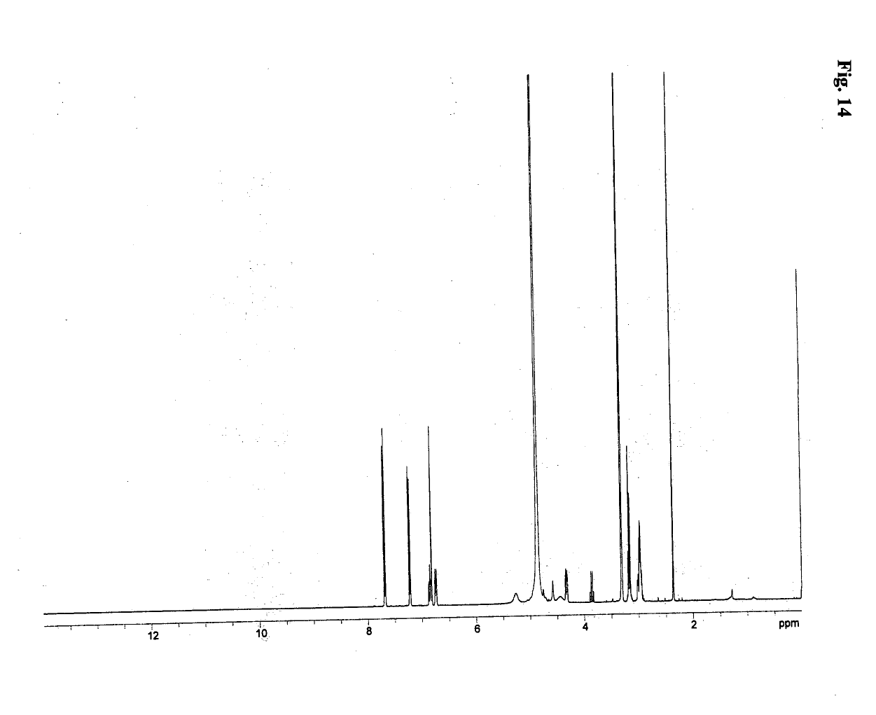

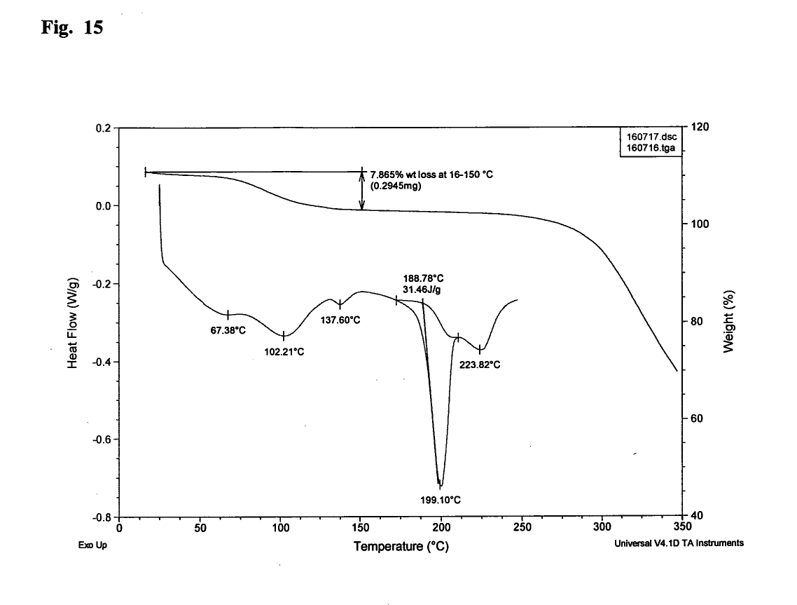

Form E of (R)-5-(2-Aminoethyl)-1-(6,8-difluorochroman-3-yl)-1,3-dihydroimidazole-2-thione tosylate may also be characterised as having the DSC thermogram as shown in FIG. 15.

In another embodiment, Form E of the tosylate salt is characterised as being in the form of a solvate of trifluoroethanol. The number of moles of trifluoroethanol per mole of Form E may range from 0.13 to 0.5. Typically, the number of moles ranges from 0.14 to 0.33. In an embodiment, there is 0.143 mole of trifluoroethanol per 1 mole of Form E.

In another embodiment, there is provided a crystal modification of (R)-5-(2-Aminoethyl)-1-(6,8-difluorochroman-3-yl)-1,3-dihydroimidazole-2-thione tosylate. This crystal modification is hereinafter referred to as crystal modification X of (R)-5-(2-Aminoethyl)-1-(6,8-difluorochroman-3-yl)-1,3-dihydroimidazole-2-thione tosylate.

Crystal modification X may be characterised as having an XRPD pattern with peaks at 4.8 and 5.4 °2θ±0.2 °2θ. The XRPD pattern may have further peaks at 15.6, 16.7 and 25.0 °2θ±0.2 °2θ.

In an embodiment, crystal modification X has an XRPD pattern with peaks at the positions listed in Table 23 below.

TABLE 23

| °2θ | d space (Å) | Intensity % (I/Io) |
|---|---|---|
| 4.8 ± 0.1 | 18.258 ± 0.385 | 100 |
| 5.4 ± 0.1 | 16.519 ± 0.314 | 61 |
| 15.6 ± 0.1 | 5.666 ± 0.036 | 95 |
| 16.7 ± 0.1 | 5.312 ± 0.032 | 41 |
| 25.0 ± 0.1 | 3.566 ± 0.014 | 61 |

In another embodiment, crystal modification X has an XRPD pattern with peaks at the positions listed in Table 24 below.

TABLE 24

| °2θ | d space (Å) | Intensity % (I/Io) |
|---|---|---|
| 2.8 ± 0.1 | 31.220 ± 1.143 | 10 |
| 3.6 ± 0.1 | 24.889 ± 0.721 | 16 |
| 4.8 ± 0.1 | 18.258 ± 0.385 | 100 |
| 5.4 ± 0.1 | 16.519 ± 0.314 | 61 |
| 8.5 ± 0.1 | 10.440 ± 0.125 | 15 |
| 9.0 ± 0.1 | 9.881 ± 0.111 | 15 |
| 10.4 ± 0.1 | 8.490 ± 0.082 | 18 |
| 13.2 ± 0.1 | 6.702 ± 0.051 | 10 |
| 14.1 ± 0.1 | 6.264 ± 0.044 | 14 |
| 15.6 ± 0.1 | 5.666 ± 0.036 | 95 |
| 16.2 ± 0.1 | 5.488 ± 0.034 | 52 |
| 16.7 ± 0.1 | 5.312 ± 0.032 | 41 |
| 18.5 ± 0.1 | 4.791 ± 0.026 | 14 |
| 19.5 ± 0.1 | 4.557 ± 0.023 | 16 |
| 25.0 ± 0.1 | 3.566 ± 0.014 | 61 |
| 25.8 ± 0.1 | 3.456 ± 0.013 | 33 |

In an embodiment, crystal modification X of (R)-5-(2-Aminoethyl)-1-(6,8-difluorochroman-3-yl)-1,3-dihydroimidazole-2-thione tosylate the XRPD pattern as shown in FIG. 6f.

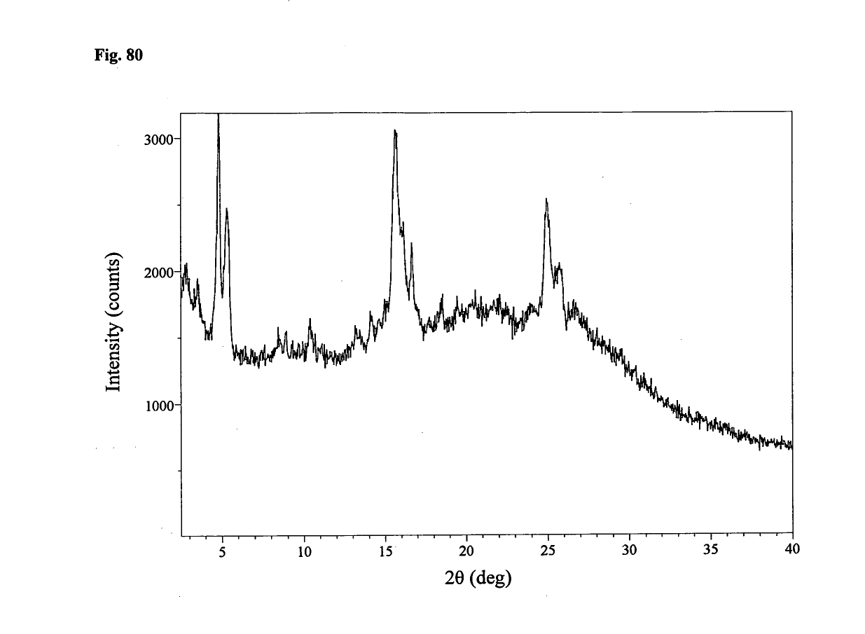

In an embodiment, crystal modification X of (R)-5-(2-Aminoethyl)-1-(6,8-difluorochroman-3-yl)-1,3-dihydroimidazole-2-thione tosylate the XRPD pattern as shown in FIG. 80.

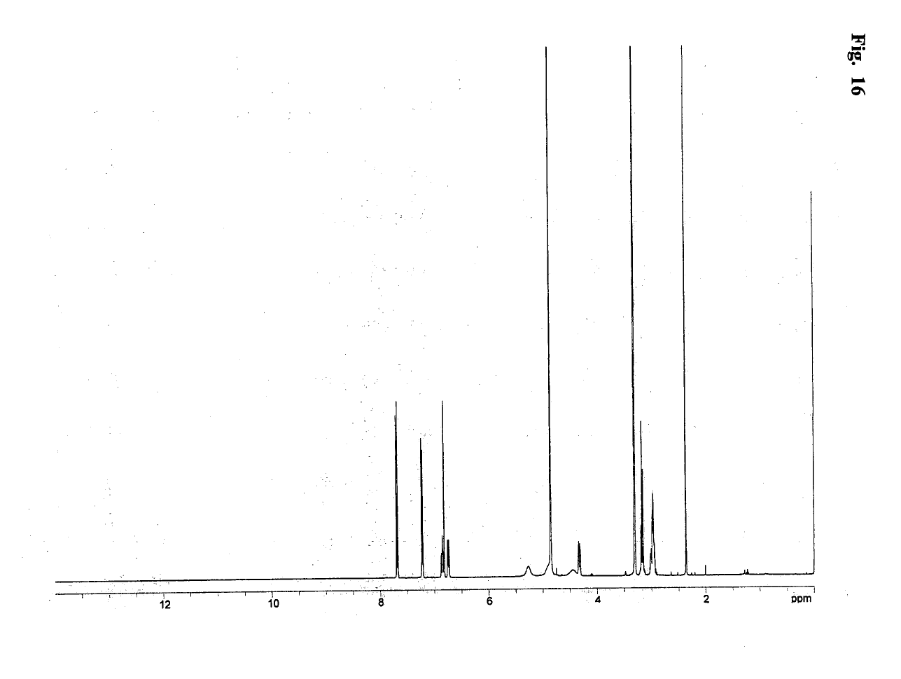

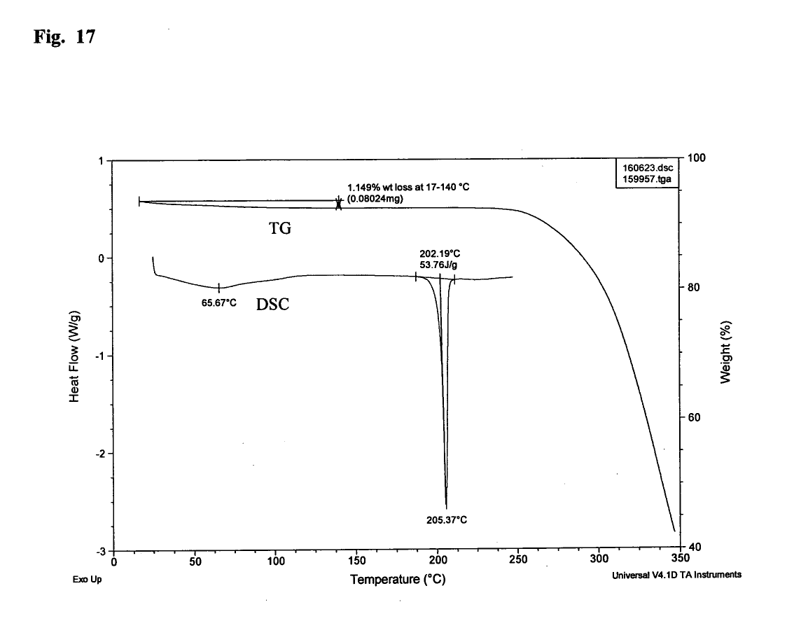

Crystal modification X of (R)-5-(2-Aminoethyl)-1-(6,8-difluorochroman-3-yl)-1,3-dihydroimidazole-2-thione tosylate may also be characterised as having the DSC thermogram as shown in FIG. 17.

In another embodiment, there is provided crystalline Form G of (R)-5-(2-Aminoethyl)-1-(6,8-difluorochroman-3-yl)-1,3-dihydroimidazole-2-thione tosylate.

Form G may be characterised as having an XRPD pattern with peaks at 3.6, 4.4, 5.3 and 14.2 °2θ±0.2 °2θ. The XRPD pattern may have further peaks at 7.1, 9.0 and 13.3 °2θ±0.2 °2θ. The XRPD pattern may have a still further peak at 15.7 °2θ±0.2°θ.

In an embodiment, Form G has an XRPD pattern with peaks at the positions listed in Table 25 below.

TABLE 25

| °2θ | d space (Å) | Intensity % (I/Io) |
|---|---|---|
| 3.6 ± 0.1 | 24.681 ± 0.709 | 69 |
| 4.4 ± 0.1 | 19.992 ± 0.463 | 27 |
| 5.3 ± 0.1 | 16.706 ± 0.322 | 88 |
| 14.2 ± 0.1 | 6.237 ± 0.044 | 38 |

In another embodiment, Form G has an XRPD pattern with peaks at the positions listed in Table 26 below.

TABLE 26

| °2θ | d space (Å) | Intensity % (I/Io) |
|---|---|---|
| 3.6 ± 0.1 | 24.681 ± 0.709 | 69 |
| 4.4 ± 0.1 | 19.992 ± 0.463 | 27 |
| 5.3 ± 0.1 | 16.706 ± 0.322 | 88 |
| 7.1 ± 0.1 | 12.468 ± 0.178 | 15 |
| 9.0 ± 0.1 | 9.881 ± 0.111 | 26 |
| 13.3 ± 0.1 | 6.657 ± 0.050 | 21 |
| 14.2 ± 0.1 | 6.237 ± 0.044 | 38 |
| 15.7 ± 0.1 | 5.655 ± 0.036 | 72 |
| 21.0 ± 0.1 | 4.228 ± 0.020 | 91 |
| 25.1 ± 0.1 | 3.545 ± 0.014 | 100 |

In yet another embodiment, Form G has an XRPD pattern with peaks at the positions listed in Table 27 below.

TABLE 27

| °2θ | d space (Å) | Intensity % (I/Io) |
|---|---|---|
| 3.6 ± 0.1 | 24.681 ± 0.709 | 69 |
| 4.4 ± 0.1 | 19.992 ± 0.463 | 27 |
| 5.3 ± 0.1 | 16.706 ± 0.322 | 88 |
| 6.1 ± 0.1 | 14.561 ± 0.244 | 10 |
| 7.1 ± 0.1 | 12.468 ± 0.178 | 15 |
| 9.0 ± 0.1 | 9.881 ± 0.111 | 26 |
| 10.7 ± 0.1 | 8.276 ± 0.078 | 15 |
| 11.1 ± 0.1 | 7.986 ± 0.073 | 12 |
| 13.3 ± 0.1 | 6.657 ± 0.050 | 21 |
| 14.2 ± 0.1 | 6.237 ± 0.044 | 38 |
| 15.0 ± 0.1 | 5.914 ± 0.040 | 33 |
| 15.7 ± 0.1 | 5.655 ± 0.036 | 72 |
| 16.3 ± 0.1 | 5.438 ± 0.033 | 59 |
| 17.7 ± 0.1 | 5.000 ± 0.028 | 16 |
| 19.2 ± 0.1 | 4.620 ± 0.024 | 18 |
| 20.1 ± 0.1 | 4.416 ± 0.022 | 32 |
| 21.0 ± 0.1 | 4.228 ± 0.020 | 91 |
| 25.1 ± 0.1 | 3.545 ± 0.014 | 100 |
| 26.6 ± 0.1 | 3.345 ± 0.012 | 22 |
| 27.2 ± 0.1 | 3.273 ± 0.012 | 26 |
| 28.1 ± 0.1 | 3.177 ± 0.011 | 14 |

In an embodiment, Form G of (R)-5-(2-Aminoethyl)-1-(6,8-difluorochroman-3-yl)-1,3-dihydroimidazole-2-thione tosylate has the XRPD pattern as shown in FIG. 6g.

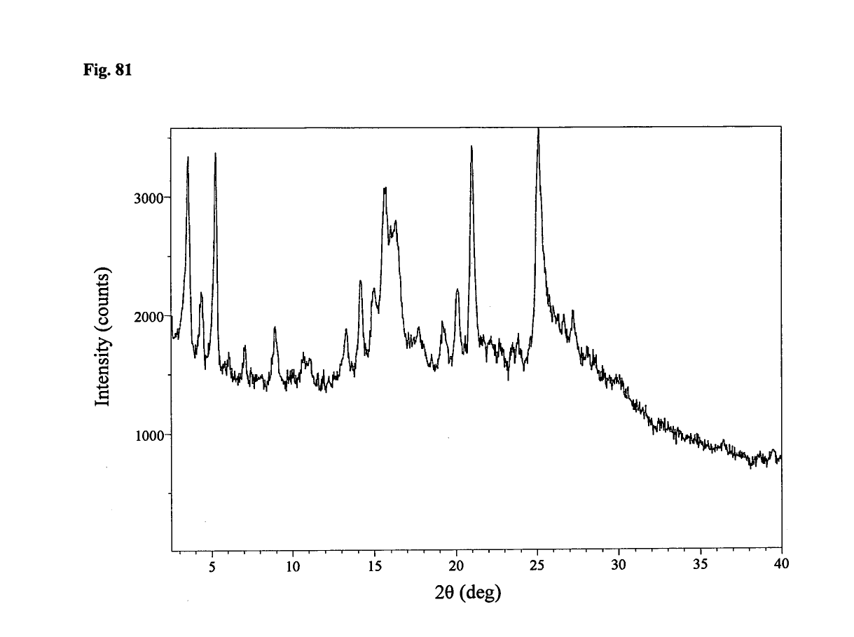

In an embodiment, Form G of (R)-5-(2-Aminoethyl)-1-(6,8-difluorochroman-3-yl)-1,3-dihydroimidazole-2-thione tosylate has the XRPD pattern as shown in FIG. 81.

In another embodiment, there is provided another crystal modification of (R)-5-(2-Aminoethyl)-1-(6,8-difluorochroman-3-yl)-1,3-dihydroimidazole-2-thione tosylate. This crystal modification is hereinafter referred to as crystal modification Y of (R)-5-(2-Aminoethyl)-1-(6,8-difluorochroman-3-yl)-1,3-dihydroimidazole-2-thione tosylate.

Crystal modification Y may be characterised as having an XRPD pattern with peaks at 4.7 and 11.8 °2θ±0.2 °2θ. The XRPD pattern may have further peaks at 17.7, 19.2, 19.9 and 20.8 °2θ±0.2 °2θ.

In an embodiment, crystal modification Y has an XRPD pattern with peaks at the positions listed in Table 28 below.

TABLE 28

| °2θ | d space (Å) | Intensity % (I/Io) |
|---|---|---|
| 4.7 ± 0.1 | 18.722 ± 0.405 | 100 |
| 11.8 ± 0.1 | 7.519 ± 0.064 | 43 |
| 17.7 ± 0.1 | 5.000 ± 0.028 | 18 |
| 19.2 ± 0.1 | 4.635 ± 0.024 | 22 |
| 19.9 ± 0.1 | 4.468 ± 0.022 | 32 |
| 20.8 ± 0.1 | 4.277 ± 0.020 | 44 |

In another embodiment, crystal modification Y has an XRPD pattern with peaks at the positions listed in Table 29 below.

TABLE 29

| °2θ | d space (Å) | Intensity % (I/Io) |
|---|---|---|
| 4.7 ± 0.1 | 18.722 ± 0.405 | 100 |
| 9.6 ± 0.1 | 9.261 ± 0.098 | 4 |
| 10.7 ± 0.1 | 8.299 ± 0.078 | 4 |
| 11.8 ± 0.1 | 7.519 ± 0.064 | 43 |
| 13.1 ± 0.1 | 6.748 ± 0.052 | 5 |
| 14.3 ± 0.1 | 6.198 ± 0.043 | 5 |
| 14.7 ± 0.1 | 6.022 ± 0.041 | 7 |
| 15.9 ± 0.1 | 5.581 ± 0.035 | 8 |
| 17.7 ± 0.1 | 5.000 ± 0.028 | 18 |
| 19.2 ± 0.1 | 4.635 ± 0.024 | 22 |
| 19.9 ± 0.1 | 4.468 ± 0.022 | 32 |
| 20.8 ± 0.1 | 4.277 ± 0.020 | 44 |
| 22.1 ± 0.1 | 4.019 ± 0.018 | 7 |
| 22.4 ± 0.1 | 3.966 ± 0.018 | 6 |
| 22.9 ± 0.1 | 3.884 ± 0.017 | 7 |
| 24.5 ± 0.1 | 3.631 ± 0.015 | 16 |
| 25.2 ± 0.1 | 3.541 ± 0.014 | 22 |
| 26.1 ± 0.1 | 3.417 ± 0.013 | 10 |
| 27.4 ± 0.1 | 3.252 ± 0.012 | 10 |
| 27.9 ± 0.1 | 3.197 ± 0.011 | 6 |
| 29.7 ± 0.1 | 3.010 ± 0.010 | 8 |

In an embodiment, crystal modification Y of (R)-5-(2-Aminoethyl)-1-(6,8-difluorochroman-3-yl)-1,3-dihydroimidazole-2-thione tosylate has the XRPD pattern as shown in FIG. 6h.

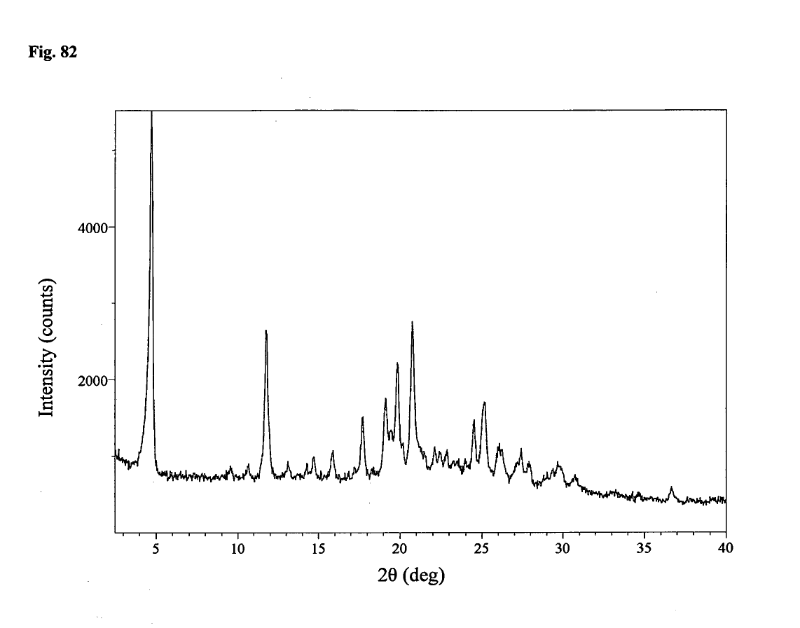

In another embodiment, crystal modification Y of (R)-5-(2-Aminoethyl)-1-(6,8-difluorochroman-3-yl)-1,3-dihydroimidazole-2-thione tosylate has the XRPD pattern as shown in FIG. 82.

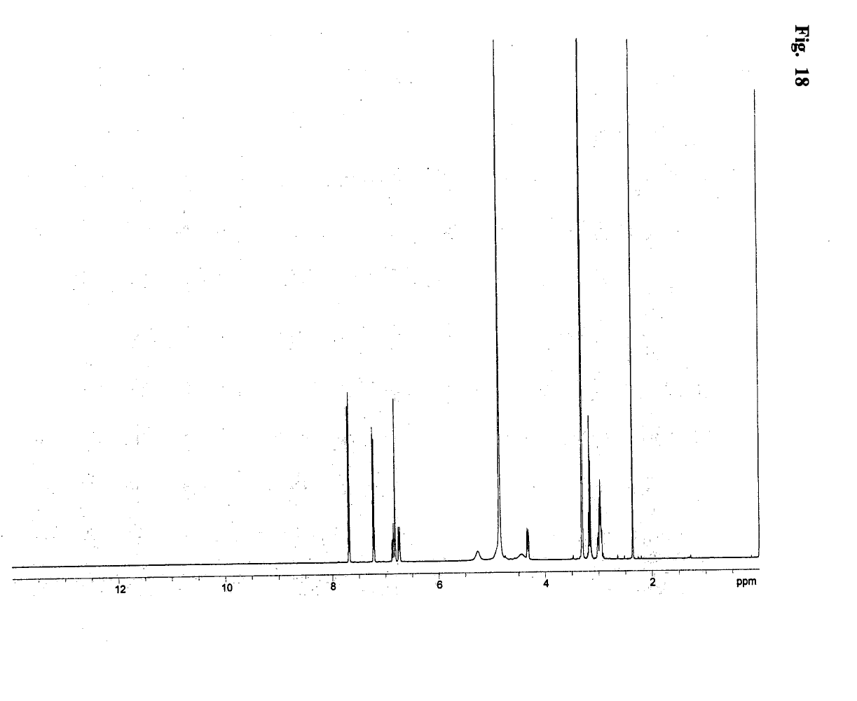

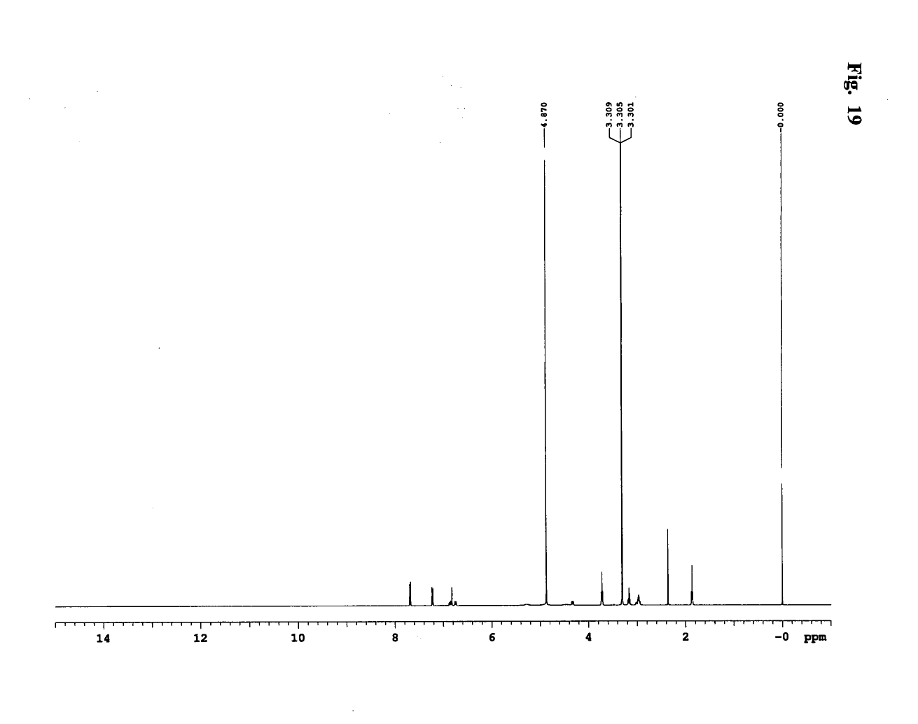

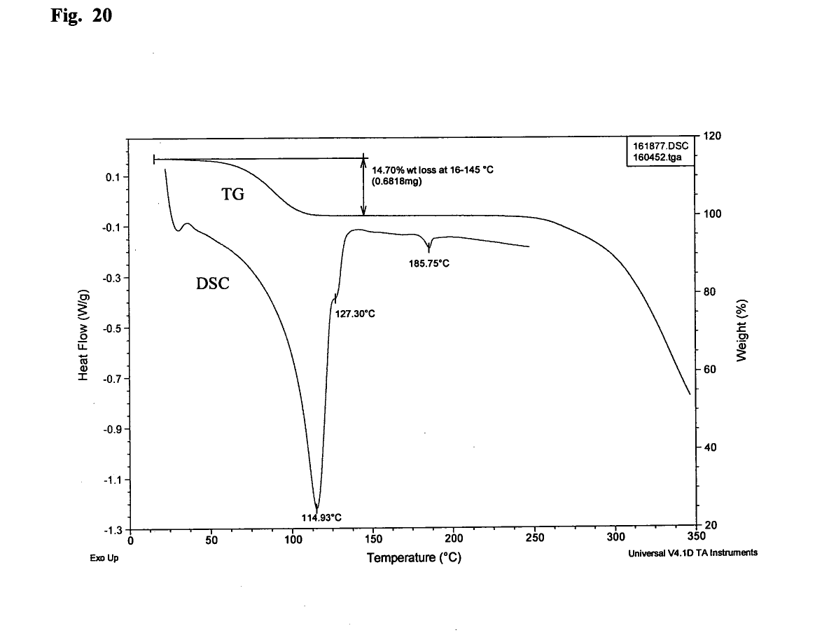

Crystal modification Y of (R)-5-(2-Aminoethyl)-1-(6,8-difluorochroman-3-yl)-1,3-dihydroimidazole-2-thione tosylate may also be characterised as having the DSC thermogram as shown in FIG. 20. In another embodiment, crystal modification Y of the tosylate salt is characterised as being in the form of a solvate of trifluoroethanol. The number of moles of trifluoroethanol per mole of crystal modification Y may range from 0.13 to 0.5. Typically, the number of moles ranges from 0.14 to 0.33. In an embodiment, there is 0.143 mole of trifluoroethanol per 1 mole of crystal modification Y.

According to another aspect of the present invention, there is provided the acetic acid salt of (R)-5-(2-Aminoethyl)-1-(6,8-difluorochroman-3-yl)-1,3-dihydroimidazole-2-thione, i.e. (R)-5-(2-Aminoethyl)-1-(6,8-difluorochroman-3-yl)-1,3-dihydroimidazole-2-thione acetate.

In an embodiment, there is provided crystalline Form 1 of (R)-5-(2-Aminoethyl)-1-(6,8-difluorochroman-3-yl)-1,3-dihydroimidazole-2-thione acetate.

Form 1 may be characterised as having an XRPD pattern with peaks at 11.0 and 12.9 °2θ±0.2 °2θ. The XRPD pattern may have further peaks at 15.2, 16.2, 19.6, 21.0, 21.8 and 22.2 °2θ±0.2 °2θ.

In an embodiment, Form 1 has an XRPD pattern with peaks at the positions listed in Table 30 below.

TABLE 30

| °2θ | d space (Å) | Intensity % (I/Io) |
|---|---|---|
| 11.0 ± 0.1 | 8.029 ± 0.073 | 32 |
| 12.9 ± 0.1 | 6.842 ± 0.053 | 100 |
| 15.2 ± 0.1 | 5.810 ± 0.038 | 20 |
| 16.2 ± 0.1 | 5.478 ± 0.034 | 62 |
| 19.6 ± 0.1 | 4.522 ± 0.023 | 46 |
| 21.0 ± 0.1 | 4.228 ± 0.020 | 46 |
| 21.8 ± 0.1 | 4.068 ± 0.018 | 37 |
| 22.2 ± 0.1 | 4.013 ± 0.018 | 54 |
| 24.8 ± 0.1 | 3.596 ± 0.014 | 65 |
| 28.9 ± 0.1 | 3.086 ± 0.010 | 67 |

In another embodiment, Form 1 has an XRPD pattern with peaks at the positions listed in Table 31 below.

TABLE 31

| °2θ | d space (Å) | Intensity % (I/Io) |
|---|---|---|
| 11.0 ± 0.1 | 8.029 ± 0.073 | 32 |
| 12.9 ± 0.1 | 6.842 ± 0.053 | 100 |
| 13.3 ± 0.1 | 6.657 ± 0.050 | 34 |
| 13.5 ± 0.1 | 6.540 ± 0.048 | 25 |
| 15.2 ± 0.1 | 5.810 ± 0.038 | 20 |
| 16.2 ± 0.1 | 5.478 ± 0.034 | 62 |
| 18.2 ± 0.1 | 4.877 ± 0.027 | 8 |
| 19.2 ± 0.1 | 4.613 ± 0.024 | 18 |
| 19.6 ± 0.1 | 4.522 ± 0.023 | 46 |
| 21.0 ± 0.1 | 4.228 ± 0.020 | 46 |
| 21.8 ± 0.1 | 4.068 ± 0.018 | 37 |
| 22.2 ± 0.1 | 4.013 ± 0.018 | 54 |
| 23.5 ± 0.1 | 3.791 ± 0.016 | 19 |
| 23.9 ± 0.1 | 3.729 ± 0.015 | 14 |
| 24.2 ± 0.1 | 3.679 ± 0.015 | 10 |
| 24.8 ± 0.1 | 3.596 ± 0.014 | 65 |
| 25.4 ± 0.1 | 3.508 ± 0.014 | 27 |
| 26.0 ± 0.1 | 3.432 ± 0.013 | 15 |
| 26.3 ± 0.1 | 3.386 ± 0.013 | 20 |
| 27.1 ± 0.1 | 3.294 ± 0.012 | 40 |
| 27.6 ± 0.1 | 3.227 ± 0.011 | 29 |
| 28.9 ± 0.1 | 3.086 ± 0.010 | 67 |
| 29.4 ± 0.1 | 3.034 ± 0.010 | 14 |
| 29.8 ± 0.1 | 2.998 ± 0.010 | 14 |

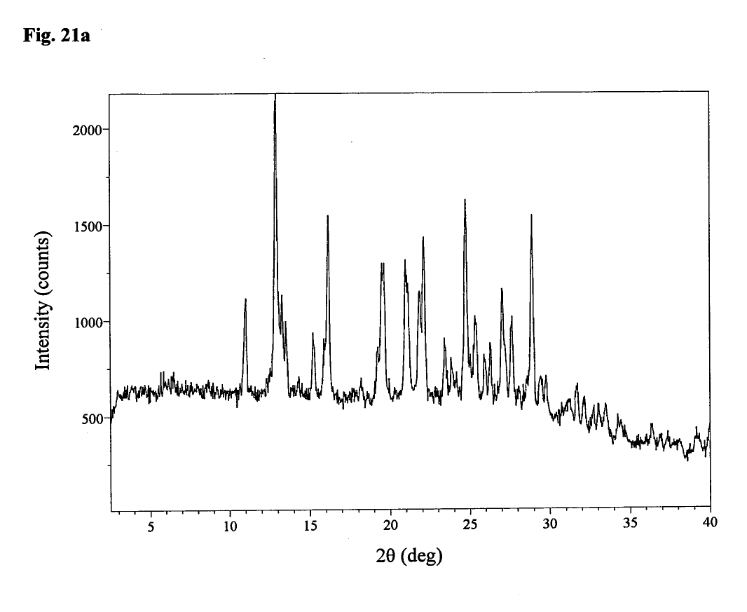

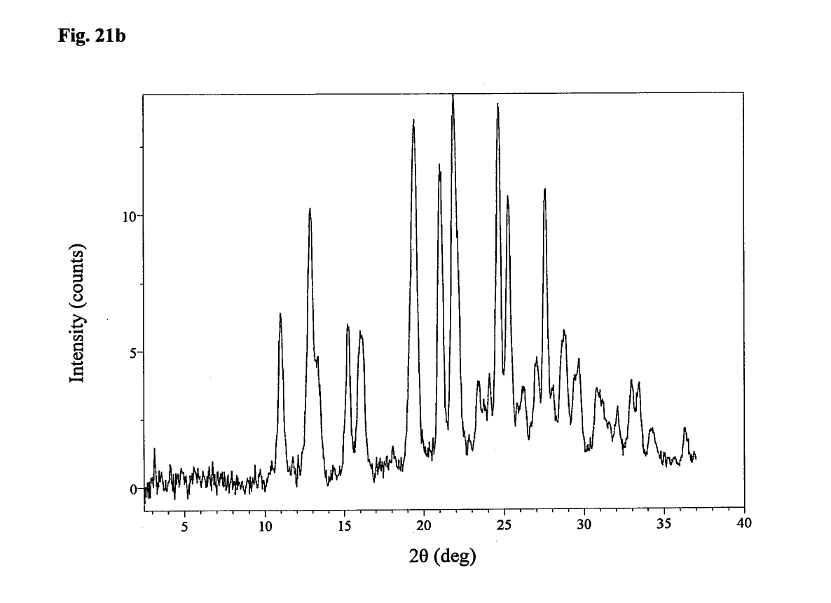

In a further embodiment, Form 1 of (R)-5-(2-Aminoethyl)-1-(6,8-difluorochroman-3-yl)-1,3-dihydroimidazole-2-thione acetate has the XRPD pattern as shown in FIG. 21a. In a yet further embodiment, Form 1 of (R)-5-(2-Aminoethyl)-1-(6,8-difluorochroman-3-yl)-1,3-dihydroimidazole-2-thione acetate has the XRPD pattern as shown in FIG. 21b.

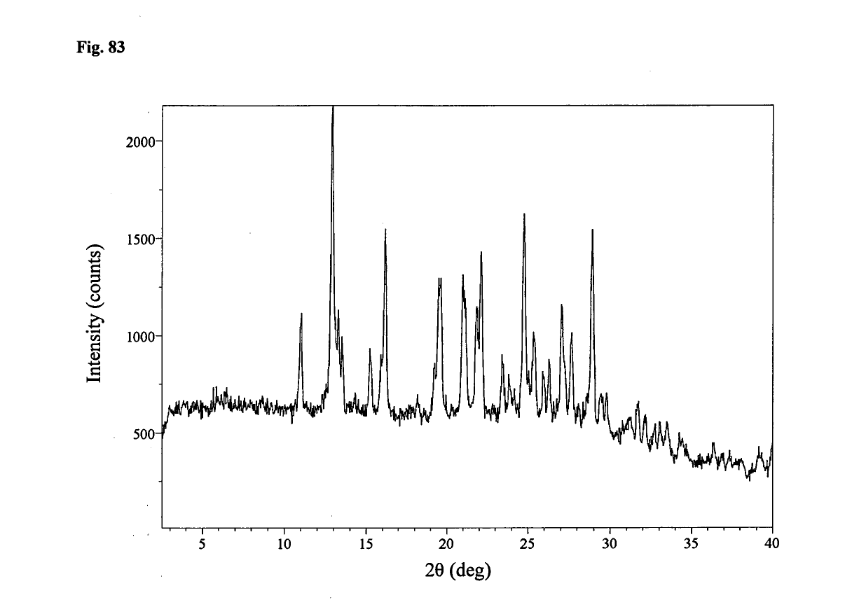

In a further embodiment, Form 1 of (R)-5-(2-Aminoethyl)-1-(6,8-difluorochroman-3-yl)-1,3-dihydroimidazole-2-thione acetate has the XRPD pattern as shown in FIG. 83.

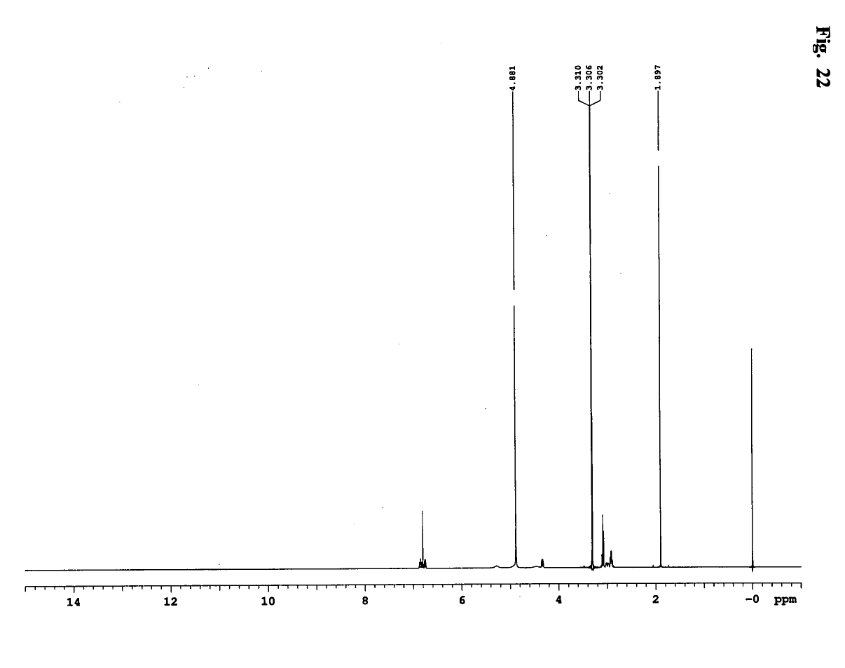

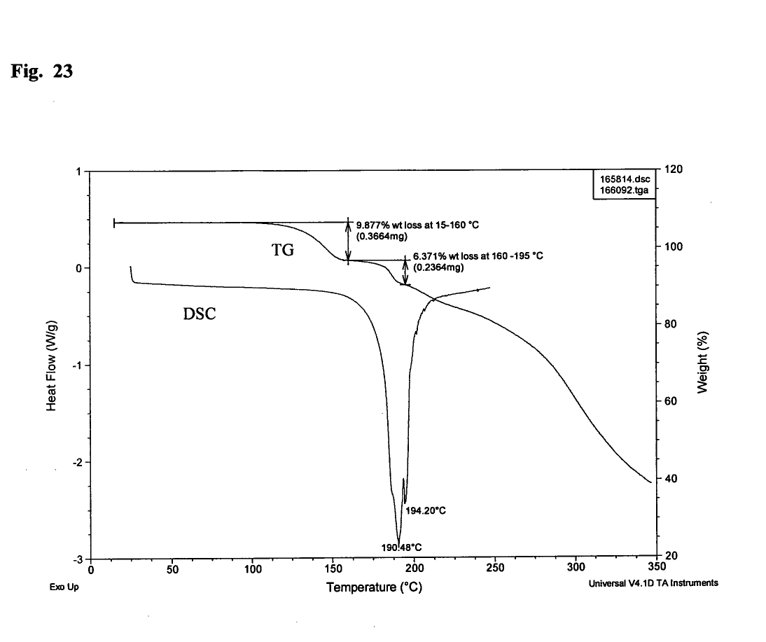

Form 1 of (R)-5-(2-Aminoethyl)-1-(6,8-difluorochroman-3-yl)-1,3-dihydroimidazole-2-thione acetate may also be characterised as having a DSC thermogram as shown in FIG. 23.

According to another aspect of the present invention, there is provided the adipic acid salt of (R)-5-(2-Aminoethyl)-1-(6,8-difluorochroman-3-yl)-1,3-dihydroimidazole-2-thione, i.e. (R)-5-(2-Aminoethyl)-1-(6,8-difluorochroman-3-yl)-1,3-dihydroimidazole-2-thione adipate.

In an embodiment, there is provided crystalline Form 1 of (R)-5-(2-Aminoethyl)-1-(6,8-difluorochroman-3-yl)-1,3-dihydroimidazole-2-thione adipate.

Form 1 may be characterised as having an XRPD pattern with a peak at 7.8 °2θ±0.2 °2θ. The XRPD pattern may have further peaks at 4.5, 12.6, 13.6 and 15.0 °2θ±0.2 °2θ. The XRPD pattern may have still further peaks at 19.6 and 21.5 °2θ±0.2°θ.

In an embodiment, Form 1 has an XRPD pattern with peaks at the positions listed in Table 32 below.

TABLE 32

| °2θ | d space (Å) | Intensity % (I/Io) |
|---|---|---|
| 7.8 ± 0.1 | 11.277 ± 0.145 | 100 |

In another embodiment, Form 1 has an XRPD pattern with peaks at the positions listed in Table 33 below.

TABLE 33

| °2θ | d space (Å) | Intensity % (I/Io) |
|---|---|---|
| 4.5 ± 0.1 | 19.593 ± 0.444 | 23 |
| 7.8 ± 0.1 | 11.277 ± 0.145 | 100 |
| 12.6 ± 0.1 | 7.020 ± 0.056 | 81 |
| 13.6 ± 0.1 | 6.497 ± 0.048 | 56 |
| 15.0 ± 0.1 | 5.891 ± 0.039 | 96 |
| 19.6 ± 0.1 | 4.536 ± 0.023 | 50 |
| 21.5 ± 0.1 | 4.129 ± 0.019 | 66 |

In yet another embodiment, Form 1 has an XRPD pattern with peaks at the positions listed in Table 34 below.

TABLE 34

| °2θ | d space (Å) | Intensity % (I/Io) |
|---|---|---|
| 4.5 ± 0.1 | 19.593 ± 0.444 | 23 |
| 7.8 ± 0.1 | 11.277 ± 0.145 | 100 |
| 10.8 ± 0.1 | 8.207 ± 0.077 | 11 |
| 12.6 ± 0.1 | 7.020 ± 0.056 | 81 |
| 13.0 ± 0.1 | 6.810 ± 0.053 | 20 |
| 13.6 ± 0.1 | 6.497 ± 0.048 | 56 |
| 14.0 ± 0.1 | 6.330 ± 0.045 | 29 |
| 14.4 ± 0.1 | 6.160 ± 0.043 | 26 |
| 15.0 ± 0.1 | 5.891 ± 0.039 | 96 |
| 15.6 ± 0.1 | 5.666 ± 0.036 | 25 |
| 16.5 ± 0.1 | 5.369 ± 0.032 | 19 |
| 19.6 ± 0.1 | 4.536 ± 0.023 | 50 |
| 20.0 ± 0.1 | 4.435 ± 0.022 | 34 |
| 20.6 ± 0.1 | 4.308 ± 0.021 | 26 |
| 21.5 ± 0.1 | 4.129 ± 0.019 | 66 |
| 22.1 ± 0.1 | 4.019 ± 0.018 | 28 |
| 22.7 ± 0.1 | 3.919 ± 0.017 | 25 |
| 23.9 ± 0.1 | 3.720 ± 0.015 | 55 |
| 24.5 ± 0.1 | 3.631 ± 0.015 | 77 |
| 25.0 ± 0.1 | 3.558 ± 0.014 | 75 |
| 25.8 ± 0.1 | 3.456 ± 0.013 | 28 |
| 27.1 ± 0.1 | 3.290 ± 0.012 | 37 |
| 27.9 ± 0.1 | 3.193 ± 0.011 | 12 |
| 29.4 ± 0.1 | 3.043 ± 0.010 | 28 |

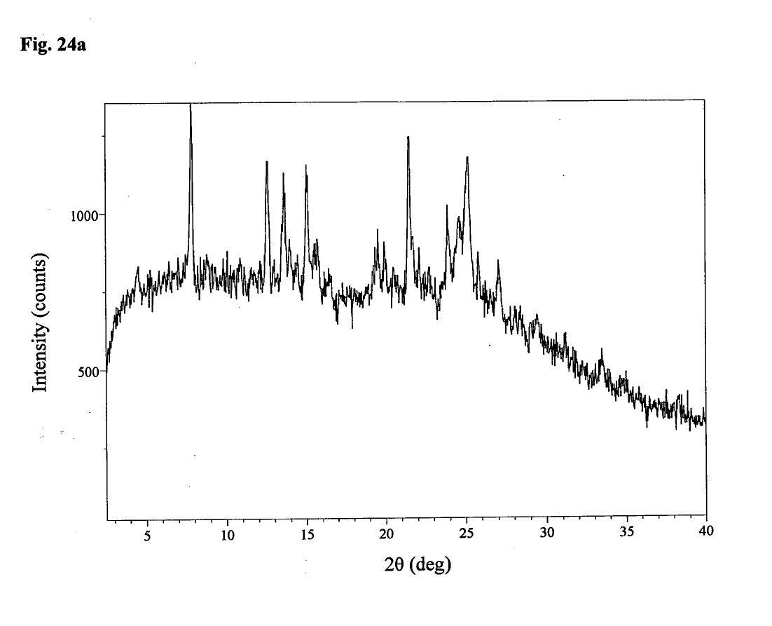

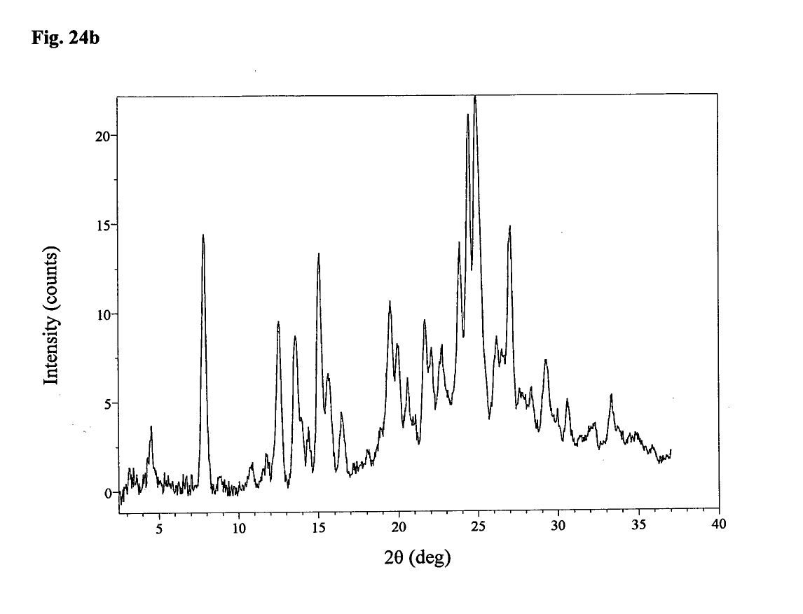

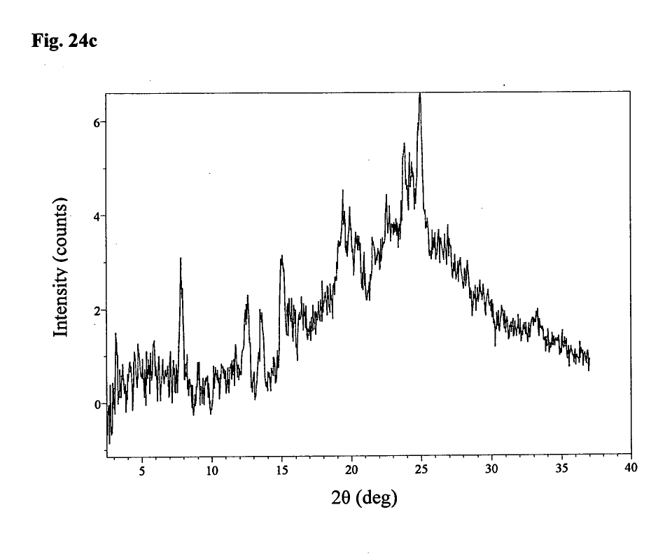

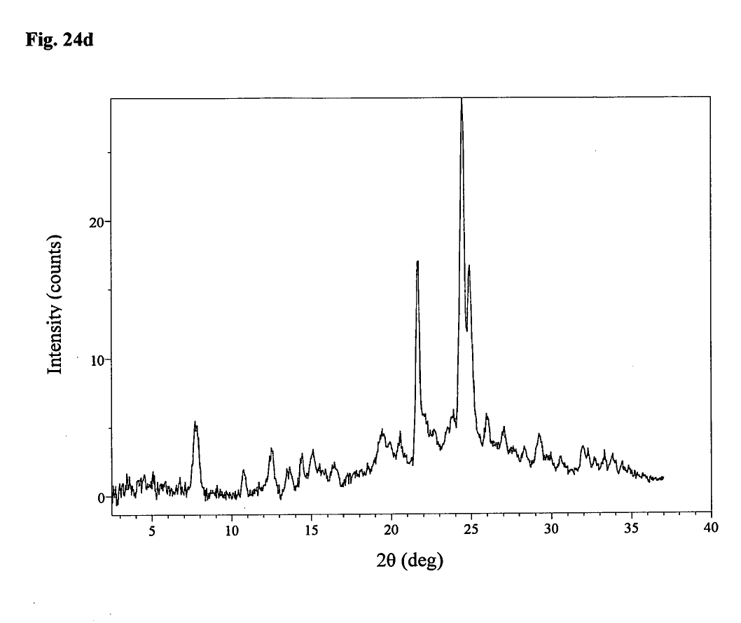

In an embodiment, Form 1 of (R)-5-(2-Aminoethyl)-1-(6,8-difluorochroman-3-yl)-1,3-dihydroimidazole-2-thione adipate has an XRPD pattern as shown in FIG. 24a. In another embodiment, Form 1 of (R)-5-(2-Aminoethyl)-1-(6,8-difluorochroman-3-yl)-1,3-dihydroimidazole-2-thione adipate has an XRPD pattern as shown in FIG. 24b.

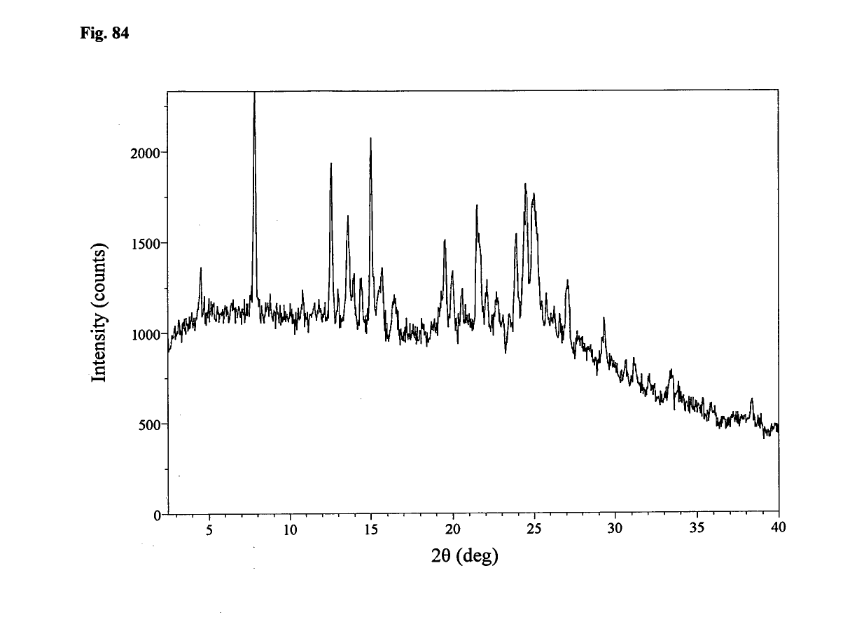

In an embodiment, Form 1 of (R)-5-(2-Aminoethyl)-1-(6,8-difluorochroman-3-yl)-1,3-dihydroimidazole-2-thione adipate has an XRPD pattern as shown in FIG. 84.

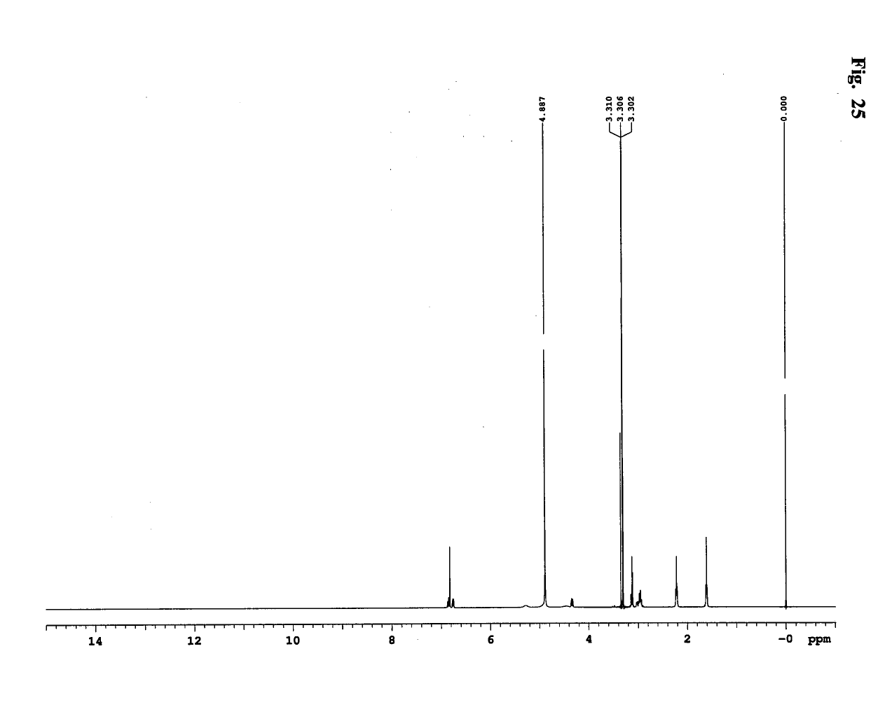

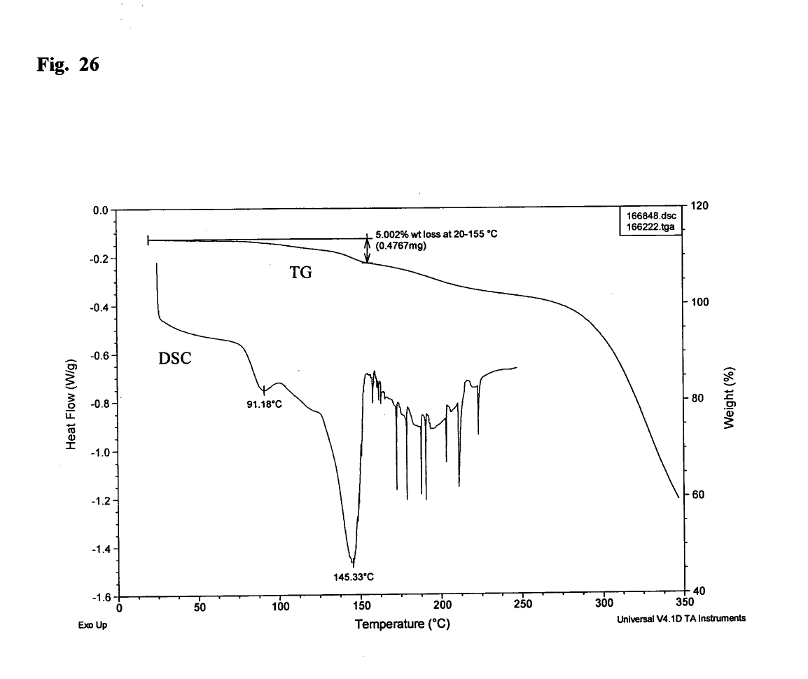

Form 1 of (R)-5-(2-Aminoethyl)-1-(6,8-difluorochroman-3-yl)-1,3-dihydroimidazole-2-thione adipate may also be characterised by having a DSC thermogram as shown in FIG. 26.

According to another aspect of the present invention, there is provided the glutaric acid salt of (R)-5-(2-Aminoethyl)-1-(6,8-difluorochroman-3-yl)-1,3-dihydroimidazole-2-thione, i.e. (R)-5-(2-Aminoethyl)-1-(6,8-difluorochroman-3-yl)-1,3-dihydroimidazole-2-thione glutarate.

In an embodiment, there is provided Form 1 of (R)-5-(2-Aminoethyl)-1-(6,8-difluorochroman-3-yl)-1,3-dihydroimidazole-2-thione glutarate.

Form 1 may be characterised as having an XRPD pattern with peaks at 4.4, 8.0, 10.7, 12.4, 13.6 and 14.2 °2θ±0.2 °2θ. The XRPD pattern may have further peaks at 15.5 and 16.1 °2θ±0.2 °2θ. The XRPD pattern may have still further peaks at 19.1 and 19.8 °2θ±0.2°θ.

In an embodiment, Form 1 has an XRPD pattern with peaks at the positions listed in Table 35 below.

TABLE 35

| °2θ | d space (Å) | Intensity % (I/Io) |
|---|---|---|
| 4.4 ± 0.1 | 19.857 ± 0.456 | 26 |
| 8.0 ± 0.1 | 11.024 ± 0.139 | 57 |
| 10.7 ± 0.1 | 8.299 ± 0.078 | 18 |
| 12.4 ± 0.1 | 7.121 ± 0.058 | 97 |
| 13.6 ± 0.1 | 6.497 ± 0.048 | 42 |
| 14.2 ± 0.1 | 6.250 ± 0.044 | 26 |
| 15.5 ± 0.1 | 5.732 ± 0.037 | 63 |
| 16.1 ± 0.1 | 5.509 ± 0.034 | 56 |
| 19.1 ± 0.1 | 4.656 ± 0.024 | 29 |
| 19.8 ± 0.1 | 4.495 ± 0.023 | 42 |

In another embodiment, Form 1 has an XRPD pattern with peaks at the positions listed in Table 36 below.

TABLE 36

| °2θ | d space (Å) | Intensity % (I/Io) |
|---|---|---|
| 4.4 ± 0.1 | 19.857 ± 0.456 | 26 |
| 8.0 ± 0.1 | 11.024 ± 0.139 | 57 |
| 8.9 ± 0.1 | 9.914 ± 0.112 | 12 |
| 10.7 ± 0.1 | 8.299 ± 0.078 | 18 |
| 11.9 ± 0.1 | 7.443 ± 0.063 | 10 |
| 12.4 ± 0.1 | 7.121 ± 0.058 | 97 |
| 13.6 ± 0.1 | 6.497 ± 0.048 | 42 |
| 14.2 ± 0.1 | 6.250 ± 0.044 | 26 |
| 15.5 ± 0.1 | 5.732 ± 0.037 | 63 |
| 16.1 ± 0.1 | 5.509 ± 0.034 | 56 |
| 19.1 ± 0.1 | 4.656 ± 0.024 | 29 |
| 19.8 ± 0.1 | 4.495 ± 0.023 | 42 |
| 20.5 ± 0.1 | 4.326 ± 0.021 | 23 |
| 21.4 ± 0.1 | 4.147 ± 0.019 | 21 |
| 22.1 ± 0.1 | 4.024 ± 0.018 | 20 |
| 22.5 ± 0.1 | 3.950 ± 0.017 | 18 |
| 22.9 ± 0.1 | 3.884 ± 0.017 | 26 |
| 23.9 ± 0.1 | 3.725 ± 0.015 | 71 |
| 25.0 ± 0.1 | 3.562 ± 0.014 | 62 |
| 25.3 ± 0.1 | 3.524 ± 0.014 | 57 |
| 25.7 ± 0.1 | 3.472 ± 0.013 | 100 |
| 26.3 ± 0.1 | 3.386 ± 0.013 | 23 |
| 27.1 ± 0.1 | 3.294 ± 0.012 | 36 |
| 27.9 ± 0.1 | 3.193 ± 0.011 | 17 |
| 28.4 ± 0.1 | 3.137 ± 0.011 | 8 |
| 29.6 ± 0.1 | 3.019 ± 0.010 | 14 |

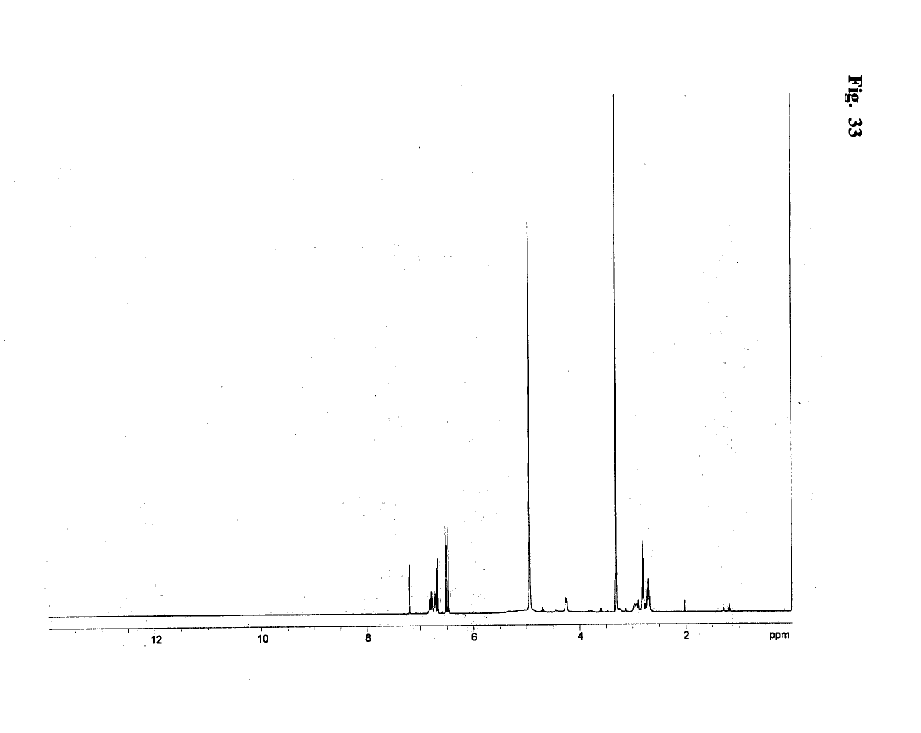

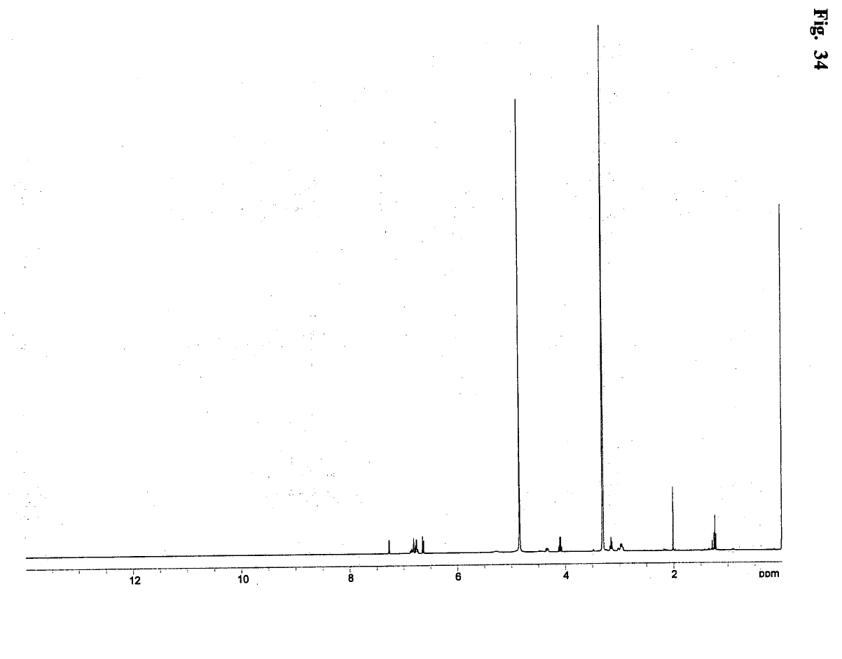

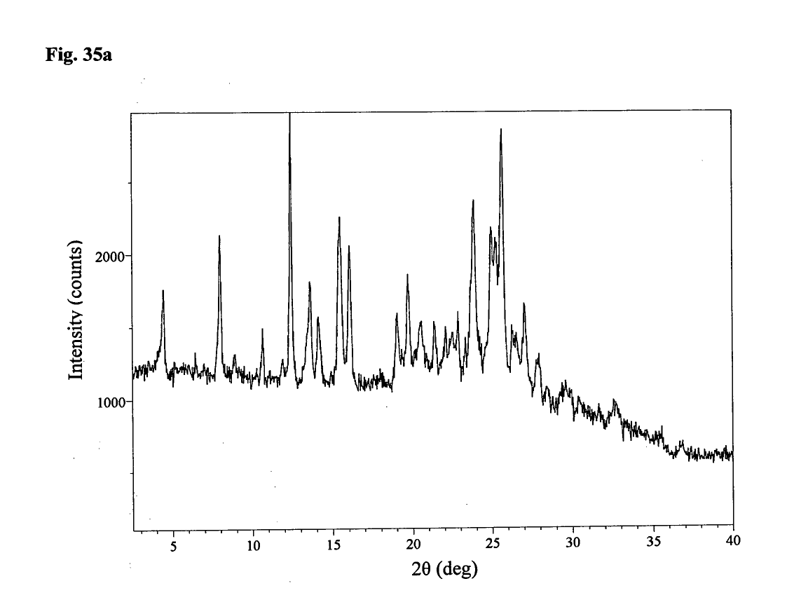

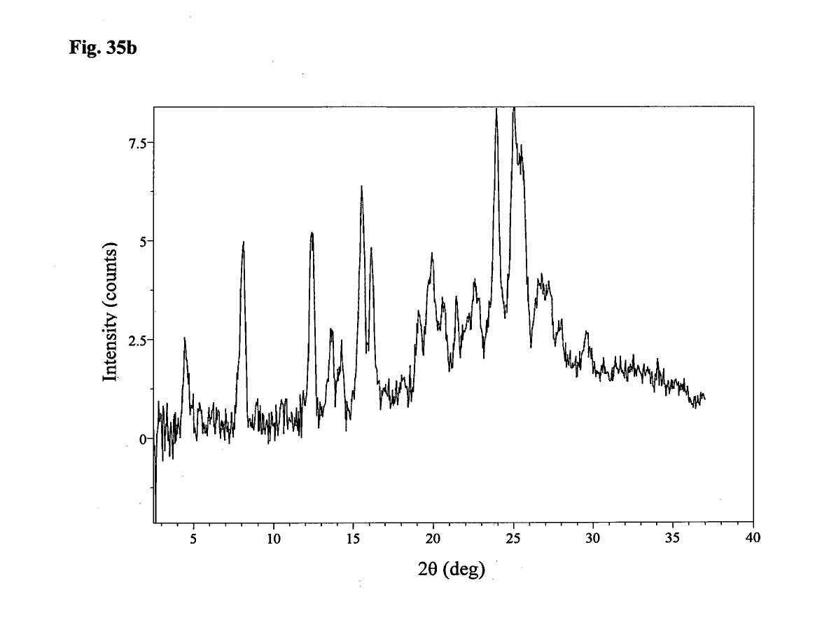

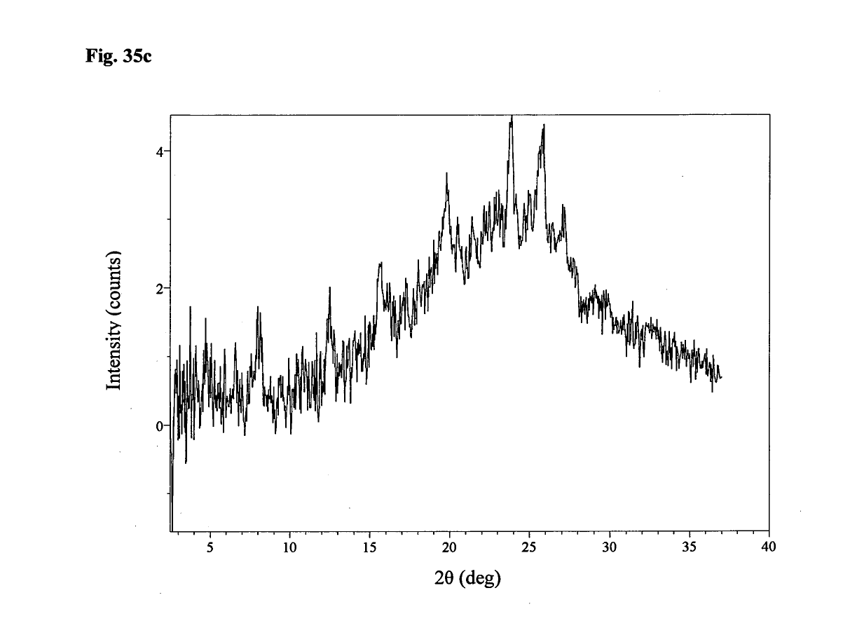

In an embodiment, Form 1 of (R)-5-(2-Aminoethyl)-1-(6,8-difluorochroman-3-yl)-1,3-dihydroimidazole-2-thione glutarate has the XRPD pattern as shown in FIG. 35a. In another embodiment, Form 1 of (R)-5-(2-Aminoethyl)-1-(6,8-difluorochroman-3-yl)-1,3-dihydroimidazole-2-thione glutarate has the XRPD pattern as shown in FIG. 35b.

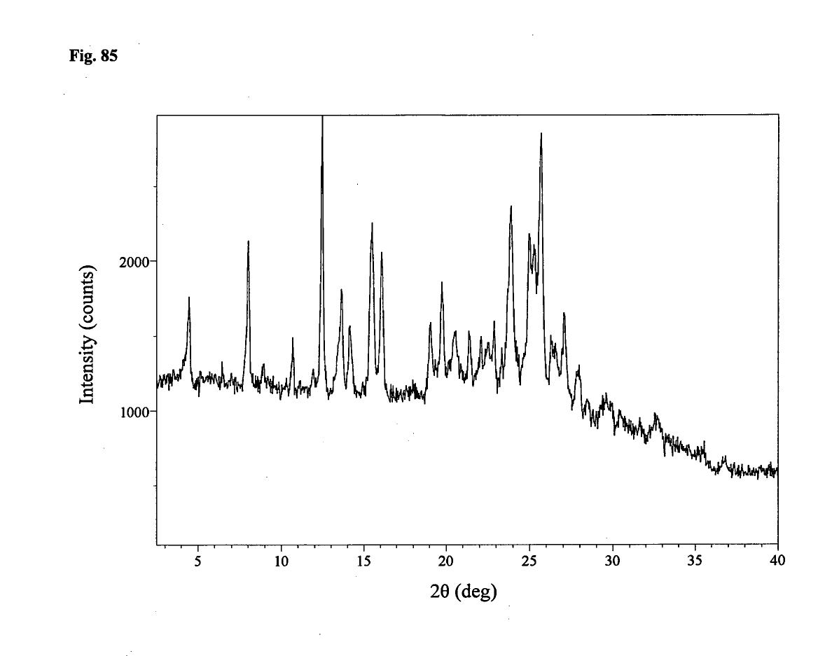

In an embodiment, Form 1 of (R)-5-(2-Aminoethyl)-1-(6,8-difluorochroman-3-yl)-1,3-dihydroimidazole-2-thione glutarate has the XRPD pattern as shown in FIG. 85.

According to another aspect of the present invention, there is provided the succinic acid salt of (R)-5-(2-Aminoethyl)-1-(6,8-difluorochroman-3-yl)-1,3-dihydroimidazole-2-thione, i.e. (R)-5-(2-Aminoethyl)-1-(6,8-difluorochroman-3-yl)-1,3-dihydroimidazole-2-thione succinate.

In an embodiment, there is provided crystalline Form 1 of (R)-5-(2-Aminoethyl)-1-(6,8-difluorochroman-3-yl)-1,3-dihydroimidazole-2-thione succinate.

Form 1 may be characterised as having an XRPD pattern with peaks at 4.6, 8.1, and 12.7 °2θ±0.2 °2θ. The XRPD pattern may have a further peak at 9.0 °2θ±0.2 °2θ. The XRPD pattern may have yet a further peak at 14.0 °2θ±0.2 °2θ. The XRPD pattern may have yet further peaks at 15.7, 20.5 and 24.7 °2θ±0.2°θ.

In an embodiment, Form 1 has an XRPD pattern with peaks at the positions listed in Table 37 below.

TABLE 37

| °2θ | d space (Å) | Intensity % (I/Io) |
|---|---|---|
| 4.6 ± 0.1 | 19.045 ± 0.419 | 36 |
| 8.1 ± 0.1 | 10.889 ± 0.136 | 36 |
| 9.0 ± 0.1 | 9.826 ± 0.110 | 14 |
| 12.7 ± 0.1 | 6.981 ± 0.055 | 46 |

In another embodiment, Form 1 has an XRPD pattern with peaks at the positions listed in Table 38 below.

TABLE 38

| °2θ | d space (Å) | Intensity % (I/Io) |
|---|---|---|
| 4.6 ± 0.1 | 19.045 ± 0.419 | 36 |
| 8.1 ± 0.1 | 10.889 ± 0.136 | 36 |
| 9.0 ± 0.1 | 9.826 ± 0.110 | 14 |
| 10.9 ± 0.1 | 8.102 ± 0.075 | 16 |
| 12.7 ± 0.1 | 6.981 ± 0.055 | 46 |
| 14.0 ± 0.1 | 6.344 ± 0.046 | 47 |
| 15.7 ± 0.1 | 5.652 ± 0.036 | 63 |
| 20.5 ± 0.1 | 4.337 ± 0.021 | 67 |
| 24.7 ± 0.1 | 3.607 ± 0.014 | 100 |

In yet another embodiment, Form 1 has an XRPD pattern with peaks at the positions listed in Table 39 below.

TABLE 39

| °2θ | d space (Å) | Intensity % (I/Io) |
|---|---|---|
| 4.6 ± 0.1 | 19.045 ± 0.419 | 36 |
| 8.1 ± 0.1 | 10.889 ± 0.136 | 36 |
| 9.0 ± 0.1 | 9.826 ± 0.110 | 14 |
| 10.9 ± 0.1 | 8.102 ± 0.075 | 16 |
| 12.7 ± 0.1 | 6.981 ± 0.055 | 46 |
| 14.0 ± 0.1 | 6.344 ± 0.046 | 47 |
| 14.7 ± 0.1 | 6.018 ± 0.041 | 14 |
| 15.7 ± 0.1 | 5.652 ± 0.036 | 63 |
| 16.8 ± 0.1 | 5.290 ± 0.032 | 14 |
| 18.5 ± 0.1 | 4.801 ± 0.026 | 13 |
| 19.7 ± 0.1 | 4.511 ± 0.023 | 26 |
| 20.5 ± 0.1 | 4.337 ± 0.021 | 67 |
| 21.9 ± 0.1 | 4.062 ± 0.018 | 23 |
| 22.8 ± 0.1 | 3.894 ± 0.017 | 38 |
| 24.7 ± 0.1 | 3.607 ± 0.014 | 100 |
| 25.1 ± 0.1 | 3.545 ± 0.014 | 84 |
| 26.0 ± 0.1 | 3.422 ± 0.013 | 46 |
| 27.1 ± 0.1 | 3.288 ± 0.012 | 50 |
| 28.5 ± 0.1 | 3.134 ± 0.011 | 30 |
| 29.0 ± 0.1 | 3.083 ± 0.010 | 30 |
| 29.8 ± 0.1 | 2.994 ± 0.010 | 28 |

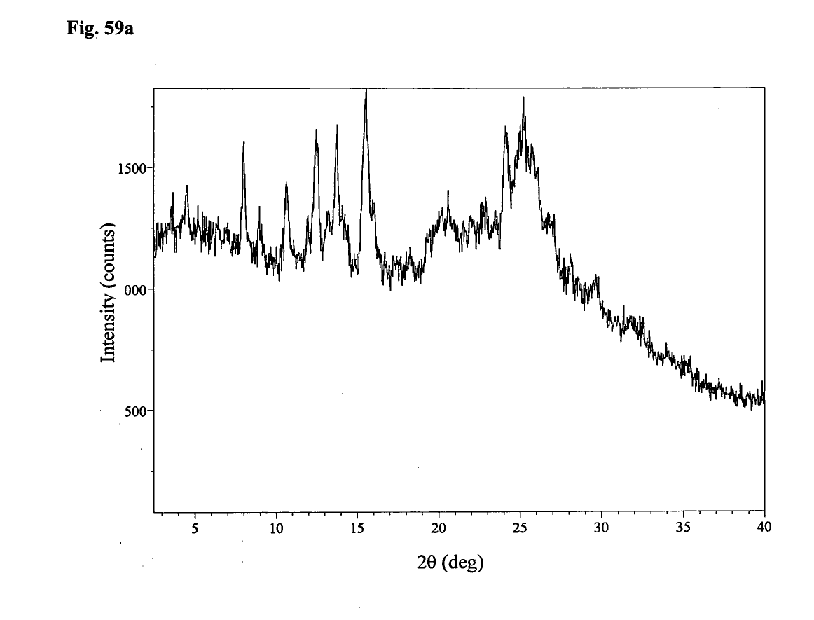

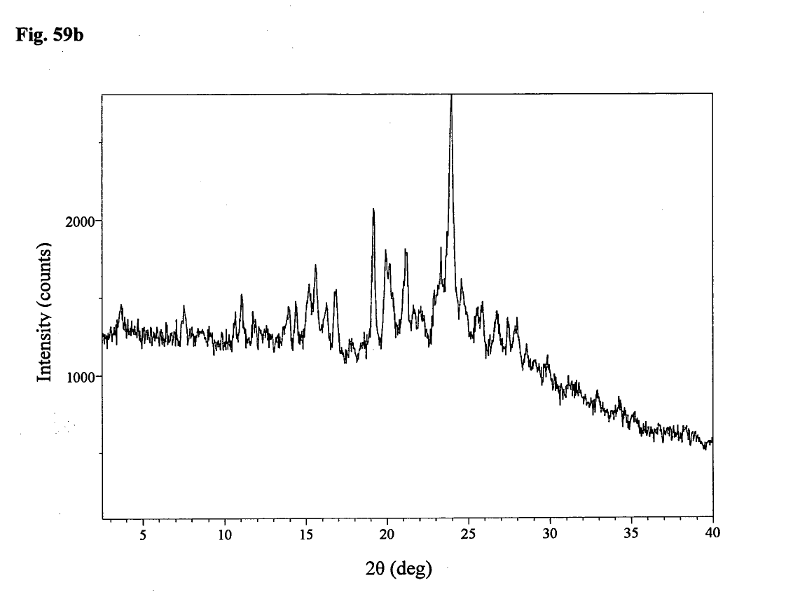

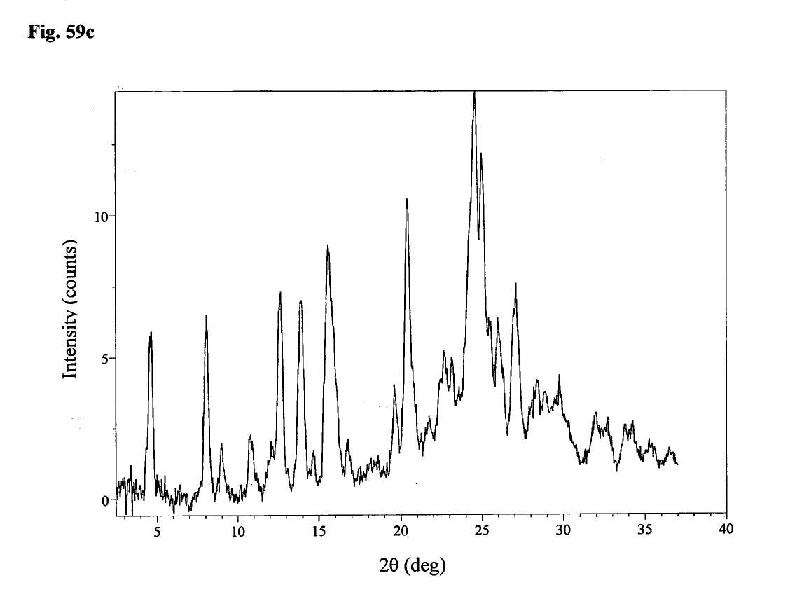

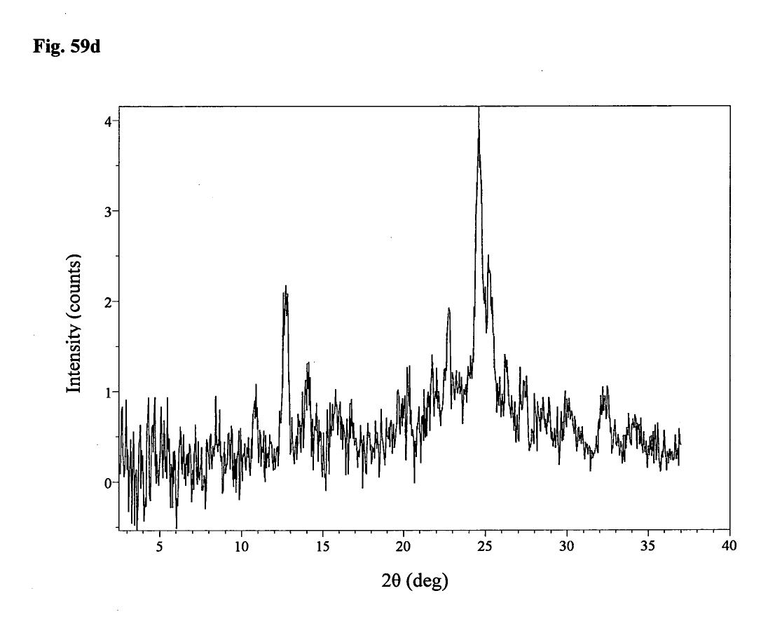

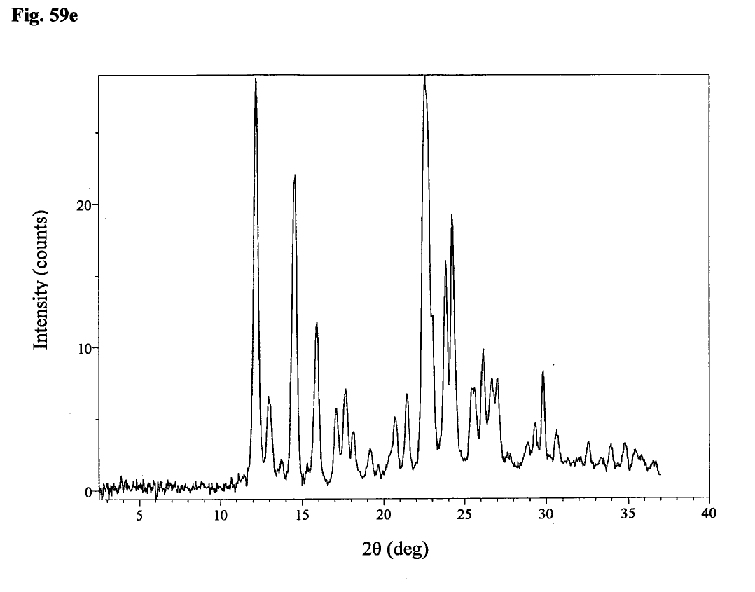

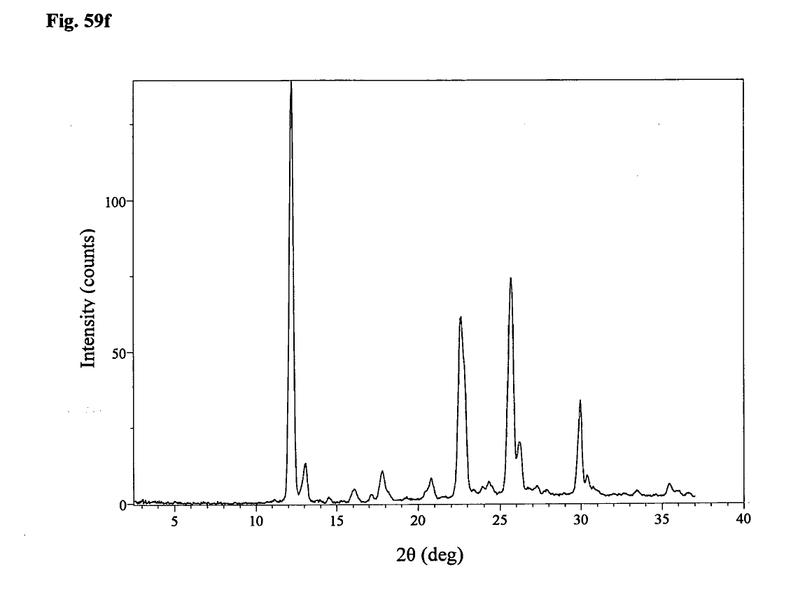

In an embodiment, Form 1 of (R)-5-(2-Aminoethyl)-1-(6,8-difluorochroman-3-yl)-1,3-dihydroimidazole-2-thione succinate is characterised as having an XRPD pattern as shown in FIG. 59.

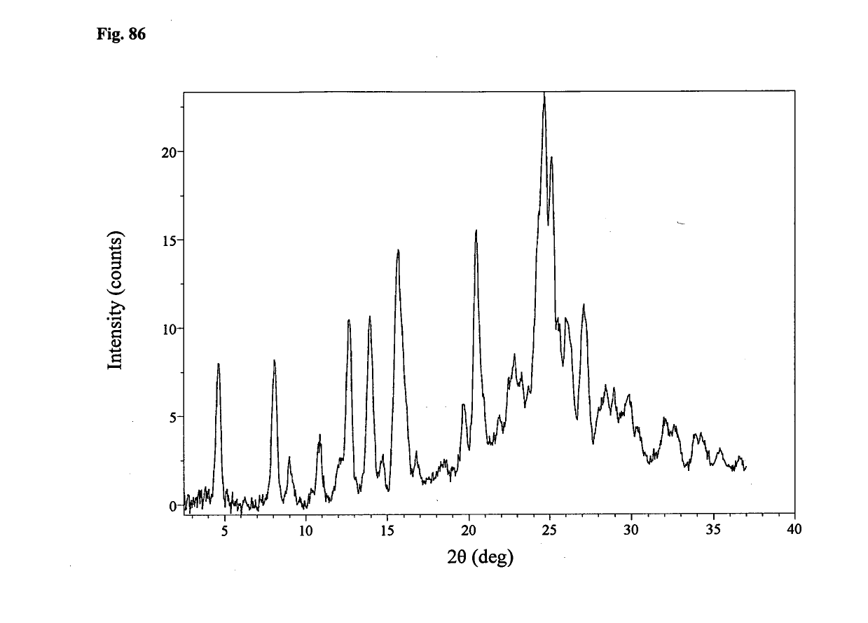

In an embodiment, Form 1 of (R)-5-(2-Aminoethyl)-1-(6,8-difluorochroman-3-yl)-1,3-dihydroimidazole-2-thione succinate is characterised as having an XRPD pattern as shown in FIG. 86.

In another embodiment, there is provided crystalline Form 2 of (R)-5-(2-Aminoethyl)-1-(6,8-difluorochroman-3-yl)-1,3-dihydroimidazole-2-thione succinate.

Form 2 may be characterised as having an XRPD pattern with a peak at 14.6 °2θ±0.2 °2θ. The XRPD pattern may have further peaks at 13.0 and 17.1 °2θ±0.2 °2θ. The XRPD pattern may have still further peaks at 12.2 and 15.9 °2θ±0.2°θ. The XRPD pattern may have still further peaks at 17.7 and 22.6 °2θ±0.2°θ.

In an embodiment, Form 2 has an XRPD pattern with peaks at the positions listed in Table 40 below.

TABLE 40

| °2θ | d space (Å) | Intensity % (I/Io) |
|---|---|---|
| 13.0 ± 0.1 | 6.831 ± 0.053 | 24 |
| 14.6 ± 0.1 | 6.084 ± 0.042 | 75 |
| 17.1 ± 0.1 | 5.192 ± 0.030 | 21 |

In another embodiment, Form 2 has an XRPD pattern with peaks at the positions listed in Table 41 below.

TABLE 41

| °2θ | d space (Å) | Intensity % (I/Io) |
|---|---|---|
| 12.2 ± 0.1 | 7.255 ± 0.060 | 99 |
| 13.0 ± 0.1 | 6.831 ± 0.053 | 24 |
| 14.6 ± 0.1 | 6.084 ± 0.042 | 75 |
| 15.9 ± 0.1 | 5.567 ± 0.035 | 42 |
| 17.1 ± 0.1 | 5.192 ± 0.030 | 21 |
| 17.7 ± 0.1 | 5.017 ± 0.028 | 26 |
| 22.6 ± 0.1 | 3.941 ± 0.017 | 100 |
| 23.8 ± 0.1 | 3.733 ± 0.015 | 56 |
| 24.2 ± 0.1 | 3.672 ± 0.015 | 67 |

In yet another embodiment, Form 2 has an XRPD pattern with peaks at the positions listed in Table 42 below.

TABLE 42

| °2θ | d space (Å) | Intensity % (I/Io) |
|---|---|---|
| 12.2 ± 0.1 | 7.255 ± 0.060 | 99 |
| 13.0 ± 0.1 | 6.831 ± 0.053 | 24 |
| 13.7 ± 0.1 | 6.454 ± 0.047 | 9 |
| 14.6 ± 0.1 | 6.084 ± 0.042 | 75 |
| 15.9 ± 0.1 | 5.567 ± 0.035 | 42 |
| 17.1 ± 0.1 | 5.192 ± 0.030 | 21 |
| 17.7 ± 0.1 | 5.017 ± 0.028 | 26 |
| 18.1 ± 0.1 | 4.896 ± 0.027 | 15 |
| 19.2 ± 0.1 | 4.632 ± 0.024 | 12 |
| 20.7 ± 0.1 | 4.287 ± 0.021 | 19 |
| 21.4 ± 0.1 | 4.145 ± 0.019 | 25 |
| 22.6 ± 0.1 | 3.941 ± 0.017 | 100 |
| 23.8 ± 0.1 | 3.733 ± 0.015 | 56 |
| 24.2 ± 0.1 | 3.672 ± 0.015 | 67 |
| 25.5 ± 0.1 | 3.496 ± 0.014 | 26 |
| 26.2 ± 0.1 | 3.407 ± 0.013 | 35 |
| 26.7 ± 0.1 | 3.341 ± 0.012 | 28 |
| 27.0 ± 0.1 | 3.298 ± 0.012 | 28 |
| 28.9 ± 0.1 | 3.092 ± 0.011 | 13 |
| 29.3 ± 0.1 | 3.046 ± 0.010 | 17 |
| 29.8 ± 0.1 | 2.994 ± 0.010 | 30 |

In an embodiment, Form 2 of (R)-5-(2-Aminoethyl)-1-(6,8-difluorochroman-3-yl)-1,3-dihydroimidazole-2-thione succinate is characterised as having an XRPD pattern as shown in FIG. 59.

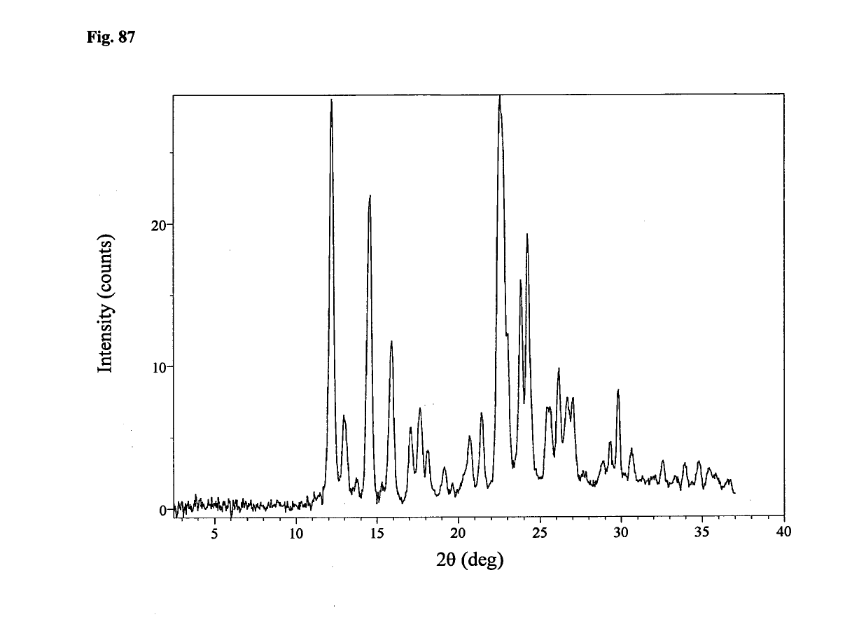

In an embodiment, Form 2 of (R)-5-(2-Aminoethyl)-1-(6,8-difluorochroman-3-yl)-1,3-dihydroimidazole-2-thione succinate is characterised as having an XRPD pattern as shown in FIG. 87.

In an embodiment, there is provided crystalline Form 3 of (R)-5-(2-Aminoethyl)-1-(6,8-difluorochroman-3-yl)-1,3-dihydroimidazole-2-thione succinate.

Form 3 may be characterised as having an XRPD pattern with a peak at 7.6 °2θ±0.2 °2θ. The XRPD pattern may have a further peak at 3.7 °2θ±0.2 °2θ. The XRPD pattern may have still further peaks at 11.1, 14.0 and 14.4 °2θ±0.2°θ. The XRPD pattern may have yet further peaks at 15.6, 19.2 and 24.0 °2θ±0.2°θ.

In an embodiment, Form 3 has an XRPD pattern with peaks at the positions listed in Table 43 below.

TABLE 43

| °2θ | d space (Å) | Intensity % (I/Io) |
|---|---|---|
| 7.6 ± 0.1 | 11.633 ± 0.155 | 14 |

In another embodiment, Form 3 has an XRPD pattern with peaks at the positions listed in Table 44 below.

TABLE 44

| °2θ | d space (Å) | Intensity % (I/Io) |
|---|---|---|
| 3.7 ± 0.1 | 24.076 ± 0.674 | 13 |
| 7.6 ± 0.1 | 11.633 ± 0.155 | 14 |

In yet another embodiment, Form 3 has an XRPD pattern with peaks at the positions listed in Table 45 below.

TABLE 45

| °2θ | d space (Å) | Intensity % (I/Io) |
|---|---|---|
| 3.7 ± 0.1 | 24.076 ± 0.674 | 13 |
| 7.6 ± 0.1 | 11.633 ± 0.155 | 14 |
| 11.1 ± 0.1 | 7.986 ± 0.073 | 23 |
| 14.0 ± 0.1 | 6.344 ± 0.046 | 18 |
| 14.4 ± 0.1 | 6.160 ± 0.043 | 19 |
| 15.2 ± 0.1 | 5.821 ± 0.038 | 28 |
| 15.6 ± 0.1 | 5.677 ± 0.036 | 35 |
| 16.3 ± 0.1 | 5.448 ± 0.033 | 20 |
| 16.8 ± 0.1 | 5.265 ± 0.031 | 26 |
| 19.2 ± 0.1 | 4.628 ± 0.024 | 56 |
| 24.0 ± 0.1 | 3.711 ± 0.015 | 100 |

In yet another embodiment, Form 3 has an XRPD pattern with peaks at the positions listed in Table 46 below.

TABLE 46

| °2θ | d space (Å) | Intensity % (I/Io) |
|---|---|---|
| 3.7 ± 0.1 | 24.076 ± 0.674 | 13 |
| 7.6 ± 0.1 | 11.633 ± 0.155 | 14 |
| 10.7 ± 0.1 | 8.299 ± 0.078 | 12 |
| 11.1 ± 0.1 | 7.986 ± 0.073 | 23 |
| 11.8 ± 0.1 | 7.519 ± 0.064 | 14 |
| 14.0 ± 0.1 | 6.344 ± 0.046 | 18 |
| 14.4 ± 0.1 | 6.160 ± 0.043 | 19 |
| 15.2 ± 0.1 | 5.821 ± 0.038 | 28 |
| 15.6 ± 0.1 | 5.677 ± 0.036 | 35 |
| 16.3 ± 0.1 | 5.448 ± 0.033 | 20 |
| 16.8 ± 0.1 | 5.265 ± 0.031 | 26 |
| 17.8 ± 0.1 | 4.983 ± 0.028 | 4 |
| 19.2 ± 0.1 | 4.628 ± 0.024 | 56 |
| 20.0 ± 0.1 | 4.448 ± 0.022 | 41 |
| 20.2 ± 0.1 | 4.396 ± 0.022 | 35 |
| 21.2 ± 0.1 | 4.187 ± 0.020 | 39 |
| 21.7 ± 0.1 | 4.096 ± 0.019 | 14 |
| 22.1 ± 0.1 | 4.030 ± 0.018 | 14 |
| 23.4 ± 0.1 | 3.810 ± 0.016 | 39 |
| 24.0 ± 0.1 | 3.711 ± 0.015 | 100 |
| 24.6 ± 0.1 | 3.617 ± 0.015 | 29 |
| 25.5 ± 0.1 | 3.488 ± 0.013 | 19 |
| 25.8 ± 0.1 | 3.448 ± 0.013 | 19 |
| 26.8 ± 0.1 | 3.330 ± 0.012 | 21 |
| 27.5 ± 0.1 | 3.248 ± 0.012 | 18 |
| 28.0 ± 0.1 | 3.190 ± 0.011 | 18 |
| 28.6 ± 0.1 | 3.124 ± 0.011 | 13 |
| 29.9 ± 0.1 | 2.989 ± 0.010 | 10 |

In an embodiment, Form 3 of (R)-5-(2-Aminoethyl)-1-(6,8-difluorochroman-3-yl)-1,3-dihydroimidazole-2-thione succinate is characterised as having an XRPD pattern as shown in FIG. 59.

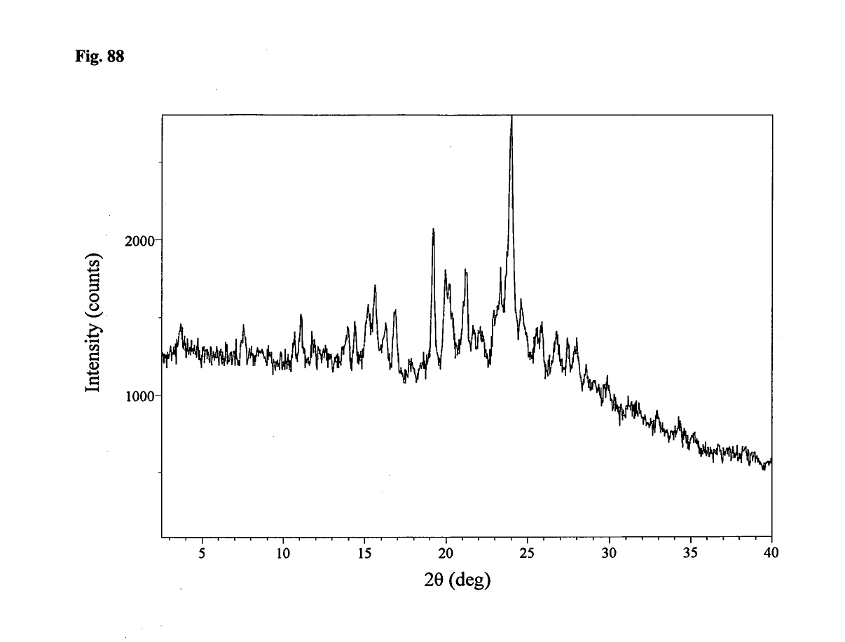

In an embodiment, Form 3 of (R)-5-(2-Aminoethyl)-1-(6,8-difluorochroman-3-yl)-1,3-dihydroimidazole-2-thione succinate is characterised as having an XRPD pattern as shown in FIG. 88.

According to another aspect of the present invention, there is provided the hydrobromide salt of (R)-5-(2-Aminoethyl)-1-(6,8-difluorochroman-3-yl)-1,3-dihydroimidazole-2-thione, i.e. (R)-5-(2-Aminoethyl)-1-(6,8-difluorochroman-3-yl)-1,3-dihydroimidazole-2-thione hydrobromide.

In an embodiment, there is provided crystalline Form 1 of (R)-5-(2-Aminoethyl)-1-(6,8-difluorochroman-3-yl)-1,3-dihydroimidazole-2-thione hydrobromide.

Form 1 may be characterised as having an XRPD pattern with a peak at 6.9 °2θ±0.2 °2θ. The XRPD pattern may have a further peak at 14.8 °2θ±0.2°2θ. The XRPD pattern may have still further peaks at 13.7, 16.5 and 18.0 °2θ±0.2°θ. The XRPD pattern may have yet further peaks at 22.0 and 27.5 °2θ±0.2°θ.

In an embodiment, Form 1 has an XRPD pattern with peaks at the positions listed in Table 47 below.

TABLE 47

| °2θ | d space (Å) | Intensity % (I/Io) |
|---|---|---|
| 6.9 ± 0.1 | 12.848 ± 0.189 | 23 |

In another embodiment, Form 1 has an XRPD pattern with peaks at the positions listed in Table 48 below.

TABLE 48

| °2θ | d space (Å) | Intensity % (I/Io) |
|---|---|---|
| 6.9 ± 0.1 | 12.848 ± 0.189 | 23 |
| 14.8 ± 0.1 | 5.970 ± 0.040 | 32 |

In yet another embodiment, Form 1 has an XRPD pattern with peaks at the positions listed in Table 49 below.

TABLE 49

| °2θ | d space (Å) | Intensity % (I/Io) |
|---|---|---|
| 6.9 ± 0.1 | 12.848 ± 0.189 | 23 |
| 13.7 ± 0.1 | 6.473 ± 0.047 | 32 |
| 14.8 ± 0.1 | 5.970 ± 0.040 | 32 |
| 16.5 ± 0.1 | 5.379 ± 0.033 | 37 |
| 18.0 ± 0.1 | 4.939 ± 0.027 | 27 |
| 20.2 ± 0.1 | 4.388 ± 0.022 | 27 |
| 21.0 ± 0.1 | 4.230 ± 0.020 | 30 |
| 22.0 ± 0.1 | 4.040 ± 0.018 | 84 |
| 27.5 ± 0.1 | 3.246 ± 0.012 | 100 |

In yet another embodiment, Form 1 has an XRPD pattern with peaks at the positions listed in Table 50 below.

TABLE 50

| °2θ | d space (Å) | Intensity % (I/Io) |
|---|---|---|
| 6.9 ± 0.1 | 12.848 ± 0.189 | 23 |
| 13.7 ± 0.1 | 6.473 ± 0.047 | 32 |
| 14.8 ± 0.1 | 5.970 ± 0.040 | 32 |
| 16.5 ± 0.1 | 5.379 ± 0.033 | 37 |
| 18.0 ± 0.1 | 4.939 ± 0.027 | 27 |
| 20.2 ± 0.1 | 4.388 ± 0.022 | 27 |
| 21.0 ± 0.1 | 4.230 ± 0.020 | 30 |
| 22.0 ± 0.1 | 4.040 ± 0.018 | 84 |
| 24.0 ± 0.1 | 3.702 ± 0.015 | 42 |
| 25.0 ± 0.1 | 3.556 ± 0.014 | 59 |
| 25.6 ± 0.1 | 3.485 ± 0.013 | 55 |
| 27.5 ± 0.1 | 3.246 ± 0.012 | 100 |

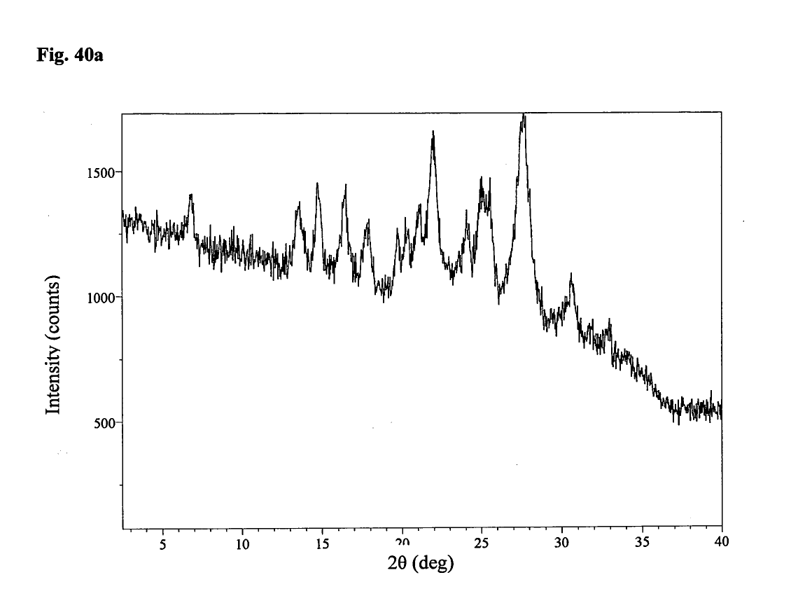

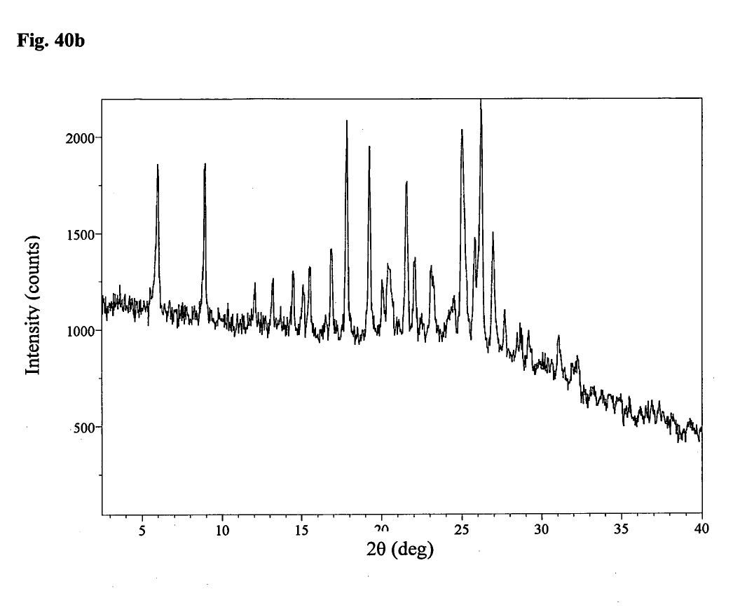

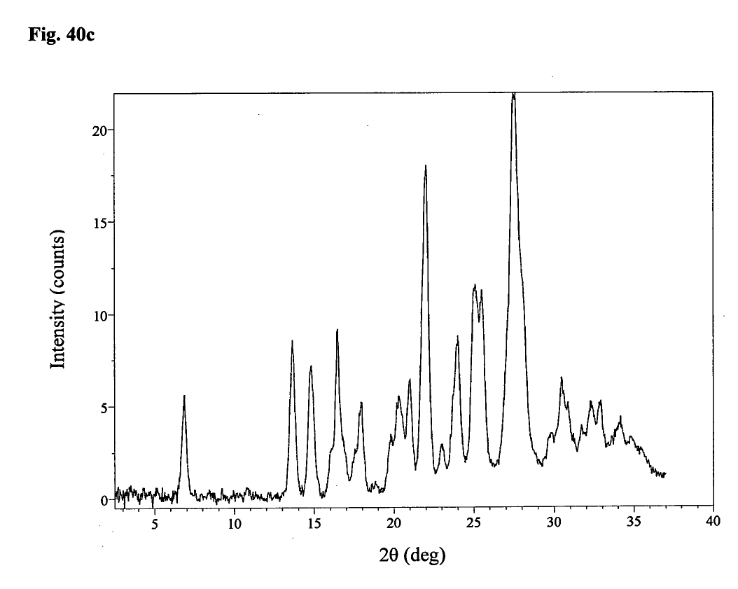

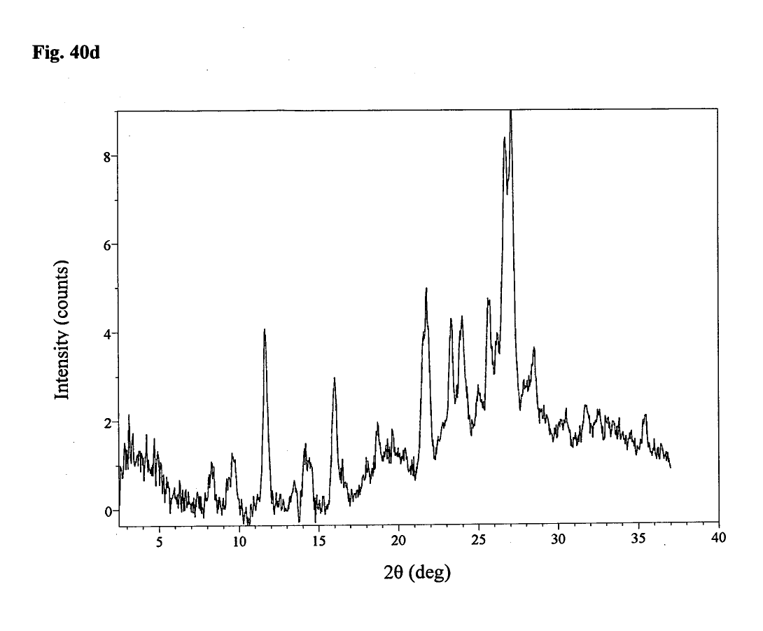

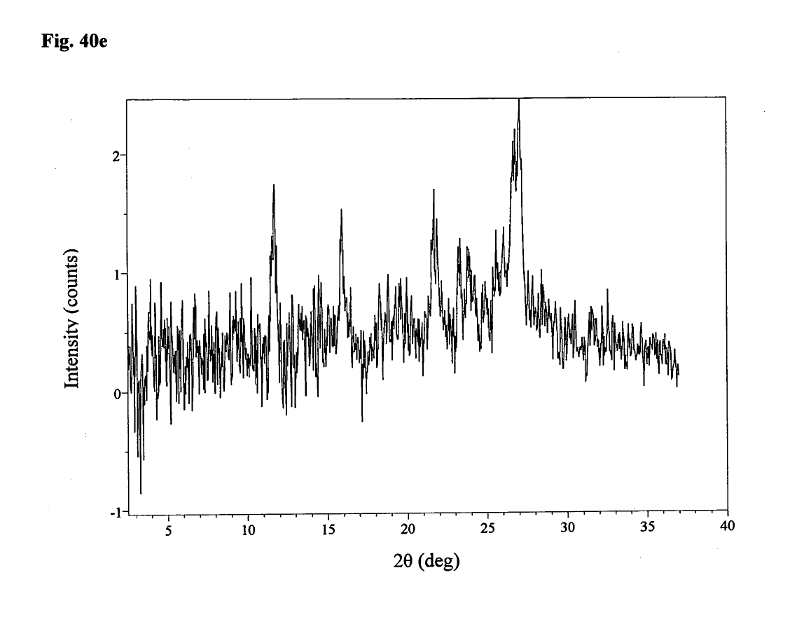

In an embodiment, Form 1 of (R)-5-(2-Aminoethyl)-1-(6,8-difluorochroman-3-yl)-1,3-dihydroimidazole-2-thione hydrobromide is characterised as having an XRPD pattern as shown in FIG. 40a. In another embodiment, Form 1 of (R)-5-(2-Aminoethyl)-1-(6,8-difluorochroman-3-yl)-1,3-dihydroimidazole-2-thione hydrobromide is characterised as having an XRPD pattern as shown in FIG. 40c.

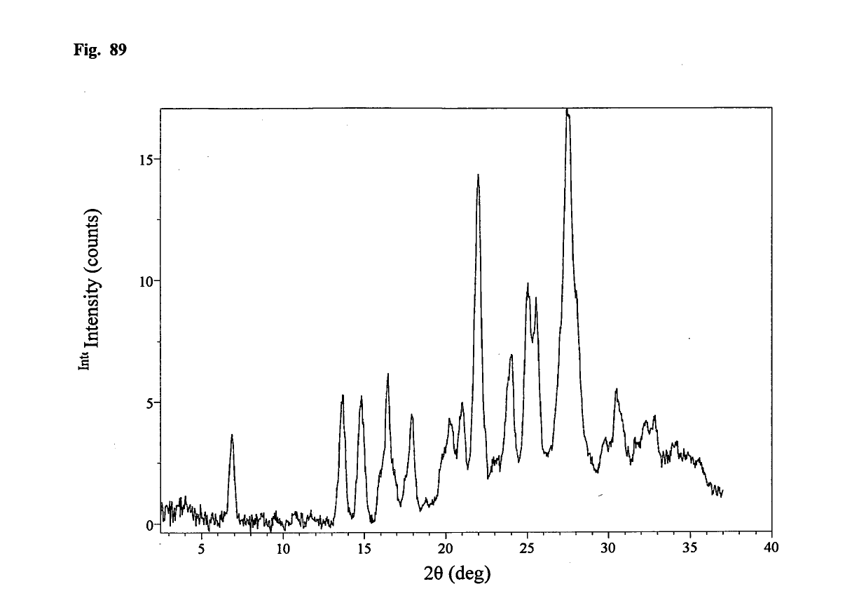

In an embodiment, Form 1 of (R)-5-(2-Aminoethyl)-1-(6,8-difluorochroman-3-yl)-1,3-dihydroimidazole-2-thione hydrobromide is characterised as having an XRPD pattern as shown in FIG. 89.

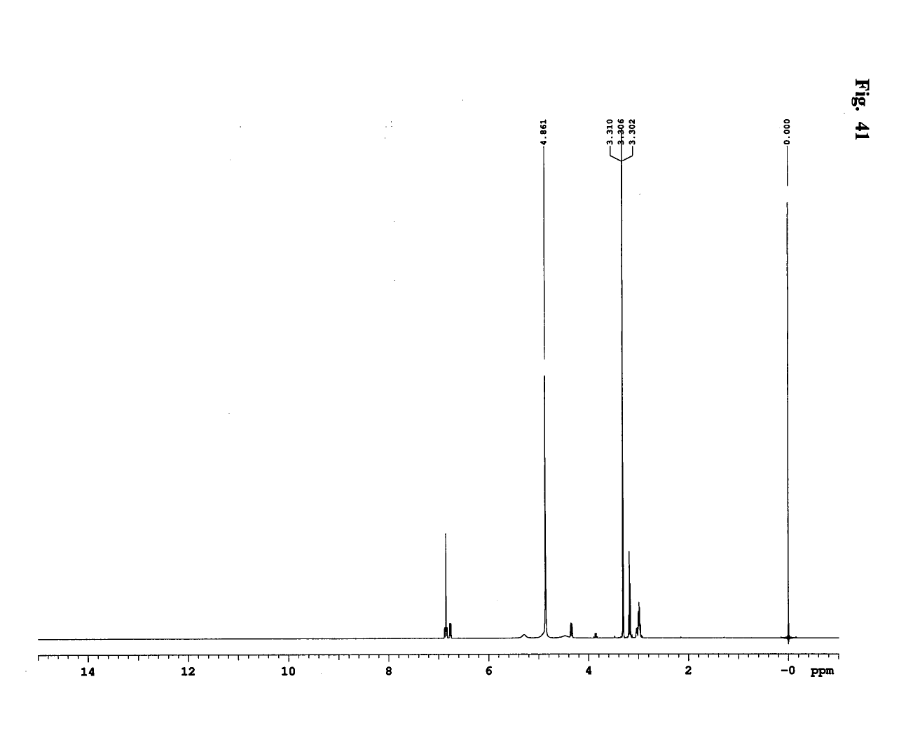

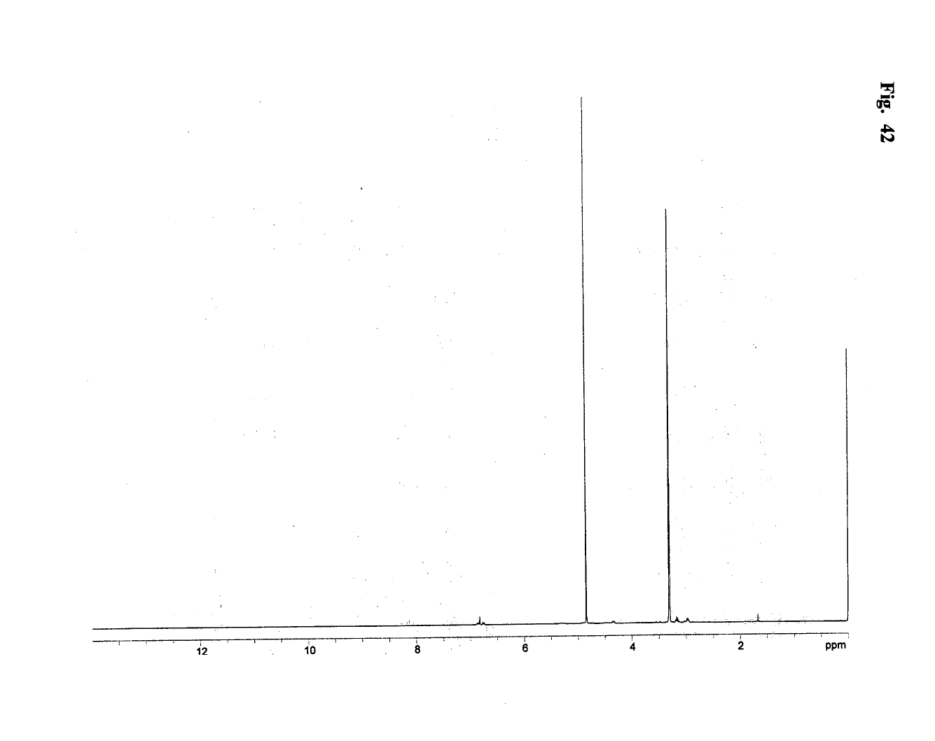

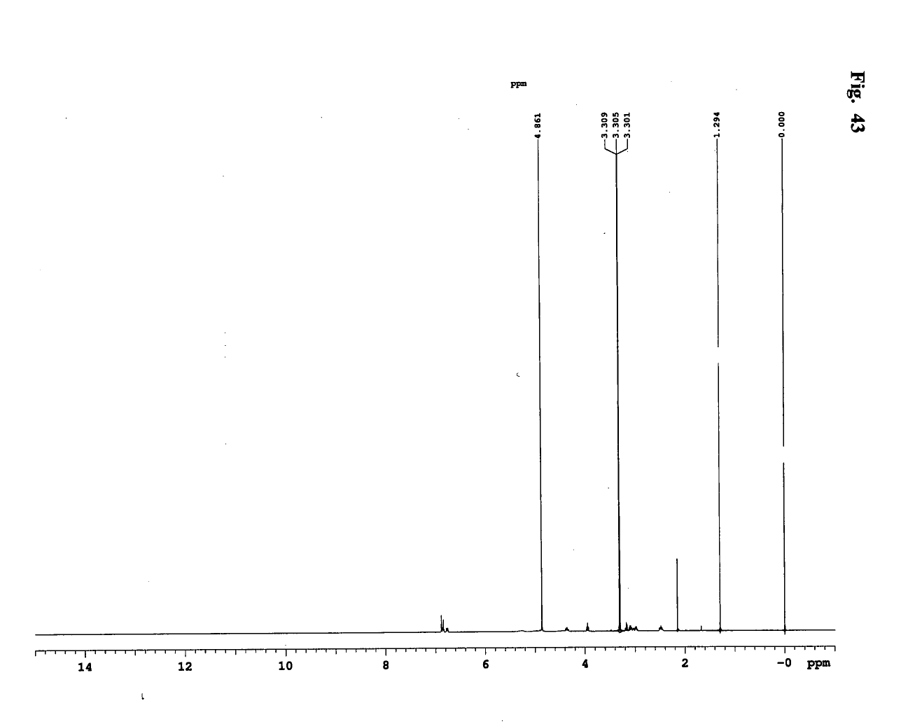

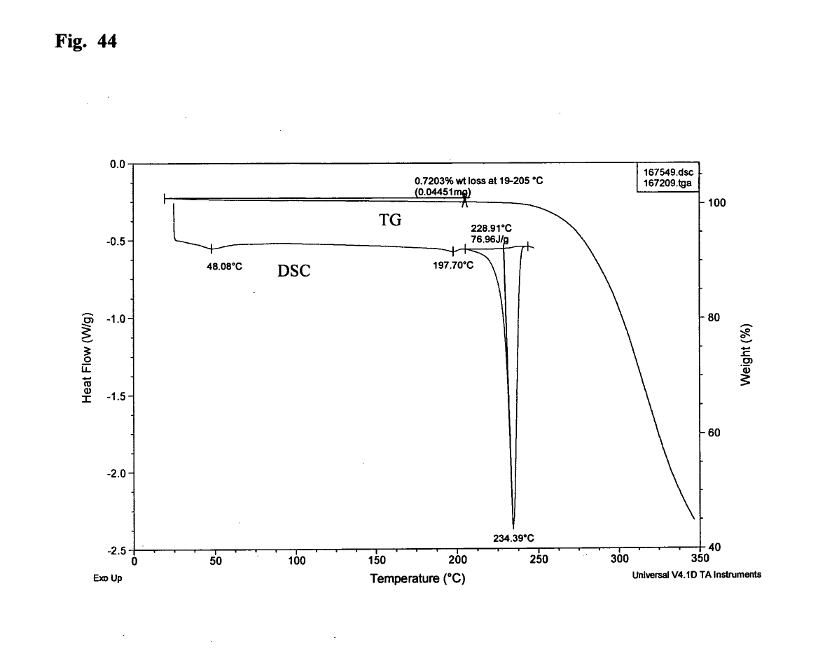

Form 1 of (R)-5-(2-Aminoethyl)-1-(6,8-difluorochroman-3-yl)-1,3-dihydroimidazole-2-thione hydrobromide may also be characterised by having a DSC thermogram as shown in FIG. 44.

In an embodiment, there is provided crystalline Form 2 of (R)-5-(2-Aminoethyl)-1-(6,8-difluorochroman-3-yl)-1,3-dihydroimidazole-2-thione hydrobromide.

Form 2 may be characterised as having an XRPD pattern with peaks at 9.7, 11.8 and 12.3 °2θ±0.2 °2θ. The XRPD pattern may have further peaks at 14.5 or 16.0 °2θ±0.2 °2θ. The XRPD pattern may have still further peaks at 18.7, 23.3 and 26.8 °2θ±0.2°θ.

In an embodiment, Form 2 has an XRPD pattern with peaks at the positions listed in Table 51 below.

TABLE 51

| °2θ | d space (Å) | Intensity % (I/Io) |
|---|---|---|
| 9.7 ± 0.1 | 9.137 ± 0.095 | 23 |
| 11.8 ± 0.1 | 7.525 ± 0.064 | 26 |
| 12.3 ± 0.1 | 7.208 ± 0.059 | 25 |

In another embodiment, Form 2 has an XRPD pattern with peaks at the positions listed in Table 52 below.

TABLE 52

| °2θ | d space (Å) | Intensity % (I/Io) |
|---|---|---|
| 9.7 ± 0.1 | 9.137 ± 0.095 | 23 |
| 11.8 ± 0.1 | 7.525 ± 0.064 | 26 |
| 12.3 ± 0.1 | 7.208 ± 0.059 | 25 |
| 14.5 ± 0.1 | 6.117 ± 0.042 | 28 |
| 16.0 ± 0.1 | 5.553 ± 0.035 | 53 |
| 18.7 ± 0.1 | 4.750 ± 0.025 | 33 |
| 22.0 ± 0.1 | 4.048 ± 0.018 | 51 |
| 23.3 ± 0.1 | 3.821 ± 0.016 | 62 |
| 26.8 ± 0.1 | 3.327 ± 0.012 | 100 |

In yet another embodiment, Form 2 has an XRPD pattern with peaks at the positions listed in Table 53 below.

TABLE 53

| °2θ | d space (Å) | Intensity % (I/Io) |
|---|---|---|
| 4.8 ± 0.1 | 18.565 ± 0.398 | 12 |
| 8.3 ± 0.1 | 10.627 ± 0.129 | 14 |
| 9.7 ± 0.1 | 9.137 ± 0.095 | 23 |
| 11.8 ± 0.1 | 7.525 ± 0.064 | 26 |
| 12.3 ± 0.1 | 7.208 ± 0.059 | 25 |
| 13.6 ± 0.1 | 6.511 ± 0.048 | 19 |
| 14.5 ± 0.1 | 6.117 ± 0.042 | 28 |
| 16.0 ± 0.1 | 5.553 ± 0.035 | 53 |
| 18.7 ± 0.1 | 4.750 ± 0.025 | 33 |
| 21.6 ± 0.1 | 4.114 ± 0.019 | 46 |
| 22.0 ± 0.1 | 4.048 ± 0.018 | 51 |
| 23.3 ± 0.1 | 3.821 ± 0.016 | 62 |
| 24.0 ± 0.1 | 3.708 ± 0.015 | 48 |
| 24.9 ± 0.1 | 3.579 ± 0.014 | 51 |
| 26.8 ± 0.1 | 3.327 ± 0.012 | 100 |
| 28.5 ± 0.1 | 3.134 ± 0.011 | 42 |

In an embodiment, Form 2 of (R)-5-(2-Aminoethyl)-1-(6,8-difluorochroman-3-yl)-1,3-dihydroimidazole-2-thione hydrobromide is characterised as having an XRPD pattern as shown in FIG. 40d.

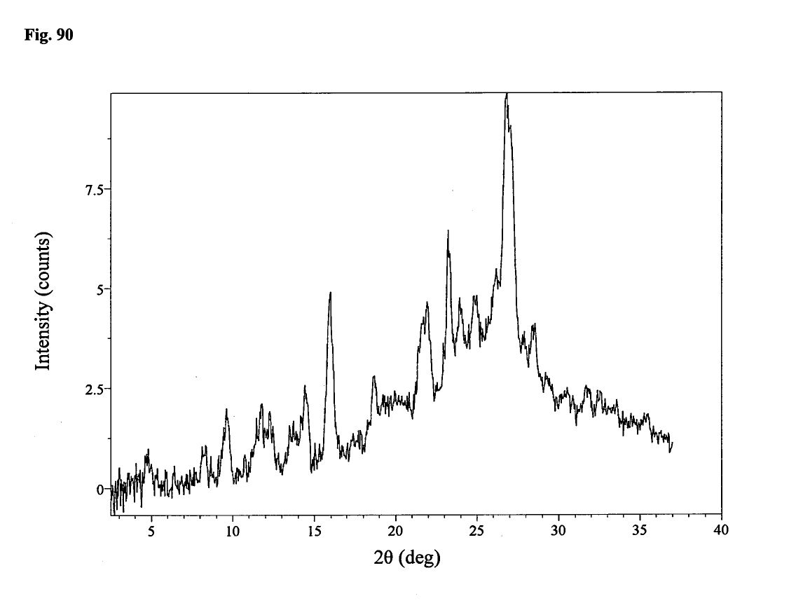

In an embodiment, Form 2 of (R)-5-(2-Aminoethyl)-1-(6,8-difluorochroman-3-yl)-1,3-dihydroimidazole-2-thione hydrobromide is characterised as having an XRPD pattern as shown in FIG. 90.

In an embodiment, there is provided crystalline Form 3 of (R)-5-(2-Aminoethyl)-1-(6,8-difluorochroman-3-yl)-1,3-dihydroimidazole-2-thione hydrobromide.

Form 3 may be characterised as having an XRPD pattern with peaks at 6.0, 8.9 and 13.2 °2θ±0.2 °2θ. The XRPD pattern may have further peaks at 15.1, 15.6 and 16.9 °2θ±0.2°2θ. The XRPD pattern may have still further peaks at 12.1 and 14.5 °2θ±0.2°θ. The XRPD pattern may have still further peaks at 17.9 and 26.2 °2θ±0.2°θ.

In an embodiment, Form 3 has an XRPD pattern with peaks at the positions listed in Table 54 below.

TABLE 54

| °2θ | d space (Å) | Intensity % (I/Io) |
|---|---|---|
| 6.0 ± 0.1 | 14.706 ± 0.249 | 63 |
| 8.9 ± 0.1 | 9.914 ± 0.112 | 64 |
| 13.2 ± 0.1 | 6.702 ± 0.051 | 23 |
| 15.1 ± 0.1 | 5.867 ± 0.039 | 21 |
| 15.6 ± 0.1 | 5.699 ± 0.037 | 29 |
| 16.9 ± 0.1 | 5.256 ± 0.031 | 37 |

In another embodiment, Form 3 has an XRPD pattern with peaks at the positions listed in Table 55 below.

TABLE 55

| °2θ | d space (Å) | Intensity % (I/Io) |
|---|---|---|
| 6.0 ± 0.1 | 14.706 ± 0.249 | 63 |
| 8.9 ± 0.1 | 9.914 ± 0.112 | 64 |
| 12.1 ± 0.1 | 7.333 ± 0.061 | 21 |
| 13.2 ± 0.1 | 6.702 ± 0.051 | 23 |
| 14.5 ± 0.1 | 6.109 ± 0.042 | 26 |
| 15.1 ± 0.1 | 5.867 ± 0.039 | 21 |
| 15.6 ± 0.1 | 5.699 ± 0.037 | 29 |
| 16.9 ± 0.1 | 5.256 ± 0.031 | 37 |
| 17.9 ± 0.1 | 4.966 ± 0.028 | 86 |
| 19.3 ± 0.1 | 4.606 ± 0.024 | 78 |
| 21.6 ± 0.1 | 4.118 ± 0.019 | 64 |
| 25.1 ± 0.1 | 3.549 ± 0.014 | 78 |
| 26.2 ± 0.1 | 3.401 ± 0.013 | 100 |

In yet another embodiment, Form 3 has an XRPD pattern with peaks at the positions listed in Table 56 below.

TABLE 56

| °2θ | d space (Å) | Intensity % (I/Io) |
|---|---|---|
| 6.0 ± 0.1 | 14.706 ± 0.249 | 63 |
| 8.9 ± 0.1 | 9.914 ± 0.112 | 64 |
| 12.1 ± 0.1 | 7.333 ± 0.061 | 21 |
| 13.2 ± 0.1 | 6.702 ± 0.051 | 23 |
| 14.5 ± 0.1 | 6.109 ± 0.042 | 26 |
| 15.1 ± 0.1 | 5.867 ± 0.039 | 21 |
| 15.6 ± 0.1 | 5.699 ± 0.037 | 29 |
| 16.9 ± 0.1 | 5.256 ± 0.031 | 37 |
| 17.9 ± 0.1 | 4.966 ± 0.028 | 86 |
| 19.3 ± 0.1 | 4.606 ± 0.024 | 78 |
| 20.1 ± 0.1 | 4.422 ± 0.022 | 23 |
| 20.4 ± 0.1 | 4.351 ± 0.021 | 30 |
| 21.6 ± 0.1 | 4.118 ± 0.019 | 64 |
| 22.1 ± 0.1 | 4.024 ± 0.018 | 33 |
| 23.1 ± 0.1 | 3.849 ± 0.016 | 31 |
| 24.4 ± 0.1 | 3.648 ± 0.015 | 14 |
| 25.1 ± 0.1 | 3.549 ± 0.014 | 78 |
| 25.8 ± 0.1 | 3.452 ± 0.013 | 45 |
| 26.2 ± 0.1 | 3.401 ± 0.013 | 100 |
| 27.0 ± 0.1 | 3.308 ± 0.012 | 49 |
| 27.7 ± 0.1 | 3.221 ± 0.011 | 18 |
| 28.7 ± 0.1 | 3.115 ± 0.011 | 16 |
| 29.2 ± 0.1 | 3.062 ± 0.010 | 17 |

In an embodiment, Form 3 of (R)-5-(2-Aminoethyl)-1-(6,8-difluorochroman-3-yl)-1,3-dihydroimidazole-2-thione hydrobromide is characterised as having an XRPD pattern as shown in FIG. 40b.

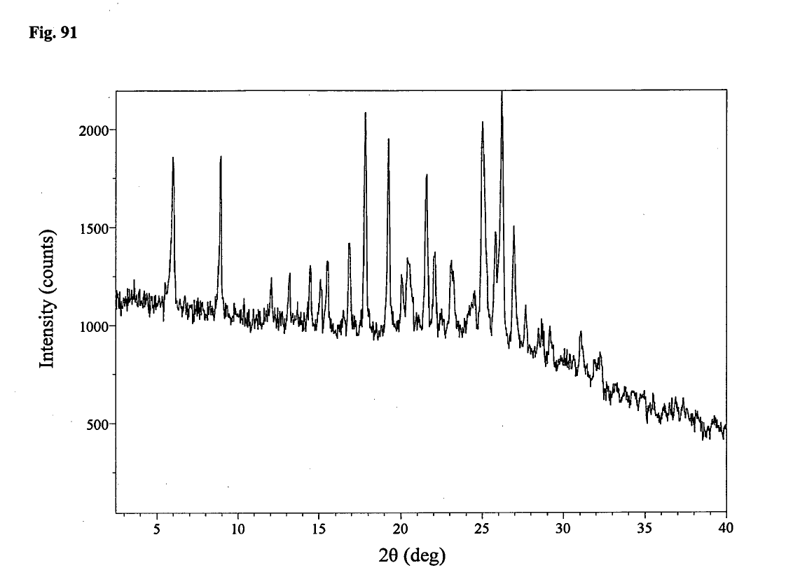

In an embodiment, Form 3 of (R)-5-(2-Aminoethyl)-1-(6,8-difluorochroman-3-yl)-1,3-dihydroimidazole-2-thione hydrobromide is characterised as having an XRPD pattern as shown in FIG. 91.

According to another aspect of the present invention, there is provided the maleic acid salt of (R)-5-(2-Aminoethyl)-1-(6,8-difluorochroman-3-yl)-1,3-dihydroimidazole-2-thione, i.e. (R)-5-(2-Aminoethyl)-1-(6,8-difluorochroman-3-yl)-1,3-dihydroimidazole-2-thione maleate.

In an embodiment, there is provided crystalline Form 1 of (R)-5-(2-Aminoethyl)-1-(6,8-difluorochroman-3-yl)-1,3-dihydroimidazole-2-thione maleate.

Form 1 may be characterised as having an XRPD pattern with peaks at 11.3, 14.1 and 14.4 °2θ±0.2°2θ. The XRPD pattern may have a further peak at 9.1 °2θ±0.2 °2θ. The XRPD pattern may have still further peaks at 15.6 and 16.4 °2θ±0.2°θ. The XRPD pattern may have yet further peaks at 19.7 and 25.2 °θ0±0.2°θ.

In an embodiment, Form 1 has an XRPD pattern with peaks at the positions listed in Table 57 below.

TABLE 57

| °2θ | d space (Å) | Intensity % (I/Io) |
|---|---|---|
| 9.1 ± 0.1 | 9.697 ± 0.107 | 14 |
| 11.3 ± 0.1 | 7.817 ± 0.069 | 34 |
| 14.1 ± 0.1 | 6.290 ± 0.045 | 30 |
| 14.4 ± 0.1 | 6.134 ± 0.043 | 31 |
| 15.6 ± 0.1 | 5.666 ± 0.036 | 24 |
| 16.4 ± 0.1 | 5.418 ± 0.033 | 56 |

In another embodiment, Form 1 has an XRPD pattern with peaks at the positions listed in Table 58 below.

TABLE 58

| °2θ | d space (Å) | Intensity % (I/Io) |
|---|---|---|
| 9.1 ± 0.1 | 9.697 ± 0.107 | 14 |
| 11.3 ± 0.1 | 7.817 ± 0.069 | 34 |
| 12.5 ± 0.1 | 7.070 ± 0.057 | 15 |
| 14.1 ± 0.1 | 6.290 ± 0.045 | 30 |
| 14.4 ± 0.1 | 6.134 ± 0.043 | 31 |
| 15.6 ± 0.1 | 5.666 ± 0.036 | 24 |
| 16.4 ± 0.1 | 5.418 ± 0.033 | 56 |
| 19.7 ± 0.1 | 4.502 ± 0.023 | 44 |
| 22.8 ± 0.1 | 3.900 ± 0.017 | 36 |
| 24.0 ± 0.1 | 3.702 ± 0.015 | 70 |
| 25.2 ± 0.1 | 3.534 ± 0.014 | 100 |

In yet another embodiment, Form 1 has an XRPD pattern with peaks at the positions listed in Table 59 below.

TABLE 59

| °2θ | d space (Å) | Intensity % (I/Io) |
|---|---|---|
| 9.1 ± 0.1 | 9.697 ± 0.107 | 14 |
| 10.6 ± 0.1 | 8.346 ± 0.079 | 9 |
| 11.3 ± 0.1 | 7.817 ± 0.069 | 34 |
| 12.5 ± 0.1 | 7.070 ± 0.057 | 15 |
| 13.4 ± 0.1 | 6.608 ± 0.049 | 12 |
| 14.1 ± 0.1 | 6.290 ± 0.045 | 30 |
| 14.4 ± 0.1 | 6.134 ± 0.043 | 31 |
| 15.6 ± 0.1 | 5.666 ± 0.036 | 24 |
| 16.4 ± 0.1 | 5.418 ± 0.033 | 56 |
| 17.2 ± 0.1 | 5.156 ± 0.030 | 15 |
| 17.7 ± 0.1 | 5.005 ± 0.028 | 14 |
| 18.6 ± 0.1 | 4.760 ± 0.025 | 11 |
| 19.7 ± 0.1 | 4.502 ± 0.023 | 44 |
| 20.6 ± 0.1 | 4.303 ± 0.021 | 19 |
| 21.0 ± 0.1 | 4.222 ± 0.020 | 16 |
| 21.7 ± 0.1 | 4.092 ± 0.019 | 21 |
| 22.8 ± 0.1 | 3.900 ± 0.017 | 36 |
| 24.0 ± 0.1 | 3.702 ± 0.015 | 70 |
| 25.2 ± 0.1 | 3.534 ± 0.014 | 100 |
| 26.2 ± 0.1 | 3.407 ± 0.013 | 35 |
| 27.2 ± 0.1 | 3.279 ± 0.012 | 44 |
| 29.1 ± 0.1 | 3.067 ± 0.010 | 20 |

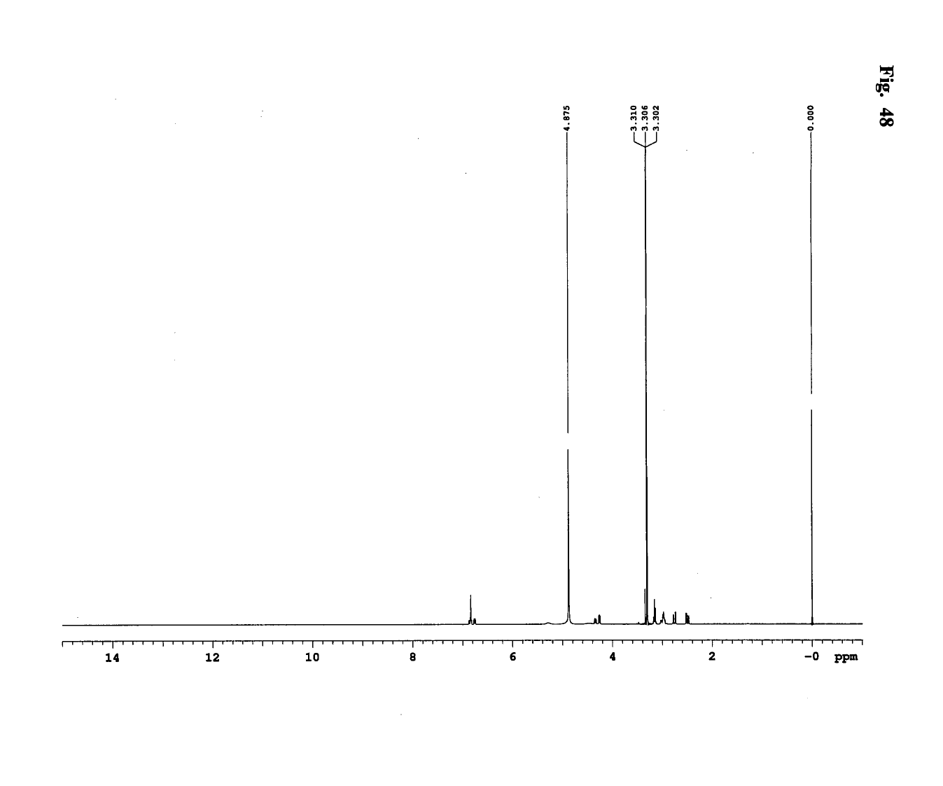

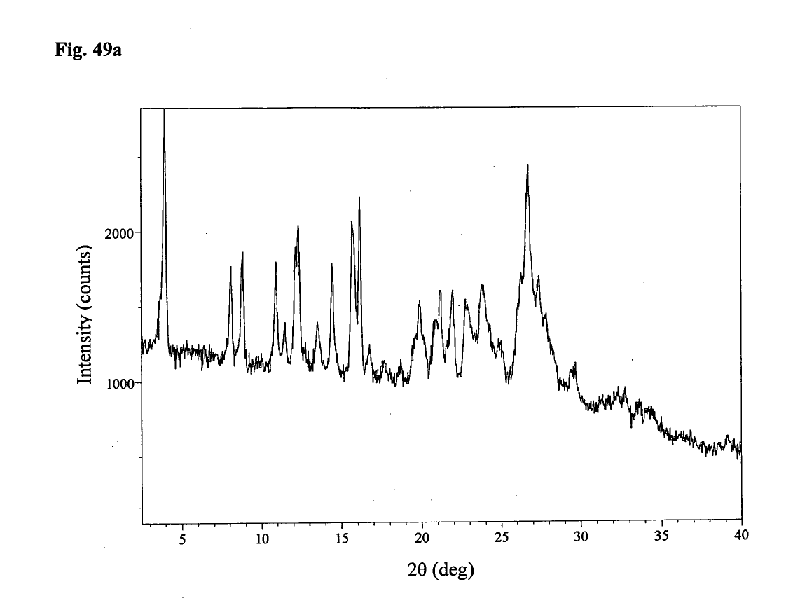

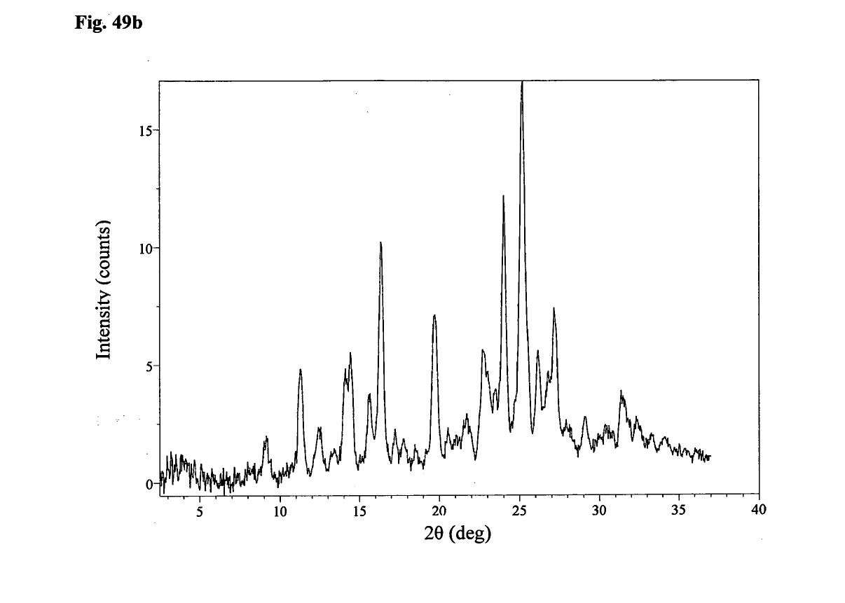

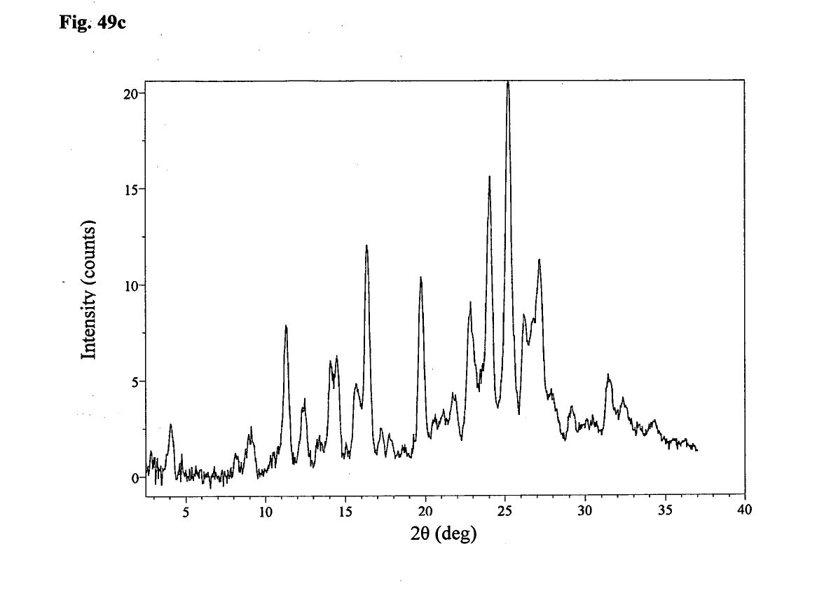

In an embodiment, Form 1 of (R)-5-(2-Aminoethyl)-1-(6,8-difluorochroman-3-yl)-1,3-dihydroimidazole-2-thione maleate is characterised as having an XRPD pattern as shown in FIG. 49b.

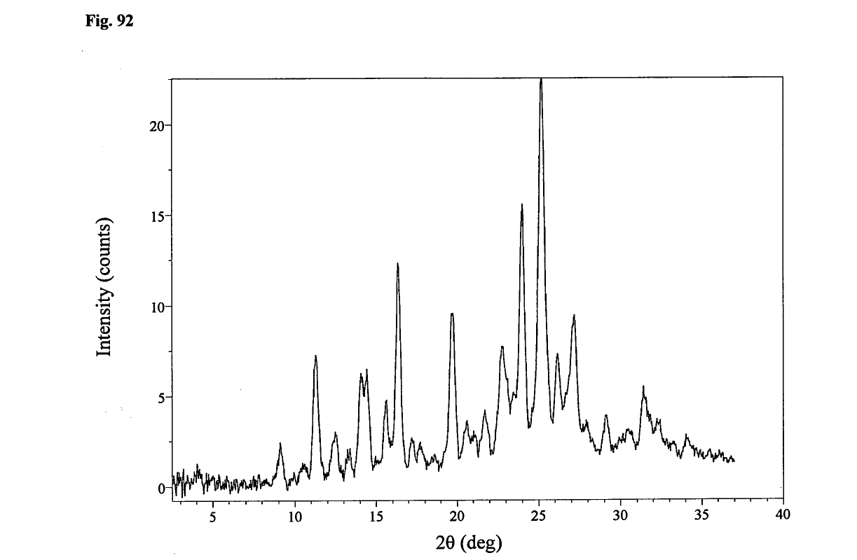

In an, embodiment, Form 1 of (R)-5-(2-Aminoethyl)-1-(6,8-difluorochroman-3-yl)-1,3-dihydroimidazole-2-thione maleate is characterised as having an XRPD pattern as shown in FIG. 92.

In an embodiment, there is provided crystalline Form 1+peaks of (R)-5-(2-Aminoethyl)-1-(6,8-difluorochroman-3-yl)-1,3-dihydroimidazole-2-thione maleate. Hereinafter, this crystalline form shall be referred to as Form 2 of (R)-5-(2-Aminoethyl)-1-(6,8-difluorochroman-3-yl)-1,3-dihydroimidazole-2-thione maleate.

Form 2 may be characterised as having an XRPD pattern with peaks at 4.0, 8.1, 8.8 and 11.0 °2θ±0.2 °2θ. The XRPD pattern may have a further peak at 16.2 °2θ±0.2 °2θ. The XRPD pattern may have still further peaks at 12.3 and 14.5 °2θ±0.2°θ. The XRPD pattern may have a yet further peak at 15.8 °2θ±0.2°θ.

In an embodiment, Form 2 has an XRPD pattern with peaks at the positions listed in Table 60 below.

TABLE 60

| °2θ | d space (Å) | Intensity % (I/Io) |
|---|---|---|
| 4.0 ± 0.1 | 22.090 ± 0.566 | 100 |
| 8.1 ± 0.1 | 10.902 ± 0.136 | 44 |
| 8.8 ± 0.1 | 10.015 ± 0.114 | 49 |
| 11.0 ± 0.1 | 8.073 ± 0.074 | 49 |
| 16.2 ± 0.1 | 5.478 ± 0.034 | 80 |

In another embodiment, Form 2 has an XRPD pattern with peaks at the positions listed in Table 61 below.

TABLE 61

| °2θ | d space (Å) | Intensity % (I/Io) |
|---|---|---|
| 4.0 ± 0.1 | 22.090 ± 0.566 | 100 |
| 8.1 ± 0.1 | 10.902 ± 0.136 | 44 |
| 8.8 ± 0.1 | 10.015 ± 0.114 | 49 |
| 11.0 ± 0.1 | 8.073 ± 0.074 | 49 |
| 12.3 ± 0.1 | 7.173 ± 0.058 | 65 |
| 14.5 ± 0.1 | 6.121 ± 0.042 | 50 |
| 15.8 ± 0.1 | 5.623 ± 0.036 | 67 |
| 16.2 ± 0.1 | 5.478 ± 0.034 | 80 |

In yet another embodiment, Form 2 has an XRPD pattern with peaks at the positions listed in Table 62 below.

TABLE 62

| °2θ | d space (Å) | Intensity % (I/Io) |
|---|---|---|
| 4.0 ± 0.1 | 22.090 ± 0.566 | 100 |
| 8.1 ± 0.1 | 10.902 ± 0.136 | 44 |
| 8.8 ± 0.1 | 10.015 ± 0.114 | 49 |
| 11.0 ± 0.1 | 8.073 ± 0.074 | 49 |
| 11.5 ± 0.1 | 7.695 ± 0.067 | 21 |
| 12.3 ± 0.1 | 7.173 ± 0.058 | 65 |
| 13.6 ± 0.1 | 6.525 ± 0.048 | 22 |
| 14.5 ± 0.1 | 6.121 ± 0.042 | 50 |
| 15.8 ± 0.1 | 5.623 ± 0.036 | 67 |
| 16.2 ± 0.1 | 5.478 ± 0.034 | 80 |
| 16.8 ± 0.1 | 5.284 ± 0.031 | 16 |
| 17.7 ± 0.1 | 5.017 ± 0.028 | 9 |
| 18.7 ± 0.1 | 4.745 ± 0.025 | 8 |
| 19.9 ± 0.1 | 4.462 ± 0.022 | 34 |
| 20.9 ± 0.1 | 4.246 ± 0.020 | 27 |
| 21.2 ± 0.1 | 4.193 ± 0.020 | 40 |
| 22.0 ± 0.1 | 4.046 ± 0.018 | 39 |
| 22.8 ± 0.1 | 3.899 ± 0.017 | 31 |
| 23.8 ± 0.1 | 3.734 ± 0.016 | 42 |
| 24.9 ± 0.1 | 3.575 ± 0.014 | 14 |
| 26.3 ± 0.1 | 3.390 ± 0.013 | 50 |
| 26.7 ± 0.1 | 3.338 ± 0.012 | 95 |
| 27.4 ± 0.1 | 3.259 ± 0.012 | 48 |
| 29.6 ± 0.1 | 3.013 ± 0.010 | 14 |

In another embodiment, Form 2 of (R)-5-(2-Aminoethyl)-1-(6,8-difluorochroman-3-yl)-1,3-dihydroimidazole-2-thione maleate is characterised as having an XRPD pattern as shown in FIG. 49a.

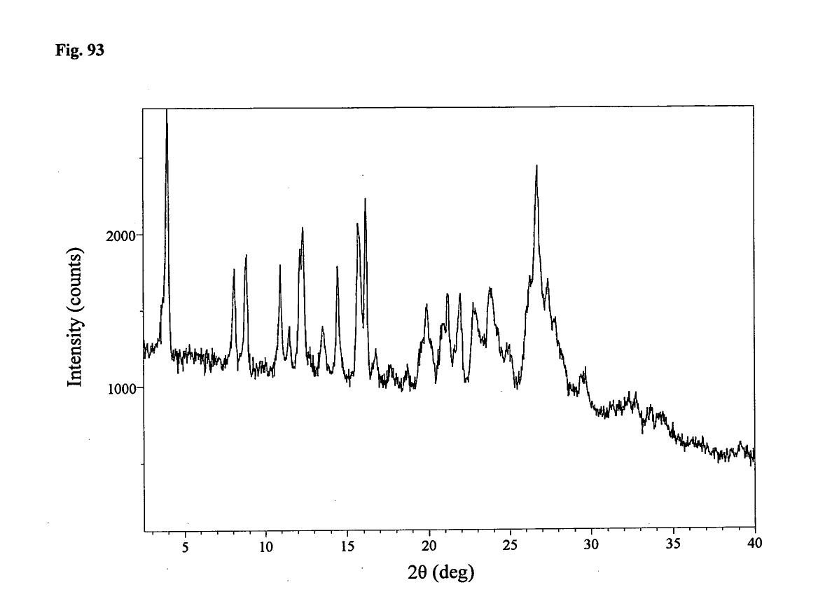

In another embodiment, Form 2 of (R)-5-(2-Aminoethyl)-1-(6,8-difluorochroman-3-yl)-1,3-dihydroimidazole-2-thione maleate is characterised as having an XRPD pattern as shown in FIG. 93.

According to another aspect of the present invention, there is provided the phosphoric acid salt of (R)-5-(2-Aminoethyl)-1-(6,8-difluorochroman-3-yl)-1,3-dihydroimidazole-2-thione, i.e. (R)-5-(2-Aminoethyl)-1-(6,8-difluorochroman-3-yl)-1,3-dihydroimidazole-2-thione phosphate.

In an embodiment, there is provided crystalline Form 1 of (R)-5-(2-Aminoethyl)-1-(6,8-difluorochroman-3-yl)-1,3-dihydroimidazole-2-thione phosphate.

Form 1 may be characterised as having an XRPD pattern with peaks at 4.6, 8.5, 9.3 and 11.0 °2θ±0.2 °2θ. The XRPD pattern may have a further peak at 16.4 °2θ±0.2°2θ. The XRPD pattern may have still further peaks at 21.0, 23.0 and 27.2 °2θ±0.2°θ.

In an embodiment, Form 1 has an XRPD pattern with peaks at the positions listed in Table 63 below.

TABLE 63

| °2θ | d space (Å) | Intensity % (I/Io) |
|---|---|---|
| 4.6 ± 0.1 | 19.210 ± 0.427 | 14 |
| 8.5 ± 0.1 | 10.378 ± 0.123 | 27 |
| 9.3 ± 0.1 | 9.530 ± 0.104 | 30 |
| 11.0 ± 0.1 | 8.073 ± 0.074 | 46 |
| 16.4 ± 0.1 | 5.392 ± 0.033 | 55 |
| 21.0 ± 0.1 | 4.238 ± 0.020 | 40 |
| 23.0 ± 0.1 | 3.874 ± 0.017 | 44 |
| 27.2 ± 0.1 | 3.283 ± 0.012 | 100 |

In another embodiment, Form 1 has an XRPD pattern with peaks at the positions listed in Table 64 below.

TABLE 64

| °2θ | d space (Å) | Intensity % (I/Io) |
|---|---|---|
| 4.6 ± 0.1 | 19.210 ± 0.427 | 14 |
| 8.5 ± 0.1 | 10.378 ± 0.123 | 27 |
| 9.3 ± 0.1 | 9.530 ± 0.104 | 30 |
| 11.0 ± 0.1 | 8.073 ± 0.074 | 46 |
| 11.6 ± 0.1 | 7.629 ± 0.066 | 12 |
| 12.3 ± 0.1 | 7.185 ± 0.059 | 18 |
| 12.8 ± 0.1 | 6.938 ± 0.055 | 16 |
| 13.8 ± 0.1 | 6.417 ± 0.047 | 15 |
| 14.3 ± 0.1 | 6.185 ± 0.043 | 19 |
| 15.3 ± 0.1 | 5.799 ± 0.038 | 19 |
| 16.4 ± 0.1 | 5.392 ± 0.033 | 55 |
| 18.1 ± 0.1 | 4.896 ± 0.027 | 19 |
| 19.4 ± 0.1 | 4.566 ± 0.023 | 14 |
| 20.0 ± 0.1 | 4.431 ± 0.022 | 20 |
| 21.0 ± 0.1 | 4.238 ± 0.020 | 40 |
| 21.7 ± 0.1 | 4.099 ± 0.019 | 22 |
| 23.0 ± 0.1 | 3.874 ± 0.017 | 44 |
| 24.2 ± 0.1 | 3.678 ± 0.015 | 22 |
| 24.8 ± 0.1 | 3.584 ± 0.014 | 32 |
| 25.7 ± 0.1 | 3.469 ± 0.013 | 25 |
| 27.2 ± 0.1 | 3.283 ± 0.012 | 100 |
| 28.7 ± 0.1 | 3.113 ± 0.011 | 40 |
| 29.7 ± 0.1 | 3.006 ± 0.010 | 16 |

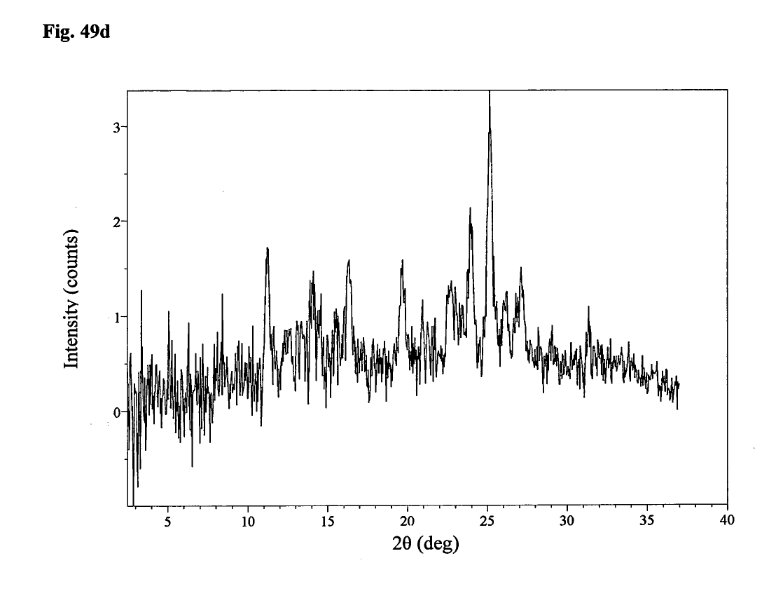

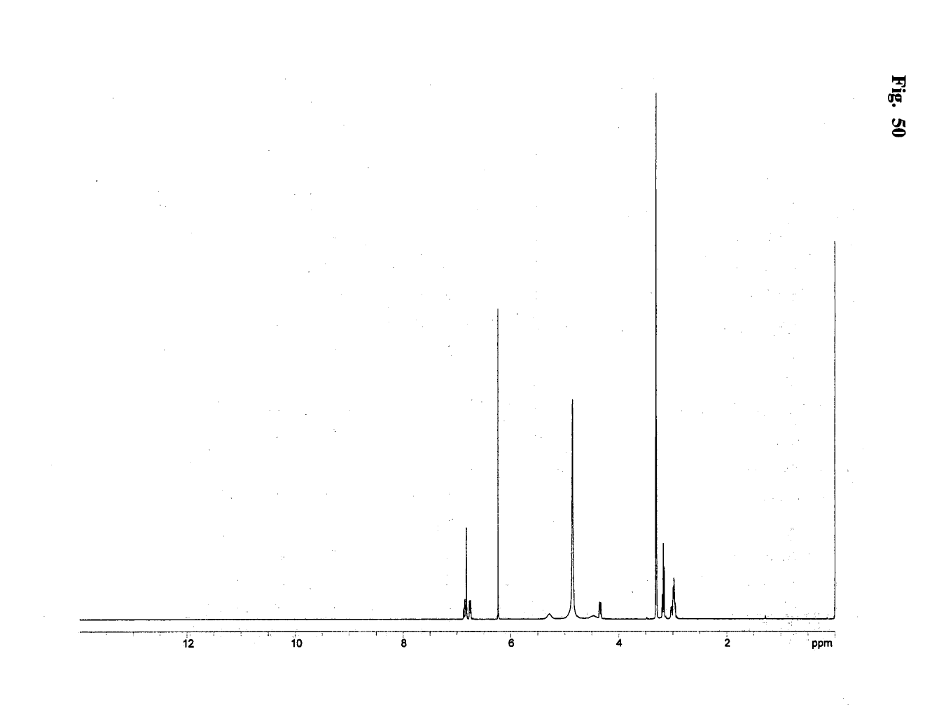

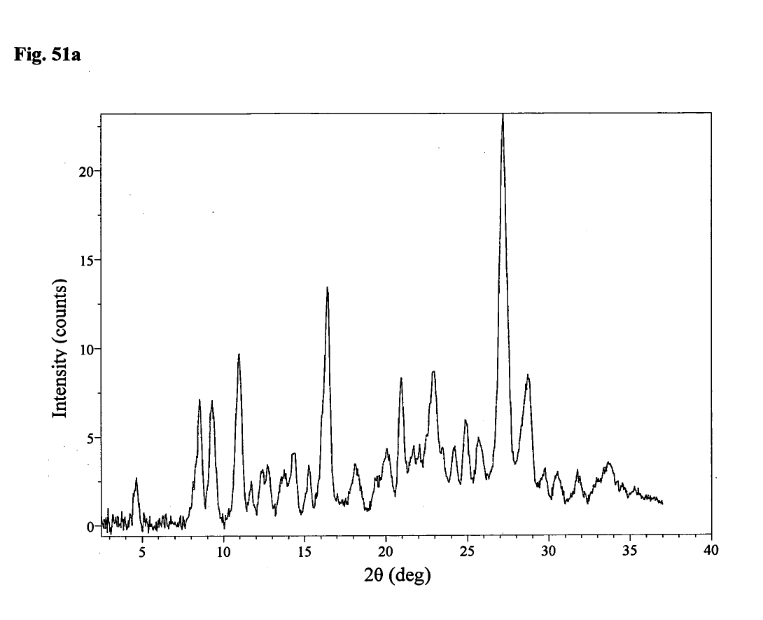

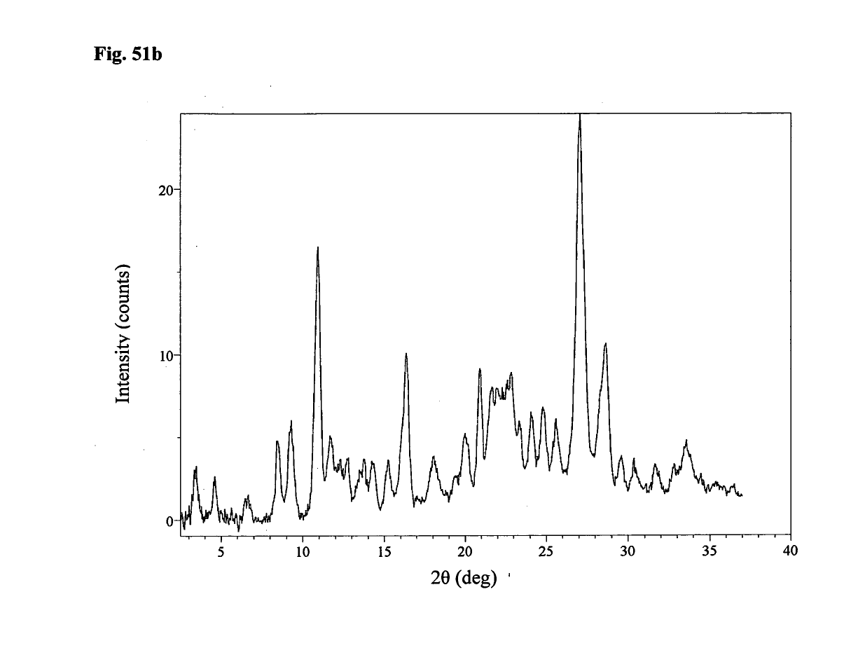

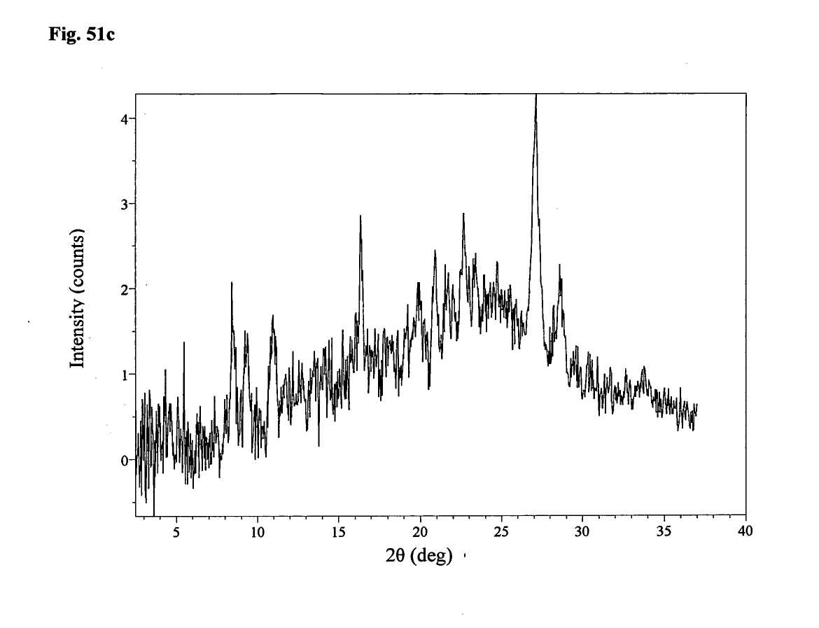

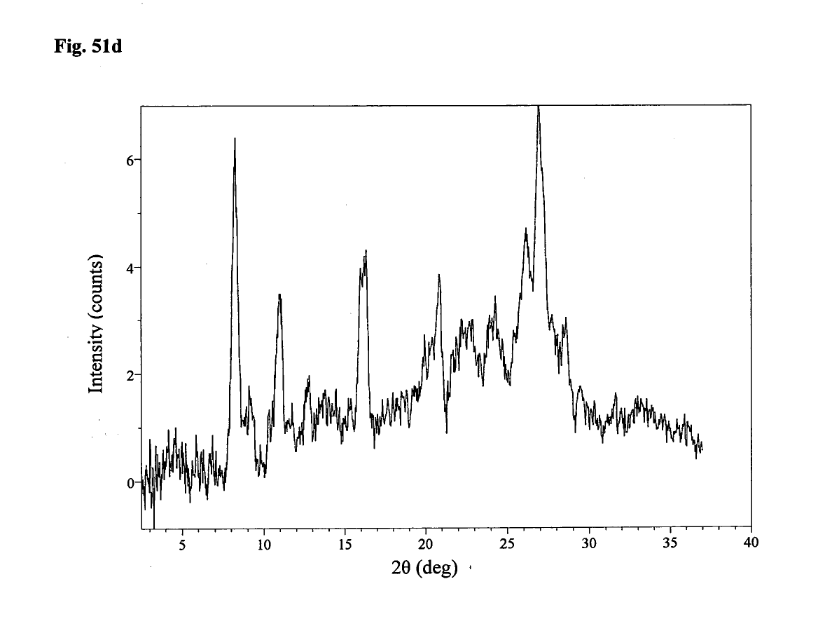

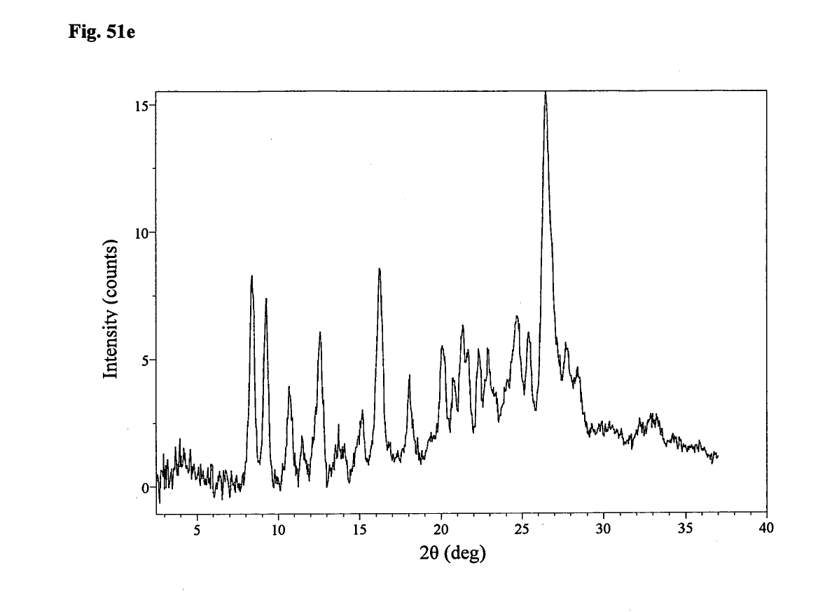

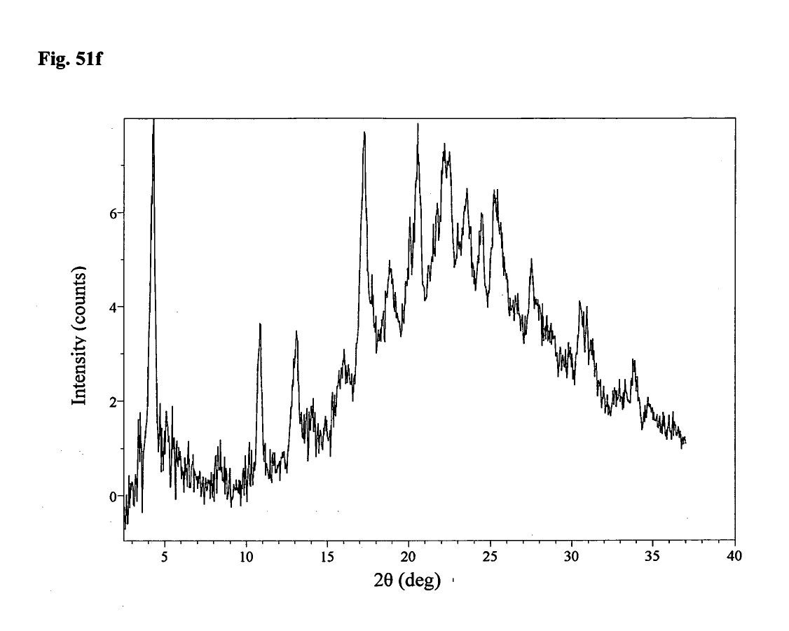

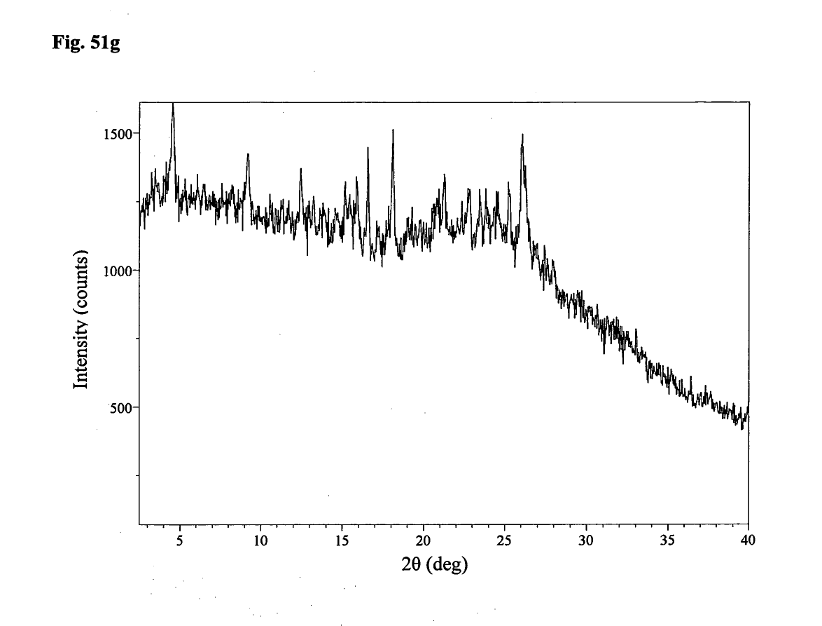

In an embodiment, Form 1 of (R)-5-(2-Aminoethyl)-1-(6,8-difluorochroman-3-yl)-1,3-dihydroimidazole-2-thione phosphate is characterised as having an XRPD pattern as shown in FIG. 51a.

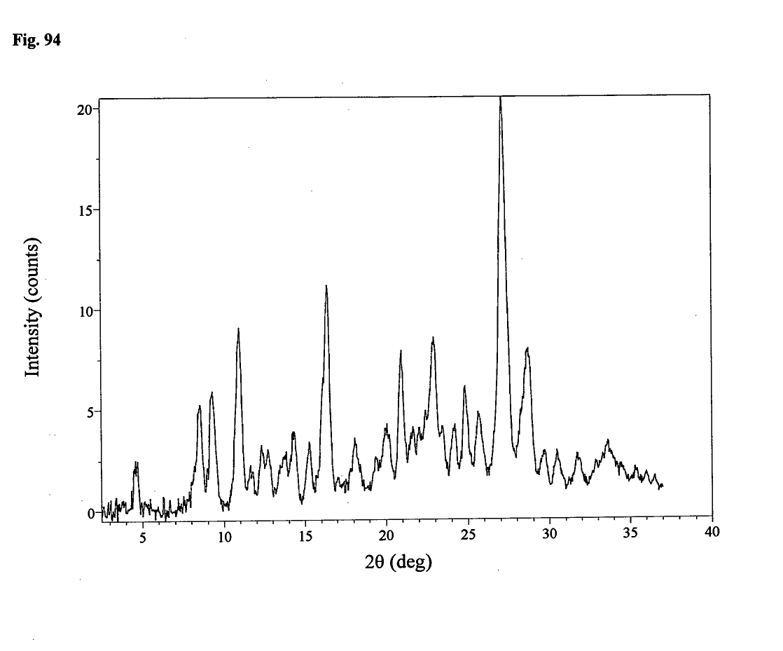

In an embodiment, Form 1 of (R)-5-(2-Aminoethyl)-1-(6,8-difluorochroman-3-yl)-1,3-dihydroimidazole-2-thione phosphate is characterised as having an XRPD pattern as shown in FIG. 94.

In an embodiment, there is provided crystalline Form 2 of (R)-5-(2-Aminoethyl)-1-(6,8-difluorochroman-3-yl)-1,3-dihydroimidazole-2-thione phosphate.

Form 2 may be characterised as having an XRPD pattern with peaks at 4.5, 8.3, 9.0, 10.4, 11.1 and 12.7 °2θ±0.2 °2θ. The XRPD pattern may have further peaks at 16.1 and 17.5 °2θ±0.2 °2θ. The XRPD pattern may have a still further peak at 20.9 °2θ±0.2°θ.

In an embodiment, Form 2 has an XRPD pattern with peaks at the positions listed in Table 65 below.

TABLE 65

| °2θ | d space (Å) | Intensity % (I/Io) |
|---|---|---|
| 4.5 ± 0.1 | 19.724 ± 0.450 | 27 |
| 8.3 ± 0.1 | 10.679 ± 0.130 | 100 |
| 9.0 ± 0.1 | 9.826 ± 0.110 | 25 |
| 10.4 ± 0.1 | 8.539 ± 0.083 | 18 |
| 11.1 ± 0.1 | 7.986 ± 0.073 | 41 |
| 12.7 ± 0.1 | 6.959 ± 0.055 | 28 |
| 16.1 ± 0.1 | 5.512 ± 0.034 | 53 |
| 17.5 ± 0.1 | 5.062 ± 0.029 | 28 |
| 20.9 ± 0.1 | 4.254 ± 0.020 | 49 |

In another embodiment, Form 2 has an XRPD pattern with peaks at the positions listed in Table 66 below.

TABLE 66

| °2θ | d space (Å) | Intensity % (I/Io) |
|---|---|---|
| 4.5 ± 0.1 | 19.724 ± 0.450 | 27 |
| 8.3 ± 0.1 | 10.679 ± 0.130 | 100 |
| 9.0 ± 0.1 | 9.826 ± 0.110 | 25 |
| 10.4 ± 0.1 | 8.539 ± 0.083 | 18 |
| 11.1 ± 0.1 | 7.986 ± 0.073 | 41 |
| 12.7 ± 0.1 | 6.959 ± 0.055 | 28 |
| 13.8 ± 0.1 | 6.436 ± 0.047 | 22 |
| 16.1 ± 0.1 | 5.512 ± 0.034 | 53 |
| 17.5 ± 0.1 | 5.062 ± 0.029 | 28 |
| 18.6 ± 0.1 | 4.771 ± 0.026 | 22 |
| 20.4 ± 0.1 | 4.353 ± 0.021 | 35 |
| 20.9 ± 0.1 | 4.254 ± 0.020 | 49 |
| 21.5 ± 0.1 | 4.129 ± 0.019 | 30 |
| 22.2 ± 0.1 | 3.997 ± 0.018 | 40 |
| 22.8 ± 0.1 | 3.894 ± 0.017 | 35 |
| 24.1 ± 0.1 | 3.696 ± 0.015 | 51 |
| 26.2 ± 0.1 | 3.407 ± 0.013 | 65 |
| 27.0 ± 0.1 | 3.298 ± 0.012 | 65 |
| 27.9 ± 0.1 | 3.196 ± 0.011 | 43 |

In an embodiment, Form 2 of (R)-5-(2-Aminoethyl)-1-(6,8-difluorochroman-3-yl)-1,3-dihydroimidazole-2-thione phosphate is characterised as having an XRPD pattern as shown in FIG. 51d.

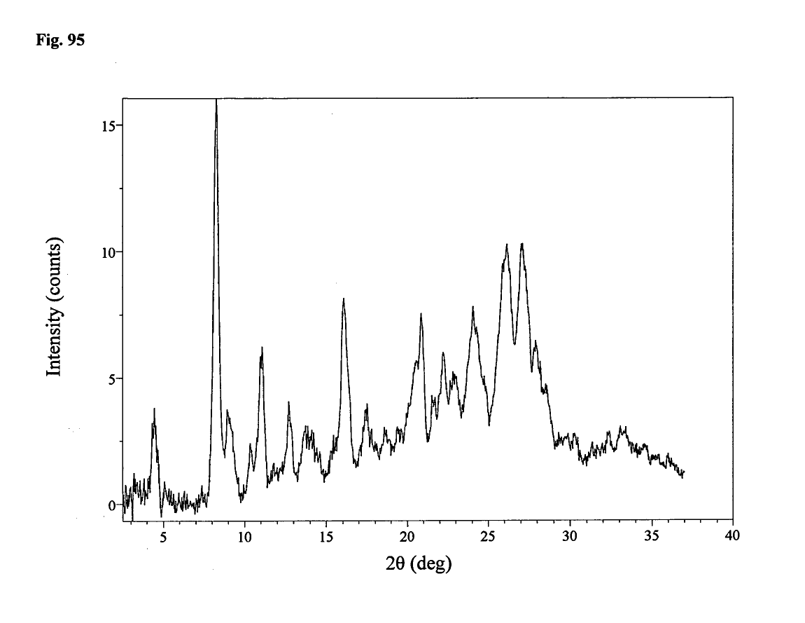

In an embodiment, Form 2 of (R)-5-(2-Aminoethyl)-1-(6,8-difluorochroman-3-yl)-1,3-dihydroimidazole-2-thione phosphate is characterised as having an XRPD pattern as shown in FIG. 95.

In an embodiment, there is provided crystalline Form 3 of (R)-5-(2-Aminoethyl)-1-(6,8-difluorochroman-3-yl)-1,3-dihydroimidazole-2-thione phosphate.

Form 3 may be characterised as having an XRPD pattern with peaks at 8.4, 9.3, 10.7 and 12.6 °2θ±0.2 °2θ. The XRPD pattern may have a further peak at 16.2 °2θ±0.2 °2θ. The XRPD pattern may have a still further peak at 26.5 °2θ±0.2°θ.

In an embodiment, Form 3 has an XRPD pattern with peaks at the positions listed in Table 67 below.

TABLE 67

| °2θ | d space (Å) | Intensity % (I/Io) |
|---|---|---|
| 8.4 ± 0.1 | 10.526 ± 0.127 | 56 |
| 9.3 ± 0.1 | 9.530 ± 0.104 | 51 |
| 10.7 ± 0.1 | 8.253 ± 0.077 | 28 |
| 12.6 ± 0.1 | 7.003 ± 0.056 | 42 |
| 16.2 ± 0.1 | 5.458 ± 0.034 | 58 |
| 26.5 ± 0.1 | 3.366 ± 0.013 | 100 |

In another embodiment, Form 3 has an XRPD pattern with peaks at the positions listed in Table 68 below.

TABLE 68

| °2θ | d space (Å) | Intensity % (I/Io) |
|---|---|---|
| 8.4 ± 0.1 | 10.526 ± 0.127 | 56 |
| 9.3 ± 0.1 | 9.530 ± 0.104 | 51 |
| 10.7 ± 0.1 | 8.253 ± 0.077 | 28 |
| 11.5 ± 0.1 | 7.708 ± 0.068 | 18 |
| 12.6 ± 0.1 | 7.003 ± 0.056 | 42 |
| 13.7 ± 0.1 | 6.454 ± 0.047 | 21 |
| 15.2 ± 0.1 | 5.829 ± 0.038 | 25 |
| 16.2 ± 0.1 | 5.458 ± 0.034 | 58 |
| 18.1 ± 0.1 | 4.907 ± 0.027 | 33 |
| 20.1 ± 0.1 | 4.422 ± 0.022 | 40 |
| 20.8 ± 0.1 | 4.271 ± 0.020 | 31 |
| 21.4 ± 0.1 | 4.160 ± 0.019 | 45 |
| 21.7 ± 0.1 | 4.099 ± 0.019 | 39 |
| 22.3 ± 0.1 | 3.983 ± 0.018 | 39 |
| 22.9 ± 0.1 | 3.880 ± 0.017 | 38 |
| 24.7 ± 0.1 | 3.602 ± 0.014 | 47 |
| 25.4 ± 0.1 | 3.501 ± 0.014 | 43 |
| 26.5 ± 0.1 | 3.366 ± 0.013 | 100 |
| 27.7 ± 0.1 | 3.218 ± 0.011 | 40 |
| 28.4 ± 0.1 | 3.138 ± 0.011 | 35 |

In an embodiment, Form 3 of (R)-5-(2-Aminoethyl)-1-(6,8-difluorochroman-3-yl)-1,3-dihydroimidazole-2-thione phosphate is characterised as having an XRPD pattern as shown in FIG. 51e.

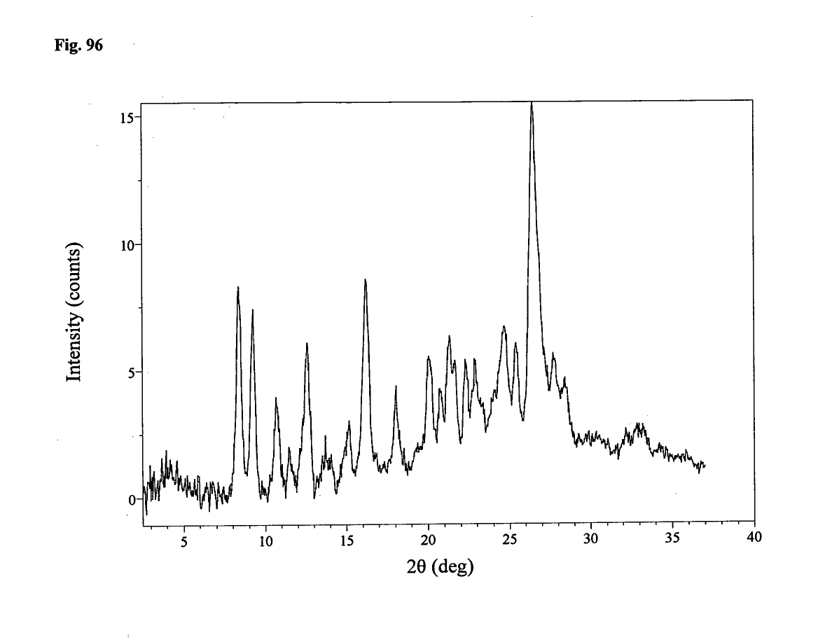

In an embodiment, Form 3 of (R)-5-(2-Aminoethyl)-1-(6,8-difluorochroman-3-yl)-1,3-dihydroimidazole-2-thione phosphate is characterised as having an XRPD pattern as shown in FIG. 96.

In an embodiment, there is provided crystalline Form 4 of (R)-5-(2-Aminoethyl)-1-(6,8-difluorochroman-3-yl)-1,3-dihydroimidazole-2-thione phosphate.

Form 4 may be characterised as having an XRPD pattern with peaks at 4.3, 10.8 and 13.1 °2θ±0.2 °2θ. The XRPD pattern may have further peaks at 17.2 and 20.5 °2θ±0.2°2θ.

In an embodiment, Form 4 has an XRPD pattern with peaks at the positions listed in Table 69 below.

TABLE 69

| °2θ | d space (Å) | Intensity % (I/Io) |
|---|---|---|
| 4.3 ± 0.1 | 20.646 ± 0.494 | 89 |
| 10.8 ± 0.1 | 8.192 ± 0.076 | 53 |
| 13.1 ± 0.1 | 6.769 ± 0.052 | 55 |
| 17.2 ± 0.1 | 5.144 ± 0.030 | 100 |
| 20.5 ± 0.1 | 4.328 ± 0.021 | 89 |

In another embodiment, Form 4 has an XRPD pattern with peaks at the positions listed in Table 70 below.

TABLE 70

| °2θ | d space (Å) | Intensity % (I/Io) |
|---|---|---|
| 4.3 ± 0.1 | 20.646 ± 0.494 | 89 |
| 10.8 ± 0.1 | 8.192 ± 0.076 | 53 |
| 13.1 ± 0.1 | 6.769 ± 0.052 | 55 |
| 15.9 ± 0.1 | 5.567 ± 0.035 | 40 |
| 17.2 ± 0.1 | 5.144 ± 0.030 | 100 |
| 17.7 ± 0.1 | 5.005 ± 0.028 | 52 |
| 18.8 ± 0.1 | 4.720 ± 0.025 | 57 |
| 20.1 ± 0.1 | 4.413 ± 0.022 | 59 |
| 20.5 ± 0.1 | 4.328 ± 0.021 | 89 |
| 21.7 ± 0.1 | 4.092 ± 0.019 | 78 |
| 22.2 ± 0.1 | 4.012 ± 0.018 | 83 |
| 22.4 ± 0.1 | 3.969 ± 0.018 | 83 |
| 23.6 ± 0.1 | 3.770 ± 0.016 | 67 |
| 24.4 ± 0.1 | 3.642 ± 0.015 | 64 |
| 25.4 ± 0.1 | 3.507 ± 0.014 | 71 |
| 27.6 ± 0.1 | 3.232 ± 0.012 | 60 |

In an embodiment, Form 4 of (R)-5-(2-Aminoethyl)-1-(6,8-difluorochroman-3-yl)-1,3-dihydroimidazole-2-thione phosphate is characterised as having an XRPD pattern as shown in FIG. 51f.

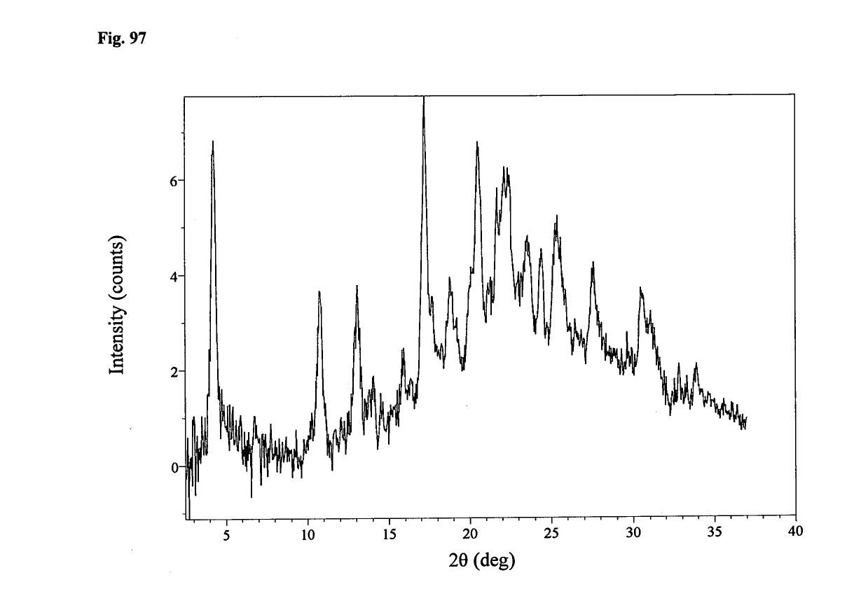

In an embodiment, Form 4 of (R)-5-(2-Aminoethyl)-1-(6,8-difluorochroman-3-yl)-1,3-dihydroimidazole-2-thione phosphate is characterised as having an XRPD pattern as shown in FIG. 97.

In an embodiment, there is provided a crystal modification of (R)-5-(2-Aminoethyl)-1-(6,8-difluorochroman-3-yl)-1,3-dihydroimidazole-2-thione phosphate. This crystal modification is hereinafter referred to as crystal modification X of (R)-5-(2-Aminoethyl)-1-(6,8-difluorochroman-3-yl)-1,3-dihydroimidazole-2-thione phosphate.

Crystal modification X may be characterised as having an XRPD pattern with peaks at 4.6, 9.2, 12.5, 15.2 and 15.9 °2θ±0.2 °2θ. The XRPD pattern may have further peaks at 16.6, 18.1 and 21.3 °2θ±0.2 °2θ. The XRPD pattern may have a still further peak at 26.1 °2θ±0.2°θ.

In an embodiment, crystal modification X has an XRPD pattern with peaks at the positions listed in Table 71 below.

TABLE 71

| °2θ | d space (Å) | Intensity % (I/Io) |
|---|---|---|
| 4.6 ± 0.1 | 19.336 ± 0.432 | 71 |
| 9.2 ± 0.1 | 9.623 ± 0.106 | 53 |
| 12.5 ± 0.1 | 7.104 ± 0.057 | 51 |
| 15.2 ± 0.1 | 5.833 ± 0.038 | 47 |
| 15.9 ± 0.1 | 5.581 ± 0.035 | 55 |
| 16.6 ± 0.1 | 5.350 ± 0.032 | 77 |
| 18.1 ± 0.1 | 4.901 ± 0.027 | 89 |
| 21.3 ± 0.1 | 4.175 ± 0.019 | 56 |
| 26.1 ± 0.1 | 3.417 ± 0.013 | 100 |

In another embodiment, crystal modification X has an XRPD pattern with peaks at the positions listed in Table 72 below.

TABLE 72

| °2θ | d space (Å) | Intensity % (I/Io) |
|---|---|---|
| 4.6 ± 0.1 | 19.336 ± 0.432 | 71 |
| 9.2 ± 0.1 | 9.623 ± 0.106 | 53 |
| 12.5 ± 0.1 | 7.104 ± 0.057 | 51 |
| 15.2 ± 0.1 | 5.833 ± 0.038 | 47 |
| 15.9 ± 0.1 | 5.581 ± 0.035 | 55 |
| 16.6 ± 0.1 | 5.350 ± 0.032 | 77 |
| 18.1 ± 0.1 | 4.901 ± 0.027 | 89 |
| 20.8 ± 0.1 | 4.265 ± 0.020 | 39 |
| 21.3 ± 0.1 | 4.175 ± 0.019 | 56 |
| 22.8 ± 0.1 | 3.894 ± 0.017 | 47 |
| 23.5 ± 0.1 | 3.791 ± 0.016 | 46 |
| 23.8 ± 0.1 | 3.734 ± 0.016 | 47 |
| 24.6 ± 0.1 | 3.622 ± 0.015 | 51 |
| 25.2 ± 0.1 | 3.529 ± 0.014 | 59 |
| 26.1 ± 0.1 | 3.417 ± 0.013 | 100 |
| 26.3 ± 0.1 | 3.394 ± 0.013 | 79 |

In an embodiment, crystal modification X of (R)-5-(2-Aminoethyl)-1-(6,8-difluorochroman-3-yl)-1,3-dihydroimidazole-2-thione phosphate is characterised as having an XRPD pattern as shown in FIG. 51g.

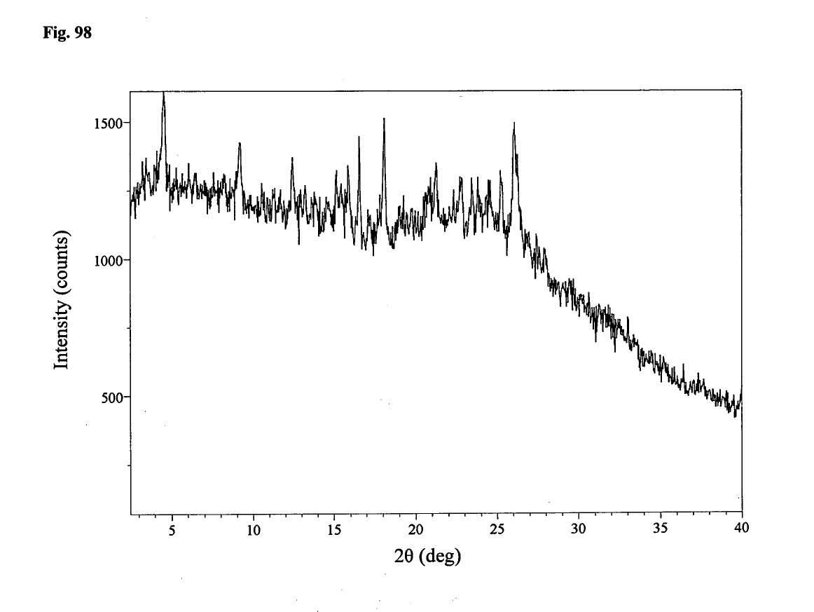

In another embodiment, crystal modification X of (R)-5-(2-Aminoethyl)-1-(6,8-difluorochroman-3-yl)-1,3-dihydroimidazole-2-thione phosphate is characterised as having an XRPD pattern as shown in FIG. 98.

In an embodiment, there is provided crystalline Form 6 of (R)-5-(2-Aminoethyl)-1-(6,8-difluorochroman-3-yl)-1,3-dihydroimidazole-2-thione phosphate.

Form 6 may be characterised as having an XRPD pattern with a peak at 6.6 °2θ±0.2 °2θ. The XRPD pattern may have a further peak at 3.3 °2θ±0.2 °2θ. The XRPD pattern may have further peaks at 11.8, 12.1 and 13.2 °2θ±0.2°2θ. The XRPD pattern may have still further peaks at 17.8, 20.1 and 22.2 °2θ±0.2°θ.

In an embodiment, Form 6 has an XRPD pattern with peaks at the positions listed in Table 73 below.

TABLE 73

| °2θ | d space (Å) | Intensity % (I/Io) |
|---|---|---|
| 6.6 ± 0.1 | 13.433 ± 0.207 | 46 |

In another embodiment, Form 6 has an XRPD pattern with peaks at the positions listed in Table 74 below.

TABLE 74

| °2θ | d space (Å) | Intensity % (I/Io) |
|---|---|---|
| 3.3 ± 0.1 | 26.454 ± 0.816 | 100 |
| 6.6 ± 0.1 | 13.433 ± 0.207 | 46 |

In yet another embodiment, Form 6 has an XRPD pattern with peaks at the positions listed in Table 75 below.

TABLE 75

| °2θ | d space (Å) | Intensity % (I/Io) |
|---|---|---|
| 3.3 ± 0.1 | 26.454 ± 0.816 | 100 |
| 6.6 ± 0.1 | 13.433 ± 0.207 | 46 |
| 11.8 ± 0.1 | 7.481 ± 0.064 | 55 |
| 12.1 ± 0.1 | 7.315 ± 0.061 | 30 |
| 13.2 ± 0.1 | 6.718 ± 0.051 | 25 |
| 17.8 ± 0.1 | 4.983 ± 0.028 | 21 |
| 20.1 ± 0.1 | 4.422 ± 0.022 | 25 |
| 22.2 ± 0.1 | 4.013 ± 0.018 | 34 |

In yet another embodiment, Form 6 has an XRPD pattern with peaks at the positions listed in Table 76 below.

TABLE 76

| °2θ | d space (Å) | Intensity % (I/Io) |
|---|---|---|
| 3.3 ± 0.1 | 26.454 ± 0.816 | 100 |
| 6.6 ± 0.1 | 13.433 ± 0.207 | 46 |
| 8.8 ± 0.1 | 10.015 ± 0.114 | 11 |
| 11.3 ± 0.1 | 7.817 ± 0.069 | 14 |
| 11.8 ± 0.1 | 7.481 ± 0.064 | 55 |
| 12.1 ± 0.1 | 7.315 ± 0.061 | 30 |
| 12.5 ± 0.1 | 7.087 ± 0.057 | 8 |
| 13.2 ± 0.1 | 6.718 ± 0.051 | 25 |
| 14.6 ± 0.1 | 6.084 ± 0.042 | 5 |
| 15.2 ± 0.1 | 5.844 ± 0.039 | 11 |
| 15.3 ± 0.1 | 5.776 ± 0.038 | 10 |
| 15.6 ± 0.1 | 5.699 ± 0.037 | 16 |
| 16.0 ± 0.1 | 5.529 ± 0.034 | 6 |
| 16.5 ± 0.1 | 5.379 ± 0.033 | 10 |
| 17.3 ± 0.1 | 5.129 ± 0.030 | 6 |
| 17.8 ± 0.1 | 4.983 ± 0.028 | 21 |
| 18.3 ± 0.1 | 4.853 ± 0.026 | 8 |
| 18.8 ± 0.1 | 4.715 ± 0.025 | 15 |
| 20.1 ± 0.1 | 4.422 ± 0.022 | 25 |
| 20.8 ± 0.1 | 4.271 ± 0.020 | 16 |
| 21.3 ± 0.1 | 4.175 ± 0.019 | 15 |
| 21.6 ± 0.1 | 4.118 ± 0.019 | 13 |
| 22.2 ± 0.1 | 4.013 ± 0.018 | 34 |
| 22.7 ± 0.1 | 3.919 ± 0.017 | 8 |
| 23.8 ± 0.1 | 3.743 ± 0.016 | 15 |
| 24.2 ± 0.1 | 3.679 ± 0.015 | 10 |
| 24.6 ± 0.1 | 3.626 ± 0.015 | 9 |
| 25.0 ± 0.1 | 3.562 ± 0.014 | 21 |
| 25.8 ± 0.1 | 3.460 ± 0.013 | 11 |
| 26.7 ± 0.1 | 3.338 ± 0.012 | 25 |
| 27.5 ± 0.1 | 3.248 ± 0.012 | 15 |
| 28.4 ± 0.1 | 3.144 ± 0.011 | 14 |
| 29.5 ± 0.1 | 3.025 ± 0.010 | 7 |

In an embodiment, Form 6 of (R)-5-(2-Aminoethyl)-1-(6,8-difluorochroman-3-yl)-1,3-dihydroimidazole-2-thione phosphate is characterised as having an XRPD pattern as shown in FIG. 51h.

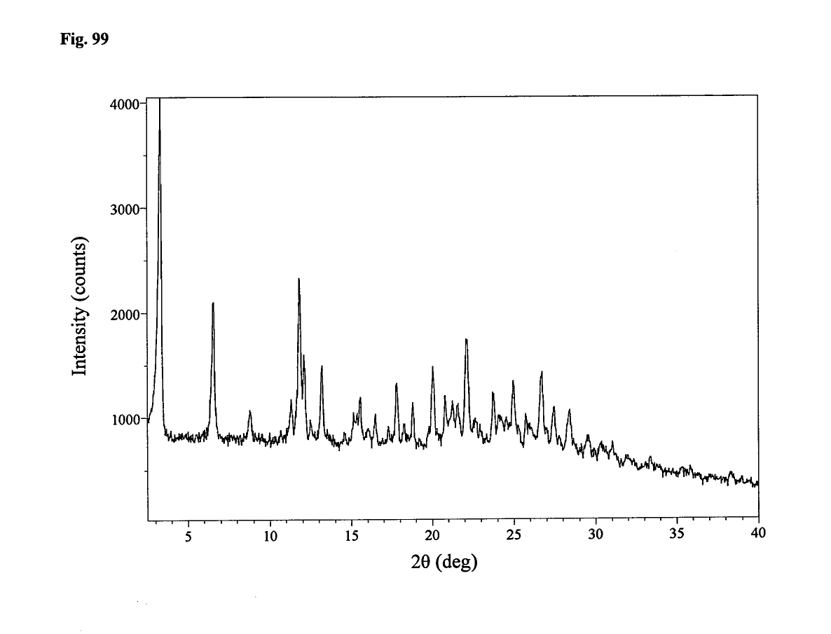

In an embodiment, Form 6 of (R)-5-(2-Aminoethyl)-1-(6,8-difluorochroman-3-yl)-1,3-dihydroimidazole-2-thione phosphate is characterised as having an XRPD pattern as shown in FIG. 99.

In an embodiment, there is provided crystalline Form 7 of (R)-5-(2-Aminoethyl)-1-(6,8-difluorochroman-3-yl)-1,3-dihydroimidazole-2-thione phosphate.

Form 7 may be characterised as having an XRPD pattern with peaks at 4.1 and 6.0 °2θ±0.2 °2θ. The XRPD pattern may have a further peak at 11.8 °2θ±0.2 °2θ. The XRPD pattern may have still further peaks at 16.6, 21.2 and 23.5 °2θ±0.2°θ.

In an embodiment, Form 7 has an XRPD pattern with peaks at the positions listed in Table 77 below.

TABLE 77

| °2θ | d space (Å) | Intensity % (I/Io) |
|---|---|---|
| 4.1 ± 0.1 | 21.604 ± 0.541 | 100 |
| 6.0 ± 0.1 | 14.633 ± 0.246 | 46 |

In another embodiment, Form 7 has an XRPD pattern with peaks at the positions listed in Table 78 below.

TABLE 78

| °2θ | d space (Å) | Intensity % (I/Io) |
|---|---|---|
| 4.1 ± 0.1 | 21.604 ± 0.541 | 100 |
| 6.0 ± 0.1 | 14.633 ± 0.246 | 46 |
| 11.8 ± 0.1 | 7.519 ± 0.064 | 97 |
| 16.6 ± 0.1 | 5.341 ± 0.032 | 76 |
| 21.2 ± 0.1 | 4.199 ± 0.020 | 77 |
| 23.5 ± 0.1 | 3.786 ± 0.016 | 80 |

In yet another embodiment, Form 7 has an XRPD pattern with peaks at the positions listed in Table 79 below.

TABLE 79

| °2θ | d space (Å) | Intensity % (I/Io) |
|---|---|---|
| 4.1 ± 0.1 | 21.604 ± 0.541 | 100 |
| 6.0 ± 0.1 | 14.633 ± 0.246 | 46 |
| 8.4 ± 0.1 | 10.477 ± 0.125 | 37 |
| 11.8 ± 0.1 | 7.519 ± 0.064 | 97 |
| 15.5 ± 0.1 | 5.732 ± 0.037 | 41 |
| 16.6 ± 0.1 | 5.341 ± 0.032 | 76 |
| 17.5 ± 0.1 | 5.068 ± 0.029 | 46 |
| 20.4 ± 0.1 | 4.351 ± 0.021 | 63 |
| 21.2 ± 0.1 | 4.199 ± 0.020 | 77 |
| 22.6 ± 0.1 | 3.940 ± 0.017 | 58 |
| 23.5 ± 0.1 | 3.786 ± 0.016 | 80 |
| 24.8 ± 0.1 | 3.592 ± 0.014 | 54 |
| 27.1 ± 0.1 | 3.290 ± 0.012 | 51 |

In an embodiment, Form 7 of (R)-5-(2-Aminoethyl)-1-(6,8-difluorochroman-3-yl)-1,3-dihydroimidazole-2-thione phosphate is characterised as having an XRPD pattern as shown in FIG. 51i.

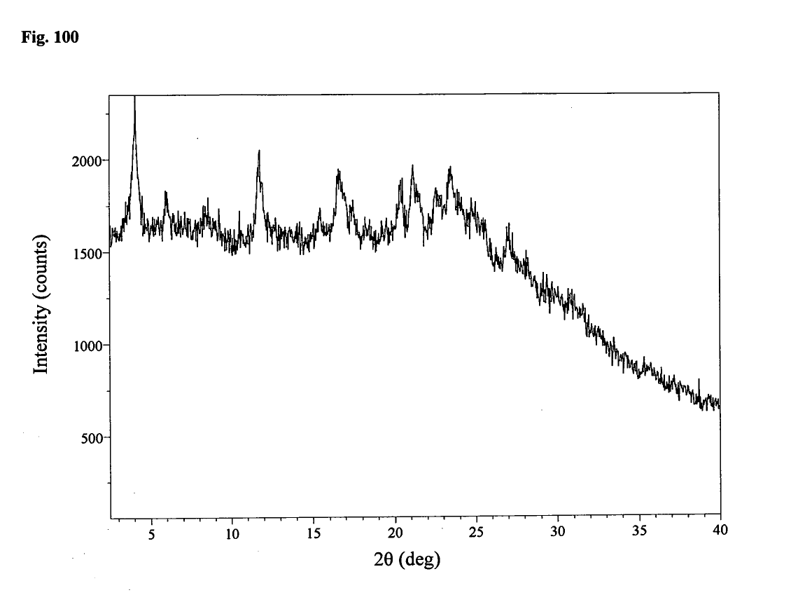

In an embodiment, Form 7 of (R)-5-(2-Aminoethyl)-1-(6,8-difluorochroman-3-yl)-1,3-dihydroimidazole-2-thione phosphate is characterised as having an XRPD pattern as shown in FIG. 100.

In an embodiment, there is provided crystalline Form 8 of (R)-5-(2-Aminoethyl)-1-(6,8-difluorochroman-3-yl)-1,3-dihydroimidazole-2-thione phosphate.

Form 8 may be characterised as having an XRPD pattern with peaks at 11.7, 12.2, 15.2 and 16.6 °2θ±0.2°2θ. The XRPD pattern may have a further peak at 18.1 °2θ±0.2 °2θ. The XRPD pattern may have still further peaks at 22.8 and 26.1 °2θ±0.2°θ.

In an embodiment, Form 8 has an XRPD pattern with peaks at the positions listed in Table 80 below.

TABLE 80

| °2θ | d space (Å) | Intensity % (I/Io) |
|---|---|---|
| 11.7 ± 0.1 | 7.557 ± 0.065 | 21 |
| 12.2 ± 0.1 | 7.225 ± 0.059 | 14 |
| 15.2 ± 0.1 | 5.833 ± 0.038 | 30 |
| 16.6 ± 0.1 | 5.341 ± 0.032 | 80 |
| 18.1 ± 0.1 | 4.901 ± 0.027 | 100 |
| 22.8 ± 0.1 | 3.899 ± 0.017 | 41 |
| 26.1 ± 0.1 | 3.417 ± 0.013 | 61 |

In another embodiment, Form 8 has an XRPD pattern with peaks at the positions listed in Table 81 below.

TABLE 81

| °2θ | d space (Å) | Intensity % (I/Io) |
|---|---|---|
| 6.4 ± 0.1 | 13.746 ± 0.217 | 9 |
| 11.7 ± 0.1 | 7.557 ± 0.065 | 21 |
| 12.2 ± 0.1 | 7.225 ± 0.059 | 14 |
| 15.2 ± 0.1 | 5.833 ± 0.038 | 30 |
| 16.6 ± 0.1 | 5.341 ± 0.032 | 80 |
| 18.1 ± 0.1 | 4.901 ± 0.027 | 100 |
| 19.0 ± 0.1 | 4.678 ± 0.025 | 11 |
| 19.3 ± 0.1 | 4.599 ± 0.024 | 14 |
| 19.8 ± 0.1 | 4.489 ± 0.023 | 23 |
| 20.6 ± 0.1 | 4.320 ± 0.021 | 9 |
| 20.8 ± 0.1 | 4.271 ± 0.020 | 8 |
| 21.3 ± 0.1 | 4.175 ± 0.019 | 28 |
| 21.7 ± 0.1 | 4.096 ± 0.019 | 22 |
| 22.4 ± 0.1 | 3.966 ± 0.018 | 7 |
| 22.8 ± 0.1 | 3.899 ± 0.017 | 41 |
| 23.5 ± 0.1 | 3.786 ± 0.016 | 25 |
| 23.9 ± 0.1 | 3.729 ± 0.015 | 38 |
| 24.6 ± 0.1 | 3.626 ± 0.015 | 28 |
| 24.9 ± 0.1 | 3.570 ± 0.014 | 9 |
| 25.3 ± 0.1 | 3.520 ± 0.014 | 33 |
| 26.1 ± 0.1 | 3.417 ± 0.013 | 61 |
| 26.5 ± 0.1 | 3.364 ± 0.013 | 21 |
| 27.6 ± 0.1 | 3.234 ± 0.012 | 13 |
| 28.0 ± 0.1 | 3.190 ± 0.011 | 17 |
| 29.2 ± 0.1 | 3.062 ± 0.010 | 7 |

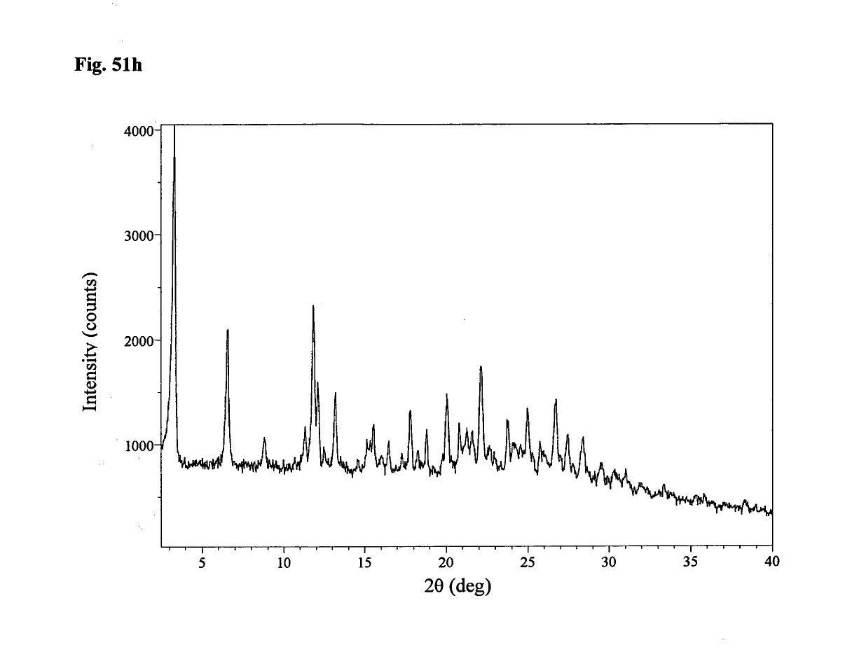

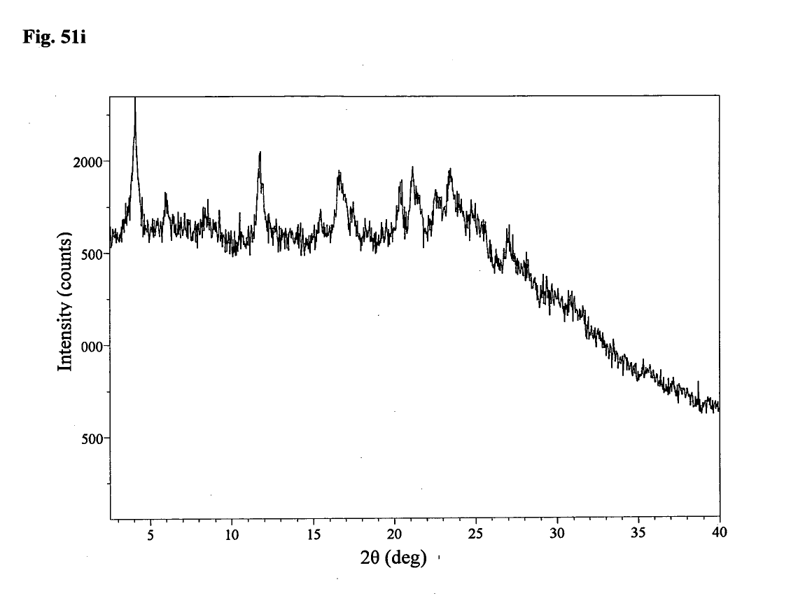

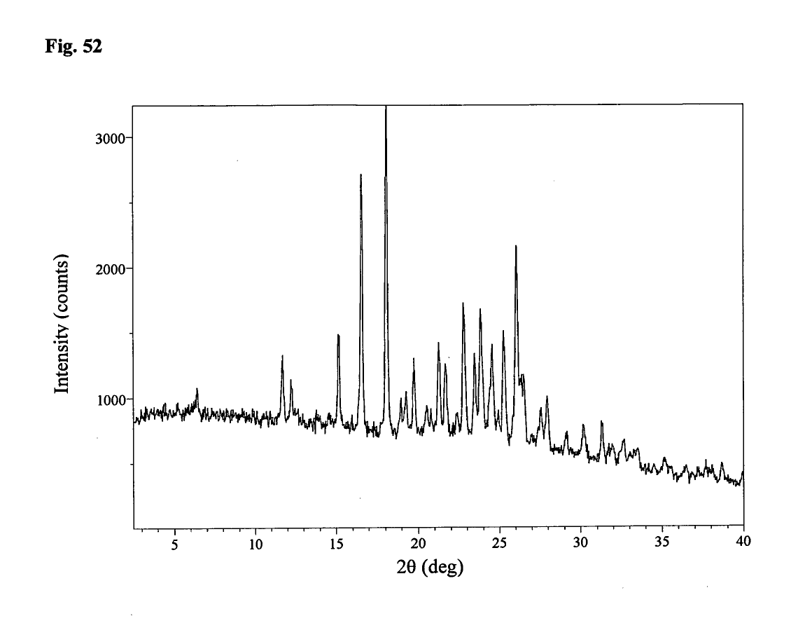

In an embodiment, Form 8 of (R)-5-(2-Aminoethyl)-1-(6,8-difluorochroman-3-yl)-1,3-dihydroimidazole-2-thione phosphate is characterised as having an XRPD pattern as shown in FIG. 52.

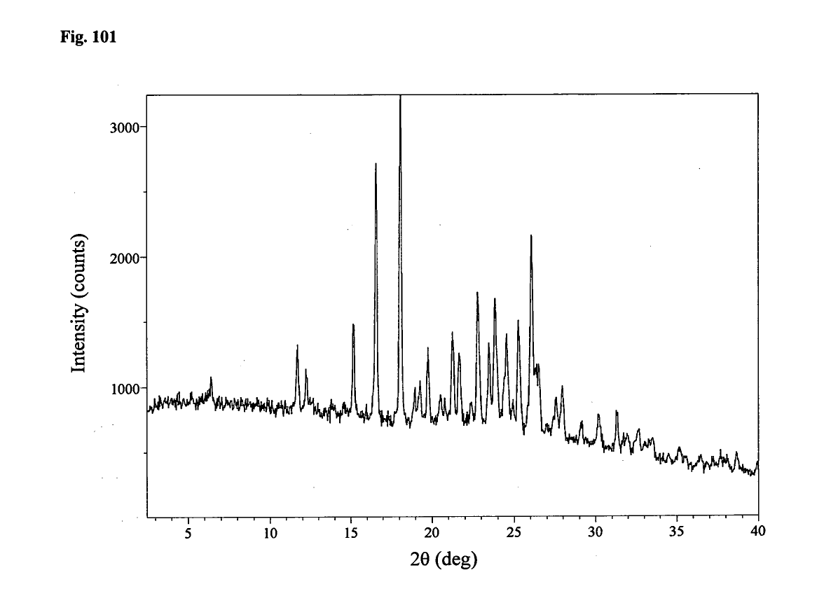

In an embodiment, Form 8 of (R)-5-(2-Aminoethyl)-1-(6,8-difluorochroman-3-yl)-1,3-dihydroimidazole-2-thione phosphate is characterised as having an XRPD pattern as shown in FIG. 101.

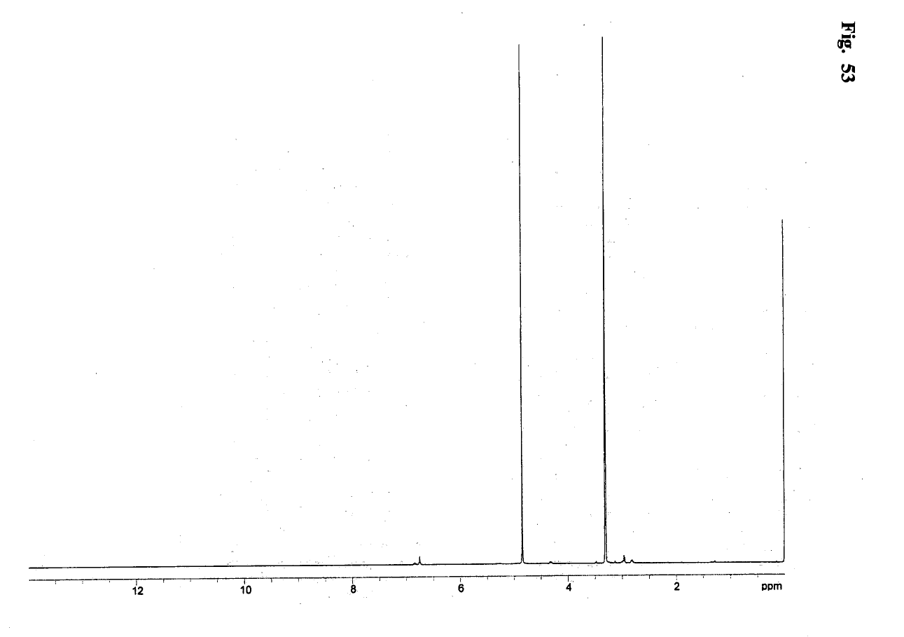

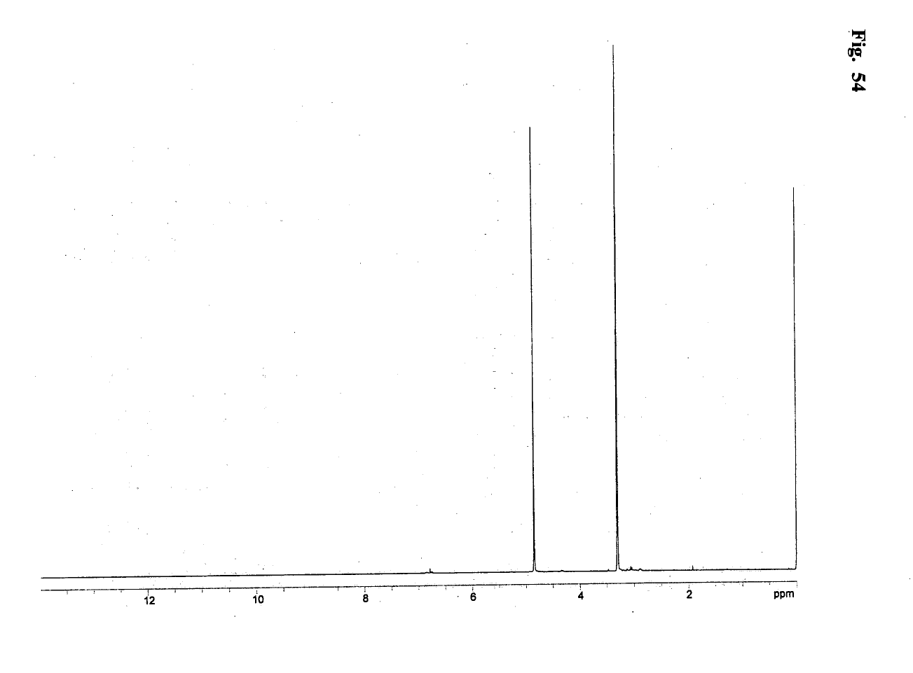

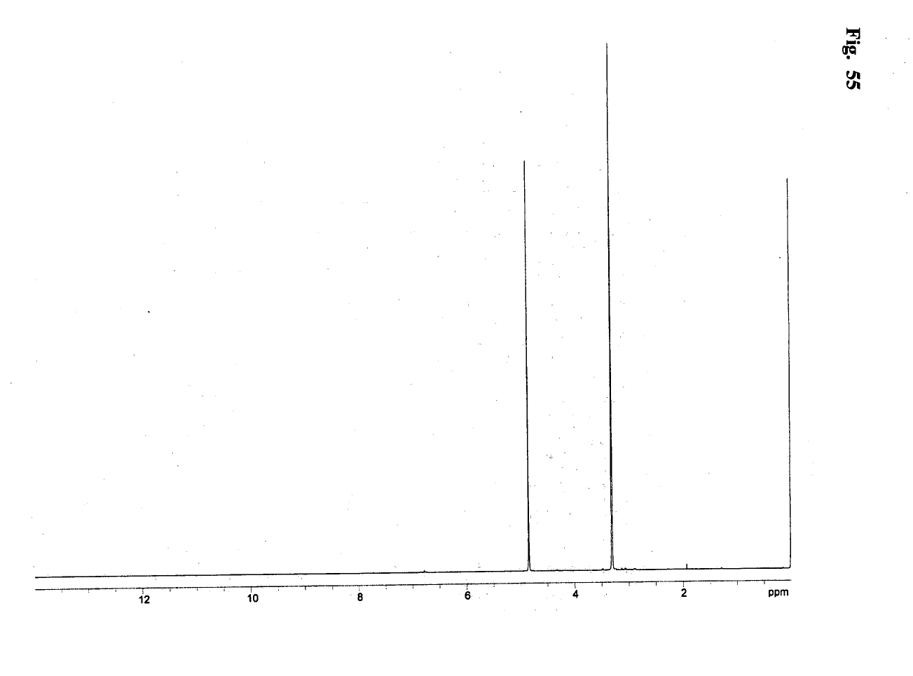

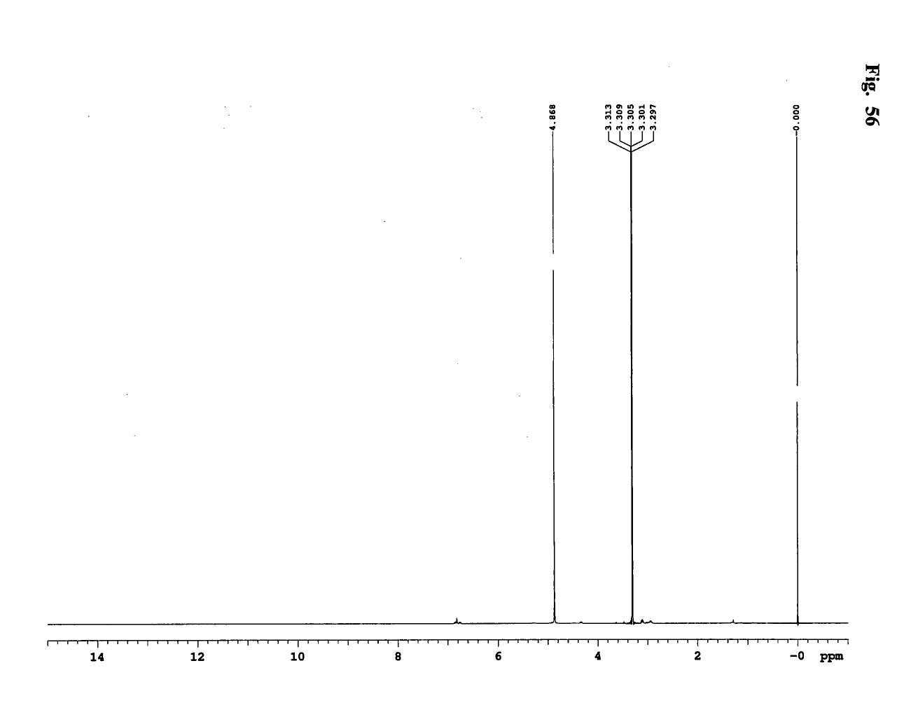

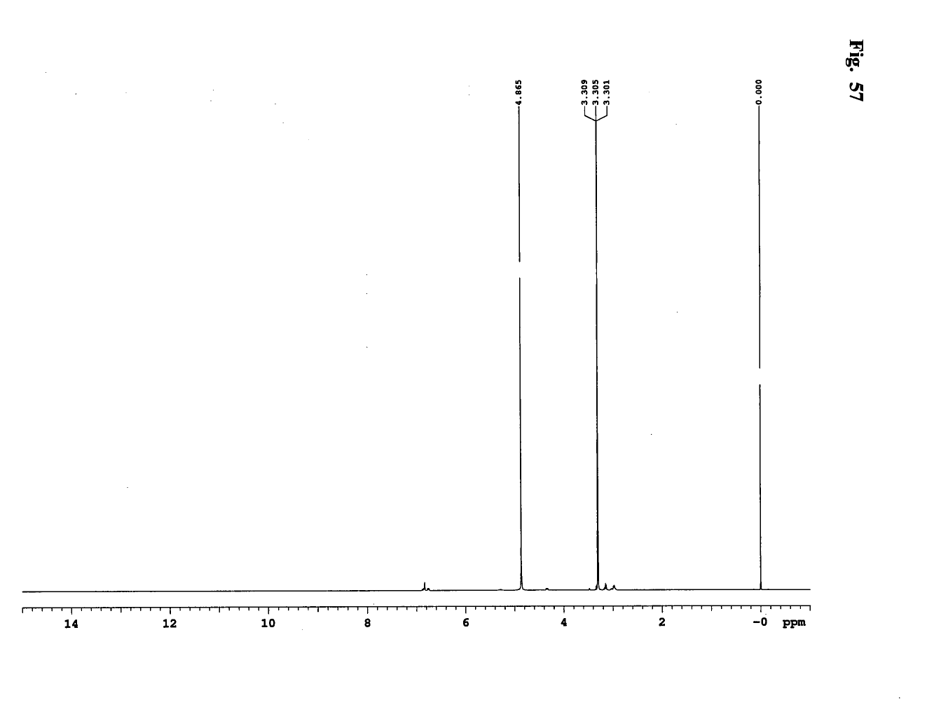

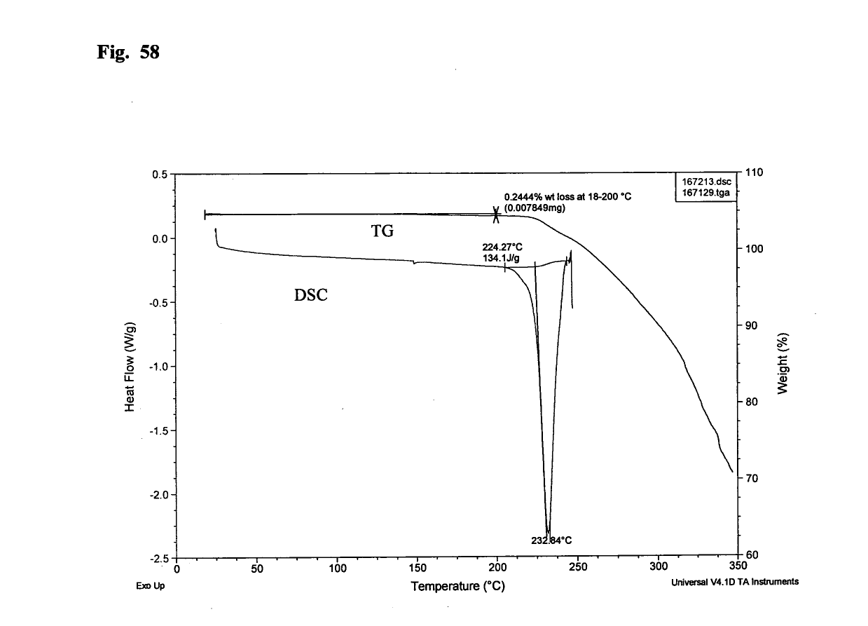

Form 8 of (R)-5-(2-Aminoethyl)-1-(6,8-difluorochroman-3-yl)-1,3-dihydroimidazole-2-thione phosphate may also be characterised by having a DSC thermogram as shown in FIG. 58.

According to another aspect of the present invention, there is provided the gentisic acid salt of (R)-5-(2-Aminoethyl)-1-(6,8-difluorochroman-3-yl)-1,3-dihydroimidazole-2-thione, i.e. (R)-5-(2-Aminoethyl)-1-(6,8-difluorochroman-3-yl)-1,3-dihydroimidazole-2-thione gentisate.

In an embodiment, there is provided crystalline Form 1 of (R)-5-(2-Aminoethyl)-1-(6,8-difluorochroman-3-yl)-1,3-dihydroimidazole-2-thione gentisate.

Form 1 may be characterised as having an XRPD pattern with peaks at 18.2 and 18.6 °2θ±0.2 °2θ. The XRPD pattern may have further peaks at 12.9 and 14.0 °2θ±0.2 °2θ. The XRPD pattern may have still further peaks at 17.1 and 21.6 °2θ±0.2°θ. The XRPD pattern may have yet further peaks at 24.8 and 25.7 °2θ±0.2°θ.

In an embodiment, Form 1 has an XRPD pattern with peaks at the positions listed in Table 82 below.

TABLE 82

| °2θ | d space (Å) | Intensity % (I/Io) |
|---|---|---|
| 18.2 ± 0.1 | 4.877 ± 0.027 | 85 |
| 18.6 ± 0.1 | 4.760 ± 0.025 | 93 |

In another embodiment, Form 1 has an XRPD pattern with peaks at the positions listed in Table 83 below.

TABLE 83

| °2θ | d space (Å) | Intensity % (I/Io) |
|---|---|---|
| 12.9 ± 0.1 | 6.842 ± 0.053 | 23 |
| 14.0 ± 0.1 | 6.317 ± 0.045 | 19 |
| 17.1 ± 0.1 | 5.192 ± 0.030 | 99 |
| 18.2 ± 0.1 | 4.877 ± 0.027 | 85 |
| 18.6 ± 0.1 | 4.760 ± 0.025 | 93 |
| 21.6 ± 0.1 | 4.118 ± 0.019 | 53 |
| 22.2 ± 0.1 | 4.008 ± 0.018 | 49 |
| 22.5 ± 0.1 | 3.945 ± 0.017 | 45 |
| 24.8 ± 0.1 | 3.583 ± 0.014 | 85 |
| 25.7 ± 0.1 | 3.468 ± 0.013 | 100 |

In yet another embodiment, Form 1 has an XRPD pattern with peaks at the positions listed in Table 84 below.

TABLE 84

| °2θ | d space (Å) | Intensity % (I/Io) |
|---|---|---|
| 4.8 ± 0.1 | 18.372 ± 0.390 | 12 |
| 9.8 ± 0.1 | 9.035 ± 0.093 | 12 |
| 12.9 ± 0.1 | 6.842 ± 0.053 | 23 |
| 13.4 ± 0.1 | 6.613 ± 0.050 | 11 |
| 14.0 ± 0.1 | 6.317 ± 0.045 | 19 |
| 14.6 ± 0.1 | 6.059 ± 0.042 | 31 |
| 15.2 ± 0.1 | 5.810 ± 0.038 | 20 |
| 16.7 ± 0.1 | 5.312 ± 0.032 | 10 |
| 17.1 ± 0.1 | 5.192 ± 0.030 | 99 |
| 18.2 ± 0.1 | 4.877 ± 0.027 | 85 |
| 18.6 ± 0.1 | 4.760 ± 0.025 | 93 |
| 20.2 ± 0.1 | 4.390 ± 0.022 | 11 |
| 20.7 ± 0.1 | 4.295 ± 0.021 | 21 |
| 21.6 ± 0.1 | 4.118 ± 0.019 | 53 |
| 22.2 ± 0.1 | 4.008 ± 0.018 | 49 |
| 22.5 ± 0.1 | 3.945 ± 0.017 | 45 |
| 23.6 ± 0.1 | 3.762 ± 0.016 | 22 |
| 23.9 ± 0.1 | 3.729 ± 0.015 | 17 |
| 24.8 ± 0.1 | 3.583 ± 0.014 | 85 |
| 25.7 ± 0.1 | 3.468 ± 0.013 | 100 |
| 26.0 ± 0.1 | 3.428 ± 0.013 | 51 |
| 26.4 ± 0.1 | 3.371 ± 0.013 | 29 |
| 26.8 ± 0.1 | 3.327 ± 0.012 | 30 |
| 28.2 ± 0.1 | 3.170 ± 0.011 | 52 |

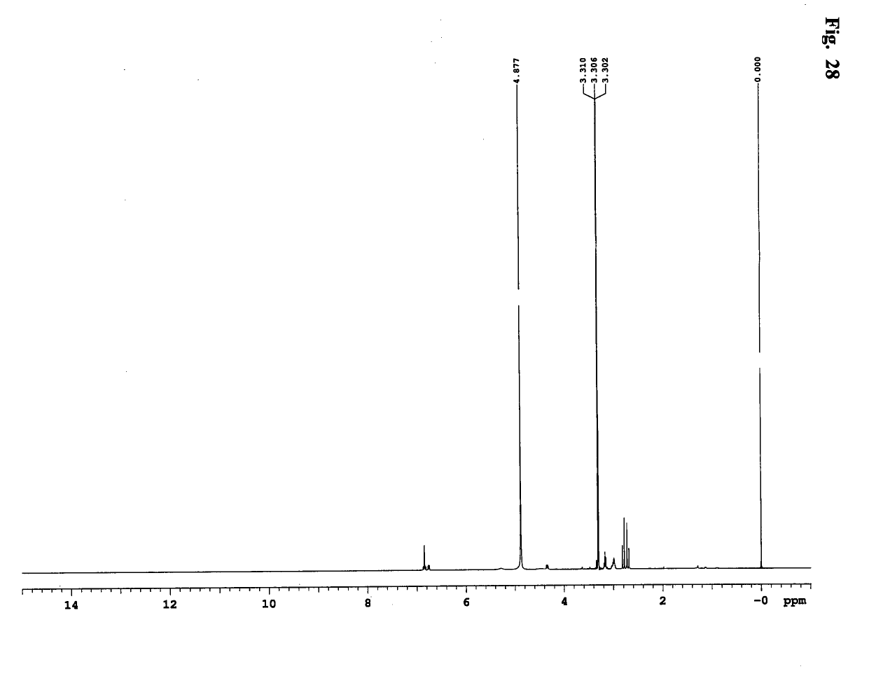

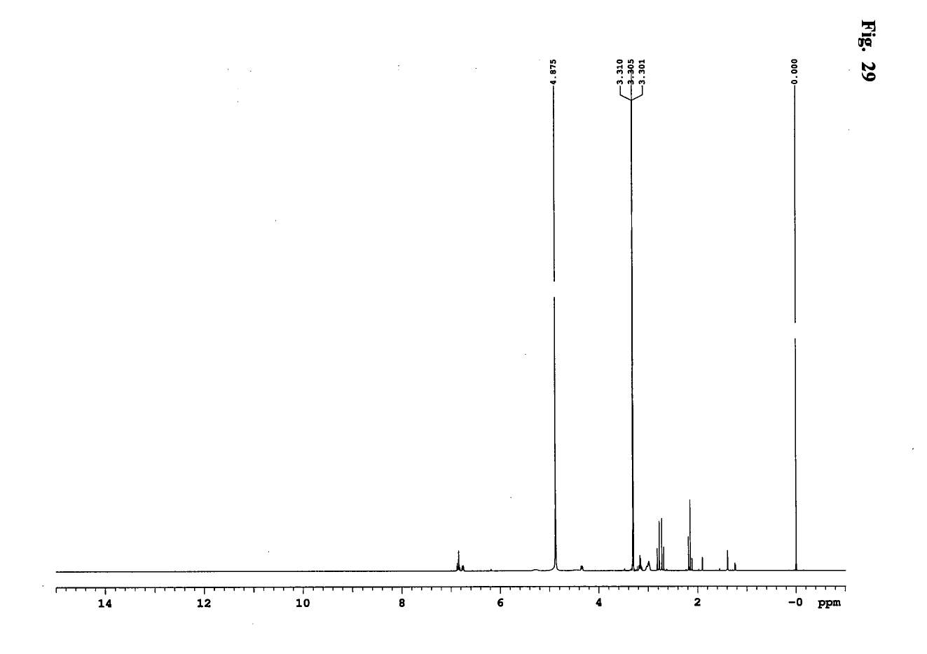

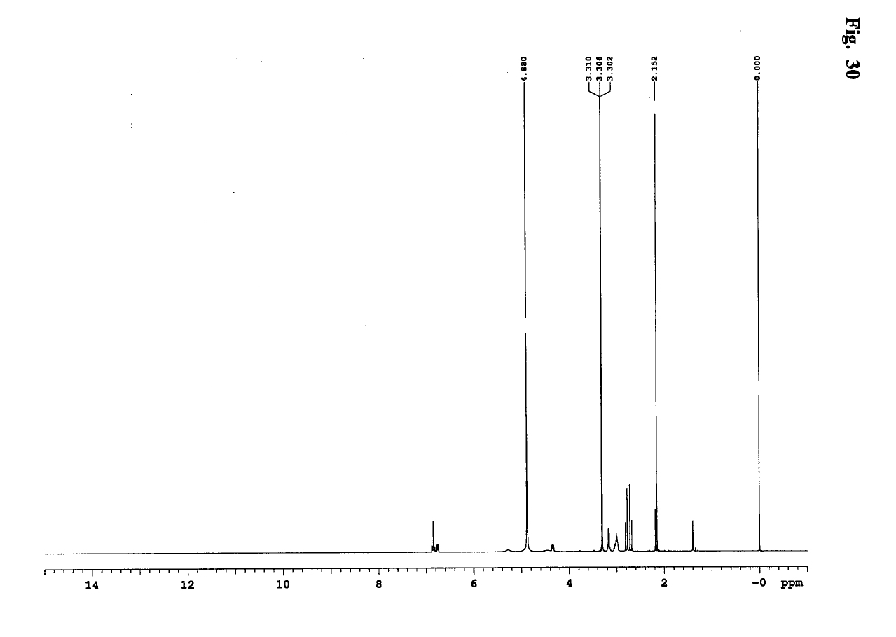

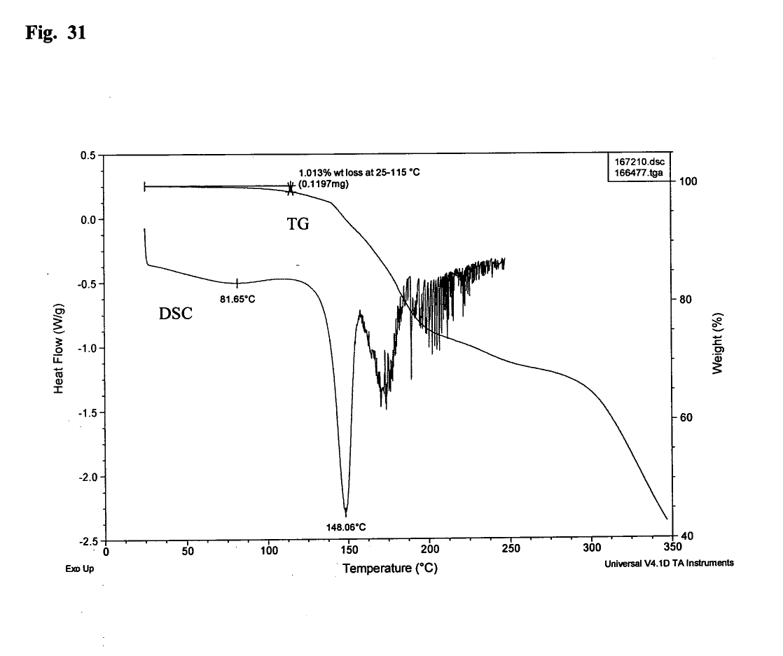

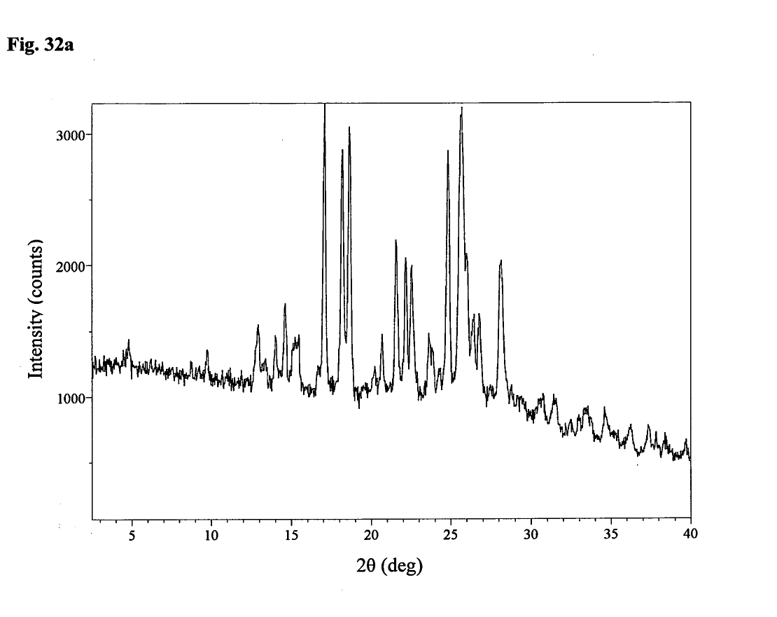

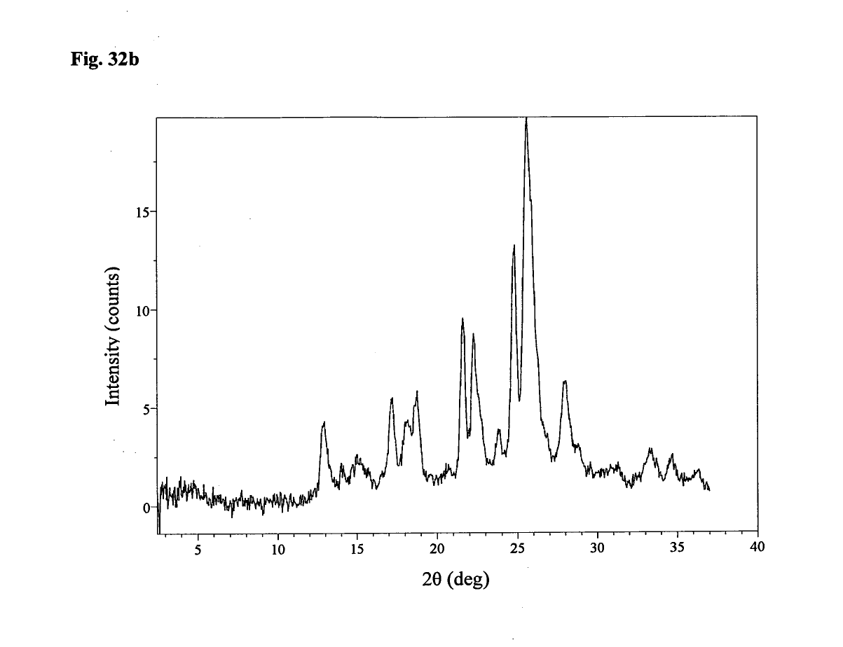

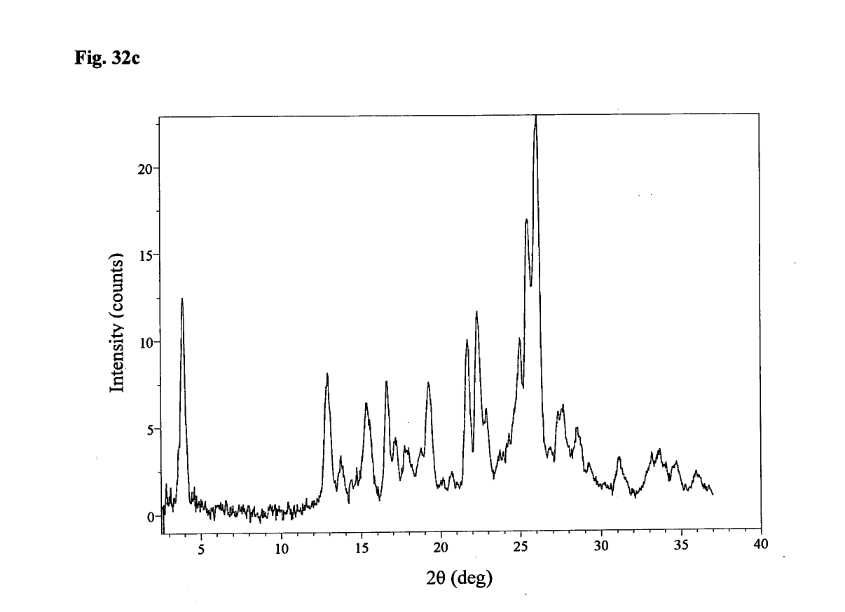

In an embodiment, Form 1 of (R)-5-(2-Aminoethyl)-1-(6,8-difluorochroman-3-yl)-1,3-dihydroimidazole-2-thione gentisate is characterised as having an XRPD pattern as shown in FIG. 32a. In another embodiment, Form 1 of (R)-5-(2-Aminoethyl)-1-(6,8-difluorochroman-3-yl)-1,3-dihydroimidazole-2-thione gentisate is characterised as having an XRPD pattern as shown in FIG. 32b.

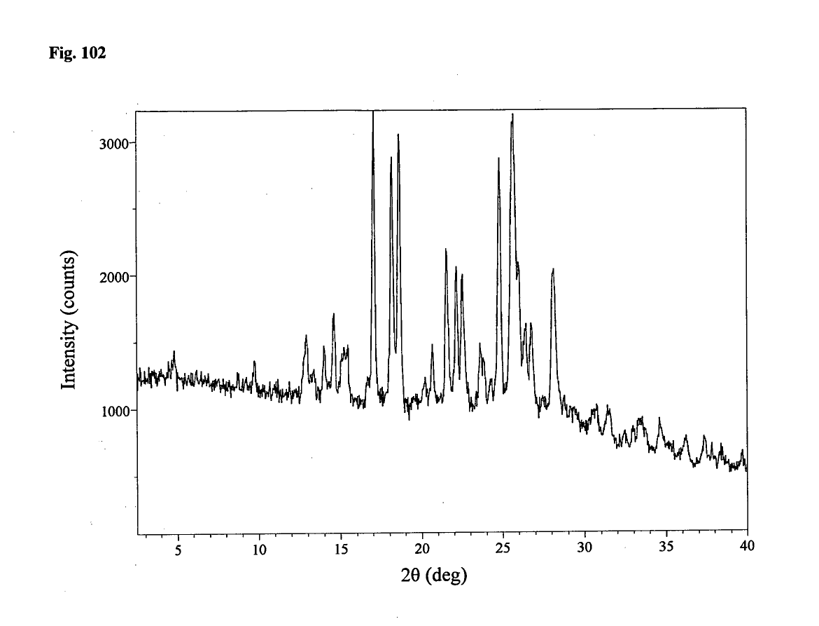

In an embodiment, Form 1 of (R)-5-(2-Aminoethyl)-1-(6,8-difluorochroman-3-yl)-1,3-dihydroimidazole-2-thione gentisate is characterised as having an XRPD pattern as shown in FIG. 102.

In an embodiment, there is provided crystalline Form 2 of (R)-5-(2-Aminoethyl)-1-(6,8-difluorochroman-3-yl)-1,3-dihydroimidazole-2-thione gentisate.

Form 2 may be characterised as having an XRPD pattern with a peak at 3.9 °2θ±0.2 °2θ. The XRPD pattern may have a further peak at 19.3 °2θ±0.2 °2θ. The XRPD pattern may have still further peaks at 12.9 and 13.7 °2θ±0.2°θ. The XRPD pattern may have yet further peaks at 15.4 and 16.6 °2θ±0.2°θ. The XRPD pattern may have still yet further peaks at 25.5 and 26.1 °2θ±0.2°θ.

In an embodiment, Form 2 has an XRPD pattern with peaks at the positions listed in Table 85 below.

TABLE 85

| °2θ | d space (Å) | Intensity % (I/Io) |
|---|---|---|
| 3.9 ± 0.1 | 22.541 ± 0.590 | 56 |
| 19.3 ± 0.1 | 4.604 ± 0.024 | 36 |

In another embodiment, Form 2 has an XRPD pattern with peaks at the positions listed in Table 86 below.

TABLE 86

| °2θ | d space (Å) | Intensity % (I/Io) |
|---|---|---|
| 3.9 ± 0.1 | 22.541 ± 0.590 | 56 |
| 12.9 ± 0.1 | 6.852 ± 0.053 | 38 |
| 13.7 ± 0.1 | 6.454 ± 0.047 | 18 |
| 15.4 ± 0.1 | 5.769 ± 0.038 | 31 |
| 16.6 ± 0.1 | 5.341 ± 0.032 | 36 |
| 19.3 ± 0.1 | 4.604 ± 0.024 | 36 |
| 21.8 ± 0.1 | 4.084 ± 0.019 | 45 |
| 22.4 ± 0.1 | 3.976 ± 0.018 | 53 |
| 25.5 ± 0.1 | 3.496 ± 0.014 | 75 |
| 26.1 ± 0.1 | 3.417 ± 0.013 | 100 |

In yet another embodiment, Form 2 has an XRPD pattern with peaks at the positions listed in Table 87 below.

TABLE 87

| °2θ | d space (Å) | Intensity % (I/Io) |
|---|---|---|
| 3.9 ± 0.1 | 22.541 ± 0.590 | 56 |
| 12.9 ± 0.1 | 6.852 ± 0.053 | 38 |
| 13.7 ± 0.1 | 6.454 ± 0.047 | 18 |
| 15.4 ± 0.1 | 5.769 ± 0.038 | 31 |
| 16.6 ± 0.1 | 5.341 ± 0.032 | 36 |
| 17.1 ± 0.1 | 5.179 ± 0.030 | 22 |
| 17.8 ± 0.1 | 4.994 ± 0.028 | 21 |
| 18.8 ± 0.1 | 4.730 ± 0.025 | 20 |
| 19.3 ± 0.1 | 4.604 ± 0.024 | 36 |
| 20.7 ± 0.1 | 4.295 ± 0.021 | 14 |
| 21.8 ± 0.1 | 4.084 ± 0.019 | 45 |
| 22.4 ± 0.1 | 3.976 ± 0.018 | 53 |
| 22.9 ± 0.1 | 3.880 ± 0.017 | 29 |
| 25.0 ± 0.1 | 3.556 ± 0.014 | 45 |
| 25.5 ± 0.1 | 3.496 ± 0.014 | 75 |
| 26.1 ± 0.1 | 3.417 ± 0.013 | 100 |
| 27.7 ± 0.1 | 3.223 ± 0.011 | 30 |
| 28.5 ± 0.1 | 3.130 ± 0.011 | 24 |

In an embodiment, Form 2 of (R)-5-(2-Aminoethyl)-1-(6,8-difluorochroman-3-yl)-1,3-dihydroimidazole-2-thione gentisate is characterised as having an XRPD pattern as shown in FIG. 32c.

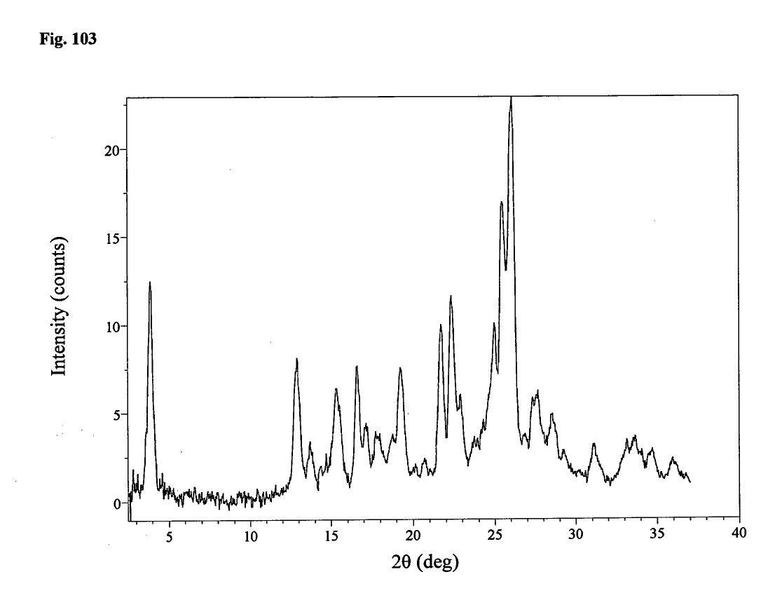

In an embodiment, Form 2 of (R)-5-(2-Aminoethyl)-1-(6,8-difluorochroman-3-yl)-1,3-dihydroimidazole-2-thione gentisate is characterised as having an XRPD pattern as shown in FIG. 103.

In another embodiment, Form 2 of the gentisate salt is characterised as being in the form of a solvate of ethyl acetate. The number of moles of ethyl acetate per mole of Form 2 may range from about 0.4 to about 1.0. Typically, the number of moles ranges from about 0.5 to about 0.9, more typically from about 0.6 to about 0.8. In an embodiment, there is 0.7 mole of ethyl acetate per 1 mole of Form 2.

According to another aspect of the present invention, there is provided the citric acid salt of (R)-5-(2-Aminoethyl)-1-(6,8-difluorochroman-3-yl)-1,3-dihydroimidazole-2-thione, i.e. (R)-5-(2-Aminoethyl)-1-(6,8-difluorochroman-3-yl)-1,3-dihydroimidazole-2-thione citrate.

In an embodiment, there is provided crystalline Form 1 of (R)-5-(2-Aminoethyl)-1-(6,8-difluorochroman-3-yl)-1,3-dihydroimidazole-2-thione citrate.

Form 1 may be characterised as having an XRPD pattern with peaks at 10.6 and 13.7 °2θ±0.2°2θ. The XRPD pattern may have a further peak at 8.9 °2θ±0.2 °2θ. The XRPD pattern may have a still further peak at 12.3 °2θ±0.2°θ. The XRPD pattern may have yet further peaks at 15.6 and 15.9 °2θ±0.2°θ. The XRPD pattern may have still yet further peaks at 23.2 and 26.4 °2θ±0.2°θ.

In an embodiment, Form 1 has an XRPD pattern with peaks at the positions listed in Table 88 below.

TABLE 88

| °2θ | d space (Å) | Intensity % (I/Io) |
|---|---|---|
| 8.9 ± 0.1 | 9.914 ± 0.112 | 18 |
| 10.6 ± 0.1 | 8.378 ± 0.080 | 37 |
| 13.7 ± 0.1 | 6.473 ± 0.047 | 38 |

In another embodiment, Form 1 has an XRPD pattern with peaks at the positions listed in Table 89 below.

TABLE 89

| °2θ | d space (Å) | Intensity % (I/Io) |
|---|---|---|
| 8.9 ± 0.1 | 9.914 ± 0.112 | 18 |
| 10.6 ± 0.1 | 8.378 ± 0.080 | 37 |
| 12.3 ± 0.1 | 7.185 ± 0.059 | 52 |
| 13.7 ± 0.1 | 6.473 ± 0.047 | 38 |
| 15.6 ± 0.1 | 5.695 ± 0.037 | 73 |
| 15.9 ± 0.1 | 5.581 ± 0.035 | 72 |
| 23.2 ± 0.1 | 3.828 ± 0.016 | 65 |
| 26.4 ± 0.1 | 3.381 ± 0.013 | 100 |

In yet another embodiment, Form 1 has an XRPD pattern with peaks at the positions listed in Table 90 below.

TABLE 90

| °2θ | d space (Å) | Intensity % (I/Io) |
|---|---|---|
| 8.9 ± 0.1 | 9.914 ± 0.112 | 18 |
| 10.6 ± 0.1 | 8.378 ± 0.080 | 37 |
| 12.3 ± 0.1 | 7.185 ± 0.059 | 52 |
| 13.0 ± 0.1 | 6.810 ± 0.053 | 26 |
| 13.7 ± 0.1 | 6.473 ± 0.047 | 38 |
| 14.7 ± 0.1 | 6.018 ± 0.041 | 21 |
| 15.6 ± 0.1 | 5.695 ± 0.037 | 73 |
| 15.9 ± 0.1 | 5.581 ± 0.035 | 72 |
| 17.0 ± 0.1 | 5.204 ± 0.030 | 22 |
| 18.6 ± 0.1 | 4.760 ± 0.025 | 29 |
| 19.4 ± 0.1 | 4.585 ± 0.024 | 43 |
| 20.8 ± 0.1 | 4.271 ± 0.020 | 43 |
| 21.3 ± 0.1 | 4.175 ± 0.019 | 38 |
| 22.3 ± 0.1 | 3.990 ± 0.018 | 35 |
| 22.6 ± 0.1 | 3.934 ± 0.017 | 36 |
| 23.2 ± 0.1 | 3.828 ± 0.016 | 65 |
| 24.0 ± 0.1 | 3.702 ± 0.015 | 51 |
| 24.6 ± 0.1 | 3.613 ± 0.014 | 54 |
| 26.4 ± 0.1 | 3.381 ± 0.013 | 100 |
| 28.6 ± 0.1 | 3.117 ± 0.011 | 30 |

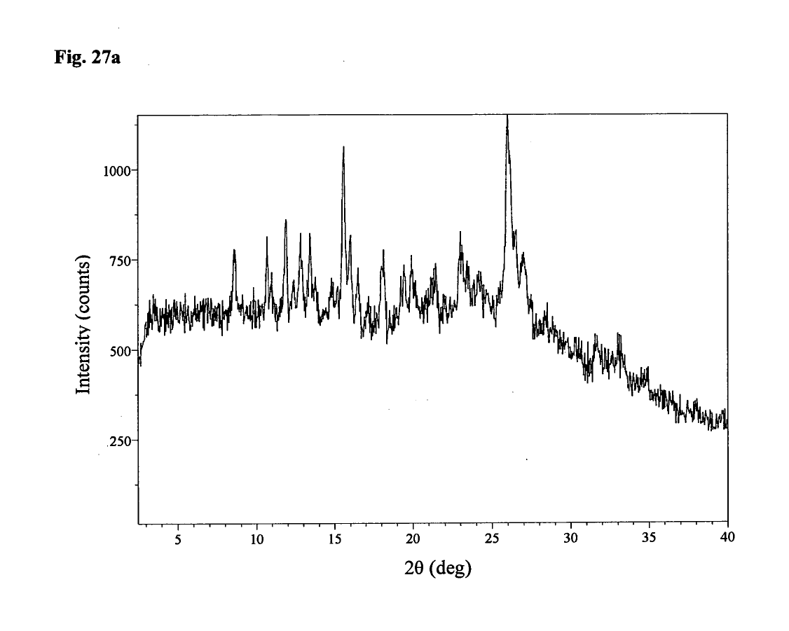

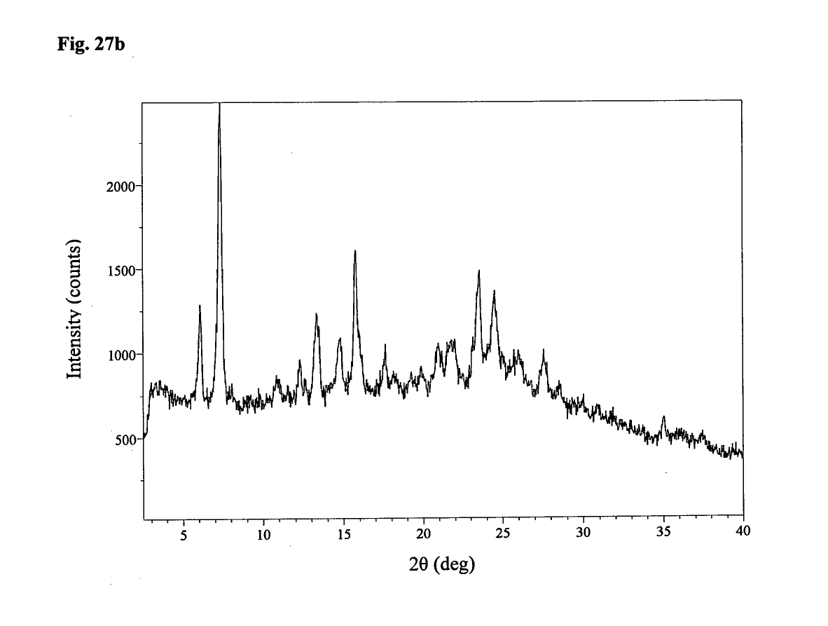

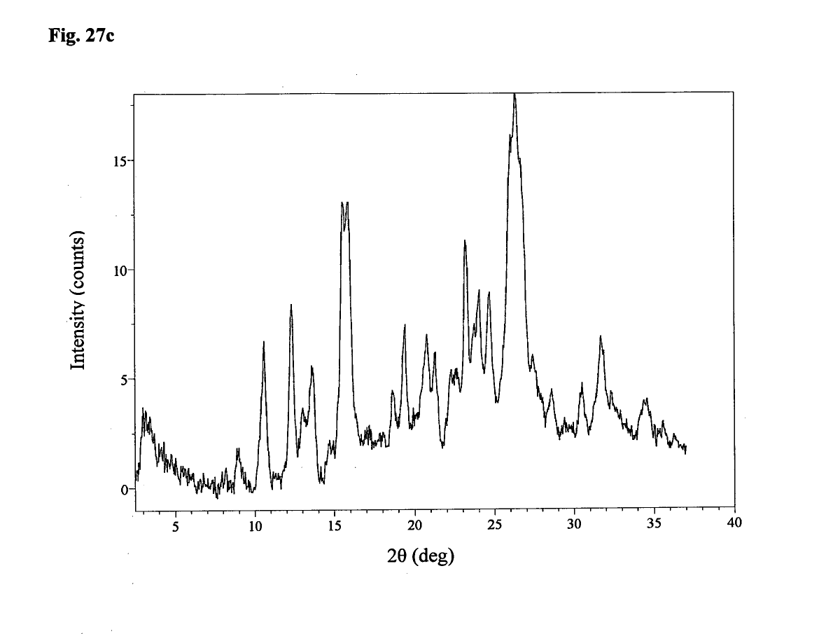

In an embodiment, Form 1 of (R)-5-(2-Aminoethyl)-1-(6,8-difluorochroman-3-yl)-1,3-dihydroimidazole-2-thione citrate is characterised as having an XRPD pattern as shown in FIG. 27a. In another embodiment, Form 1 of (R)-5-(2-Aminoethyl)-1-(6,8-difluorochroman-3-yl)-1,3-dihydroimidazole-2-thione citrate is characterised as having an XRPD pattern as shown in FIG. 27c.

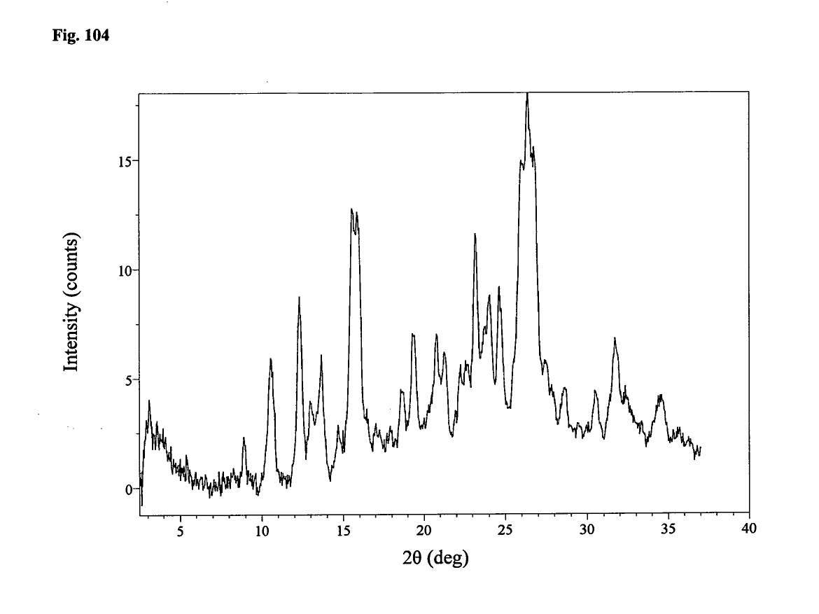

In an embodiment, Form 1 of (R)-5-(2-Aminoethyl)-1-(6,8-difluorochroman-3-yl)-1,3-dihydroimidazole-2-thione citrate is characterised as having an XRPD pattern as shown in FIG. 104.

In another embodiment, there is provided crystalline Form 2 of (R)-5-(2-Aminoethyl)-1-(6,8-difluorochroman-3-yl)-1,3-dihydroimidazole-2-thione citrate.

Form 2 may be characterised as having an XRPD pattern with peaks at 6.1 and 7.4 °2θ±0.2 °2θ. The XRPD pattern may have further peaks at 13.4 and 14.7 °2θ±0.2 °2θ. The XRPD pattern may have a still further peak at 15.7 °2θ±0.2°θ.

In an embodiment, Form 2 has an XRPD pattern with peaks at the positions listed in Table 91 below.

TABLE 91

| °2θ | d space (Å) | Intensity % (I/Io) |
|---|---|---|
| 6.1 ± 0.1 | 14.561 ± 0.244 | 25 |
| 7.4 ± 0.1 | 12.011 ± 0.165 | 100 |

In another embodiment, Form 2 has an XRPD pattern with peaks at the positions listed in Table 100 below.

TABLE 100

| °2θ | d space (Å) | Intensity % (I/Io) |
|---|---|---|
| 6.1 ± 0.1 | 14.561 ± 0.244 | 25 |
| 7.4 ± 0.1 | 12.011 ± 0.165 | 100 |
| 13.4 ± 0.1 | 6.583 ± 0.049 | 27 |
| 14.7 ± 0.1 | 6.010 ± 0.041 | 29 |
| 15.7 ± 0.1 | 5.634 ± 0.036 | 35 |

In yet another embodiment, Form 2 has an XRPD pattern with peaks at the positions listed in Table 101 below.

TABLE 101

| °2θ | d space (Å) | Intensity % (I/Io) |
|---|---|---|
| 6.1 ± 0.1 | 14.561 ± 0.244 | 25 |
| 7.4 ± 0.1 | 12.011 ± 0.165 | 100 |
| 8.0 ± 0.1 | 10.983 ± 0.138 | 5 |
| 10.8 ± 0.1 | 8.162 ± 0.076 | 9 |
| 12.3 ± 0.1 | 7.208 ± 0.059 | 10 |
| 13.4 ± 0.1 | 6.583 ± 0.049 | 27 |
| 14.7 ± 0.1 | 6.010 ± 0.041 | 29 |
| 15.7 ± 0.1 | 5.634 ± 0.036 | 35 |
| 16.0 ± 0.1 | 5.539 ± 0.035 | 18 |
| 17.6 ± 0.1 | 5.042 ± 0.029 | 9 |
| 18.2 ± 0.1 | 4.861 ± 0.027 | 6 |
| 19.0 ± 0.1 | 4.664 ± 0.024 | 4 |
| 19.9 ± 0.1 | 4.468 ± 0.022 | 7 |
| 20.8 ± 0.1 | 4.271 ± 0.020 | 13 |
| 21.6 ± 0.1 | 4.107 ± 0.019 | 19 |
| 23.2 ± 0.1 | 3.839 ± 0.016 | 20 |
| 23.6 ± 0.1 | 3.776 ± 0.016 | 30 |
| 24.4 ± 0.1 | 3.648 ± 0.015 | 31 |
| 26.0 ± 0.1 | 3.432 ± 0.013 | 18 |
| 27.4 ± 0.1 | 3.259 ± 0.012 | 18 |
| 28.5 ± 0.1 | 3.134 ± 0.011 | 6 |

In an embodiment, Form 2 of (R)-5-(2-Aminoethyl)-1-(6,8-difluorochroman-3-yl)-1,3-dihydroimidazole-2-thione citrate is characterised as having an XRPD pattern as shown in FIG. 27b.

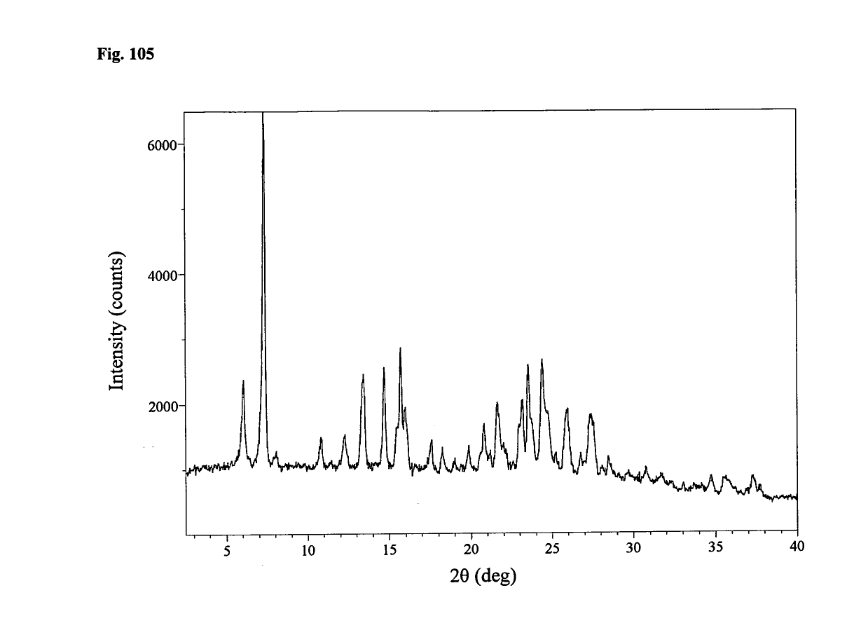

In an embodiment, Form 2 of (R)-5-(2-Aminoethyl)-1-(6,8-difluorochroman-3-yl)-1,3-dihydroimidazole-2-thione citrate is characterised as having an XRPD pattern as shown in FIG. 105.

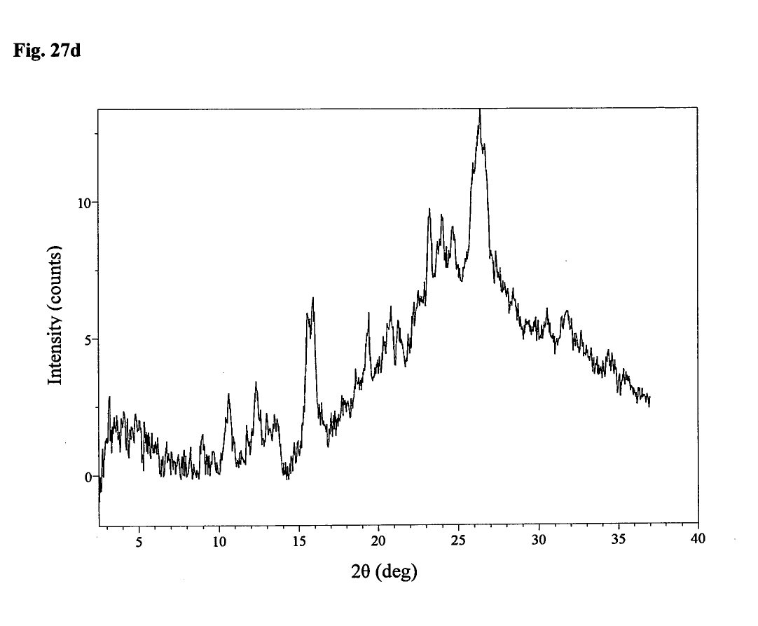

Form 2 of (R)-5-(2-Aminoethyl)-1-(6,8-difluorochroman-3-yl)-1,3-dihydroimidazole-2-thione phosphate may also be characterised by having a DSC thermogram as shown in FIG. 31.

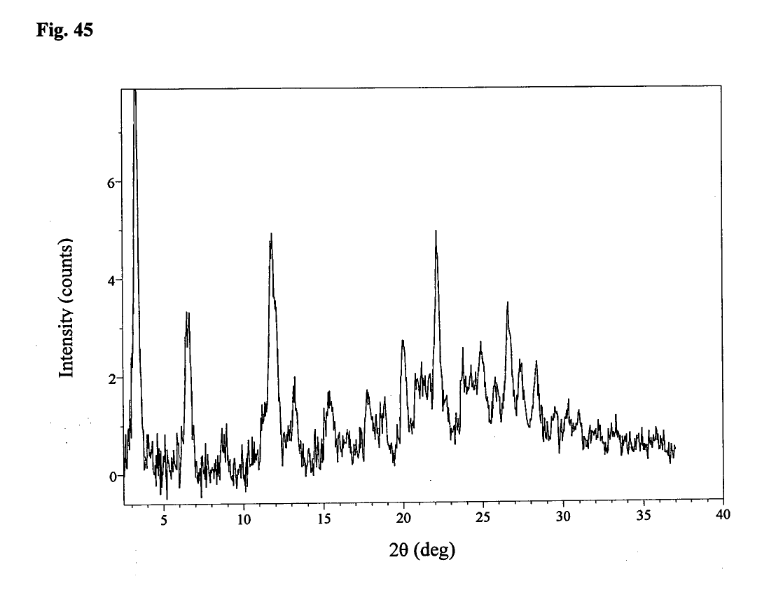

According to another aspect of the present invention, there is provided the lactic acid salt of (R)-5-(2-Aminoethyl)-1-(6,8-difluorochroman-3-yl)-1,3-dihydroimidazole-2-thione, i.e. (R)-5-(2-Aminoethyl)-1-(6,8-difluorochroman-3-yl)-1,3-dihydroimidazole-2-thione lactate. In another embodiment, there is provided crystalline (R)-5-(2-Aminoethyl)-1-(6,8-difluorochroman-3-yl)-1,3-dihydroimidazole-2-thione lactate. Crystalline (R)-5-(2-Aminoethyl)-1-(6,8-difluorochroman-3-yl)-1,3-dihydroimidazole-2-thione lactate may be characterised by having an XRPD pattern as shown in FIG. 45.

According to another aspect of the present invention, there is provided the L-malic acid salt of (R)-5-(2-Aminoethyl)-1-(6,8-difluorochroman-3-yl)-1,3-dihydroimidazole-2-thione, i.e. (R)-5-(2-Aminoethyl)-1-(6,8-difluorochroman-3-yl)-1,3-dihydroimidazole-2-thione L-malate.

In an embodiment, there is provided crystalline Form 1 of (R)-5-(2-Aminoethyl)-1-(6,8-difluorochroman-3-yl)-1,3-dihydroimidazole-2-thione L-malate.

Form 1 may be characterised as having an XRPD pattern with peaks at 8.0, 9.0, 10.7, 12.0, 12.6 and 13.9 °2θ±0.2 °2θ. The XRPD pattern may have further peaks at 15.6 and 20.2 °2θ±0.2 °2θ. The XRPD pattern may have a still further peak at 20.8 °2θ±0.2°θ.

In an embodiment, Form 1 has an XRPD pattern with peaks at the positions listed in Table 102 below.

TABLE 102

| °2θ | d space (Å) | Intensity % (I/Io) |
|---|---|---|
| 8.0 ± 0.1 | 10.983 ± 0.138 | 37 |
| 9.0 ± 0.1 | 9.848 ± 0.111 | 32 |
| 10.7 ± 0.1 | 8.276 ± 0.078 | 30 |
| 12.0 ± 0.1 | 7.351 ± 0.061 | 27 |
| 12.6 ± 0.1 | 7.053 ± 0.056 | 92 |
| 13.9 ± 0.1 | 6.385 ± 0.046 | 63 |
| 15.6 ± 0.1 | 5.677 ± 0.036 | 100 |
| 20.2 ± 0.1 | 4.390 ± 0.022 | 79 |
| 20.8 ± 0.1 | 4.277 ± 0.020 | 46 |

In another embodiment, Form 1 has an XRPD pattern with peaks at the positions listed in Table 103 below.

TABLE 103

| °2θ | d space (Å) | Intensity % (I/Io) |
|---|---|---|
| 4.5 ± 0.1 | 19.464 ± 0.438 | 20 |
| 8.0 ± 0.1 | 10.983 ± 0.138 | 37 |
| 9.0 ± 0.1 | 9.848 ± 0.111 | 32 |
| 9.8 ± 0.1 | 9.007 ± 0.092 | 6 |
| 10.7 ± 0.1 | 8.276 ± 0.078 | 30 |
| 12.0 ± 0.1 | 7.351 ± 0.061 | 27 |
| 12.6 ± 0.1 | 7.053 ± 0.056 | 92 |
| 13.9 ± 0.1 | 6.385 ± 0.046 | 63 |
| 15.6 ± 0.1 | 5.677 ± 0.036 | 100 |
| 15.8 ± 0.1 | 5.591 ± 0.035 | 59 |
| 16.1 ± 0.1 | 5.509 ± 0.034 | 27 |
| 16.5 ± 0.1 | 5.369 ± 0.032 | 19 |
| 17.9 ± 0.1 | 4.966 ± 0.028 | 14 |
| 19.5 ± 0.1 | 4.550 ± 0.023 | 30 |
| 19.8 ± 0.1 | 4.482 ± 0.023 | 22 |
| 20.2 ± 0.1 | 4.390 ± 0.022 | 79 |
| 20.8 ± 0.1 | 4.277 ± 0.020 | 46 |
| 21.6 ± 0.1 | 4.124 ± 0.019 | 24 |
| 22.4 ± 0.1 | 3.960 ± 0.017 | 30 |
| 23.4 ± 0.1 | 3.805 ± 0.016 | 22 |
| 23.7 ± 0.1 | 3.753 ± 0.016 | 26 |
| 24.2 ± 0.1 | 3.670 ± 0.015 | 79 |
| 24.5 ± 0.1 | 3.631 ± 0.015 | 92 |
| 25.0 ± 0.1 | 3.562 ± 0.014 | 99 |
| 25.5 ± 0.1 | 3.492 ± 0.014 | 26 |
| 26.0 ± 0.1 | 3.425 ± 0.013 | 35 |
| 26.8 ± 0.1 | 3.330 ± 0.012 | 32 |
| 27.1 ± 0.1 | 3.294 ± 0.012 | 30 |
| 27.6 ± 0.1 | 3.227 ± 0.011 | 16 |
| 28.4 ± 0.1 | 3.147 ± 0.011 | 26 |
| 29.8 ± 0.1 | 2.995 ± 0.010 | 15 |

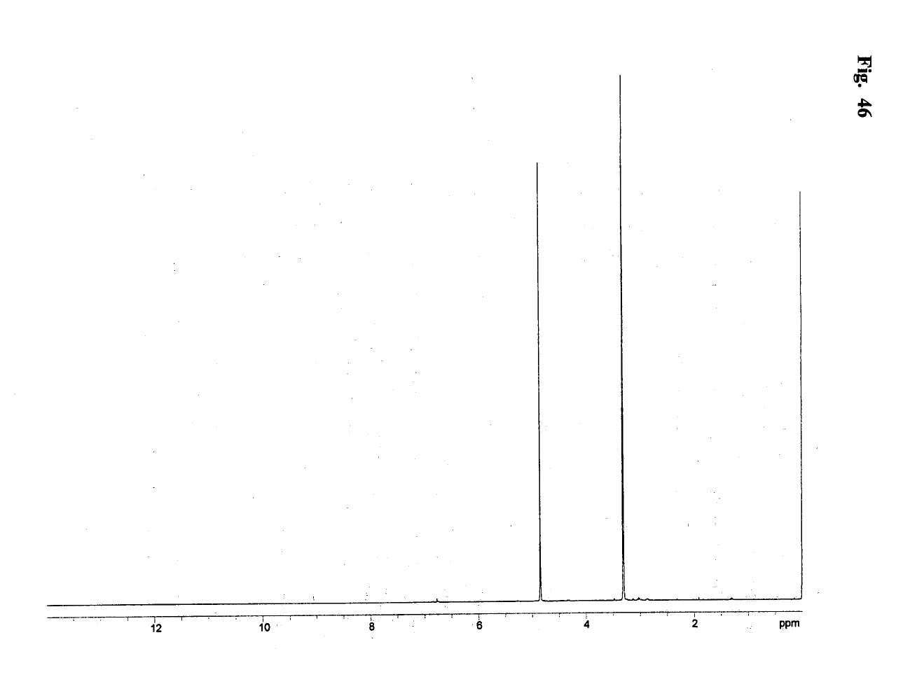

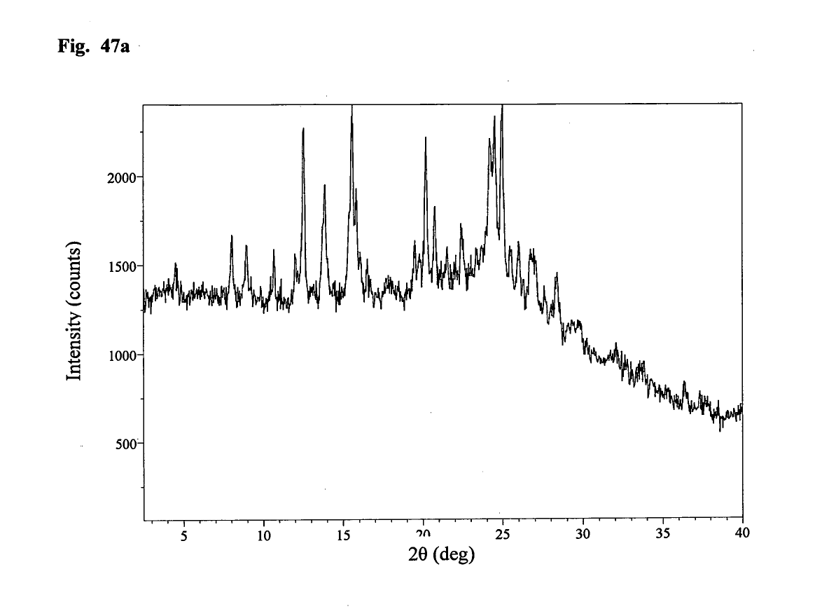

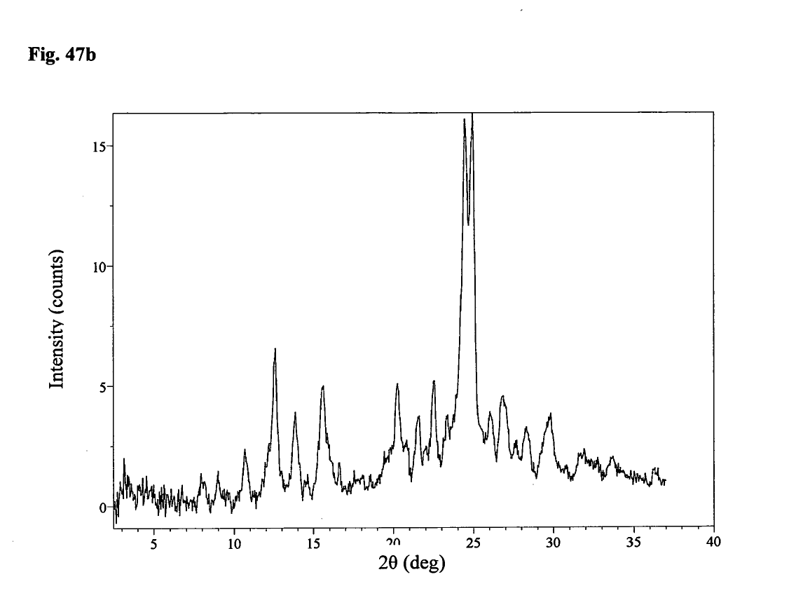

In an embodiment, Form 1 of (R)-5-(2-Aminoethyl)-1-(6,8-difluorochroman-3-yl)-1,3-dihydroimidazole-2-thione malate is characterised as having an XRPD pattern as shown in FIG. 47a. In another embodiment, Form 1 of (R)-5-(2-Aminoethyl)-1-(6,8-difluorochroman-3-yl)-1,3-dihydroimidazole-2-thione malate is characterised as having an XRPD pattern as shown in FIG. 47b.

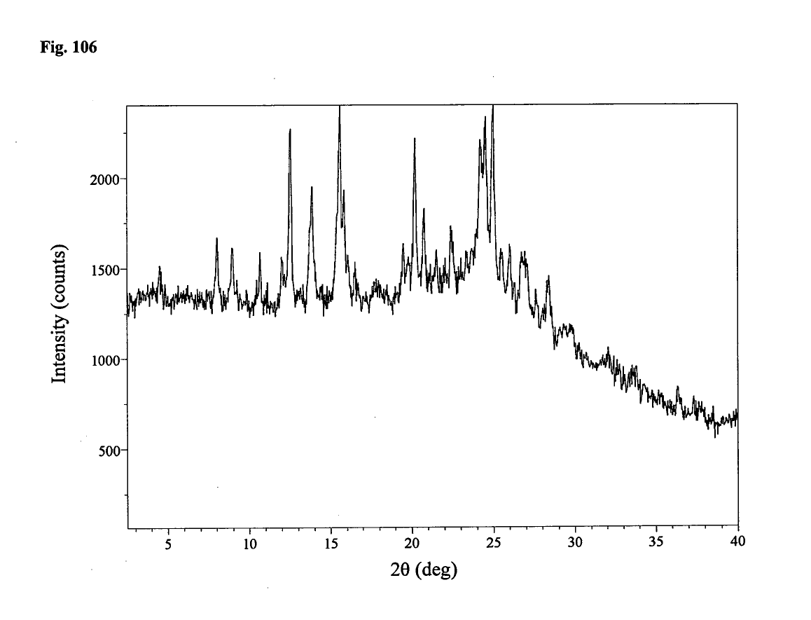

In an embodiment, Form 1 of (R)-5-(2-Aminoethyl)-1-(6,8-difluorochroman-3-yl)-1,3-dihydroimidazole-2-thione malate is characterised as having an XRPD pattern as shown in FIG. 106.

According to another aspect of the present invention, there is provided the glycolic acid salt of (R)-5-(2-Aminoethyl)-1-(6,8-difluorochroman-3-yl)-1,3-dihydroimidazole-2-thione, i.e. (R)-5-(2-Aminoethyl)-1-(6,8-difluorochroman-3-yl)-1,3-dihydroimidazole-2-thione glycolate.

In an embodiment, there is provided crystalline Form 1 of (R)-5-(2-Aminoethyl)-1-(6,8-difluorochroman-3-yl)-1,3-dihydroimidazole-2-thione glycolate.

Form 1 may be characterised as having an XRPD pattern with peaks at 5.2, 11.8, and 12.9 °2θ±0.2 °2θ. The XRPD pattern may have a further peak at 14.8 °2θ±0.2 °2θ. The XRPD pattern may have still further peaks at 15.2, 16.7, 17.1, 17.6 and 18.5 °2θ±0.2°θ.

In an embodiment, Form 1 has an XRPD pattern with peaks at the positions listed in Table 104 below.

TABLE 104

| °2θ | d space (Å) | Intensity % (I/Io) |
|---|---|---|
| 5.2 ± 0.1 | 17.093 ± 0.337 | 43 |
| 11.8 ± 0.1 | 7.519 ± 0.064 | 95 |
| 12.9 ± 0.1 | 6.873 ± 0.054 | 62 |
| 14.8 ± 0.1 | 5.986 ± 0.040 | 23 |
| 15.2 ± 0.1 | 5.833 ± 0.038 | 28 |
| 16.7 ± 0.1 | 5.321 ± 0.032 | 66 |
| 17.1 ± 0.1 | 5.182 ± 0.030 | 68 |
| 17.6 ± 0.1 | 5.051 ± 0.029 | 43 |
| 18.5 ± 0.1 | 4.791 ± 0.026 | 49 |
| 21.6 ± 0.1 | 4.124 ± 0.019 | 44 |
| 22.9 ± 0.1 | 3.879 ± 0.017 | 32 |
| 23.6 ± 0.1 | 3.762 ± 0.016 | 40 |
| 24.9 ± 0.1 | 3.579 ± 0.014 | 88 |
| 25.3 ± 0.1 | 3.516 ± 0.014 | 100 |

In another embodiment, Form 1 has an XRPD pattern with peaks at the positions listed in Table 105 below.

TABLE 105

| °2θ | d space (Å) | Intensity % (I/Io) |
|---|---|---|
| 5.2 ± 0.1 | 17.093 ± 0.337 | 43 |
| 11.8 ± 0.1 | 7.519 ± 0.064 | 95 |
| 12.9 ± 0.1 | 6.873 ± 0.054 | 62 |
| 14.8 ± 0.1 | 5.986 ± 0.040 | 23 |
| 15.2 ± 0.1 | 5.833 ± 0.038 | 28 |
| 15.5 ± 0.1 | 5.710 ± 0.037 | 9 |
| 16.7 ± 0.1 | 5.321 ± 0.032 | 66 |
| 17.1 ± 0.1 | 5.182 ± 0.030 | 68 |
| 17.6 ± 0.1 | 5.051 ± 0.029 | 43 |
| 18.5 ± 0.1 | 4.791 ± 0.026 | 49 |
| 18.7 ± 0.1 | 4.738 ± 0.025 | 29 |
| 20.1 ± 0.1 | 4.409 ± 0.022 | 10 |
| 21.1 ± 0.1 | 4.205 ± 0.020 | 19 |
| 21.6 ± 0.1 | 4.124 ± 0.019 | 44 |
| 21.8 ± 0.1 | 4.079 ± 0.019 | 13 |
| 22.9 ± 0.1 | 3.879 ± 0.017 | 32 |
| 23.4 ± 0.1 | 3.805 ± 0.016 | 13 |
| 23.6 ± 0.1 | 3.762 ± 0.016 | 40 |
| 24.9 ± 0.1 | 3.579 ± 0.014 | 88 |
| 25.3 ± 0.1 | 3.516 ± 0.014 | 100 |
| 26.2 ± 0.1 | 3.401 ± 0.013 | 27 |
| 26.4 ± 0.1 | 3.379 ± 0.013 | 28 |
| 27.2 ± 0.1 | 3.276 ± 0.012 | 18 |
| 28.2 ± 0.1 | 3.163 ± 0.011 | 47 |
| 28.4 ± 0.1 | 3.141 ± 0.011 | 63 |
| 29.9 ± 0.1 | 2.992 ± 0.010 | 22 |

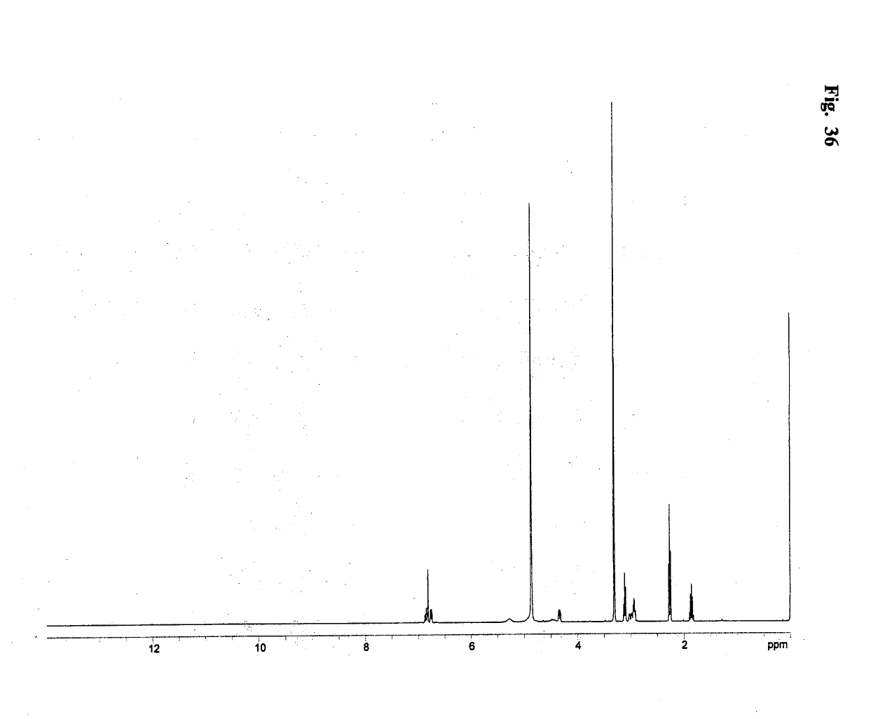

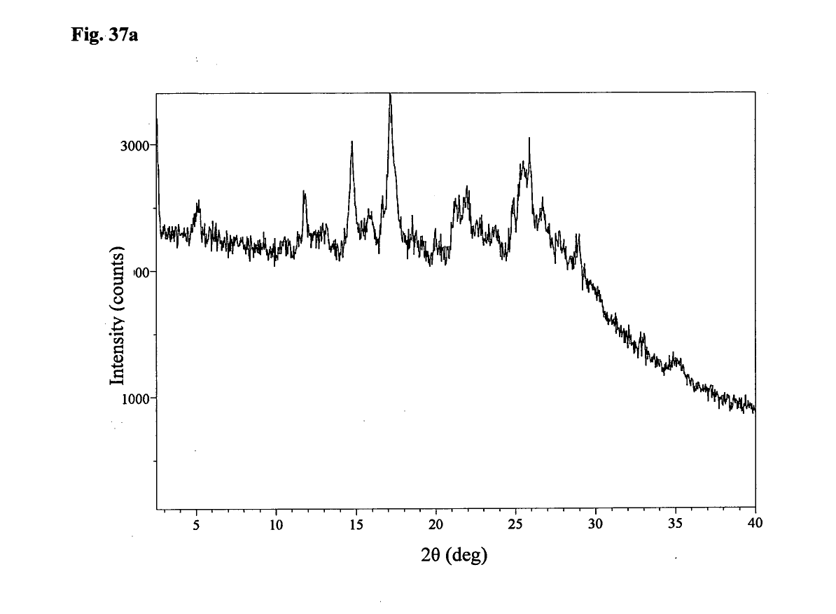

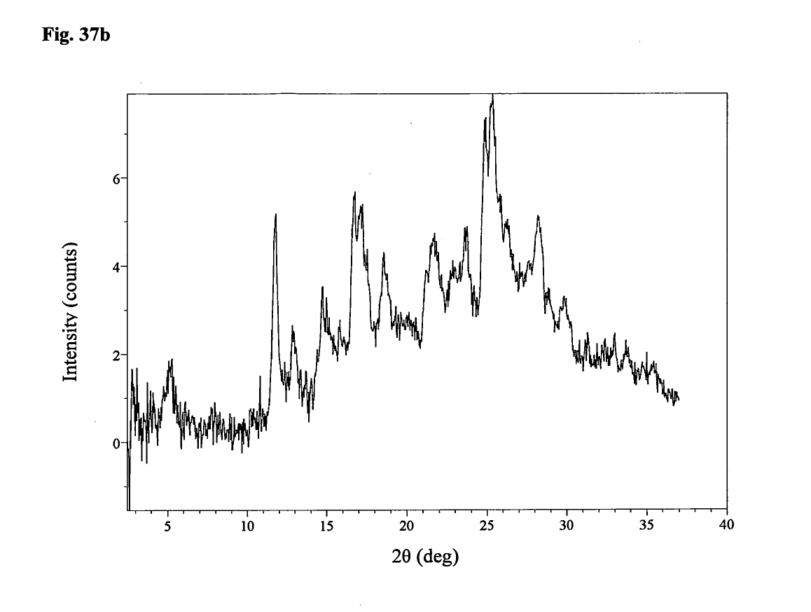

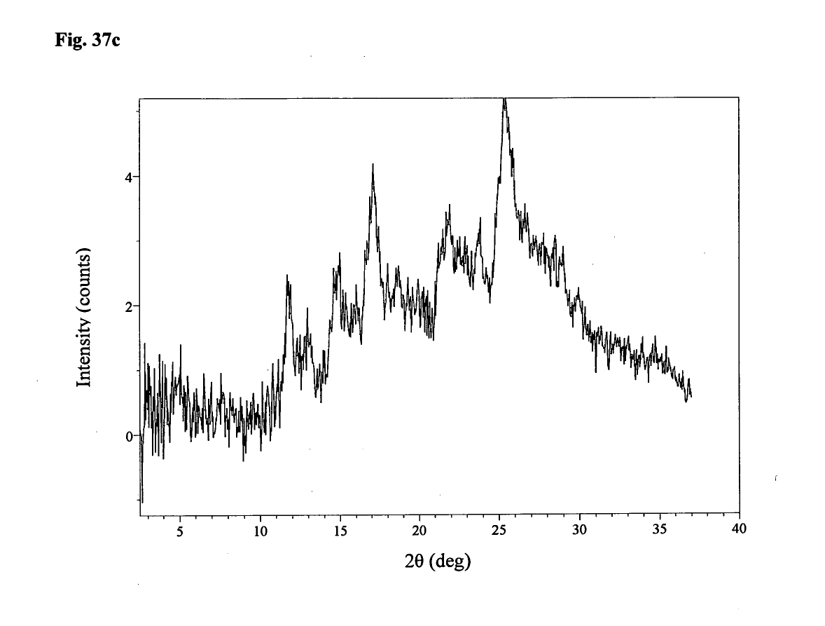

In an embodiment, Form 1 of (R)-5-(2-Aminoethyl)-1-(6,8-difluorochroman-3-yl)-1,3-dihydroimidazole-2-thione glycolate is characterised as having an XRPD pattern as shown in FIG. 37a. In another embodiment, Form 1 of (R)-5-(2-Aminoethyl)-1-(6,8-difluorochroman-3-yl)-1,3-dihydroimidazole-2-thione glycolate is characterised as having an XRPD pattern as shown in FIG. 37b.

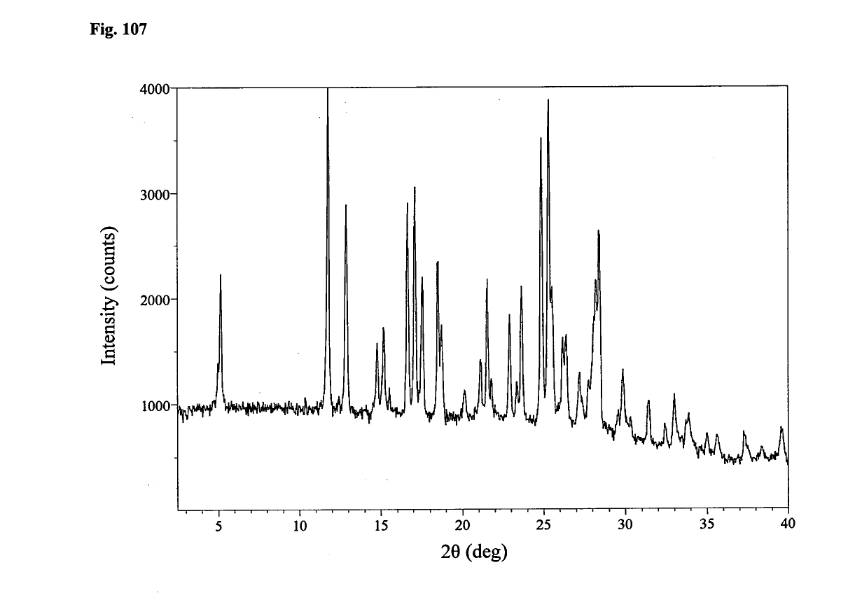

In an embodiment, Form 1 of (R)-5-(2-Aminoethyl)-1-(6,8-difluorochroman-3-yl)-1,3-dihydroimidazole-2-thione glycolate is characterised as having an XRPD pattern as shown in FIG. 107.

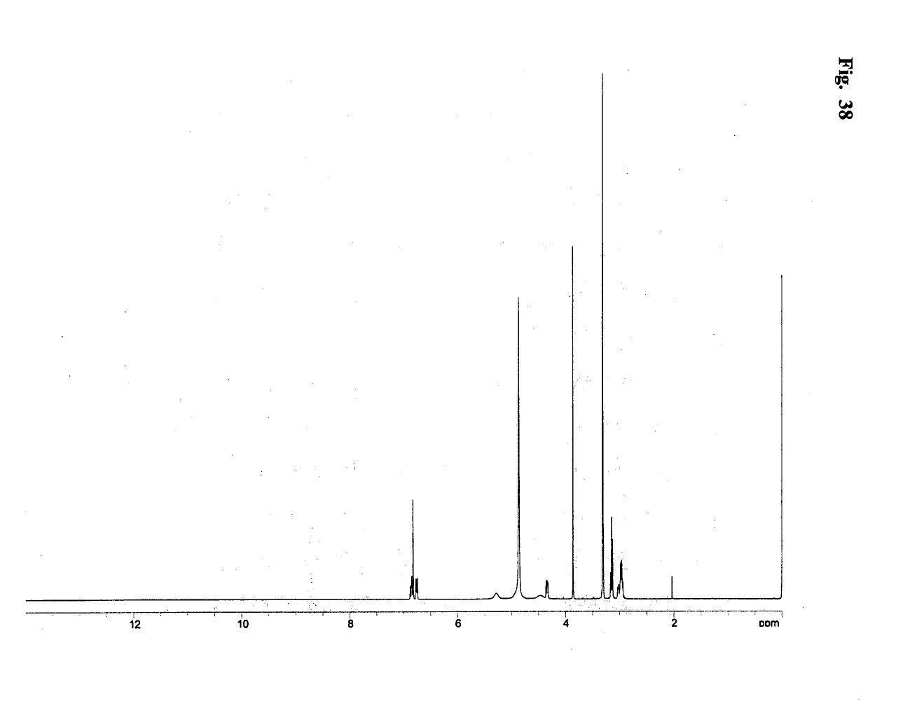

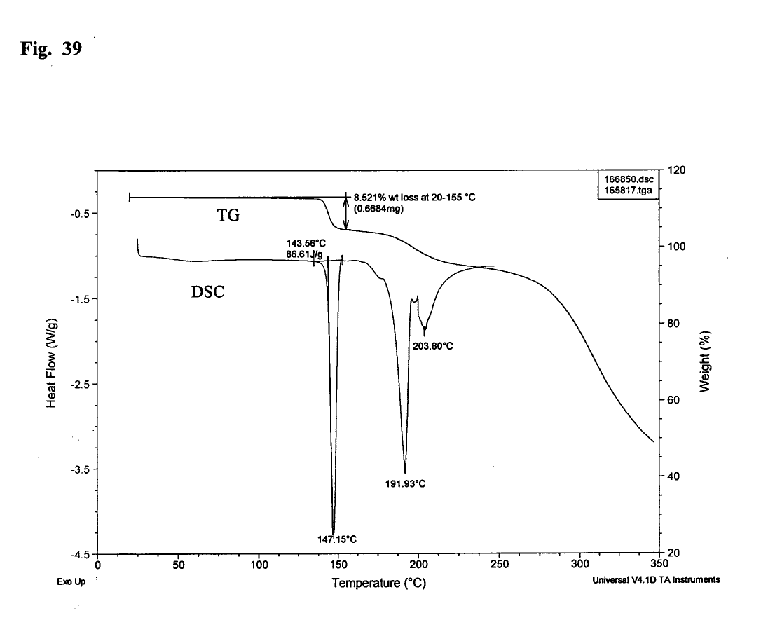

Form 1 of (R)-5-(2-Aminoethyl)-1-(6,8-difluorochroman-3-yl)-1,3-dihydroimidazole-2-thione glycolate may also be characterised by having a DSC thermogram as shown in FIG. 39.

According to another aspect of the present invention, there is provided (R)-5-(2-Aminoethyl)-1-(6,8-difluorochroman-3-yl)-1,3-dihydroimidazole-2-thione sulfate.

In an embodiment, there is provided crystalline Form 1 of (R)-5-(2-Aminoethyl)-1-(6,8-difluorochroman-3-yl)-1,3-dihydroimidazole-2-thione sulfate.

Form 1 may be characterised as having an XRPD pattern with a peak at 8.9 °2θ±0.2 °2θ. The XRPD pattern may have a further peak at 17.7 °2θ±0.2 °2θ. The XRPD pattern may have still further peaks at 11.0, 12.4, 12.7 and 13.7 °2θ±0.2°θ. The XRPD pattern may have yet further peaks at 16.0, 17.0 and 22.1 °2θ±0.2°θ.

In an embodiment, Form 1 has an XRPD pattern with peaks at the positions listed in Table 106 below.

TABLE 106

| °2θ | d space (Å) | Intensity % (I/Io) |
|---|---|---|
| 8.9 ± 0.1 | 9.947 ± 0.113 | 11 |
| 17.7 ± 0.1 | 5.000 ± 0.028 | 53 |

In another embodiment, Form 1 has an XRPD pattern with peaks at the positions listed in Table 107 below.

TABLE 107

| °2θ | d space (Å) | Intensity % (I/Io) |
|---|---|---|
| 8.9 ± 0.1 | 9.947 ± 0.113 | 11 |
| 11.0 ± 0.1 | 8.007 ± 0.073 | 28 |
| 12.4 ± 0.1 | 7.156 ± 0.058 | 14 |
| 12.7 ± 0.1 | 6.970 ± 0.055 | 24 |
| 13.7 ± 0.1 | 6.483 ± 0.048 | 26 |
| 16.0 ± 0.1 | 5.550 ± 0.035 | 59 |
| 17.0 ± 0.1 | 5.210 ± 0.031 | 38 |
| 17.7 ± 0.1 | 5.000 ± 0.028 | 53 |
| 22.1 ± 0.1 | 4.019 ± 0.018 | 100 |

In yet another embodiment, Form 1 has an XRPD pattern with peaks at the positions listed in Table 108 below.

TABLE 108

| °2θ | d space (Å) | Intensity % (I/Io) |
|---|---|---|
| 7.3 ± 0.1 | 12.160 ± 0.169 | 7 |
| 8.9 ± 0.1 | 9.947 ± 0.113 | 11 |
| 11.0 ± 0.1 | 8.007 ± 0.073 | 28 |
| 12.4 ± 0.1 | 7.156 ± 0.058 | 14 |
| 12.7 ± 0.1 | 6.970 ± 0.055 | 24 |
| 13.4 ± 0.1 | 6.583 ± 0.049 | 14 |
| 13.7 ± 0.1 | 6.483 ± 0.048 | 26 |
| 14.6 ± 0.1 | 6.084 ± 0.042 | 4 |
| 15.2 ± 0.1 | 5.844 ± 0.039 | 5 |
| 16.0 ± 0.1 | 5.550 ± 0.035 | 59 |
| 17.0 ± 0.1 | 5.210 ± 0.031 | 38 |
| 17.7 ± 0.1 | 5.000 ± 0.028 | 53 |
| 19.1 ± 0.1 | 4.649 ± 0.024 | 12 |
| 20.3 ± 0.1 | 4.370 ± 0.021 | 6 |
| 21.5 ± 0.1 | 4.129 ± 0.019 | 28 |
| 22.1 ± 0.1 | 4.019 ± 0.018 | 100 |
| 22.7 ± 0.1 | 3.919 ± 0.017 | 19 |
| 23.4 ± 0.1 | 3.795 ± 0.016 | 22 |
| 23.6 ± 0.1 | 3.762 ± 0.016 | 21 |
| 24.0 ± 0.1 | 3.706 ± 0.015 | 10 |
| 24.5 ± 0.1 | 3.631 ± 0.015 | 29 |
| 24.9 ± 0.1 | 3.570 ± 0.014 | 38 |
| 26.4 ± 0.1 | 3.375 ± 0.013 | 15 |
| 27.1 ± 0.1 | 3.290 ± 0.012 | 9 |
| 27.6 ± 0.1 | 3.238 ± 0.012 | 27 |
| 28.2 ± 0.1 | 3.163 ± 0.011 | 4 |
| 28.9 ± 0.1 | 3.093 ± 0.011 | 10 |
| 29.3 ± 0.1 | 3.049 ± 0.010 | 30 |
| 29.7 ± 0.1 | 3.004 ± 0.010 | 8 |

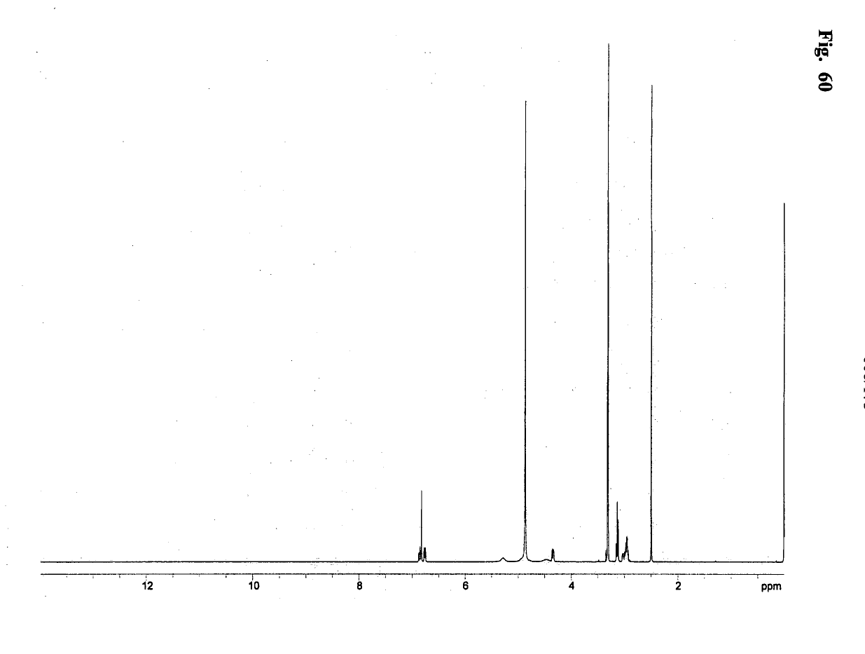

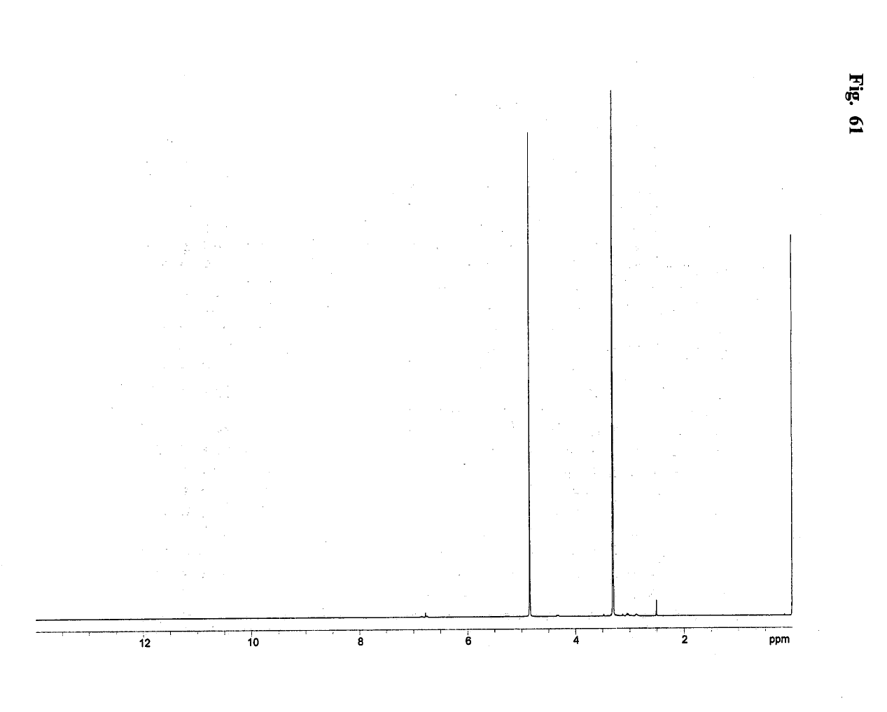

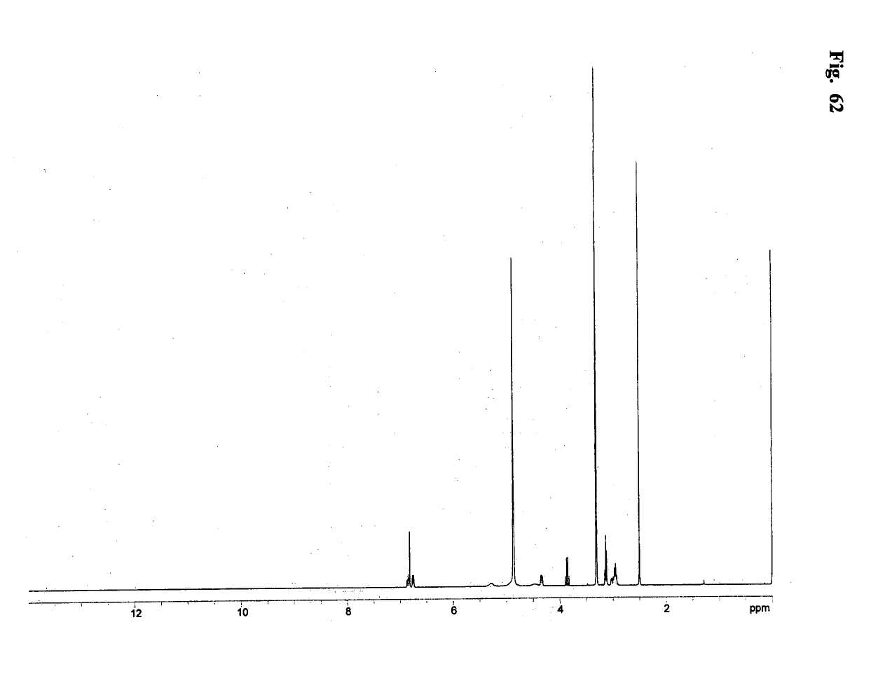

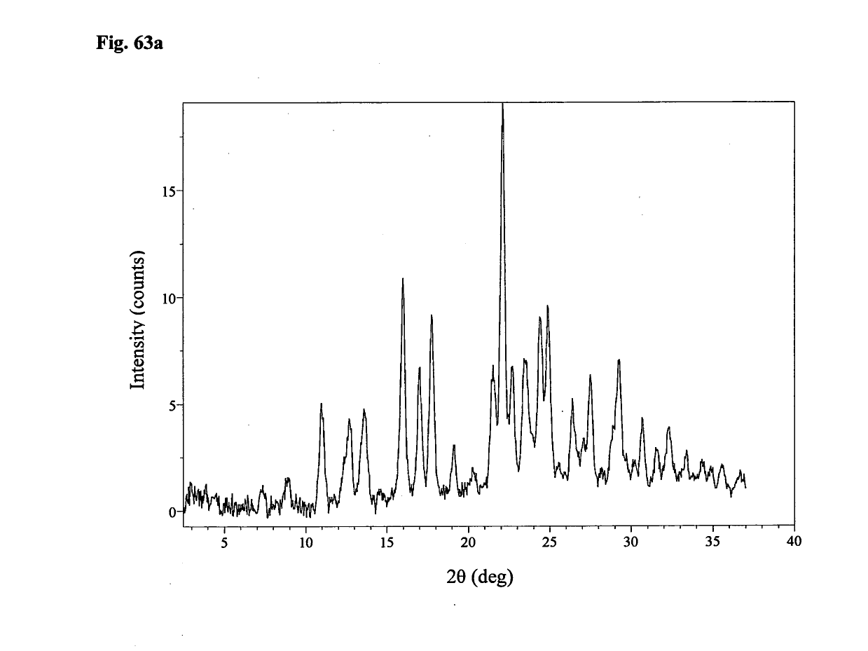

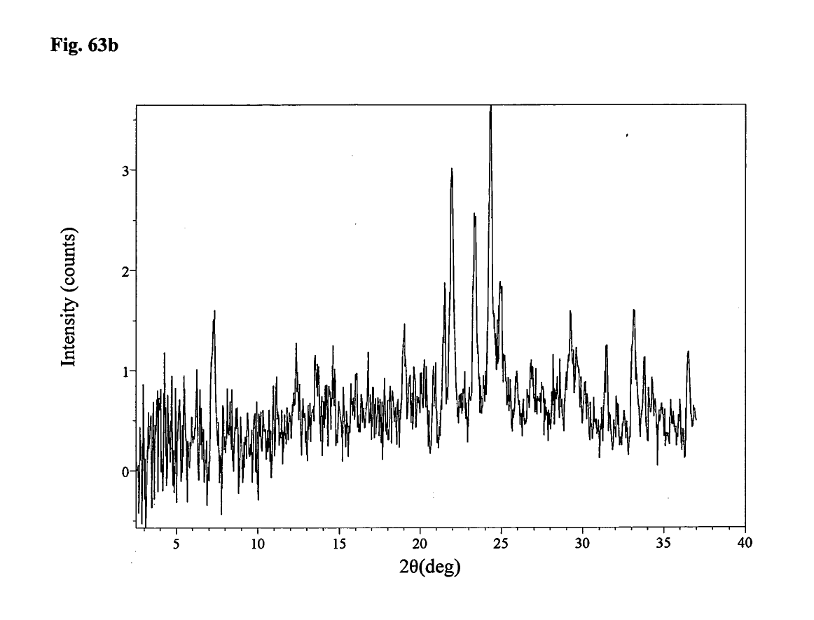

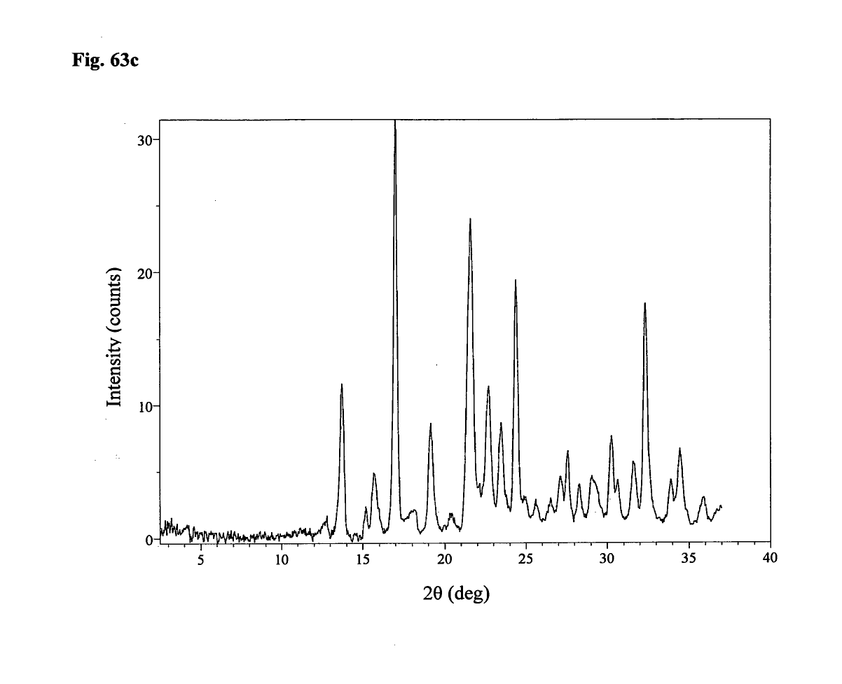

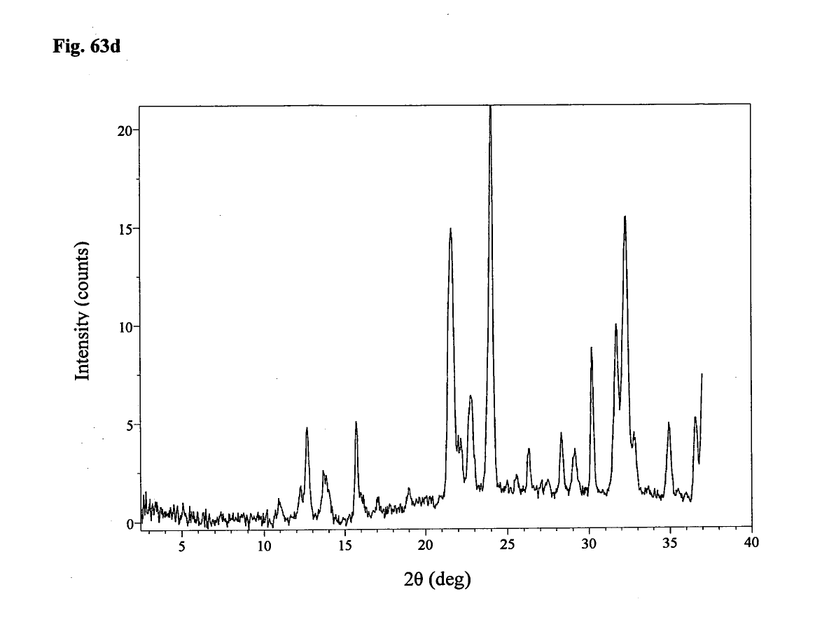

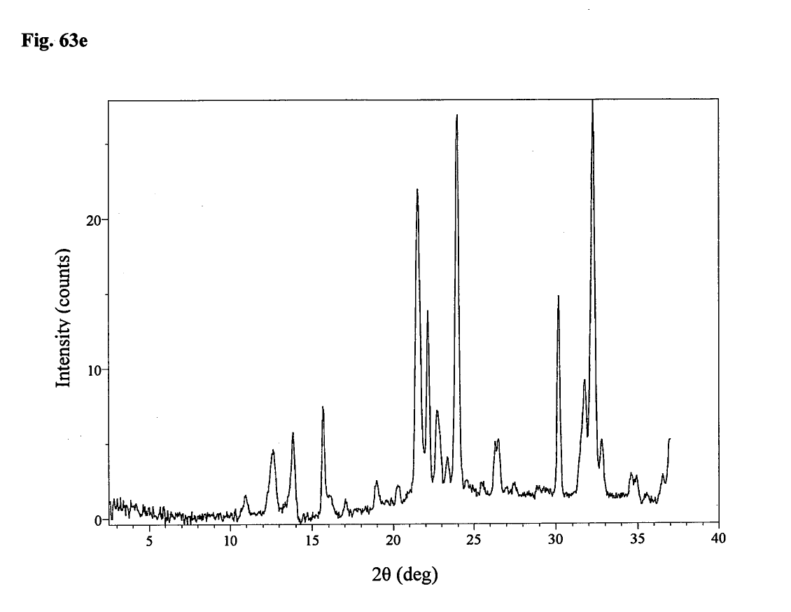

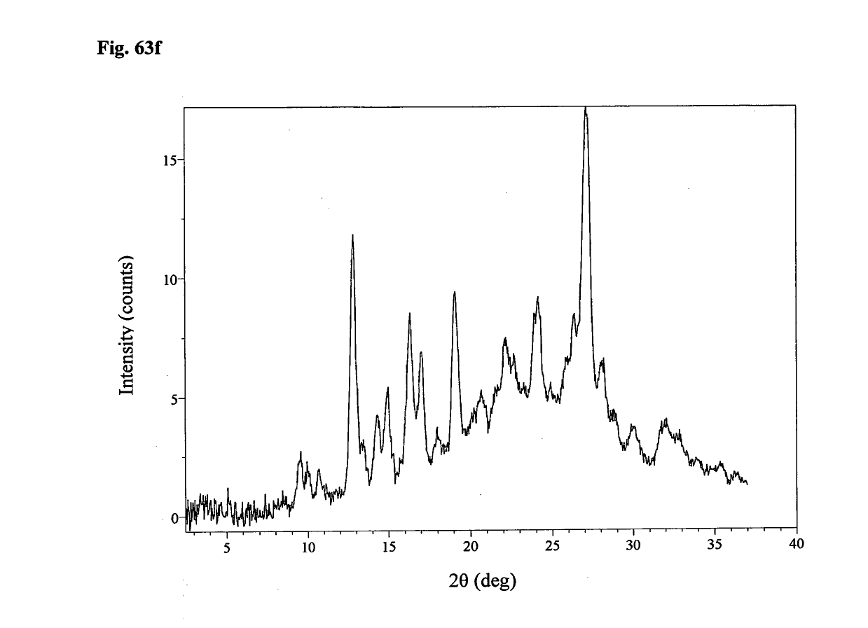

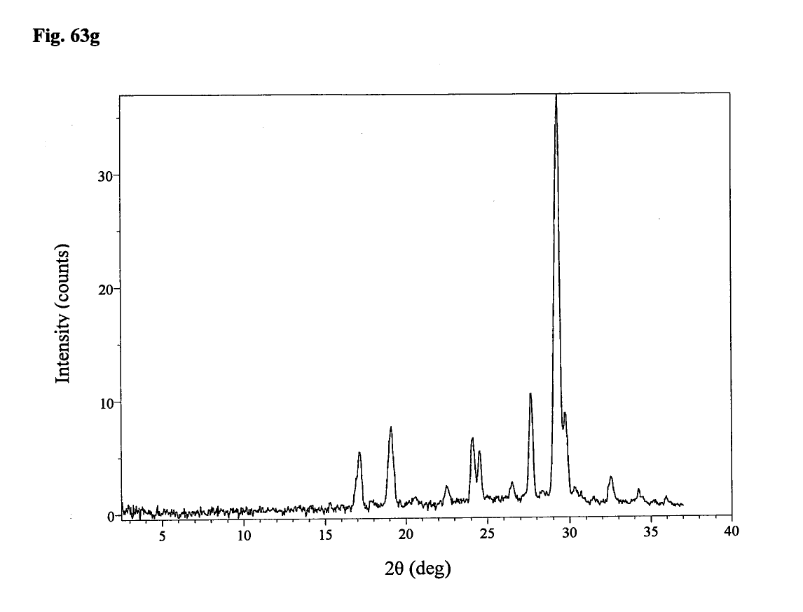

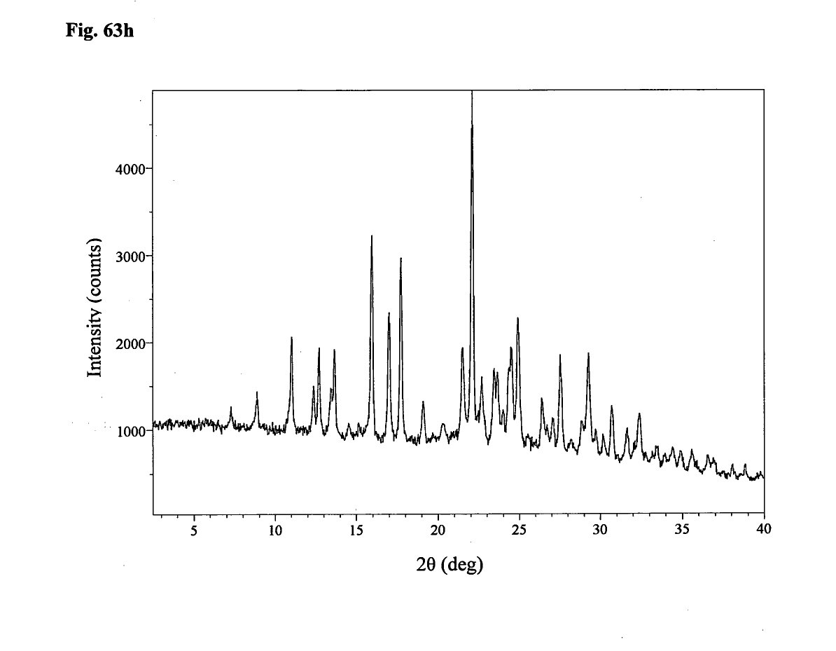

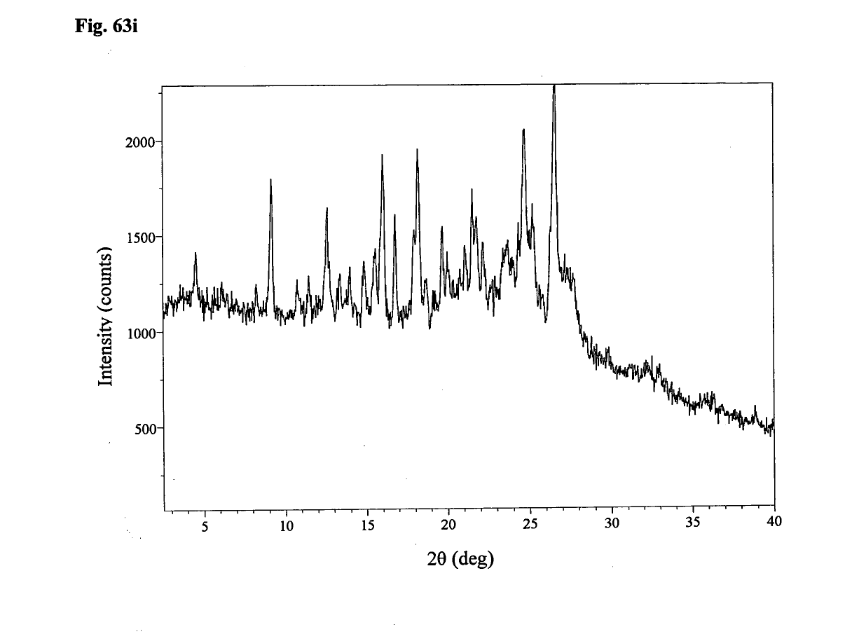

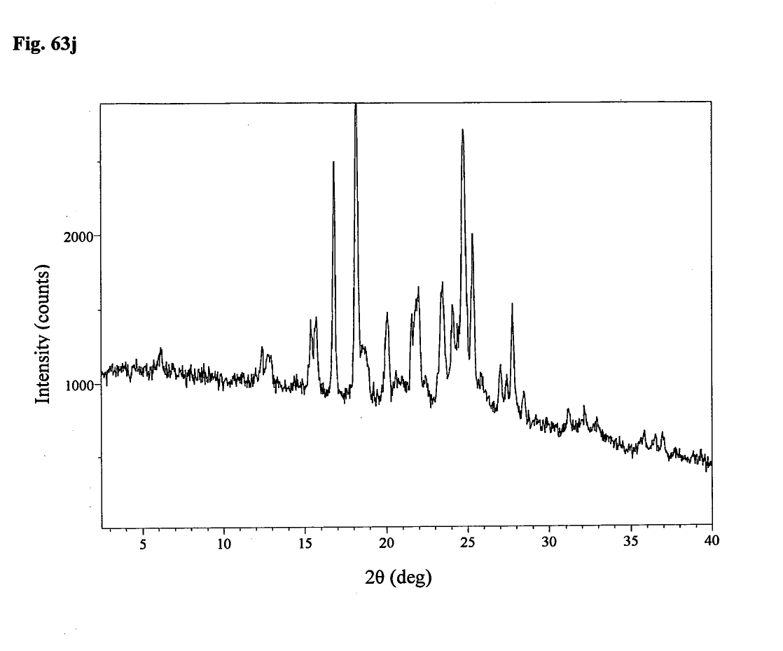

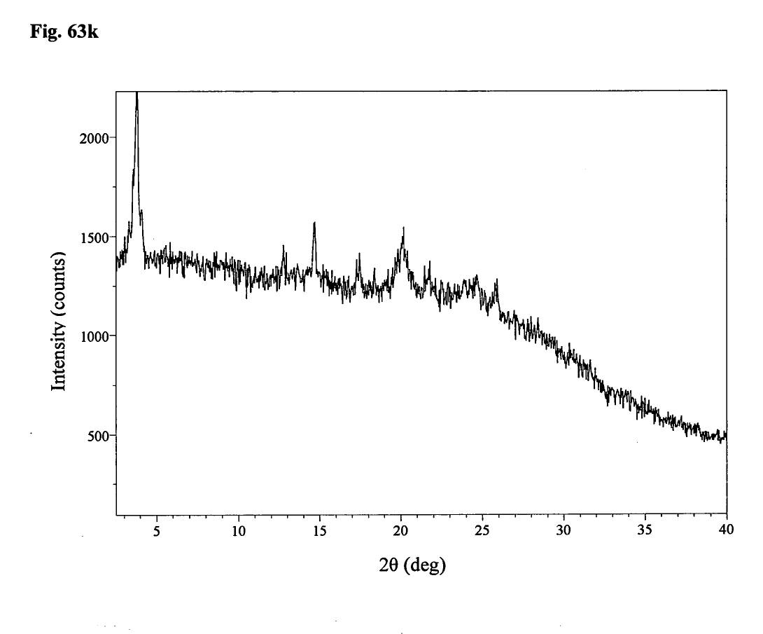

In an embodiment, Form 1 of (R)-5-(2-Aminoethyl)-1-(6,8-difluorochroman-3-yl)-1,3-dihydroimidazole-2-thione sulfate is characterised as having an XRPD pattern as shown in FIG. 63a. In another embodiment, Form 1 of (R)-5-(2-Aminoethyl)-1-(6,8-difluorochroman-3-yl)-1,3-dihydroimidazole-2-thione sulfate is characterised as having an XRPD pattern as shown in FIG. 63h.

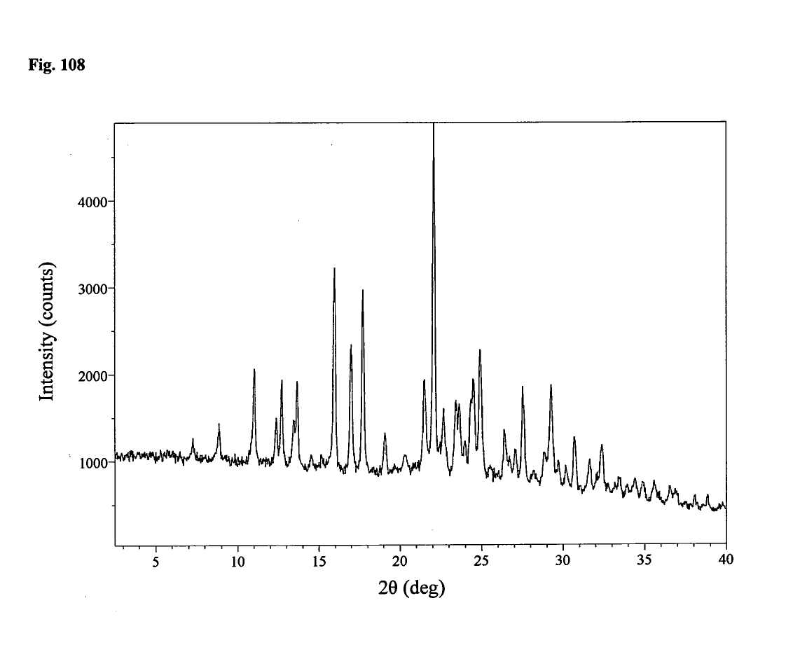

In an embodiment, Form 1 of (R)-5-(2-Aminoethyl)-1-(6,8-difluorochroman-3-yl)-1,3-dihydroimidazole-2-thione sulfate is characterised as having an XRPD pattern as shown in FIG. 108.

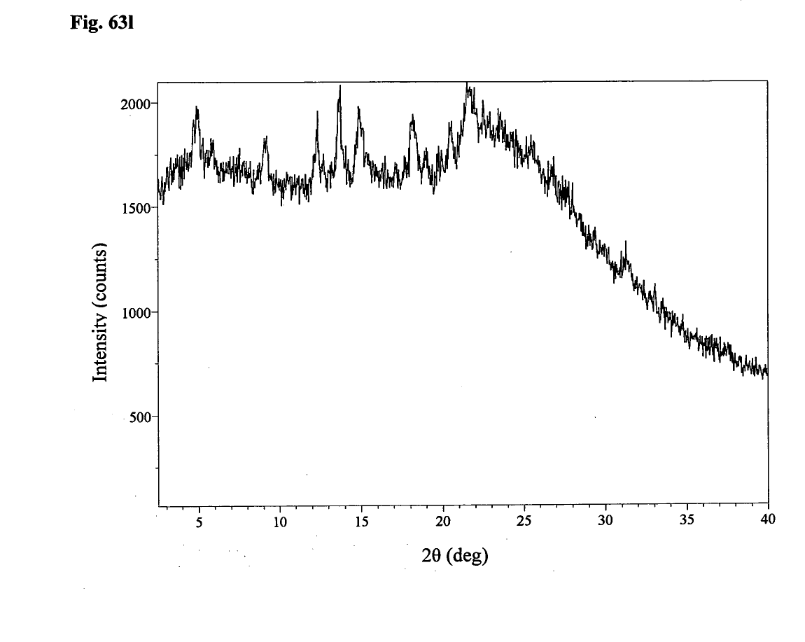

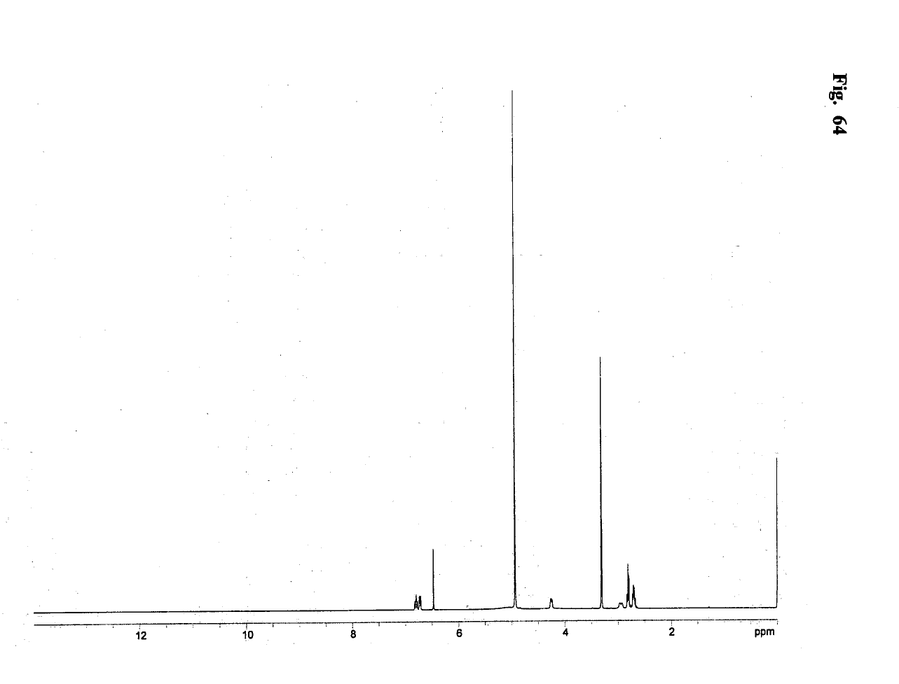

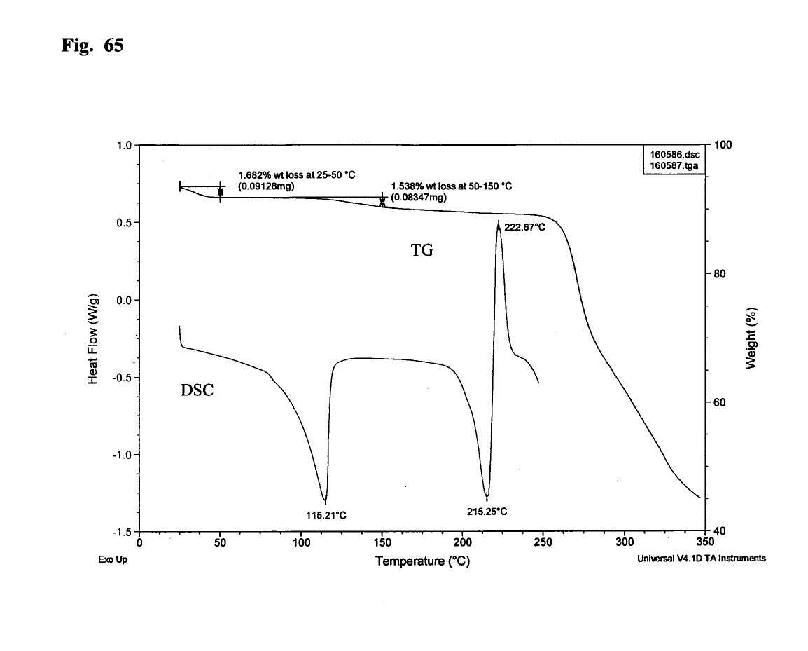

Form 1 of (R)-5-(2-Aminoethyl)-1-(6,8-difluorochroman-3-yl)-1,3-dihydroimidazole-2-thione sulfate may also be characterised by having a DSC thermogram as shown in FIG. 65.

In an embodiment, there is provided a crystal modification of (R)-5-(2-Aminoethyl)-1-(6,8-difluorochroman-3-yl)-1,3-dihydroimidazole-2-thione sulfate. This crystal modification is hereinafter referred to as crystal modification X of (R)-5-(2-Aminoethyl)-1-(6,8-difluorochroman-3-yl)-1,3-dihydroimidazole-2-thione sulfate.

Crystal modification X may be characterised as having an XRPD pattern with peaks at 12.7 and 15.8 °2θ±0.2 °2θ. The XRPD pattern may have still further peaks at 21.6 and 24.1 °2θ±0.2°θ.

In an embodiment, crystal modification X has an XRPD pattern with peaks at the positions listed in Table 109 below.

TABLE 109

| °2θ | d space (Å) | Intensity % (I/Io) |
|---|---|---|
| 12.7 ± 0.1 | 6.981 ± 0.055 | 24 |
| 15.8 ± 0.1 | 5.623 ± 0.036 | 25 |
| 21.6 ± 0.1 | 4.107 ± 0.019 | 71 |
| 24.1 ± 0.1 | 3.696 ± 0.015 | 100 |

In another embodiment, crystal modification X has an XRPD pattern with peaks at the positions listed in Table 110 below.

TABLE 110

| °2θ | d space (Å) | Intensity % (I/Io) |
|---|---|---|
| 10.9 ± 0.1 | 8.102 ± 0.075 | 7 |
| 12.3 ± 0.1 | 7.208 ± 0.059 | 10 |
| 12.7 ± 0.1 | 6.981 ± 0.055 | 24 |
| 13.7 ± 0.1 | 6.454 ± 0.047 | 13 |
| 15.8 ± 0.1 | 5.623 ± 0.036 | 25 |
| 17.1 ± 0.1 | 5.192 ± 0.030 | 6 |
| 19.0 ± 0.1 | 4.671 ± 0.024 | 10 |
| 21.6 ± 0.1 | 4.107 ± 0.019 | 71 |
| 22.0 ± 0.1 | 4.033 ± 0.018 | 22 |
| 22.8 ± 0.1 | 3.900 ± 0.017 | 31 |
| 24.1 ± 0.1 | 3.696 ± 0.015 | 100 |
| 25.6 ± 0.1 | 3.480 ± 0.013 | 12 |
| 26.3 ± 0.1 | 3.386 ± 0.013 | 19 |
| 27.5 ± 0.1 | 3.246 ± 0.012 | 11 |
| 28.3 ± 0.1 | 3.151 ± 0.011 | 22 |
| 29.2 ± 0.1 | 3.063 ± 0.010 | 19 |

In an embodiment, crystal modification X of (R)-5-(2-Aminoethyl)-1-(6,8-difluorochroman-3-yl)-1,3-dihydroimidazole-2-thione sulfate is characterised as having an XRPD pattern as shown in FIG. 63d.

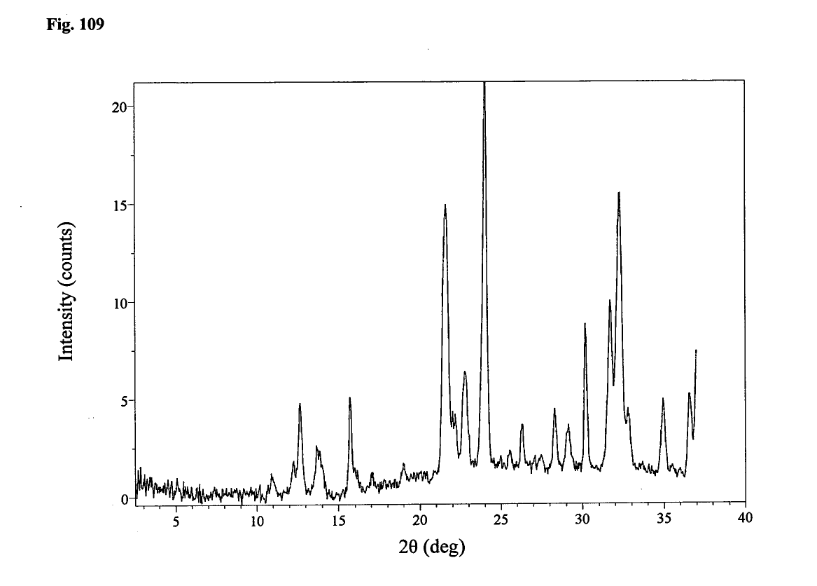

In another embodiment, crystal modification X of (R)-5-(2-Aminoethyl)-1-(6,8-difluorochroman-3-yl)-1,3-dihydroimidazole-2-thione sulfate is characterised as having an XRPD pattern as shown in FIG. 109.

In an embodiment, there is provided crystalline Form 3 of (R)-5-(2-Aminoethyl)-1-(6,8-difluorochroman-3-yl)-1,3-dihydroimidazole-2-thione sulfate.

Form 3 may be characterised as having an XRPD pattern with a peak at 9.6 °2θ±0.2 °2θ. The XRPD pattern may have further peaks at 16.4 °2θ±0.2 °2θ. The XRPD pattern may have a still further peak at 12.8 °2θ±0.2°θ. The XRPD pattern may have yet further peaks at 17.0, 19.1 and 27.1 °2θ±0.2°θ.

In an embodiment, Form 3 has an XRPD pattern with peaks at the positions listed in Table 112 below.

TABLE 112

| °2θ | d space (Å) | Intensity % (I/Io) |
|---|---|---|
| 9.6 ± 0.1 | 9.252 ± 0.098 | 19 |
| 16.4 ± 0.1 | 5.418 ± 0.033 | 51 |

In another embodiment, Form 3 has an XRPD pattern with peaks at the positions listed in Table 113 below.

TABLE 113

| °2θ | d space (Å) | Intensity % (I/Io) |
|---|---|---|
| 9.6 ± 0.1 | 9.252 ± 0.098 | 19 |
| 12.8 ± 0.1 | 6.895 ± 0.054 | 70 |
| 16.4 ± 0.1 | 5.418 ± 0.033 | 51 |
| 17.0 ± 0.1 | 5.204 ± 0.030 | 42 |
| 19.1 ± 0.1 | 4.652 ± 0.024 | 56 |
| 27.1 ± 0.1 | 3.288 ± 0.012 | 100 |

In yet another embodiment, Form 3 has an XRPD pattern with peaks at the positions listed in Table 114 below.

TABLE 114

| °2θ | d space (Å) | Intensity % (I/Io) |
|---|---|---|
| 9.6 ± 0.1 | 9.252 ± 0.098 | 19 |
| 10.0 ± 0.1 | 8.846 ± 0.089 | 14 |
| 10.7 ± 0.1 | 8.284 ± 0.078 | 15 |
| 12.8 ± 0.1 | 6.895 ± 0.054 | 70 |
| 13.4 ± 0.1 | 6.588 ± 0.049 | 21 |
| 14.3 ± 0.1 | 6.203 ± 0.044 | 27 |
| 15.0 ± 0.1 | 5.922 ± 0.040 | 33 |
| 16.4 ± 0.1 | 5.418 ± 0.033 | 51 |
| 17.0 ± 0.1 | 5.204 ± 0.030 | 42 |
| 18.0 ± 0.1 | 4.928 ± 0.027 | 24 |
| 19.1 ± 0.1 | 4.652 ± 0.024 | 56 |
| 20.7 ± 0.1 | 4.295 ± 0.021 | 33 |
| 22.2 ± 0.1 | 4.012 ± 0.018 | 44 |
| 22.7 ± 0.1 | 3.921 ± 0.017 | 42 |
| 24.2 ± 0.1 | 3.684 ± 0.015 | 55 |
| 26.4 ± 0.1 | 3.381 ± 0.013 | 51 |
| 27.1 ± 0.1 | 3.288 ± 0.012 | 100 |
| 28.0 ± 0.1 | 3.182 ± 0.011 | 39 |

In an embodiment, Form 3 of (R)-5-(2-Aminoethyl)-1-(6,8-difluorochroman-3-yl)-1,3-dihydroimidazole-2-thione sulfate is characterised as having an XRPD pattern as shown in FIG. 63f.

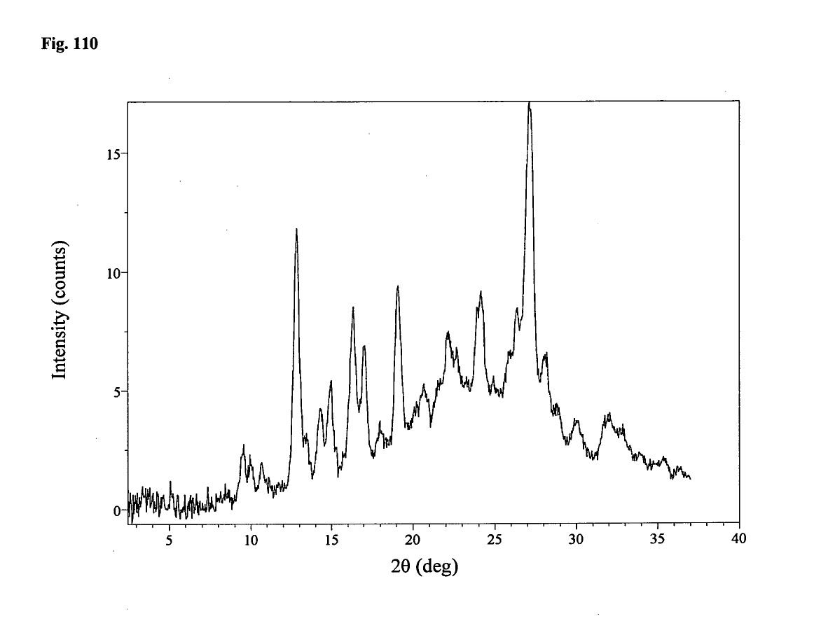

In an embodiment, Form 3 of (R)-5-(2-Aminoethyl)-1-(6,8-difluorochroman-3-yl)-1,3-dihydroimidazole-2-thione sulfate is characterised as having an XRPD pattern as shown in FIG. 110.

In an embodiment, there is provided another crystal modification of (R)-5-(2-Aminoethyl)-1-(6,8-difluorochroman-3-yl)-1,3-dihydroimidazole-2-thione sulfate. This crystal modification is hereinafter referred to as crystal modification Y of (R)-5-(2-Aminoethyl)-1-(6,8-difluorochroman-3-yl)-1,3-dihydroimidazole-2-thione sulfate.

Crystal modification Y may be characterised as having an XRPD pattern with peaks at 17.2 and 19.1 °2θ±0.2 °2θ. The XRPD pattern may have further peaks at 24.1, 24.6, 27.7 and 29.3 °2θ±0.2°2θ.

In an embodiment, crystal modification Y has an XRPD pattern with peaks at the positions listed in Table 115 below.

TABLE 115

| °2θ | d space (Å) | Intensity % (I/Io) |
|---|---|---|
| 17.2 ± 0.1 | 5.167 ± 0.030 | 16 |
| 19.1 ± 0.1 | 4.642 ± 0.024 | 22 |
| 24.1 ± 0.1 | 3.690 ± 0.015 | 18 |
| 24.6 ± 0.1 | 3.625 ± 0.015 | 16 |
| 27.7 ± 0.1 | 3.223 ± 0.011 | 29 |
| 29.3 ± 0.1 | 3.046 ± 0.010 | 100 |

In another embodiment, crystal modification Y has an XRPD pattern with peaks at the positions listed in Table 116 below.

TABLE 116

| °2θ | d space (Å) | Intensity % (I/Io) |
|---|---|---|
| 17.2 ± 0.1 | 5.167 ± 0.030 | 16 |
| 19.1 ± 0.1 | 4.642 ± 0.024 | 22 |
| 22.5 ± 0.1 | 3.948 ± 0.017 | 8 |
| 24.1 ± 0.1 | 3.690 ± 0.015 | 18 |
| 24.6 ± 0.1 | 3.625 ± 0.015 | 16 |
| 26.5 ± 0.1 | 3.361 ± 0.012 | 8 |
| 27.7 ± 0.1 | 3.223 ± 0.011 | 29 |
| 29.3 ± 0.1 | 3.046 ± 0.010 | 100 |
| 29.8 ± 0.1 | 3.002 ± 0.010 | 25 |

In an embodiment, crystal modification Y of (R)-5-(2-Aminoethyl)-1-(6,8-difluorochroman-3-yl)-1,3-dihydroimidazole-2-thione sulfate is characterised as having an XRPD pattern as shown in FIG. 63g.

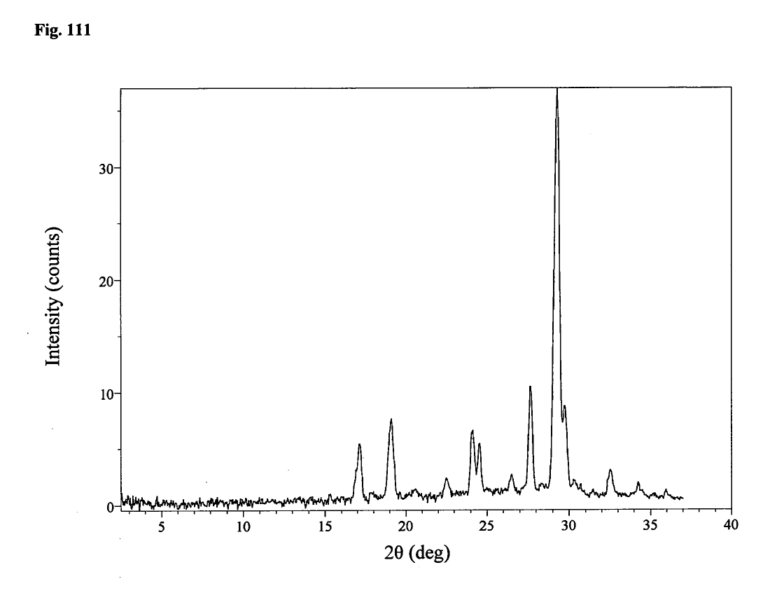

In another embodiment, crystal modification Y of (R)-5-(2-Aminoethyl)-1-(6,8-difluorochroman-3-yl)-1,3-dihydroimidazole-2-thione sulfate is characterised as having an XRPD pattern as shown in FIG. 111.

In an embodiment, there is provided crystalline Form 6 of (R)-5-(2-Aminoethyl)-1-(6,8-difluorochroman-3-yl)-1,3-dihydroimidazole-2-thione sulfate.

Form 6 may be characterised as having an XRPD pattern with peaks at 6.2 and 12.7 °2θ±0.2 °2θ. The XRPD pattern may have further peaks at 15.5, 16.8 and 18.3 °2θ±0.2°2θ. The XRPD pattern may have still further peaks at 21.7, 24.7 and 25.4 °2θ±0.2°θ.

In an embodiment, Form 6 has an XRPD pattern with peaks at the positions listed in Table 117 below.

TABLE 117

| °2θ | d space (Å) | Intensity % (I/Io) |
|---|---|---|
| 6.2 ± 0.1 | 14.210 ± 0.232 | 12 |
| 12.7 ± 0.1 | 6.987 ± 0.055 | 19 |
| 15.5 ± 0.1 | 5.710 ± 0.037 | 31 |
| 16.8 ± 0.1 | 5.274 ± 0.031 | 66 |
| 18.3 ± 0.1 | 4.838 ± 0.026 | 100 |
| 21.7 ± 0.1 | 4.101 ± 0.019 | 56 |
| 24.7 ± 0.1 | 3.609 ± 0.014 | 71 |
| 25.4 ± 0.1 | 3.512 ± 0.014 | 56 |

In another embodiment, Form 6 has an XRPD pattern with peaks at the positions listed in Table 118 below.

TABLE 118

| °2θ | d space (Å) | Intensity % (I/Io) |
|---|---|---|
| 6.2 ± 0.1 | 14.210 ± 0.232 | 12 |
| 12.4 ± 0.1 | 7.156 ± 0.058 | 11 |
| 12.7 ± 0.1 | 6.987 ± 0.055 | 19 |
| 14.3 ± 0.1 | 6.211 ± 0.044 | 5 |
| 15.5 ± 0.1 | 5.710 ± 0.037 | 31 |
| 16.8 ± 0.1 | 5.274 ± 0.031 | 66 |
| 18.3 ± 0.1 | 4.838 ± 0.026 | 100 |
| 18.7 ± 0.1 | 4.738 ± 0.025 | 25 |
| 20.0 ± 0.1 | 4.435 ± 0.022 | 24 |
| 20.6 ± 0.1 | 4.314 ± 0.021 | 15 |
| 21.2 ± 0.1 | 4.193 ± 0.020 | 11 |
| 21.7 ± 0.1 | 4.101 ± 0.019 | 56 |
| 22.2 ± 0.1 | 4.003 ± 0.018 | 13 |
| 23.4 ± 0.1 | 3.810 ± 0.016 | 34 |
| 23.6 ± 0.1 | 3.772 ± 0.016 | 32 |
| 24.0 ± 0.1 | 3.702 ± 0.015 | 24 |
| 24.3 ± 0.1 | 3.661 ± 0.015 | 22 |
| 24.7 ± 0.1 | 3.609 ± 0.014 | 71 |
| 25.4 ± 0.1 | 3.512 ± 0.014 | 56 |
| 27.0 ± 0.1 | 3.305 ± 0.012 | 9 |
| 27.7 ± 0.1 | 3.217 ± 0.011 | 32 |
| 28.5 ± 0.1 | 3.128 ± 0.011 | 9 |

In an embodiment, Form 6 of (R)-5-(2-Aminoethyl)-1-(6,8-difluorochroman-3-yl)-1,3-dihydroimidazole-2-thione sulfate is characterised as having an XRPD pattern as shown in FIG. 63j.

In an embodiment, Form 6 of (R)-5-(2-Aminoethyl)-1-(6,8-difluorochroman-3-yl)-1,3-dihydroimidazole-2-thione sulfate is characterised as having an XRPD pattern as shown in FIG. 112.

In an embodiment, there is provided crystalline Form 7 of (R)-5-(2-Aminoethyl)-1-(6,8-difluorochroman-3-yl)-1,3-dihydroimidazole-2-thione sulfate.

Form 7 may be characterised as having an XRPD pattern with a peak at 3.8 °2θ±0.2 °2θ. The XRPD pattern may have a further peak at 17.5 °2θ±0.2°2θ. The XRPD pattern may have still further peaks at 12.8 and 14.7 °2θ±0.2°θ. The XRPD pattern may have a yet further peak at 20.2 °2θ±0.2°θ.

In an embodiment, Form 7 has an XRPD pattern with peaks at the positions listed in Table 119 below.

TABLE 119

| °2θ | d space (Å) | Intensity % (I/Io) |
|---|---|---|
| 3.8 ± 0.1 | 23.131 ± 0.622 | 100 |
| 17.5 ± 0.1 | 5.076 ± 0.029 | 34 |

In another embodiment, Form 7 has an XRPD pattern with peaks at the positions listed in Table 120 below.

TABLE 120

| °2θ | d space (Å) | Intensity % (I/Io) |
|---|---|---|
| 3.8 ± 0.1 | 23.131 ± 0.622 | 100 |
| 12.8 ± 0.1 | 6.938 ± 0.055 | 34 |
| 14.7 ± 0.1 | 6.034 ± 0.041 | 53 |
| 17.5 ± 0.1 | 5.076 ± 0.029 | 34 |
| 20.2 ± 0.1 | 4.396 ± 0.022 | 54 |

In yet another embodiment, Form 7 has an XRPD pattern with peaks at the positions listed in Table 121 below.

TABLE 121

| °2θ | d space (Å) | Intensity % (I/Io) |
|---|---|---|
| 3.8 ± 0.1 | 23.131 ± 0.622 | 100 |
| 12.8 ± 0.1 | 6.938 ± 0.055 | 34 |
| 14.7 ± 0.1 | 6.034 ± 0.041 | 53 |
| 17.5 ± 0.1 | 5.076 ± 0.029 | 34 |
| 20.2 ± 0.1 | 4.396 ± 0.022 | 54 |
| 21.8 ± 0.1 | 4.079 ± 0.019 | 31 |
| 24.7 ± 0.1 | 3.609 ± 0.014 | 33 |
| 25.9 ± 0.1 | 3.436 ± 0.013 | 32 |

In an embodiment, Form 7 of (R)-5-(2-Aminoethyl)-1-(6,8-difluorochroman-3-yl)-1,3-dihydroimidazole-2-thione sulfate is characterised as having an XRPD pattern as shown in FIG. 63k.

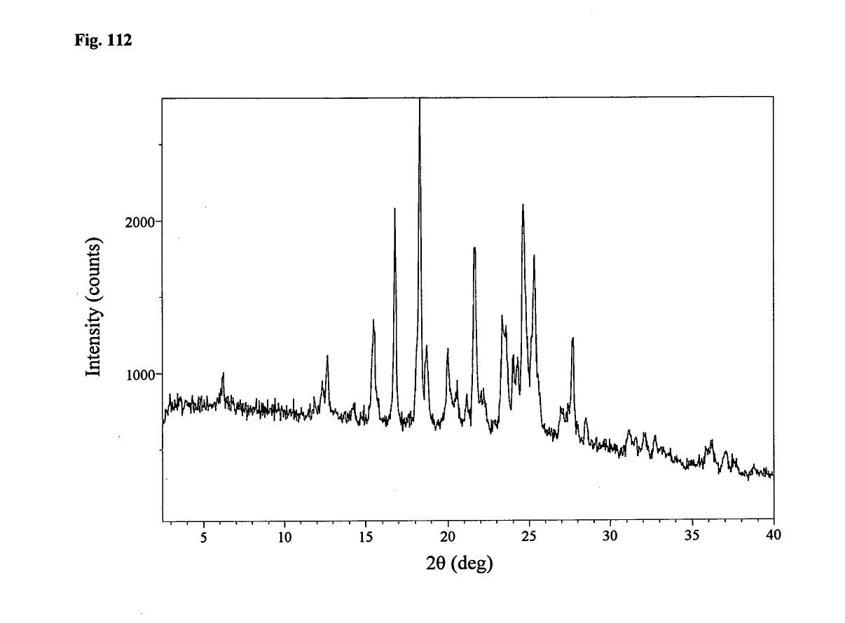

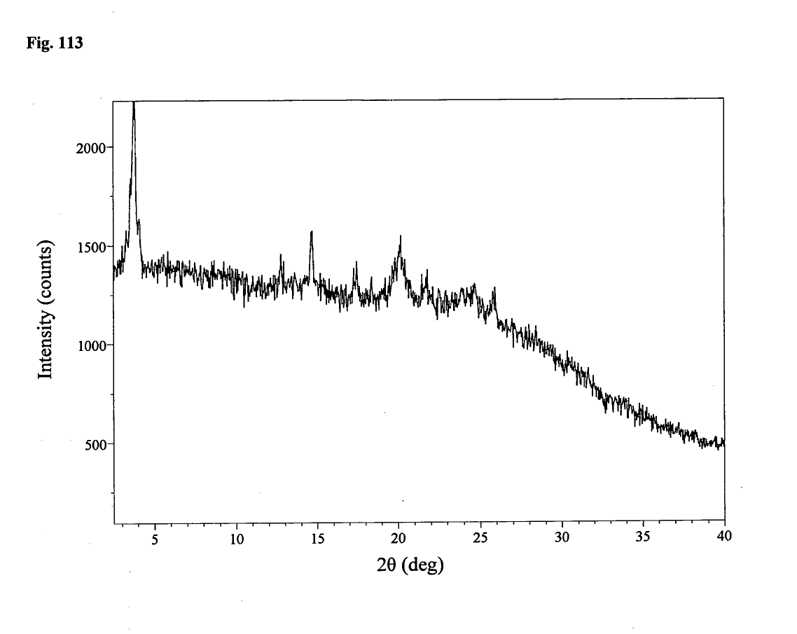

In an embodiment, Form 7 of (R)-5-(2-Aminoethyl)-1-(6,8-difluorochroman-3-yl)-1,3-dihydroimidazole-2-thione sulfate is characterised as having an XRPD pattern as shown in FIG. 113.

In an embodiment, there is provided crystalline Form 8 of (R)-5-(2-Aminoethyl)-1-(6,8-difluorochroman-3-yl)-1,3-dihydroimidazole-2-thione sulfate.

Form 8 may be characterised as having an XRPD pattern with a peak at 4.9 °2θ±0.2 °2θ. The XRPD pattern may have further peaks at 9.2, 12.4, 13.8 and 14.9 °2θ±0.2 °2θ. The XRPD pattern may have still further peaks at 18.2 and 21.5 °2θ±0.2°θ.

In an embodiment, Form 8 has an XRPD pattern with peaks at the positions listed in Table 122 below.

TABLE 122

| °2θ | d space (Å) | Intensity % (I/Io) |
|---|---|---|
| 4.9 ± 0.1 | 18.035 ± 0.375 | 68 |

In another embodiment, Form 8 has an XRPD pattern with peaks at the positions listed in Table 123 below.

TABLE 123

| °2θ | d space (Å) | Intensity % (I/Io) |
|---|---|---|
| 4.9 ± 0.1 | 18.035 ± 0.375 | 68 |
| 9.2 ± 0.1 | 9.592 ± 0.105 | 57 |
| 12.4 ± 0.1 | 7.156 ± 0.058 | 76 |
| 13.8 ± 0.1 | 6.440 ± 0.047 | 100 |
| 14.9 ± 0.1 | 5.950 ± 0.040 | 77 |
| 18.2 ± 0.1 | 4.869 ± 0.027 | 70 |
| 21.5 ± 0.1 | 4.129 ± 0.019 | 94 |

In yet another embodiment, Form 8 has an XRPD pattern with peaks at the positions listed in Table 124 below.

TABLE 124

| °2θ | d space (Å) | Intensity % (I/Io) |
|---|---|---|
| 4.9 ± 0.1 | 18.035 ± 0.375 | 68 |
| 9.2 ± 0.1 | 9.592 ± 0.105 | 57 |
| 12.4 ± 0.1 | 7.156 ± 0.058 | 76 |
| 13.8 ± 0.1 | 6.440 ± 0.047 | 100 |
| 14.9 ± 0.1 | 5.950 ± 0.040 | 77 |
| 18.2 ± 0.1 | 4.869 ± 0.027 | 70 |
| 20.6 ± 0.1 | 4.314 ± 0.021 | 56 |
| 21.5 ± 0.1 | 4.129 ± 0.019 | 94 |

In an embodiment, Form 8 of (R)-5-(2-Aminoethyl)-1-(6,8-difluorochroman-3-yl)-1,3-dihydroimidazole-2-thione sulfate is characterised as having an XRPD pattern as shown in FIG. 631.

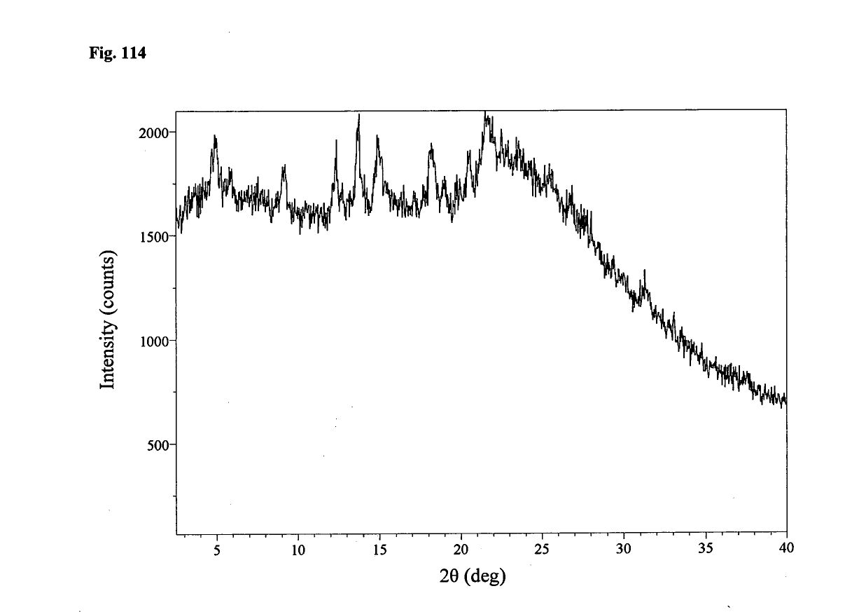

In an embodiment, Form 8 of (R)-5-(2-Aminoethyl)-1-(6,8-difluorochroman-3-yl)-1,3-dihydroimidazole-2-thione sulfate is characterised as having an XRPD pattern as shown in FIG. 114.

According to another aspect of the present invention, there is provided the hydrosulfate salt of (R)-5-(2-Aminoethyl)-1-(6,8-difluorochroman-3-yl)-1,3-dihydroimidazole-2-thione, i.e. (R)-5-(2-Aminoethyl)-1-(6,8-difluorochroman-3-yl)-1,3-dihydroimidazole-2-thione hydrosulfate.

In an embodiment, the (R)-5-(2-Aminoethyl)-1-(6,8-difluorochroman-3-yl)-1,3-dihydroimidazole-2-thione hydrosulfate is in crystalline form. The crystalline forms of the hydrosulfate salt were found in the experiments on the sulfate salt. The sulfate salt designated the number “crystalline 2 minus peaks” (FIG. 63e) was found to be the hydrosulfate salt, not the sulfate salt. This crystalline Form of the hydrosulfate form is hereinafter designated “crystalline Form A” of (R)-5-(2-Aminoethyl)-1-(6,8-difluorochroman-3-yl)-1,3-dihydroimidazole-2-thione hydrosulfate. The sulfate salt designated the number “crystalline 5” (FIG. 63i) was found to be the hydrosulfate salt, not the sulfate salt. This crystalline Form of the hydrosulfate form is hereinafter designated “crystalline Form B” of (R)-5-(2-Aminoethyl)-1-(6,8-difluorochroman-3-yl)-1,3-dihydroimidazole-2-thione hydrosulfate.

In an embodiment, Form A of (R)-5-(2-Aminoethyl)-1-(6,8-difluorochroman-3-yl)-1,3-dihydroimidazole-2-thione hydrosulfate has an XRPD pattern with a peak at a °2θ value between 29.8 and 30.5 and a peak at a °2θ value between 32.0 and 32.8. The XRPD of Form A of (R)-5-(2-Aminoethyl)-1-(6,8-difluorochroman-3-yl)-1,3-dihydroimidazole-2-thione hydrosulfate may have a further peak at a °2θ value between 13.5 and 14.2. The XRPD of Form A of (R)-5-(2-Aminoethyl)-1-(6,8-difluorochroman-3-yl)-1,3-dihydroimidazole-2-thione hydrosulfate may have a still further peak at a °2θ value between 21.2 and 21.8, a still further peak at a ° 20 value between 21.9 and 22.5 and a still further peak at a °2θ value between 23.6 and 24.3. The XRPD of Form A of (R)-5-(2-Aminoethyl)-1-(6,8-difluorochroman-3-yl)-1,3-dihydroimidazole-2-thione hydrosulfate may have a yet further peak at a °2θ value between 12.2 and 12.8 and a yet further peak at a °2θ value between 15.5 and 16.1. In one embodiment, crystalline Form A of (R)-5-(2-Aminoethyl)-1-(6,8-difluorochroman-3-yl)-1,3-dihydroimidazole-2-thione hydrosulfate is characterised as having an XRPD pattern as shown in FIG. 63e.

In an embodiment, there is provided crystalline Form B of (R)-5-(2-Aminoethyl)-1-(6,8-difluorochroman-3-yl)-1,3-dihydroimidazole-2-thione hydrosulfate

Form B may be characterised as having an XRPD pattern with peaks at 4.6, 9.2 and 12.6 °2θ±0.2 °2θ. The XRPD pattern may have further peaks at 16.0 and 18.2 °2θ±0.2 °2θ. The XRPD pattern may have still further peaks at 13.4, 14.0 and 14.9 °2θ±0.2°θ.

In an embodiment, Form B has an XRPD pattern with peaks at the positions listed in Table 125 below.

TABLE 125

| °2θ | d space (Å) | Intensity % (I/Io) |
|---|---|---|
| 4.6 ± 0.1 | 19.336 ± 0.432 | 23 |
| 9.2 ± 0.1 | 9.623 ± 0.106 | 57 |
| 12.6 ± 0.1 | 7.020 ± 0.056 | 46 |
| 16.0 ± 0.1 | 5.529 ± 0.034 | 66 |
| 18.2 ± 0.1 | 4.869 ± 0.027 | 67 |

In another embodiment, Form 5 has an XRPD pattern with peaks at the positions listed in Table 126 below.

TABLE 126

| °2θ | d space (Å) | Intensity % (I/Io) |
|---|---|---|
| 4.6 ± 0.1 | 19.336 ± 0.432 | 23 |
| 8.3 ± 0.1 | 10.705 ± 0.131 | 15 |
| 9.2 ± 0.1 | 9.623 ± 0.106 | 57 |
| 10.8 ± 0.1 | 8.230 ± 0.077 | 18 |
| 11.5 ± 0.1 | 7.715 ± 0.068 | 19 |
| 12.6 ± 0.1 | 7.020 ± 0.056 | 46 |
| 12.7 ± 0.1 | 6.954 ± 0.055 | 23 |
| 13.4 ± 0.1 | 6.613 ± 0.050 | 20 |
| 14.0 ± 0.1 | 6.330 ± 0.045 | 22 |
| 14.9 ± 0.1 | 5.962 ± 0.040 | 25 |
| 15.6 ± 0.1 | 5.688 ± 0.037 | 30 |
| 16.0 ± 0.1 | 5.529 ± 0.034 | 66 |
| 16.8 ± 0.1 | 5.274 ± 0.031 | 44 |
| 18.0 ± 0.1 | 4.934 ± 0.027 | 37 |
| 18.2 ± 0.1 | 4.869 ± 0.027 | 67 |
| 18.7 ± 0.1 | 4.745 ± 0.025 | 17 |
| 19.7 ± 0.1 | 4.502 ± 0.023 | 38 |
| 20.0 ± 0.1 | 4.435 ± 0.022 | 24 |
| 21.1 ± 0.1 | 4.211 ± 0.020 | 28 |
| 21.6 ± 0.1 | 4.124 ± 0.019 | 49 |
| 21.8 ± 0.1 | 4.073 ± 0.019 | 39 |
| 22.2 ± 0.1 | 4.003 ± 0.018 | 29 |
| 23.7 ± 0.1 | 3.748 ± 0.016 | 30 |
| 24.4 ± 0.1 | 3.653 ± 0.015 | 36 |
| 24.7 ± 0.1 | 3.600 ± 0.014 | 77 |
| 25.2 ± 0.1 | 3.533 ± 0.014 | 45 |
| 26.6 ± 0.1 | 3.356 ± 0.012 | 100 |
| 27.5 ± 0.1 | 3.245 ± 0.012 | 24 |

In another embodiment, crystalline Form B of (R)-5-(2-Aminoethyl)-1-(6,8-difluorochroman-3-yl)-1,3-dihydroimidazole-2-thione hydrosulfate is characterised as having an XRPD pattern as shown in FIG. 63i.

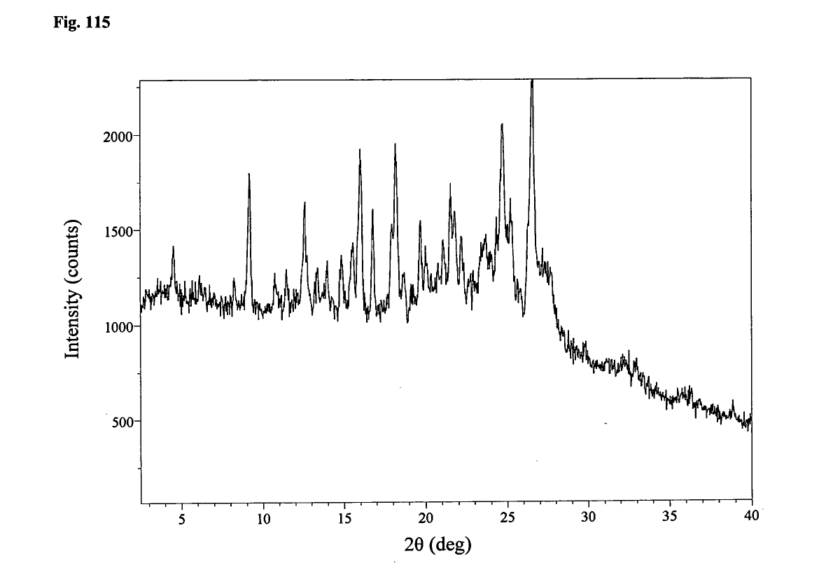

In an embodiment, Form B of (R)-5-(2-Aminoethyl)-1-(6,8-difluorochroman-3-yl)-1,3-dihydroimidazole-2-thione sulfate is characterised as having an XRPD pattern as shown in FIG. 115.

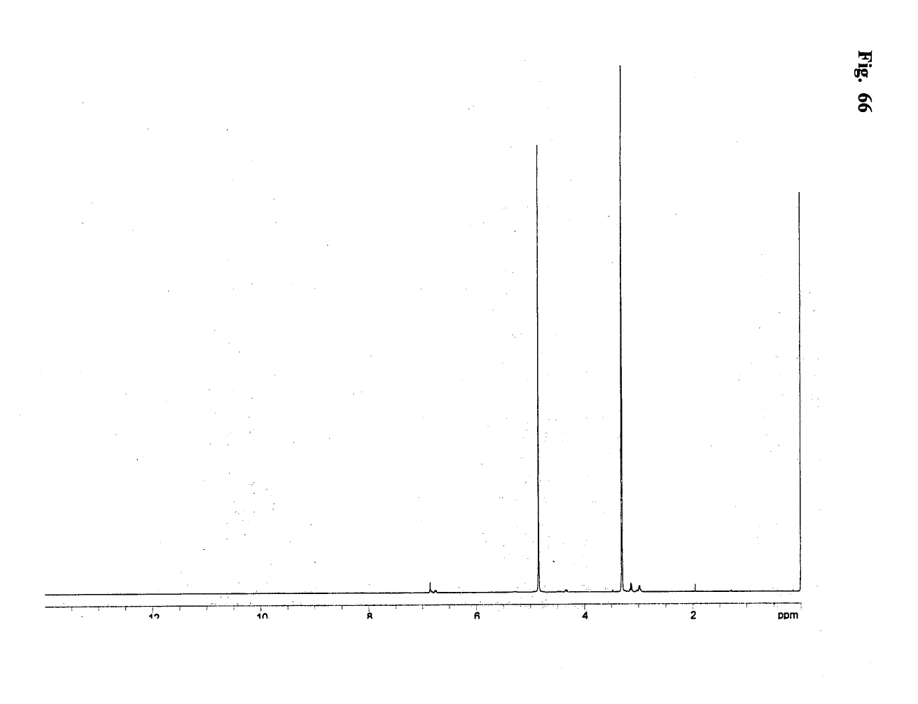

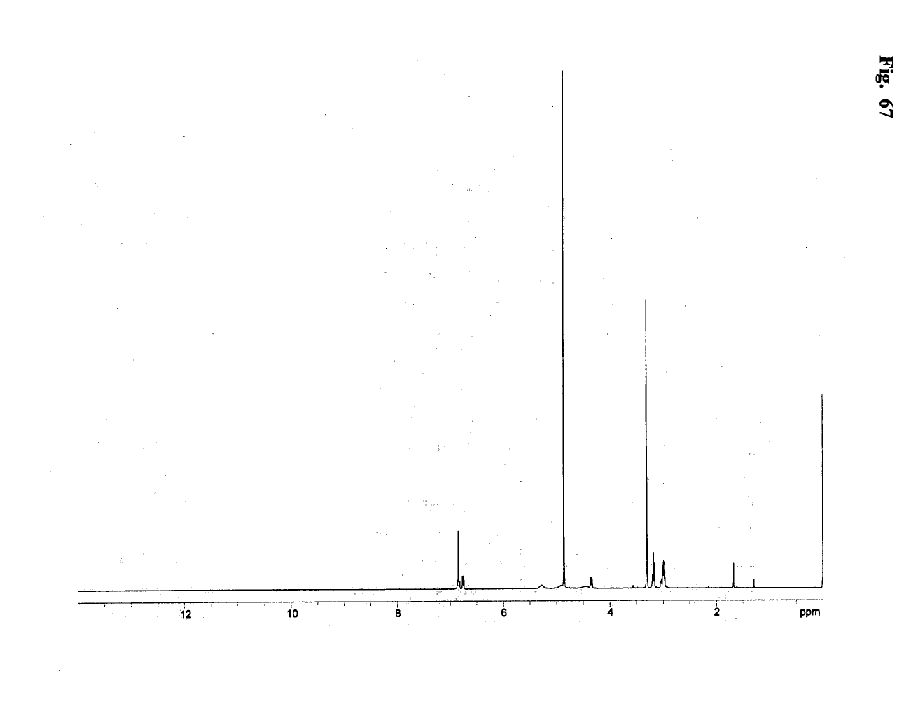

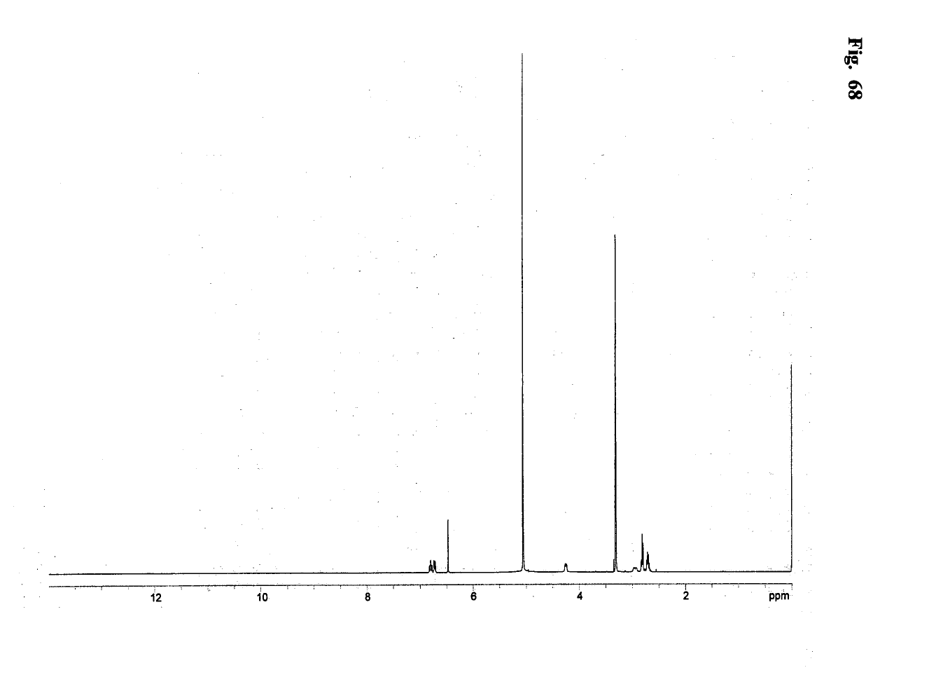

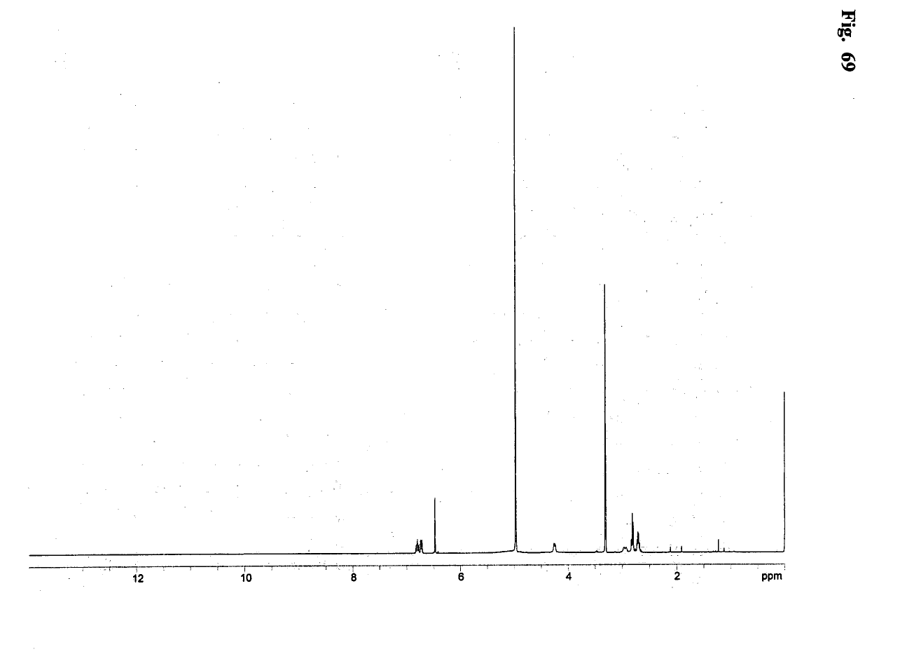

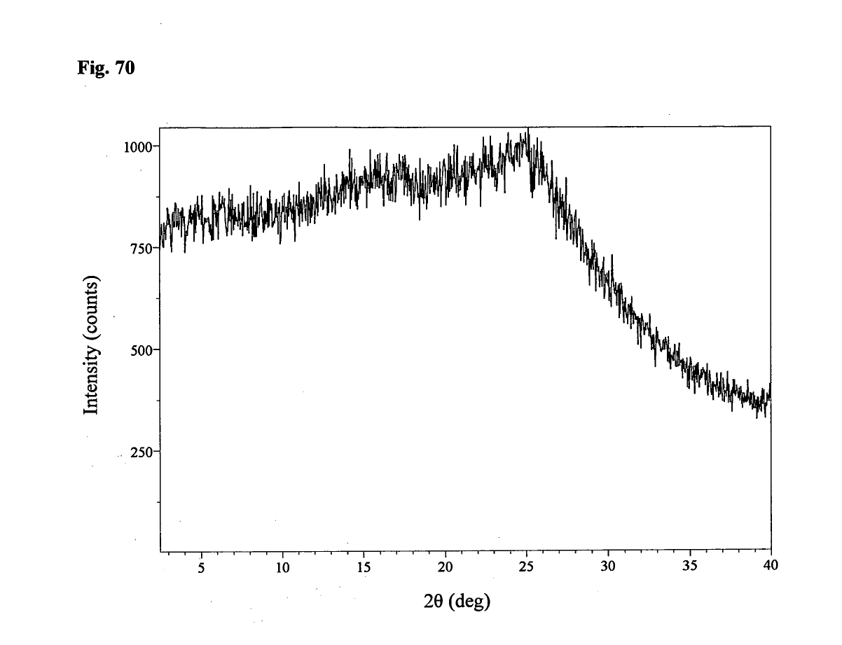

According to another aspect of the present invention, there is provided compound 2 in amorphous form, i.e. (R)-5-(2-Aminoethyl)-1-(6,8-difluorochroman-3-yl)-1,3-dihydroimidazole-2-thione in amorphous form. In an embodiment, the amorphous form of (R)-5-(2-Aminoethyl)-1-(6,8-difluorochroman-3-yl)-1,3-dihydroimidazole-2-thione is characterised as having an XRPD pattern as shown in FIG. 70.

According to another aspect of the present invention, there is provided processes for preparing the salts and polymorphs described above. Each of the processes detailed in the Experimental represent alternative embodiments of the processes of the present invention.

According to another aspect of the present invention, there is provided a pharmaceutical composition comprising a salt or polymorph as described above together with one or more pharmaceutical excipients. The pharmaceutical compositions may be as described in WO2004/033447.

In this specification, crystalline and low crystalline forms of the same polymorph are described. For example, the adipate salt exists in crystalline Form 1, as well as low crystalline Form 1. Forms having the same number but specified as being either crystalline or low crystalline refer to the same polymorph. Reasons for XRPD patterns showing the form as a low crystalline form are well known to those skilled in the art.

In this specification, the term “compound 2” refers to (R)-5-(2-Aminoethyl)-1-(6,8-difluorochroman-3-yl)-1,3-dihydroimidazole-2-thione free base.

Reference is made to the accompanying Figures, which show:

FIG. 1a XRPD pattern of L-tartrate

FIG. 1b XRPD pattern of Malonate

FIG. 1c XRPD pattern of Tosylate, Form A

FIG. 1d XRPD pattern of (1R)-10-Camphorsulfonate

FIG. 1e XRPD pattern of Fumarate

FIG. 2 DSC and TG data for malonate salt

FIG. 3a XRPD pattern of L-tartrate salt: Form A

FIG. 3b XRPD pattern of L-tartrate salt: Form B

FIG. 4 Proton NMR of tartrate salt, Form A

FIG. 5 Proton NMR of tartrate salt, Form B

FIG. 6a XRPD pattern of tosylate salt: Form A (same as FIG. 1c)

FIG. 6b XRPD pattern of tosylate salt: Form B

FIG. 6c XRPD pattern of tosylate salt: Form C

FIG. 6d XRPD pattern of tosylate salt: Form D

FIG. 6e XRPD pattern of tosylate salt: Form E

FIG. 6f XRPD pattern of tosylate salt: Form F (also called crystal modification X)

FIG. 6g XRPD pattern of tosylate salt: Form G

FIG. 6h XRPD pattern of tosylate salt: Form H (also called crystal modification Y)

FIG. 7 Proton NMR of tosylate salt, Form A

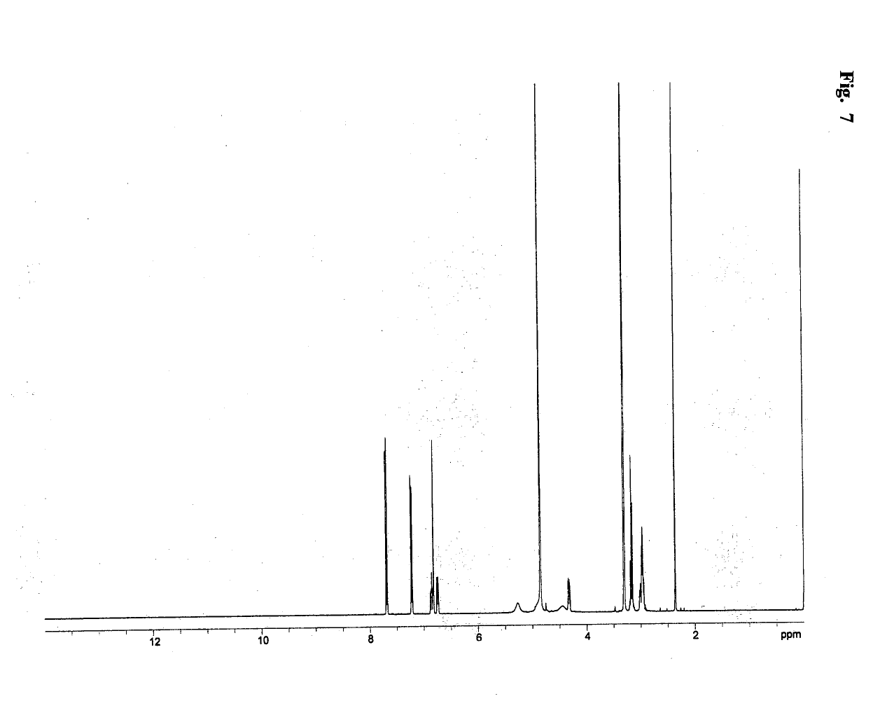

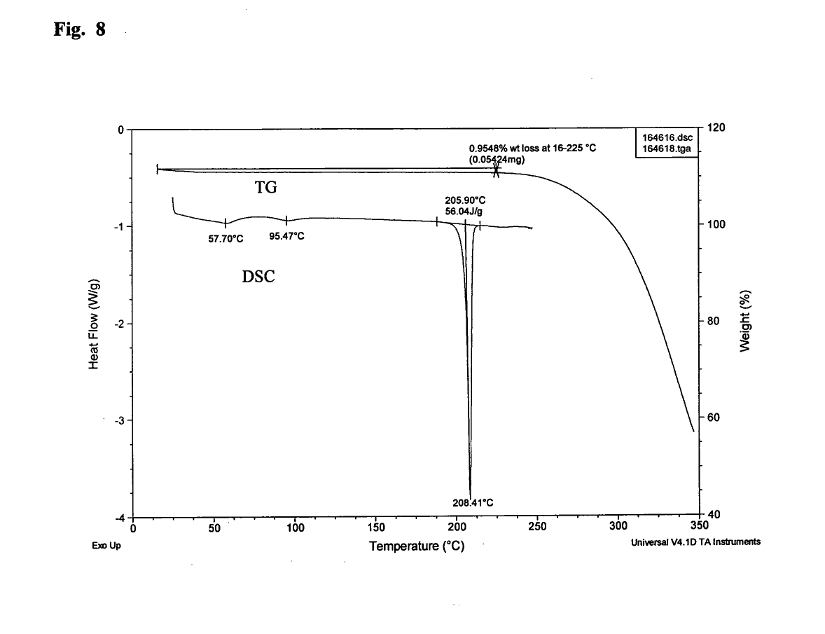

FIG. 8 DSC and TG data for the tosylate salt, Form A

FIG. 9 Proton NMR of tosylate salt, Form B

FIG. 10 DSC and TG data for tosylate salt, Form B

FIG. 11 Proton NMR of tosylate salt, Form C

FIG. 12 DSC and TG data for tosylate salt, Form C

FIG. 13 Proton NMR of tosylate salt, Form D

FIG. 14 Proton NMR of tosylate salt, Form E

FIG. 15 DSC and TG data for tosylate salt, Form E

FIG. 16 Proton NMR of tosylate salt, Form F (also called crystal modification X)

FIG. 17 DSC and TG data for tosylate salt, Form F

FIG. 18 Proton NMR of tosylate salt, Form G

FIG. 19 Proton NMR of tosylate salt, Form H (also called crystal modification Y)

FIG. 20 DSC and TG data for tosylate salt, Form H

FIG. 21a XRPD pattern of acetate salt: crystalline 1, scale-up

FIG. 21b XRPD pattern of acetate salt: crystalline 1, wellplate, well no. A3

FIG. 22 Proton NMR of acetate salt

FIG. 23 DSC and TG data for the acetate salt

FIG. 24a XRPD pattern of adipate salt: crystalline 1, scale-up

FIG. 24b XRPD pattern of adipate salt: crystalline 1, well plate, well no. B2

FIG. 24c XRPD pattern of adipate salt: low crystalline 1, well plate, well no. B1

FIG. 24d XRPD pattern of adipate salt: crystalline 1-peaks, well plate, well no. B6

FIG. 25 Proton NMR of adipate salt

FIG. 26 DSC and TG data for the adipate salt

FIG. 27a XRPD pattern of citrate salt: crystalline 1, scale-up

FIG. 27b XRPD pattern of citrate salt: crystalline 2, scale-up

FIG. 27c XRPD pattern of citrate salt: crystalline 1, well plate, well no. C3

FIG. 27d XRPD pattern of citrate salt: low crystalline 1, well plate, well no. C4

FIG. 28 Proton NMR of citrate salt, crystalline 1

FIG. 29 Proton NMR of citrate salt, crystalline 2

FIG. 30 Proton NMR of citrate salt, crystalline 2

FIG. 31 DSC and TG data for the citrate salt, crystalline 2

FIG. 32a XRPD pattern of gentisate salt: crystalline 1, scale-up

FIG. 32b XRPD pattern of gentisate salt: crystalline 1, well plate, well no. D5

FIG. 32c XRPD pattern of gentisate salt: crystalline 2, well plate, well no. D6

FIG. 33 Proton NMR of gentisate salt, crystalline 1

FIG. 34 Proton NMR of gentisate salt, crystalline 2

FIG. 35a XRPD pattern of glutarate salt: crystalline 1, scale-up

FIG. 35b XRPD pattern of glutarate salt: crystalline 1, well plate, well no. E1

FIG. 35c XRPD pattern of glutarate salt: low crystalline 1, well plate, well no. E3

FIG. 36 Proton NMR of glutarate salt

FIG. 37a XRPD pattern of glycolate salt: crystalline 1, scale-up

FIG. 37b XRPD pattern of glycolate salt: crystalline 1, well plate, well no. F1

FIG. 37c XRPD pattern of glycolate salt: low crystalline 1, well plate, well no. F2

FIG. 38 Proton NMR of glycolate salt

FIG. 39 DSC and TG data for the glycolate salt

FIG. 40a XRPD pattern of hydrobromide salt: crystalline 1, scale-up

FIG. 40b XRPD pattern of hydrobromide salt: crystalline 3, scale-up

FIG. 40c XRPD pattern of hydrobromide salt: crystalline 1, well plate, well no. All

FIG. 40d XRPD pattern of hydrobromide salt: crystalline 2, well plate, well no. A9

FIG. 40e XRPD pattern of hydrobromide salt: low crystalline 2, well plate, well no. A2

FIG. 41 Proton NMR of hydrobromide salt, crystalline 1

FIG. 42 Proton NMR of hydrobromide salt, crystalline 2

FIG. 43 Proton NMR of hydrobromide salt, crystalline 3

FIG. 44 DSC and TG data for the hydrobromide salt, crystalline 1

FIG. 45 XRPD pattern of lactate salt: crystalline 1, well plate, well no. B12

FIG. 46 Proton NMR of lactate salt

FIG. 47a XRPD pattern of L-malate salt: crystalline 1, scale-up

FIG. 47b XRPD pattern of L-malate salt: crystalline 1, well plate, well no. G6

FIG. 48 Proton NMR of L-malate salt

FIG. 49a XRPD pattern of maleate salt: crystalline 1+peaks, scale-up

FIG. 49b XRPD pattern of maleate salt: crystalline 1, well plate, well no. C5

FIG. 49c XRPD pattern of maleate salt: crystalline 1+one peak, well plate, well no. C11

FIG. 49d XRPD pattern of maleate salt: low crystalline 1, well plate, well no. C11

FIG. 50 Proton NMR of maleate salt

FIG. 51a XRPD pattern of phosphate salt: crystalline 1, well plate, well no. G11

FIG. 51b XRPD pattern of phosphate salt: crystalline 1+peaks, well plate, well no. G6

FIG. 51c XRPD pattern of phosphate salt: low crystalline 1, well plate, well no. G5

FIG. 51d XRPD pattern of phosphate salt: crystalline 2, wellplate, well no. G1

FIG. 51e XRPD pattern of phosphate salt: crystalline 3, wellplate, well no. G7

FIG. 51f XRPD pattern of phosphate salt: crystalline 4, wellplate, well no. G8

FIG. 51g XRPD pattern of phosphate salt: crystalline 5 (also called crystal modification X), scale-up

FIG. 51h XRPD pattern of phosphate salt: crystalline 6, scale-up

FIG. 51i XRPD pattern of phosphate salt: low crystalline 7, scale-up

FIG. 52 XRPD pattern of phosphate salt: crystalline 8, scale-up

FIG. 53 Proton NMR of phosphate salt, crystalline 2

FIG. 54 Proton NMR of phosphate salt, crystalline 3

FIG. 55 Proton NMR of phosphate salt; crystalline 4

FIG. 56 Proton NMR of phosphate salt, crystalline 5 (also called crystal modification X)

FIG. 57 Proton NMR data for the phosphate salt, crystalline 8

FIG. 58 DSC and TG data for the phosphate salt, crystalline 8

FIG. 59 XRPD patterns of succinate salt (top to bottom)

FIG. 60 Proton NMR of succinate salt, crystalline 1

FIG. 61 Proton NMR of succinate salt, crystalline 2

FIG. 62 Proton NMR of succinate salt, crystalline 3

FIG. 63a XRPD pattern of sulfate salt: crystalline 1, well plate, well no. F2

FIG. 63b XRPD pattern of sulfate salt: low crystalline 1, well plate 95730, well no. F4

FIG. 63d XRPD pattern of sulfate salt: crystal modification X (also referred to as crystalline 2), well plate 95730, well no. F6

FIG. 63e XRPD pattern of hydrosulfate salt: Form A (also referred to as crystalline 2 minus peaks), well plate 96343, well no. F6

FIG. 63f XRPD pattern of sulfate salt: crystalline 3, well plate, well no. F1

FIG. 63g XRPD pattern of sulfate salt: crystal modification Y (also referred to as crystalline 4), well plate, well no. F5

FIG. 63h XRPD pattern of sulfate salt: crystalline 1, scale-up

FIG. 63i XRPD pattern of hydrosulfate salt: Form B (also referred to as crystalline 5), scale-up

FIG. 63j XRPD pattern of sulfate salt: crystalline 6, scale-up

FIG. 63k XRPD pattern of sulfate salt: crystalline 7, scale-up

FIG. 631 XRPD pattern of sulfate salt: low crystalline 8, scale-up

FIG. 64 Proton NMR of sulfate salt, crystalline 1

FIG. 65 DSC and TG data for sulfate salt, crystalline 1

FIG. 66 Proton NMR of hydrosulfate salt, Form A (also referred to as crystalline 2 minus peaks)

FIG. 67 Proton NMR of hydrosulfate salt, Form B (also referred to as crystalline 5)

FIG. 68 Proton NMR of sulfate salt, crystalline 6

FIG. 69 Proton NMR of sulfate salt, crystalline 7

FIG. 70 XRPD pattern of amorphous form of compound 2

FIG. 71 XRPD pattern of Form A of (R)-5-(2-Aminoethyl)-1-(6,8-difluorochroman-3-yl)-1,3-dihydroimidazole-2-thione L-tartrate.

FIG. 72 XRPD pattern of Form B of (R)-5-(2-Aminoethyl)-1-(6,8-difluorochroman-3-yl)-1,3-dihydroimidazole-2-thione L-tartrate

FIG. 73 XRPD pattern of Form A of (R)-5-(2-Aminoethyl)-1-(6,8-difluorochroman-3-yl)-1,3-dihydroimidazole-2-thione malonate

FIG. 74 XRPD pattern of Form A of (R)-5-(2-Aminoethyl)-1-(6,8-difluorochroman-3-yl)-1,3-dihydroimidazole-2-thione camsylate

FIG. 75 XRPD pattern of Form A of (R)-5-(2-Aminoethyl)-1-(6,8-difluorochroman-3-yl)-1,3-dihydroimidazole-2-thione fumarate FIG. 76 XRPD pattern of Form A of (R)-5-(2-Aminoethyl)-1-(6,8-difluorochroman-3-yl)-1,3-dihydroimidazole-2-thione tosylate

FIG. 77 XRPD pattern of Form B of (R)-5-(2-Aminoethyl)-1-(6,8-difluorochroman-3-yl)-1,3-dihydroimidazole-2-thione tosylate

FIG. 78 XRPD pattern of Form C of (R)-5-(2-Aminoethyl)-1-(6,8-difluorochroman-3-yl)-1,3-dihydroimidazole-2-thione tosylate

FIG. 79 XRPD pattern of Form E of (R)-5-(2-Aminoethyl)-1-(6,8-difluorochroman-3-yl)-1,3-dihydroimidazole-2-thione tosylate

FIG. 80 XRPD pattern of crystal modification X of (R)-5-(2-Aminoethyl)-1-(6,8-difluorochroman-3-yl)-1,3-dihydroimidazole-2-thione tosylate

FIG. 81 XRPD pattern of Form G of (R)-5-(2-Aminoethyl)-1-(6,8-difluorochroman-3-yl)-1,3-dihydroimidazole-2-thione tosylate

FIG. 82 XRPD pattern of crystal modification Y of (R)-5-(2-Aminoethyl)-1-(6,8-difluorochroman-3-yl)-1,3-dihydroimidazole-2-thione tosylate

FIG. 83 XRPD pattern of Form 1 of (R)-5-(2-Aminoethyl)-1-(6,8-difluorochroman-3-yl)-1,3-dihydroimidazole-2-thione acetate

FIG. 84 XRPD pattern of Form 1 of (R)-5-(2-Aminoethyl)-1-(6,8-difluorochroman-3-yl)-1,3-dihydroimidazole-2-thione adipate FIG. 85 XRPD pattern of Form 1 of (R)-5-(2-Aminoethyl)-1-(6,8-difluorochroman-3-yl)-1,3-dihydroimidazole-2-thione glutarate FIG. 86 XRPD pattern of Form 1 of (R)-5-(2-Aminoethyl)-1-(6,8-difluorochroman-3-yl)-1,3-dihydroimidazole-2-thione succinate

FIG. 87 XRPD pattern of Form 2 of (R)-5-(2-Aminoethyl)-1-(6,8-difluorochroman-3-yl)-1,3-dihydroimidazole-2-thione succinate

FIG. 88 XRPD pattern of Form 3 of (R)-5-(2-Aminoethyl)-1-(6,8-difluorochroman-3-yl)-1,3-dihydroimidazole-2-thione succinate

FIG. 89 XRPD pattern of Form 1 of (R)-5-(2-Aminoethyl)-1-(6,8-difluorochroman-3-yl)-1,3-dihydroimidazole-2-thione hydrobromide

FIG. 90 XRPD pattern of Form 2 of (R)-5-(2-Aminoethyl)-1-(6,8-difluorochroman-3-yl)-1,3-dihydroimidazole-2-thione hydrobromide

FIG. 91 XRPD pattern of Form 3 of (R)-5-(2-Aminoethyl)-1-(6,8-difluorochroman-3-yl)-1,3-dihydroimidazole-2-thione hydrobromide

FIG. 92 XRPD pattern of Form 1 of (R)-5-(2-Aminoethyl)-1-(6,8-difluorochroman-3-yl)-1,3-dihydroimidazole-2-thione maleate

FIG. 93 XRPD pattern of Form 2 of (R)-5-(2-Aminoethyl)-1-(6,8-difluorochroman-3-yl)-1,3-dihydroimidazole-2-thione maleate

FIG. 94 XRPD pattern of Form 1 of (R)-5-(2-Aminoethyl)-1-(6,8-difluorochroman-3-yl)-1,3-dihydroimidazole-2-thione phosphate

FIG. 95 XRPD pattern of Form 2 of (R)-5-(2-Aminoethyl)-1-(6,8-difluorochroman-3-yl)-1,3-dihydroimidazole-2-thione phosphate

FIG. 96 XRPD pattern of Form 3 of (R)-5-(2-Aminoethyl)-1-(6,8-difluorochroman-3-yl)-1,3-dihydroimidazole-2-thione phosphate

FIG. 97 XRPD pattern of Form 4 of (R)-5-(2-Aminoethyl)-1-(6,8-difluorochroman-3-yl)-1,3-dihydroimidazole-2-thione phosphate

FIG. 98 XRPD pattern of crystal modification X of (R)-5-(2-Aminoethyl)-1-(6,8-difluorochroman-3-yl)-1,3-dihydroimidazole-2-thione phosphate

FIG. 99. XRPD pattern of Form 6 of (R)-5-(2-Aminoethyl)-1-(6,8-difluorochroman-3-yl)-1,3-dihydroimidazole-2-thione phosphate

FIG. 100 XRPD pattern of Form 7 of (R)-5-(2-Aminoethyl)-1-(6,8-difluorochroman-3-yl)-1,3-dihydroimidazole-2-thione phosphate

FIG. 101 XRPD pattern of Form 8 of (R)-5-(2-Aminoethyl)-1-(6,8-difluorochroman-3-yl)-1,3-dihydroimidazole-2-thione phosphate

FIG. 102 XRPD pattern of Form 1 of (R)-5-(2-Aminoethyl)-1-(6,8-difluorochroman-3-yl)-1,3-dihydroimidazole-2-thione gentisate

FIG. 103 XRPD pattern of Form 2 of (R)-5-(2-Aminoethyl)-1-(6,8-difluorochroman-3-yl)-1,3-dihydroimidazole-2-thione gentisate

FIG. 104 XRPD pattern of Form 1 of (R)-5-(2-Aminoethyl)-1-(6,8-difluorochroman-3-yl)-1,3-dihydroimidazole-2-thione citrate

FIG. 105 XRPD pattern of Form 2 of (R)-5-(2-Aminoethyl)-1-(6,8-difluorochroman-3-yl)-1,3-dihydroimidazole-2-thione citrate

FIG. 106 XRPD pattern of Form 1 of (R)-5-(2-Aminoethyl)-1-(6,8-difluorochroman-3-yl)-1,3-dihydroimidazole-2-thione malate

FIG. 107 XRPD pattern of Form 1 of (R)-5-(2-Aminoethyl)-1-(6,8-difluorochroman-3-yl)-1,3-dihydroimidazole-2-thione glycolate

FIG. 108 XRPD pattern of Form 1 of (R)-5-(2-Aminoethyl)-1-(6,8-difluorochroman-3-yl)-1,3-dihydroimidazole-2-thione sulfate

FIG. 109 XRPD pattern of crystal modification X of (R)-5-(2-Aminoethyl)-1-(6,8-difluorochroman-3-yl)-1,3-dihydroimidazole-2-thione sulfate

FIG. 110 XRPD pattern of Form 3 of (R)-5-(2-Aminoethyl)-1-(6,8-difluorochroman-3-yl)-1,3-dihydroimidazole-2-thione sulfate

FIG. 111 XRPD pattern of crystal modification Y of (R)-5-(2-Aminoethyl)-1-(6,8-difluorochroman-3-yl)-1,3-dihydroimidazole-2-thione sulfate

FIG. 112 XRPD pattern of Form 6 of (R)-5-(2-Aminoethyl)-1-(6,8-difluorochroman-3-yl)-1,3-dihydroimidazole-2-thione sulfate

FIG. 113 XRPD pattern of Form 7 of (R)-5-(2-Aminoethyl)-1-(6,8-difluorochroman-3-yl)-1,3-dihydroimidazole-2-thione sulfate

FIG. 114 XRPD pattern of Form 8 of (R)-5-(2-Aminoethyl)-1-(6,8-difluorochroman-3-yl)-1,3-dihydroimidazole-2-thione sulfate

FIG. 115 XRPD pattern of Form B of (R)-5-(2-Aminoethyl)-1-(6,8-difluorochroman-3-yl)-1,3-dihydroimidazole-2-thione hydrosulfate

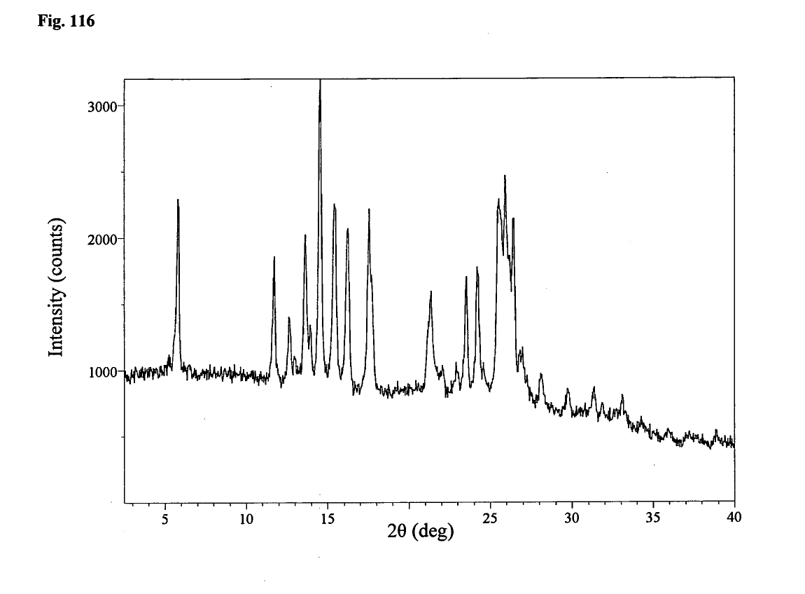

FIG. 116 XRPD pattern of compound 2

### EXPERIMENTAL DETAILS

A salt and polymorph screen was undertaken which involved various crystallisation techniques, as explained below.

1. Solvent-Based Crystallization Techniques

a. Fast Evaporation (FE)

Solutions of compound 2 were prepared in various solvents in which samples were vortexed or sonicated between aliquot additions. Once a mixture reached complete dissolution, as judged by visual observation, the solution was filtered through a 0.2-μm nylon filter. The filtered solution was allowed to evaporate at ambient conditions in an open vial. The solids were isolated and analyzed.

b. Slow Evaporation (SE)

Solutions of compound 2 were prepared in various solvents in which samples were vortexed or sonicated between aliquot additions. Once a mixture reached complete dissolution, as judged by visual observation, the solution was filtered through a 0.2-μm nylon filter. The filtered solution was allowed to evaporate at ambient conditions in a vial covered with a loose cap or perforated aluminum foil. The solids were isolated and analyzed.

c. Slurry Experiments

Solutions of compound 2 were prepared by adding enough solids to a given solvent at ambient conditions so that undissolved solids were present. The mixture was then loaded on a rotary wheel or an orbit shaker in a sealed vial at either ambient or elevated temperature for a certain period of time, typically 7 days. The solids were isolated by vacuum filtration or by drawing off or decanting the liquid phase and allowing the solids to air dry at ambient conditions prior to analysis.

d. Crash Precipiation

Solutions of compound 2 were prepared in various solvents in which samples were agitated or sonicated to facilitate dissolution. The resulting solutions (sometimes filtered) were transferred into vials containing a known volume of antisolvent and/or aliquots of antisolvent were added to the solutions until precipitation persisted. If precipitation was insufficient, some samples were left at ambient temperature. The solids were isolated by decanting the liquid phase and allowing the solids to air dry at ambient conditions prior to analysis.

e. Slow Cool

Solutions of compound 2 were prepared in various solvents in which samples were heated with agitation to facilitate dissolution. The solutions were cooled by shutting off the heat source. If precipitation was insufficient, samples were refrigerated or evaporated. The solids were isolated by vacuum filtration.

2. Well Plate Crystallization Techniques

a. Wellplate Salt Preparations

Preparation of salts was carried out in 96-well polypropylene plates using the following general procedure. API solutions were prepared by dissolving compound 2 free base in acetone, methanol, methyl ethyl ketone, tetrahydrofuran or 2,2,2-trifluoroethanol at approximately 10 mg/mL, adding 0.1 mL of these solutions per well. Dilute acid solutions were added (methanol solutions, generally 0.1M) to the wells at slightly more than one molar equivalent with respect to the API. Each API/acid combination was prepared in triplicate and wells with only the API solutions: were also prepared for comparison. The plates were covered with a self-adhesive aluminum foil cover and allowed to mix at approximately 25 RPM on an ambient-temperature orbital shaker for 8 or 11 days. Some evaporation occurred during mixing. The plates were observed after 3 days by optical microscopy and returned to the shaker. Upon removal from the shaker, they were observed visually for color under standard laboratory lighting. The plates were left uncovered to complete evaporation under ambient conditions for final microscopic evaluation and XRPD analysis.

b. General Salt Preparation procedure

To a glass vial of compound 2 dissolved in various solvents, slightly more than one molar equivalent of various counterion solutions were added. Samples were allowed to slurry and/or evaporate at ambient temperature in a laboratory fume hood. Often, antisolvent was added to precipitate solids. The resulting solids were isolated by filtration or solvent decantation (often preceded by centrifugation), examined by polarized light microscopy and generally submitted for XRPD analysis.

c. Fast Evaporation

A well plate containing various solutions was allowed to stand, uncovered, at ambient conditions to allow the solutions to evaporate. The solids were analyzed in the well plate.

d. Recrystallization Techniques

Solutions were prepared by dispensing 75 μL of methanol into each well of a well plate containing solids from previous experiments. The well plate was then covered and attached to an orbit shaker for 30 minutes to 1 hour. An equal volume (75 μL) of various antisolvents was added to each well, and the solutions were allowed to fast evaporate at ambient conditions. The solids were analyzed in the well plate.

Instrumental Techniques

The characterisation of the polymorphs involved various analytical techniques, as explained below.

A. X-Ray Powder Diffraction (XRPD)

Shimadzu XRD-6000 Diffractometer

Analyses were carried out on a Shimadzu XRD-6000 X-ray powder diffractometer using Cu Kα radiation. The instrument is equipped with a long fine focus X-ray tube. The tube voltage and amperage were set at 40 kV and 40 mA, respectively. The divergence and scattering slits were set at 1° and the receiving slit was set at 0.15 mm. Diffracted radiation was detected by a NaI scintillation detector. A theta-two theta continuous scan at 3°/min (0.4 sec/0.02° step) from 2.5 to 40 °2θ was used. A silicon standard was analyzed each day to check the instrument alignment. Samples were analyzed in an aluminum sample holder with a silicon well.

Inel XRG-3000 Diffractometer

X-ray powder diffraction (XRPD) analyses were performed using an Inel XRG-3000 diffractometer equipped with a CPS (Curved Position Sensitive) detector with a 20 range of 120°. Real time data were collected using Cu-Kα radiation starting at approximately 4 °2θ at a resolution of 0.03 °2θ. The tube voltage and amperage were set to 40 kV and 30 mA, respectively. The monochromator slit was set at 5 mm by 160 μm. The pattern is displayed from 2.5-40 °2. Samples were prepared for analysis by packing them into thin-walled glass capillaries. Each capillary was mounted onto a goniometer head that is motorized to permit spinning of the capillary during data acquisition. The samples were analyzed for 5 or 10 min. Instrument calibration was performed using a silicon reference standard.

Bruker D-8 Discover Diffractometer

XRPD patterns were collected with a Bruker D-8 Discover diffractometer and Bruker's General Area Diffraction Detection System (GADDS, v. 4.1.20). An incident beam of Cu Kα radiation was produced using a fine-focus tube (40 kV, 40 mA), a Göbel mirror, and a 0.5 mm double-pinhole collimator. The samples were positioned for analysis by securing the well plate to a translation stage and moving each sample to intersect the incident beam. The samples were analyzed using a transmission geometry. The incident beam was scanned and rastered over the sample during the analysis to optimize orientation statistics. A beam-stop was used to minimize air scatter from the incident beam at low angles. Diffraction patterns were collected using a Hi-Star area detector located 15 cm from the sample and processed using GADDS. The intensity in the GADDS image of the diffraction pattern was integrated using a step size of 0.04 °2θ. The integrated patterns display diffraction intensity as a function of 2θ. Prior to the analysis a silicon standard was analyzed to verify the Si 111 peak position. The instrument was operated under non-cGMP conditions, and the results are non-cGMP.

PatternMatch 2.4.0 software, combined with visual inspection, was used to identify peak positions for each form. “Peak position” means the maximum intensity of a peaked intensity profile. Where data collected on the INEL diffractometer was used, it was first background-corrected using PatternMatch 2.4.0.

PatternMatch 2.4.0 was used for all peak identification. Peak positions were reproducible to within 0.1 °2θ. Therefore, all peak positions reported in tables used this precision as indicated by the number following the ± in the 2θ column. All peak positions have been converted to (wavelength-independent) d space using a wavelength of 1.541874 Å and the precision at each position is indicated as well (note that the precision is not constant in d space). It will be noted that the precision of within 0.1 °2θ was used to determine reproducability of peak positions. It will be appreciated that peak positions may vary to a small extent depending on which apparatus is used to analyse a sample. Therefore, all definitions of the polymorphs which refer to peak positions at °2θ values are understood to be subject to variation of ±0.2 °2θ. Unless otherwise stated (for example in the Tables with ±values), the °2θ values of the peak positions are ±0.2 °2θ.

B. Differential Scanning Calorimetry (DSC)

Differential scanning calorimetry (DSC) was performed using a TA Instruments differential scanning calorimeter 2920 and Q1000. The sample was placed into an aluminum DSC pan, and the weight accurately recorded. The pan was covered with a lid and then crimped or non-crimped pan configuration was used. The sample cell was equilibrated at 25° C. and heated under a nitrogen purge at a rate of 10° C./min, up to a final temperature of 250, or 300° C. Indium metal was used as the calibration standard. Reported temperatures are at the transition maxima.

C. Thermogravimetry (TG)

Thermogravimetric (TG) analyses were performed using a TA Instruments 2950 thermogravimetric analyzer. Each sample was placed in an aluminum sample pan and inserted into the TG furnace. The furnace was either equilibrated at 25° C. or directly heated under nitrogen at a rate of 10° C./min, up to a final temperature of 350° C. Nickel and Alumel™ were used as the calibration standards.

D. NMR Spectroscopy

Solution 1D ¹H NMR Spectroscopy

Solution ¹H NMR spectra were acquired at ambient temperature with a Varian ^{UNITY}INOVA-400 spectrometer at a ¹H Larmor frequency of 399.795 MHz. The sample was dissolved in MeOH-d₄. The spectrum was acquired with a ¹H pulse width of 8.2, 8.4, 8.5 or 10 μs, a 2.50 second acquisition time, a 5 second delay between scans, a spectral width of 6400 Hz with 32000 data points, and 40 co-added scans. The free induction decay (FID) was processed using Varian VNMR 6.1C software with 32000 points. The residual peak from incompletely deuterated methanol is at approximately 3.3 ppm. The relatively broad peak at approximately 4.88 ppm is due to water. The spectrum was referenced to internal tetramethylsilane (TMS) at 0.0 ppm.

Solution 1D ¹H NMR Spectroscopy (SDS, Inc.)

The solution ¹H NMR spectrum was acquired by Spectral Data Services of Champaign, Ill. at 25° C. with a Varian ^{UNITY}INOVA-400 spectrometer at a ¹H Larmor frequency of 399.798 MHz. The sample was dissolved in methanol-d₄. The spectrum was acquired with a ¹H pulse width of 7.0 μs, a 5 second delay between scans, a spectral width of 7000 Hz with 35K data points, and 40 co-added scans. The free induction decay (FID) was processed with 64K points and an exponential line broadening factor of 0.2 Hz to improve the signal-to-noise ratio. The residual peak from incompletely deuterated methanol is at approximately 3.3 ppm.

Results—Solvent-Based Crystallization Screen

Camsylate Salt

The initial lot of the camsylate salt was prepared as follows.

To a suspension of compound 2 (0.93 g, 3 mmol) in MeOH (20 ml) was added a solution of (1R)-(−)-camphorsulfonic acid (0.70 g, 3 mmol) in MeOH (5 ml) at 50° C. with stirring. The mixture was heated to reflux, allowed to cool naturally to 20-25° C. with stirring, aged at 20-25° C. for 2 h. The precipitate was collected, washed with MeOH (10 ml), dried in vacuum at 45° C. to a constant weight. Yield 1.39 g (85%).

A polymorph screen was carried out on the (1R)-10-camphorsulfonate salt (camsylate salt) of compound 2 using slurry and slow evaporation experiments (Table 1A). The XRPD pattern of the camsylate salt is shown in FIG. 1d. No other forms were found in the screen.

TABLE 1A
Polymorph Screen of (1R)-10-Camphorsulfonate salt

| Solvent | Conditions^{a} | XRPD Result |
|---|---|---|
| acetone | slurry | camsylate |
| acetonitrile | slurry | camsylate |
| 1,4-dioxane | slurry | camsylate |
| ethanol | slurry | camsylate |
| ethyl acetate | slurry | camsylate |
| iso-propanol | slurry | camsylate |
| methanol | SE | camsylate |
| methyl ethyl ketone | slurry | camsylate |
| tetrahydrofuran (THF) | slurry | camsylate |
| toluene | slurry | camsylate |
| 2,2,2-trifluoroethanol | SE | camsylate |
| water | slurry | camsylate |

^{a}SE = slow evaporation

Fumarate Salt

The initial lot of the fumarate salt was prepared as follows.

Compound 2 (0.93 g, 3 mmol) was dissolved in a mixture of MeOH (20 ml) and DCM (5 ml) with heating to 40-45° C. and stirring. To the resulting clear solution fumaric acid (0.35 g, 3 mmol) in MeOH (10 ml) was added, the mixture was allowed to cool naturally to 20-25° C. with stirring (crystallisation occurred). The mixture was aged in ice for 1 h, the precipitate was collected, washed with MeOH (5 ml), dried in vacuum at 45° C. to a constant weight. Yield 0.82 g (74%).

A polymorph screen was carried out on the fumarate salt of compound 2 using slurry and fast evaporation experiments (Table 2A). The XRPD pattern of the fumarate salt is shown in FIG. 1e. No other forms were found in the screen.

TABLE 2A
Fumarate salt

| Solvent | Conditions^{a} | Habit/Description | XRPD Result^{b} |
|---|---|---|---|
| acetone | slurry, | white, morphology unknown, | fumarate |
| | 7 days | birefringent | |
| | FE (liquid phase | yellow plates and needles, | fumarate |
| | from slurry) | birefringent | |
| acetonitrile | slurry, | white, morphology unknown, | fumarate |
| | 7 days | birefringent | |
| | FE (liquid phase | clear glassy film, not | — |
| | from slurry) | birefringent | |
| 1,4-dioxane | slurry, | white plates, birefringent | fumarate |
| | 7 days | | |
| | FE (liquid phase | clear glassy film, not | — |
| | from slurry) | birefringent | |
| ethanol | slurry, | white, morphology unknown, | fumarate |
| | 7 days | birefringent | |
| | FE (liquid phase | light yellow needles and | — |
| | from slurry) | blades, birefringent | |
| ethyl acetate | slurry, | white, morphology unknown, | fumarate |
| | 7 days | birefringent | |
| | FE (liquid phase | clear, morphology unknown, | — |
| | from slurry) | birefringent | |
| iso-propanol | slurry, | white, morphology unknown, | fumarate |
| | 7 days | birefringent | |
| | FE (liquid phase | clear needles, birefringent; | — |
| | from slurry) | clear glassy film, not | |
| | | birefringent | |
| methanol | slurry, | white, morphology unknown, | fumarate |
| | 7 days | birefringent | |
| | FE (liquid phase | yellow plates and morphology | fumarate |
| | from slurry) | unknown, birefringent | |
| methyl ethyl ketone | slurry, | white, morphology unknown, | fumarate |
| | 7 days | birefringent | |
| | FE (liquid phase | clear fibers and morphology | — |
| | from slurry) | unknown, birefringent | |
| tetrahydrofuran | slurry, | white plates and morphology | fumarate |
| (THF) | 7 days | unknown, birefringent | |
| | FE (liquid phase | clear fibers, birefringent | — |
| | from slurry) | | |
| toluene | slurry, | white, morphology unknown, | fumarate |
| | 7 days | birefringent | |
| | FE (liquid phase | clear fibers, birefringent | — |
| | from slurry) | | |
| 2,2,2- | slurry, | white, morphology unknown, | fumarate, l.c. |
| trifluoroethanol | 7 days | birefringent | |
| | FE (liquid phase | white, morphology unknown, | fumarate |
| | from slurry) | birefringent | |
| water | FE | white, dendridic formations, | fumarate |
| | | birefringent | |

^{a}FE = fast evaporation
^{b}l.c. = low crystallinity

Malonate Salt

The initial lot of the malonate salt was prepared as follows.

To a suspension of compound 2 (0.93 g, 3 mmol) in MeOH (10 ml) was added a solution of malonic acid (0.31 g, 3 mmol) in MeOH (5 ml) at 50° C. with stirring. The mixture was heated to reflux to obtain a clear solution, allowed to cool naturally to 20-25° C. with stirring (crystallisation occurred), aged in ice for 30 min. The precipitate was collected, washed with MeOH (3 ml), dried in vacuum at 45° C. to a constant weight. Yield 1.12 g (90%).

A polymorph screen of the malonate salt was carried out using slurry and fast evaporation crystallization techniques (Table 3A). The XRPD pattern of the initial lot of the malonate salt is shown in FIG. 1b. No new forms were found in the abbreviated polymorph screen.

TABLE 3A
Polymorph Screen of Malonate Salt

| Solvent | Conditions^{a} | Habit/Description | XRPD Result |
|---|---|---|---|
| acetone | slurry, | clear solution | — |
| | 7 days | | |
| | FE | yellow, morphology | malonate |
| | | unknown, partially | |
| | | birefringent | |
| acetonitrile | slurry, | white, morphology unknown, | malonate |
| | 7 days | birefringent | |
| | FE (liquid phase | white needles and blades, | — |
| | from slurry) | birefringent | |
| 1,4-dioxane | slurry, | white, morphology unknown, | malonate |
| | 7 days | birefringent | |
| | FE (liquid phase | clear glassy film, not | — |
| | from slurry) | birefringent | |
| ethanol | slurry, | white, morphology unknown, | malonate |
| | 7 days | birefringent | |
| | FE (liquid phase | white, morphology unknown, | malonate |
| | from slurry) | partially birefringent | |
| ethyl | slurry, | white, morphology unknown, | malonate |
| acetate | 7 days | birefringent | |
| | FE (liquid phase | clear oily film, not | — |
| | from slurry) | birefringent | |
| iso- | slurry, | white, morphology unknown, | malonate |
| propanol | 7 days | birefringent | |
| | FE (liquid phase | translucent glassy film, not | — |
| | from slurry) | birefringent; white, | |
| | | morphology unknown, | |
| | | birefringent | |
| methanol | FE | white, morphology unknown, | malonate |
| | | birefringent | |
| methyl | slurry, | white, morphology unknown, | malonate |
| ethyl ketone | 7 days | birefringent | |
| | FE (liquid phase | yellow oily film, not | — |
| | from slurry) | birefringent | |
| tetrahydrofuran | slurry, | clear glassy film, not | amorphous + peaks from |
| (THF) | 7 days | birefringent; clear plates, | malonate |
| | | birefringent | |
| | FE (liquid phase | clear fibers, birefringent | — |
| | from slurry) | | |
| toluene | slurry, | white, morphology unknown, | malonate |
| | 7 days | birefringent | |
| | FE (liquid phase | white fibers, birefringent | — |
| | from slurry) | | |
| 2,2,2- | FE | white fibers, birefringent | malonate |
| trifluoroethanol | | | |
| water | FE | white blades, birefringent | malonate |

^{a}FE = fast evaporation

The malonate salt was characterized using thermal techniques (Table 4A, FIG. 2). A weight loss of approximately 0.3% was observed in the range of 16 to 180° C. A sharp endotherm at approximately 201° C. in DSC accompanied by approx. 25% weight loss was probably due to simultaneous melt/decomposition.

TABLE 4A
Characterization of Malonate Salt

| Technique | Analysis/Result |
|---|---|
| XRPD | A |
| DSC^{a} | endo 201 (266 J/g) |
| TGA^{b} | 0.30 @ 16-180 |
| | 24.95 @ 180-215 |

a. endo=endotherm, temperatures (C°) reported are transition maxima. Temperatures are rounded to the nearest degree.
b. weight loss (%) at a certain temperature; weight changes (%) are rounded to 2 decimal places; temperatures are rounded to the nearest degree

L-Tartrate Salt

The initial lot of the L-tartrate salt was prepared as follows.

Compound 2 (0.93 g, 3 mmol) was dissolved in a mixture of MeOH (20 ml) and DCM (5 ml) with heating to 40-45° C. and stirring. To the resulting clear solution L-tartaric acid (0.45 g, 3 mmol) in MeOH (10 ml) was added, the solution was concentrated under reduced pressure to half of the initial volume and diluted with 2-propanol (20 ml) (crystallisation occurred). The suspension was cooled in ice to 0-5° C., aged for 30 min, the precipitate was collected, washed with 2-propanol (5 ml), dried in vacuum at 45° C. to a constant weight. Yield 1.08 g (78%).

A polymorph screen of the L-tartrate salt was carried out using slurry and fast evaporation crystallization techniques (Table 5A). The XRPD pattern of the initial lot of the L-tartrate salt exhibited an amorphous character (FIG. 1a).

TABLE 5A
Polymorph Screen L-Tartrate Salt

| Solvent | Conditions^{a} | Habit/Description | XRPD Result^{b} |
|---|---|---|---|
| acetone | FE | white and yellow, | amorphous |
| | | morphology unknown, | |
| | | partially birefringent | |
| acetonitrile | slurry, | white, morphology unknown, | low crystalline Form A |
| | 7 days | partially birefringent | |
| | FE (filtrate from | clear glassy film, not | — |
| | slurry) | birefringent | |
| | slurry, | white, morphology unknown, | crystalline, possibly A + |
| | 7 days | not birefringent | peaks |
| | (scale up) | | |
| 1,4-dioxane | slurry, | yellow glassy film, not | amorphous |
| | 7 days | birefringent | |
| | FE (liquid phase | clear oily film, not | — |
| | from slurry) | birefringent | |
| ethanol | slurry, | white, morphology unknown, | IS |
| | 7 days | not birefringent; clear glassy | |
| | | film, not birefringent | |
| | FE (liquid phase | yellow, morphology | amorphous + peaks |
| | from slurry) | unknown, birefringent | |
| ethyl acetate | slurry, | white, morphology unknown, | Form B |
| | 7 days | not birefringent | |
| | FE (filtrate from | clear glassy film, not | — |
| | slurry) | birefringent | |
| | slurry, | white, morphology unknown, | B minus peaks |
| | 7 days | partially birefringent | |
| | (scale up) | | |
| iso-propanol | slurry, | light yellow, morphology | amorphous |
| | 7 days | unknown, not birefringent | |
| | FE (filtrate from | clear glassy film, not | — |
| | slurry) | birefringent; white, | |
| | | morphology unknown, | |
| | | birefringent | |
| methanol | FE | white, morphology unknown, | amorphous |
| | | birefringent | |
| methyl ethyl | slurry, | light brown, morphology | amorphous |
| ketone | 7 days | unknown, not birefringent | |
| | FE (filtrate from | yellow oily film, not | — |
| | slurry) | birefringent; clear | |
| | | morphology unknown, | |
| | | birefringent | |
| tetrahydrofuran | slurry, | white, morphology unknown, | amorphous |
| (THF) | 7 days | not birefringent | |
| | FE (filtrate from | clear fibers, birefringent | — |
| | slurry) | | |
| toluene | slurry, | white, morphology unknown, | amorphous |
| | 7 days | not birefringent | |
| | liquid phase from | clear glassy film, not | — |
| | slurry, FE | birefringent | |
| 2,2,2- | slurry, | clear solution with one white | — |
| trifluoroethanol | 3 days | float | |
| | FE | white, morphology unknown, | amorphous |
| | | not birefringent | |
| water | FE | yellow flakes, birefringent | amorphous |

^{a}FE = fast evaporation
^{b}IS = insufficient sample

A low crystalline Form A and crystalline Form B resulted from slurry experiments in acetonitrile and ethyl acetate, respectively (Table 6A and Table 7A). The XRPD patterns of both forms are presented in FIGS. 3a and 3b. The proton NMR spectra for Forms A and B are shown in FIG. 4 and FIG. 5, respectively. Based on NMR, low crystalline Form A contained residual amounts of acetonitrile, whereas crystalline Form B was likely an ethyl acetate mono-solvate.

TABLE 6A
Characterization of L-Tartrate Salt, low crystalline Form A

| Technique | Analysis/Result |
|---|---|
| XRPD | low crystalline Form A |
| ¹H NMR | 0.16 mole of CH₃CN per 1 mole of |
| | compound |

TABLE 7A
Characterization of L-Tartrate Salt, Form B

| Technique | Analysis/Result |
|---|---|
| XRPD | crystalline Form B |
| ¹H NMR | 0.91 mole of EtOAc per 1 mole of |
| | compound |

Tosylate Salt

The initial lot of the tosylate salt was prepared as follows.

To a suspension of compound (0.93 g, 3 mmol) in MeOH (10 ml) was added a solution of p-toluenesulfonic acid monohydrate (0.57 g, 3 mmol) in MeOH (5 ml) at 50° C. with stirring. The mixture was heated to reflux to obtain a clear solution, allowed to cool naturally to 20-25° C. with stirring (crystallisation occurred), aged in ice for 30 min. The precipitate was collected, washed with MeOH (3 ml), dried in vacuum at 45° C. to a constant weight. Yield 1.07 g (74%)

A polymorph screen of the tosylate salt was carried out using slurry and fast evaporation crystallization techniques (Table 8A). The initial lot of the tosylate salt was designated as Form A (FIG. 1c). Seven new crystalline forms were obtained and designated alphabetically from B through H (FIGS. 6a to 6h). The materials exhibiting new crystalline XRPD patterns were characterized by proton NMR and the NMR spectra were consistent with the compound structure, except for the spectrum of Form D. Forms B, C, E, F, and H were additionally characterized using thermal techniques.

TABLE 8A
Polymorph Screen of Tosylate salt

| Solvent | Conditions^{a} | Habit/Description | XRPD Result |
|---|---|---|---|
| acetone | FE | clear, broken glass, | amorphous |
| | | birefringent | |
| acetonitrile | slurry, | white solid | B |
| | 7 day | | |
| 1,4-dioxane | FE, vac. oven | clear glassy solid, not | — |
| | | birefringent | |
| ethanol | FE | white, dendridic formations, | A + peaks |
| | | birefringent | |
| ethyl acetate | slurry, | white solid | F |
| | 7 days | | |
| | slurry, 1 day | — | amorphous halo + |
| | | | peaks |
| | slurry, 4 days | white solid | F |
| | slurry, 4 days | white solid | — |
| iso-propanol | slurry, | white solid | C |
| | 7 days | | |
| | slurry, 1 day | — | amorphous + E |
| | | | peaks |
| | slurry, 4 days | white solid | C |
| | slurry, 4 days | white solid | — |
| methanol | FE | white solid, broken glass, not | A + peaks |
| | | birefringent and long needles, | |
| | | birefringent | |
| methyl ethyl | FE | dark red viscous liquid | — |
| ketone | | | |
| tetrahydrofuran | slurry, | white solid | D |
| (THF) | 7 days | | |
| | slurry, 1 day | — | amorphous halo + |
| | | | peaks |
| | slurry, 4 days | white solid | H |
| | slurry, 7 days | white solid | H |
| toluene | slurry, | white solid | B |
| | 7 days | | |
| | slurry, 1 day | white solid | B |
| | slurry, 1 day, dried | white solid | — |
| | under N₂, 3 days | | |
| 2,2,2- | FE | white, dendridic formations, | E |
| trifluoroethanol | | birefringent | |
| | FE | white, dendridic formations, | E |
| | | birefringent | |
| water | FE | white spherulites, birefringent | G |
| | FE | tiny white spherulites of | G |
| | | needles, birefringent; white, | |
| | | morphology unknown, not | |
| | | birefringent | |

^{a}FE = fast evaporation
b. Sample analyzed in capillary as slurry

Form A was analyzed by NMR and thermal techniques (Table 9A, FIG. 7, FIG. 8). A weight loss of approximately 0.95% was observed in TG between 16 and 225° C. The DSC exhibited two small broad endotherms at approximately 58 and 95° C., probably due to loss of residual solvent, followed by a sharp endotherm at approximately 208° C., probably due to the melt.

TABLE 9A
Characterization of Tosylate Salt Form A

| Technique | Analysis/Result |
|---|---|
| XRPD | A |
| ¹H NMR | consistent w/structure |
| DSC^{a} | endo 58 (broad), 95 |
| | (broad) 208 (56 J/g) |
| TGA^{b} | 0.95 @ 16-225 |

^{a}endo = endotherm, temperatures (C.°) reported are transition maxima. Temperatures are rounded to the nearest degree.
^{b}weight loss (%) at a certain temperature; weight changes (%) are rounded to 2 decimal places; temperatures are rounded to the nearest degree

Form B resulted from fast evaporation in acetonitrile. No solvent was present in the material based on the proton NMR spectrum (FIG. 9). The thermal data for Form B are included in Table 10A and shown in FIG. 10. The DSC thermogram exhibited a broad endotherm at approximately 63° C. followed by a sharp endotherm at approximately 205° C. most likely due to the melt (FIG. 10). The broad endotherm was probably due to dehydration and was accompanied by a weight loss of approximately 1.65% between 18 to 100° C. in TG, which was calculated to be approximately 0.45 mmol of water.

TABLE 10A
Characterization of Tosylate Salt, Form B

| Technique | Analysis/Result |
|---|---|
| XRPD | B |
| ¹H NMR | consistent w/structure |
| DSC^{a} | endo 63 (broad), 205 (52 J/g) |
| TGA^{b} | 1.65 @ 18-100 |

^{a}endo = endotherm, temperatures (C.°) reported are transition maxima. Temperatures are rounded to the nearest degree.
^{b}weight loss (%) at a certain temperature; weight changes (%) are rounded to 2 decimal places; temperatures are rounded to the nearest degree

Form C was obtained in slurry experiments in isopropanol after four and seven days. The thermal data for Form C are included in Table 11A and shown in FIG. 12. The DSC thermogram exhibited a broad endotherm at approximately 124° C. with a shoulder at 113° C. followed by an exotherm at approximately 165° C. and an endotherm at approximately 196° C., possibly due to the melt. The broad endotherm at 124° C. was accompanied by a stepwise weight loss of 13.11% in the range of 18 to 140° C. The weight loss was due to desolvation and corresponded to approximately 1.2 mmol of isopropanol. Approximately one mole of isopropanol per one mole of the compound was found based on the ¹H NMR spectrum (FIG. 11).

TABLE 11A
Characterization of Tosylate Salt, Form C

| Technique | Analysis/Result |
|---|---|
| XRPD | C |
| ¹H NMR | 0.91 mole of isopropanol per 1 mole of |
| | compound |
| DSC^{b} | shoulder 113, endo 124, exo 165, endo 196 |
| TGA^{c} | 13.11@ 18-140 |

^{b}endo = endotherm, exo = exotherm, temperatures (C.°) reported are transition maxima. Temperatures are rounded to the nearest degree.
^{c}weight loss (%) at a certain temperature; weight changes (%) are rounded to 2 decimal places; temperatures are rounded to the nearest degree

Form D resulted from a slurry experiment in tetrahydrofuran after seven days. The characterization data for Form D are summarized in Table 12A. Peak shifts in the proton NMR indicated a different structure that was, nonetheless, related to the structure of the tosylate salt (FIG. 13). The amount of material was insufficient for further characterization. Form D was not reproduced in a scale-up experiment.

TABLE 12A
Characterization of Tosylate Salt, Form D

| Technique | Analysis/Result |
|---|---|
| XRPD | D |
| ¹H NMR | different structure |

Form E was obtained in a fast evaporation experiment in 2,2,2-trifluoroethanol. The thermal data for Form E are included in Table 13A and shown in FIG. 15. The DSC thermogram exhibited three broad endotherms at approximately 67, 102, and 138° C. followed by a sharper intensive endotherm at approximately 199° C., likely due to the melt, and a small broad endotherm at 224° C. The first three endotherms were accompanied by a stepwise weight loss of 7.87% between 16 and 150° C. A residual amount of trifluoroethanol, approximately 0.143 mole per one mole of the compound, was found in the ¹H NMR spectrum (FIG. 14, Table 13A). The observed weight loss was probably due to both desolvation and dehydration (calculated to be approximately 0.4 mmol of 2,2,2-trifluoroethanol).

TABLE 13A
Characterization of Tosylate Salt, Form E

| Technique | Analysis/Result |
|---|---|
| XRPD | E |
| ¹H NMR | 0.143 mole of TFE^{a} per 1 mole of |
| | compound |
| DSC^{b} | endo 67 (broad), 102, 138, 199, 224 |
| TGA^{c} | 7.87 @ 16-150 |

^{a}TFE = 2,2,2-trifluoroethanol
^{b}endo = endotherm, temperatures (C.°) reported are transition maxima. Temperatures are rounded to the nearest degree.
^{c}weight loss (%) at a certain temperature; weight changes (%) are rounded to 2 decimal places; temperatures are rounded to the nearest degree

Form F (also referred to as crystal modification X) was produced in slurry experiments in ethyl acetate after four and seven days. No solvent was present in the material based on the ¹H NMR spectrum (FIG. 16). The thermal data for Form F are included in Table 14A and shown in FIG. 17. The DSC thermogram exhibited a broad endotherm at approximately 66° C. followed by a sharp endotherm at approximately 205° C., likely due to the melt. The broad endotherm accompanied by a weight loss of approximately 1.15% in the range of 17 to 100° C. in TG was possibly due to dehydration. The weight loss was calculated to be approximately 0.3 mmol of water.

TABLE 14A
Characterization of Tosylate Salt, Form F

| Technique | Analysis/Result |
|---|---|
| XRPD | F |
| ¹H NMR | consistent w/structure |
| DSC^{a} | endo 66 (broad), 205 (54 J/g) |
| TGA^{b} | 1.15 @ 17-140 |

^{a}endo = endotherm, temperatures (C.°) reported are transition maxima. Temperatures are rounded to the nearest degree.
^{b}weight loss (%) at a certain temperature; weight changes (%) are rounded to 2 decimal places; temperatures are rounded to the nearest degree

Form G obtained from fast evaporation in water was likely a hydrate. The XRPD and proton NMR data for Form G are summarized in Table 15A (structure confirmed by NMR, FIG. 18).

TABLE 15A
Characterization of Tosylate Salt, Form G

| Technique | Analysis/Result |
|---|---|
| XRPD | G |
| ¹H NMR | consistent w/structure |

Form H (also called crystal modification Y) was produced in a slurry experiment in tetrahydrofuran after four and seven days. The thermal data for Form H are included in Table 16A and shown in FIG. 20. The DSC thermogram exhibited a broad endotherm at approximately 115° C. with a shoulder at 127° C. followed by a small endotherm at approximately 186° C. The endotherm at 115° C. was accompanied by a stepwise weight loss of approximately 14.70% in the range of 16 to 145° C., probably due to desolvation (corresponded to approximately 1.15 mmol of tetrahydrofuran,). Approximately 0.7 mole of tetrahydrofuran per one mole of compound was found by ¹H NMR (FIG. 19).

TABLE 16A
Characterization of Tosylate Salt, Form H

| Technique | Analysis/Result |
|---|---|
| XRPD | H |
| ¹H NMR | 0.7 mole of THF per 1 mole of compound |
| DSC^{b} | endo at 115, shoulder at 127, endo at 186 (small) |
| TGA^{c} | 14.70 @ 16-145 |

^{b}endo = endotherm, temperatures (C.°) reported are transition maxima. Temperatures are rounded to the nearest degree.
^{c}weight loss (%) at a certain temperature; weight changes (%) are rounded to 2 decimal places; temperatures are rounded to the nearest degree

Results—Wellplate Salt Screen

Wellplate 1

Salt preparation results for wellplate 1 are summarized in Table 17A and Table 18A. The following acids were used in the screen:

acetic,

adipic,

citric,

gentisic,

glutaric,

glycolic,

L-malic.

The acids were dissolved in methanol and added to solutions of the freebase dissolved in acetone, methanol, methyl ethyl ketone, and tetrahydrofuran. Solids were obtained from slurry/fast evaporation experiments in the wells.

The free base (i.e. compound 2) was also dissolved in acetone, MeOH, MEK and THF) and solids obtained (well plate numbers H1, H2, H4, H5, H7, H8, H10 and H11 Table 17A). These experiments resulted in the amorphous form of compound 2.

TABLE 17A
Wellplate Salt Preparation Attempts from Compound 2
Plate 1; acids dissolved in methanol; ambient-temperature mix; 1:1equivalents acid/API
with excess ac

| | | Observations^{b} | | | | | |
|---|---|---|---|---|---|---|---|
| | API | | | 11 days (sat 6 | | Well | XRPD |
| Acid | Solvent^{a} | 3 days | B/E | days/evaporated) | B/E | No. | Results |
| citric | acetone | — | — | irregular plates | Y | C1 | low |
| | | | | (caramel) | | | crystalline 1 |
| | | | | irregular plates | Y | C2 | crystalline 1 |
| | | | | (caramel) | | | |
| | | | | unknown morphology | Y | C3 | crystalline 1 |
| | | | | (caramel) | | | |
| | MeOH | — | — | wisps (caramel) | Y | C4 | low |
| | | | | | | | crystalline 1 |
| | | | | unknown morphology | N | C5 | low |
| | | | | (yellow) | | | crystalline 1 |
| | | | | unknown morphology | N | C6 | low |
| | | | | (white) | | | crystalline 1 |
| | MEK | — | — | unknown morphology | N | C7 | low |
| | | | | (red) | | | crystalline 1 |
| | | | | | | C8 | low |
| | | | | | | | crystalline 1 |
| | | | | | | C9 | low |
| | | | | | | | crystalline 1 |
| | THF | — | — | needles (caramel) | Y | C10 | amorphous |
| | | | | unknown morphology | Y | C11 | amorphous |
| | | | | (caramel) | | | with peaks |
| | | | | unknown morphology | Y | C12 | amorphous |
| | | | | (caramel) | | | |
| gentisic | acetone | — | — | needles (caramel) | Y | D1 | amorphous |
| | | | | | | D2 | amorphous |
| | | | | | | D3 | amorphous |
| | MeOH | dark | N | (yellow) | N | D4 | amorphous |
| | | rings | | | | | |
| | | — | — | | | D5 | amorphous |
| | | — | — | | | D6 | amorphous |
| | MEK | — | — | unknown morphology | Y | D7 | amorphous |
| | | | | (orange) | | | |
| | | | | unknown morphology | Y | D8 | amorphous |
| | | | | (red) | | | |
| | | | | needles (black, red) | Y | D9 | amorphous |
| | THF | — | — | needles (caramel) | Y | D10 | amorphous |
| | | glass | N | needles (caramel) | Y | D11 | amorphous |
| | | glass | N | unknown morphology | Y | D12 | amorphous |
| | | | | (caramel) | | | |

^{a}MeOH = methanol, MEK = methyl ethyl ketone, THF = tetrahydrofuran.
^{b}B = birefringence, E = extinction; samples observed under microscope with
crossed polarized light; Y = yes, N = no. All wells exhibited dark rings upon
final observation. Visual observations for color are given in parentheses.

| | | | | | | | |
|---|---|---|---|---|---|---|---|
| acetic | acetone | — | — | (caramel) | N | A1 | amorphous |
| | | | | unknown morphology | Y | A2 | amorphous |
| | | | | (brown, caramel) | | | |
| | | | | wisps (brown) | Y | A3 | crystalline 1 |
| | MeOH | — | — | few needles (caramel) | Y | A4 | amorphous |
| | | | | few wisps (yellow) | Y | A5 | amorphous |
| | | | | few needles (caramel) | Y | A6 | crystalline 1 |
| | MEK | — | — | unknown morphology | Y | A7 | amorphous |
| | | | | (red) | | | |
| | | | | unknown morphology | Y | A8 | amorphous |
| | | | | (red) | | | |
| | | | | needles (red) | Y | A9 | amorphous |
| | THF | — | — | needles (caramel) | Y | A10 | amorphous |
| | | | | | | A11 | amorphous |
| | | | | | | A12 | amorphous |
| adipic | acetone | — | — | irregular plates | Y | B1 | low |
| | | | | (brown, caramel) | | | crystalline 1 |
| | | | | irregular plates | Y | B2 | crystalline 1 |
| | | | | (brown) | | | |
| | | | | irregular plates | Y | B3 | crystalline 1 |
| | | | | (brown) | | | |
| | MeOH | — | — | unknown morphology | Y | B4 | amorphous |
| | | | | (caramel) | | | |
| | | | | unknown morphology | Y | B5 | amorphous |
| | | | | (yellow) | | | |
| | | | | few needles (yellow) | Y | B6 | crystalline |
| | | | | | | | 1 minus |
| | | | | | | | peaks |
| | MEK | — | — | wisps (red) | Y | B7 | amorphous |
| | | | | | | B8 | amorphous |
| | | | | | | B9 | amorphous |
| | THF | — | — | unknown morphology | Y | B10 | amorphous |
| | | | | (caramel) | | B11 | amorphous |
| | | | | | | B12 | amorphous |

^{a}MeOH = methanol, MEK = methyl ethyl ketone, THF = tetrahydrofuran.
^{b}B = birefringence, E = extinction; samples observed under microscope with
crossed polarized light; Y = yes, N = no. All wells exhibited dark rings upon final
observation. Visual observations for color are given in parentheses.

| | | | | | | | |
|---|---|---|---|---|---|---|---|
| glutaric | acetone | — | — | irregular plates | Y | E1 | amorphous |
| | | | | (brown, caramel) | | | with peaks |
| | | | | unknown morphology | Y | E2 | amorphous |
| | | | | (caramel) | | | |
| | | | | unknown morphology | Y | E3 | amorphous |
| | | | | (caramel) | | | |
| | MeOH | — | — | (caramel) | N | E4 | amorphous |
| | | | | (caramel) | N | E5 | amorphous |
| | | | | (yellow) | N | E6 | amorphous |
| | MEK | — | — | (caramel) | N | E7 | amorphous |
| | | | | needles (orange) | Y | E8 | amorphous |
| | | | | wisps (red) | Y | E9 | amorphous |
| | THF | — | — | wisps (caramel) | Y | E10 | amorphous |
| | | | | wisps (caramel) | Y | E11 | amorphous |
| | | | | unknown morphology | Y | E12 | amorphous |
| | | | | (caramel) | | | |
| glycolic | acetone | — | — | unknown morphology | Y | F1 | low |
| | | | | (brown, caramel) | | | crystalline 1 |
| | | | | wisps (caramel) | Y | F2 | amorphous |
| | | | | few irregular plates | Y | F3 | amorphous |
| | | | | (caramel) | | | |
| | MeOH | — | — | (caramel) | N | F4 | amorphous |
| | | | | unknown morphology | Y | F5 | amorphous |
| | | | | (yellow) | | | |
| | | | | unknown morphology | Y | F6 | amorphous |
| | | | | (yellow) | | | |
| | MEK | — | — | unknown morphology | Y | F7 | amorphous |
| | | | | (red) | | | with peaks |
| | | | | unknown morphology | Y | F8 | amorphous |
| | | | | (orange) | | | |
| | | | | unknown morphology | Y | F9 | amorphous |
| | | | | (red) | | | with peaks |
| | THF | glass | N | needles (caramel) | Y | F10 | amorphous |
| | | | | | | | with peaks |
| | | | | | | F11 | amorphous |
| | | | | | | | with peaks |
| | | | | | | F12 | amorphous |

^{a}MeOH = methanol, MEK = methyl ethyl ketone, THF = tetrahydrofuran.
^{b}B = birefringence, E = extinction; samples observed under microscope with
crossed polarized light; Y = yes, N = no. All wells exhibited dark rings upon final
observation. Visual observations for color are given in parentheses.

| | | | | | | | |
|---|---|---|---|---|---|---|---|
| L-malic | acetone | — | — | unknown | Y | G1 | amorphous |
| | | | | morphology (brown, | | | |
| | | | | caramel) | | | |
| | | | | unknown | Y | G2 | amorphous |
| | | | | morphology (brown, | | | with peaks |
| | | | | caramel) | | | |
| | | | | needles (brown, | Y | G3 | amorphous |
| | | | | caramel) | | | with peaks |
| | MeOH | — | — | few wisps (caramel) | Y | G4 | amorphous |
| | | dark | N | prisms, needles | Y | G5 | crystalline 1 |
| | | rings | | (caramel) | | | |
| | | — | — | unknown | Y | G6 | crystalline 1 |
| | | | | morphology, needles | | | |
| | | | | (red) | | | |
| | MEK | — | — | (red) | N | G7 | amorphous |
| | | | | unknown | Y | G8 | amorphous |
| | | | | morphology (red) | | | |
| | | | | prisms (singles), | Y | G9 | amorphous |
| | | | | needles (red) | | | with peaks |
| | THF | glass | N | wisps (caramel) | Y | G10 | amorphous |
| | | | | unknown | Y | G11 | amorphous |
| | | | | morphology | | | with peaks |
| | | | | (caramel) | | | |
| | | | | wisps (caramel) | Y | G12 | amorphous |
| none | acetone | — | — | unknown | Y | H1 | amorphous |
| | | | | morphology | | | with peaks |
| | | | | (caramel) | | | |
| | | | | needles (caramel) | Y | H2 | amorphous |
| | MeOH | — | — | needles (brown, | Y | H4 | amorphous |
| | | | | caramel) | | | |
| | | | | (yellow) | N | H5 | amorphous |
| | | | | | | | with peaks |
| | MEK | — | — | needles (red, | Y | H7 | amorphous |
| | | | | caramel) | | | with peaks |
| | | | | few needles | Y | H8 | amorphous |
| | | | | (red, caramel) | | | |
| | THF | — | — | unknown | Y | H10 | amorphous |
| | | | | morphology | | H11 | amorphous |
| | | | | (caramel) | | | |

^{a}MeOH = methanol, MEK = methyl ethyl ketone, THF = tetrahydrofuran.
^{b}B = birefringence, E = extinction; samples observed under microscope with
crossed polarized light; Y = yes, N = no. Singles = well contained particles
suitable for structure determination submission. All wells exhibited dark rings upon final
observation. Visual observations for color are given in parentheses.

TABLE 18A
Summary of Well Plate Crystalline Forms

| Acid | Solvent System^{b} | Well No. | XRPD Result |
|---|---|---|---|
| acetic | acetone, MeOH^{a} | A3 | crystalline 1 |
| | MeOH | A6 | |
| | MeOH:ACN 1:1 | A3 | |
| | MeOH:EtOAc 1:1 | A6 | |
| adipic | acetone, MeOH^{a} | B2 | crystalline 1 |
| | | B3 | |
| | MeOH:ACN 1:1 | B1 | |
| | | B2 | |
| | | B3 | |
| | MeOH:EtOAc 1:1 | B5 | |
| | acetone, MeOH^{a} | B1 | low crystalline 1 |
| | MeOH | B6 | crystalline 1 minus |
| | MeOH:EtOAc 1:1 | B6 | peaks |
| citric | acetone, MeOH^{a} | C2 | crystalline 1 |
| | | C3 | |
| | MeOH:ACN 1:1 | C1 | |
| | | C3 | |
| | MeOH:EtOAc 1:1 | C4 | |
| | | C5 | |
| | | C6 | |
| | acetone, MeOH^{a} | C1 | low crystalline 1 |
| | MeOH | C4 | |
| | | C5 | |
| | | C6 | |
| | MEK, MeOH^{a} | C7 | |
| | | C8 | |
| | | C9 | |
| gentisic | MeOH:EtOAc 1:1 | D5 | crystalline 1 |
| | | D6 | crystalline 2 |
| glutaric | MeOH:ACN 1:1 | E1 | crystalline 1 |
| | | E2 | |
| | MeOH:EtOAc 1:1 | E4 | |
| | | E5 | |
| | | E6 | |
| | MeOH:ACN 1:1 | E3 | low crystalline 1 |
| glycolic | MeOH:ACN 1:1 | F1 | crystalline 1 |
| | acetone, MeOH^{a} | F1 | low crystalline 1 |
| | MeOH:ACN 1:1 | F2 | |
| | | F3 | |
| HBr | TFE, MeOH^{a} | A10 | crystalline 1 |
| | | A11 | |
| | | A12 | |
| | MeOH:EtOAc 1:1 | A5 | |
| | | A6 | |
| | MeOH:IPA 1:1 | A8 | |
| | MeOH:toluene 1:1 | A10 | |
| | | A11 | |
| | | A12 | |
| | acetone, MeOH^{a} | A2 | crystalline 2 |
| | MEK, MeOH^{a} | A7 | |
| | | A8 | |
| | | A9 | |
| | MeOH:ACN 1:1 | A1 | |
| | | A3 | |
| | MeOH:IPA 1:1 | A9 | |
| | MeOH:ACN 1:1 | A2 | low crystalline 2 |
| lactic | MeOH:toluene 1:1 | B12 | crystalline 1 |
| maleic | acetone, MeOH^{a} | C1 | crystalline 1 |
| | | C2 | |
| | MeOH | C4 | |
| | | C5 | |
| | MeOH:ACN 1:1 | C2 | |
| | MeOH:EtOAc 1:1 | C5 | |
| | acetone, MeOH^{a} | C3 | crystalline 1 + one peak |
| | MeOH | C6 | |
| | MeOH:ACN 1:1 | C1 | |
| | | C3 | |
| | MeOH:EtOAc 1:1 | C4 | |
| | | C6 | |
| | MeOH:toluene 1:1 | C10 | |
| | | C11 | |
| | | C12 | |
| | TFE, MeOH^{a} | C11 | low crystalline 1 |
| L-malic | MeOH | G5 | crystalline 1 |
| | | G6 | |
| | MeOH:ACN 1:1 | G1 | |
| | | G3 | |
| phosphoric | MeOH | G4 | crystalline 1 |
| | | G6 | |
| | TFE, MeOH^{a} | G10 | |
| | | G11 | |
| | | G12 | |
| | MeOH:ACN 1:1 | G2 | |
| | | G3 | |
| | MeOH:EtOAc 1:1 | G4 | |
| | | G5 | |
| | MeOH:toluene 1:1 | G10 | |
| | | G11 | |
| | | G12 | |
| | acetone, MeOH^{a} | G3 | crystalline 1 + peaks |
| | MeOH:EtOAc 1:1 | G6 | |
| | MeOH | G5 | low crystalline 1 |
| | acetone, MeOH^{a} | G1 | crystalline 2 |
| | | G2 | |
| | MeOH:ACN 1:1 | G1 | |
| | MEK, MeOH^{a} | G7 | crystalline 3 |
| | MeOH:IPA 1:1 | G7 | |
| | MEK, MeOH^{a} | G8 | crystalline 4 |
| | MeOH:IPA 1:1 | G8 | |
| succinic | acetone, MeOH^{a} | E1 | crystalline 1 |
| | | E2 | |
| | MeOH | E4 | |
| | | E5 | |
| | | E6 | |
| | TFE, MeOH^{a} | E12 | |
| | MeOH:ACN 1:1 | E1 | |
| | | E2 | |
| | | E3 | |
| | MeOH:EtOAc 1:1 | E4 | |
| | | E5 | |
| | | E6 | |
| | acetone, MeOH^{a} | E3 | low crystalline 1 |
| | TFE, MeOH^{a} | E10 | crystalline 2 |
| | MeOH:toluene 1:1 | E10 | |
| | | E12 | |
| | | E11 | crystalline 2 minus |
| | | | peaks |
| sulfuric | acetone, MeOH^{a} | F2 | crystalline 1 |
| | | F3 | |
| | MEK, MeOH^{a} | F8 | |
| | | F9 | |
| | TFE, MeOH^{a} | F10 | |
| | | F11 | |
| | MeOH:ACN 1:1 | F1 | |
| | | F2 | |
| | | F3 | |
| | MeOH:IPA 1:1 | F7 | |
| | | F9 | |
| | MeOH:toluene 1:1 | F10 | |
| | | F11 | |
| | | F12 | |
| | MeOH | F4 | low crystalline 1 |
| | MEK, MeOH^{a} | F7 | |
| | MeOH:EtOAc 1:1 | F4 | crystalline 1 minus |
| | | F5 | peaks |
| | MeOH:IPA 1:1 | F8 | |
| | MeOH | F6 | crystalline 2 |
| | MeOH:EtOAc 1:1 | F6 | crystalline 2 minus |
| | | | peaks |
| | acetone, MeOH^{a} | F1 | crystalline 3 |
| | MeOH | F5 | crystalline 4 |

^{a}Acids were dissolved in methanol then added to a solution containing freebase. The solvent that dissolved the freebase was the major component in the mixture.
^{b}ACN = acetonitrile, EtOAc = ethyl acetate, IPA = isopropanol, MeOH = methanol, MEK = methyl ethyl ketone, TFE = 2,2,2-trifluoroethanol.

Wellplate 2

Salt preparation results for wellplate 2 are summarized in Table 19A and Table 18A above. The following acids were used in the screen:

hydrobromic,

lactic,

maleic,

methanesulfonic,

succinic,

sulfuric,

phosphoric.

The acids were dissolved in methanol and added to solutions of compound 2 dissolved in acetone, methanol, methyl ethyl ketone, and 2,2,2-trifluoroethanol. Solids were obtained from slurry/fast evaporation experiments in the wells.

TABLE 19A
Wellplate Salt Preparation Attempts from Compound 2
Acids dissolved in methanol; ambient-temperature mix, 1:1 equivalents acid/API with excess
acid (non-GMP)

| | API | Observations^{b} | | | | Well | XRPD |
|---|---|---|---|---|---|---|---|
| Acid | Solvent^{a} | 3 days | B/E | 8 days | B/E | No. | Results |
| HBr | acetone | DR | N | yw, needles | Y | A1 | amorphous |
| | | (clear at 8 d) | | yw, UM | Y | A2 | crystalline 2 |
| | | | | white fibers | Y | A3 | amorphous |
| | | | | UM | N | | |
| | MeOH | | | white fibers | N | A4 | amorphous |
| | | | | white needles | Y | A5 | amorphous |
| | | | | white, UM | Y | A6 | amorphous |
| | MEK^{c} | DR (yw) | N | OR needles | Y | A7 | crystalline 2 |
| | | | | UM | N | | |
| | | | | OR oil | N | A8 | crystalline 2 |
| | | | | OR, UM | N | A9 | crystalline 2 |
| | TFE | DR, dark | N | off-white, UM | partial | A10 | crystalline 1 |
| | | chunks of | | | partial | A11 | crystalline 1 |
| | | UM | | | N | A12 | crystalline 1 |
| | | (white at | | | | | |
| | | 8 d) | | | | | |
| lactic | acetone | DR, few | Y | yw fibers, UM | Y | B1 | amorphous |
| | | platy | | yw irregular | Y | B2 | amorphous |
| | | particles | | plates | | | |
| | | (yw) | | | | | |
| | | DR, platy | Y | yw, UM | Y | B3 | amorphous |
| | | particles, | | | | | |
| | | specks (yw) | | | | | |
| | MeOH | DR (clear at | N | off-white glass | N | B4 | amorphous |
| | | 8 d) | | UM | Y | | with peaks |
| | | | | clear oil | N | B5 | amorphous |
| | | | | clear fibers, UM | Y | B6 | amorphous |
| | MEK | DR (yw) | N | OR glass | N | B7 | amorphous |
| | | | | fibers | Y | | |
| | | | | OR glass, UM | N | B8 | amorphous |
| | | DR (yw at | | OR oil | N | B9 | amorphous |
| | | 3 d, OR at | | | | | |
| | | 8 d) | | | | | |
| | TFE | DR (clear at | N | clear glass, UM | N | B10 | amorphous |
| | | 8 d) | | one fiber | Y | | |
| | | | | clear, UM | Y | B11 | amorphous |
| | | | | glass | N | | |
| | | | | clear fibers | Y | B12 | amorphous |
| | | | | glass, UM | N | | |

^{a}MeOH = methanol, MEK = methyl ethyl ketone, TFE = 2,2,2-trifluoroethanol.
^{b}B = birefringence, E = extinction; samples observed under microscope with crossed polarized
light; DR = dark rings, d = days, yw = yellow, OR = orange, clear = colorless, UM = unknown
morphology, Y = yes, N = no. Singles = well contained particles suitable for structure
determination submission. All wells exhibited dark rings upon final observation. Visual
observations for color.
^{c}Violet solution produced upon acid addition

| | | | | | | | |
|---|---|---|---|---|---|---|---|
| maleic | acetone | DR (yw) | N | yw, UM | N | C1 | crystalline 1 |
| | | | | fibers, UM | Y | | |
| | | | | yw spherulites | Y | C2 | crystalline 1 |
| | | | | | | C3 | crystalline |
| | | | | | | | 1 + one |
| | | | | | | | peak |
| | MeOH | DR (clear at | N | yw spherulites, one | Y | C4 | crystalline 1 |
| | | 8 d) | | fiber | | | |
| | | | | white, UM | N | C5 | crystalline 1 |
| | | | | clear spherulites, | Y | C6 | crystalline 1 |
| | | | | one fiber | | | 1 + one |
| | | | | | | | peak |
| | MEK | DR (yw) | N | OR glass, UM | N | C7 | amorphous |
| | | DR, dark | N | OR oil | N | C8 | amorphous |
| | | specks (yw) | | UM | Y | | with peaks |
| | | DR, oil | N | OR oil | N | C9 | amorphous |
| | | (yw/pink) | | UM | Y | | |
| | | (OR at 8 d) | | | | | |
| | TFE | DR (white | N | pink spherulites | Y | C10 | amorphous |
| | | at 8 d) | | white spherulites | Y | C11 | low |
| | | | | | | | crystalline 1 |
| | | | | white spherulites, | Y | C12 | amorphous |
| | | | | needles | | | |
| methane- | acetone | DR (clear at | N | clear glass, UM | N | D1 | amorphous |
| sulfonic | | 8 d) | | fibers | Y | | |
| | | | | yw fibers | Y | D2 | amorphous |
| | | | | | | D3 | amorphous |
| | MeOH | | | clear glass, UM | N | D4 | amorphous |
| | | | | clear fibers, needles | Y | D5 | amorphous |
| | | | | clear glass | N | D6 | amorphous |
| | | | | UM | Y | | with peaks |
| | MEK^{c} | DR (yw) | N | yw oil | N | D7 | amorphous |
| | | | | needles | Y | | |
| | | DR, oil (yw | N | violet oil | N | D8 | amorphous |
| | | at 8 d) | | | | | |
| | | DR, dark | N | brown oil | N | D9 | amorphous |
| | | specks | | UM | Y | | |
| | | (pink at 8 d) | | | | | |
| | TFE | DR (clear at | N | yw oil | N | D10 | amorphous |
| | | 8 d) | | fibers, UM | Y | | |
| | | | | yw oil, UM | N | D11 | amorphous |
| | | | | | | D12 | amorphous |

^{a}MeOH = methanol, MEK = methyl ethyl ketone, TFE = 2,2,2-trifluoroethanol.
^{b}B = birefringence, E = extinction; samples observed under microscope with crossed polarized
light; DR = dark rings, d = days, yw = yellow, OR = orange, clear = colorless, UM = unknown
morphology, Y = yes, N = no. Singles = well contained particles suitable for structure
determination submission. All wells exhibited dark rings upon final observation. Visual
observations for color.
^{c}Violet solution produced upon acid addition

| | | | | | | | |
|---|---|---|---|---|---|---|---|
| succinic | acetone | DR, (yw) | N | caramel-colored, | N | E1 | crystalline 1 |
| | | (OR, yw at | | UM | | | |
| | | 8 d) | | | | | |
| | | DR (yw) | N | | | E2 | crystalline 1 |
| | | DR (yw) | N | caramel-colored, | Y | E3 | low |
| | | (OR, yw at | | fibers, UM | | | crystalline 1 |
| | | 8 d) | | | | | |
| | MeOH | DR (clear at | N | yw, UM | N | E4 | crystalline 1 |
| | | 8 d) | | needles | Y | | |
| | | | | off-white blades | Y | E5 | crystalline 1 |
| | | | | pink blades | Y | E6 | crystalline 1 |
| | MEK | DR (yw) | N | red, UM | N | E7 | amorphous |
| | | | | fibers | Y | | |
| | | DR, oil (yw) | N | red oil | N | E8 | amorphous |
| | | | | UM | Y | | |
| | | DR, oil | N | red oil | N | E9 | amorphous |
| | | (yw/pink) | | UM | Y | | |
| | | (OR at 8 d) | | | | | |
| | TFE | DR (pink, | N | pink spherulites, | Y | E10 | crystalline 2 |
| | | off-white at | | needles | | | |
| | | 8 d) | | | | | |
| | | DR (off- | N | white spherulites | Y | E11 | low |
| | | white at 8 d) | | of very fine fibers | | | crystalline 1 |
| | | DR (clear at | N | white, UM | N | E12 | crystalline 1 |
| | | 8 d) | | | | | |
| H₂SO₄ | acetone | DR (yw) | N | OR, UM | partial | F1 | crystalline 3 |
| | | DR, few | Y | yw, UM | N | F2 | crystalline 1 |
| | | large | | | | | |
| | | hexagonal | | | | | |
| | | plates | | | | | |
| | | (singles) | | | | | |
| | | (yw) | | | | | |
| | | DR (yw) | N | yw irregular | Y | F3 | crystalline 1 |
| | | | | plates | | | |
| | MeOH | DR | N | clear, UM | Y | F4 | low |
| | | (clear at 8 d) | | | | | crystalline 1 |
| | | | | | partial | F5 | crystalline 4 |
| | | | | | Y | F6 | crystalline 2 |
| | MEK | DR (yw) | N | OR, UM | Y | F7 | low |
| | | | | | | | crystalline 1 |
| | | DR, oil (yw) | N | brown needles, | Y | F8 | crystalline 1 |
| | | | | UM | | | |
| | | DR, oil | N | OR, UM | Y | F9 | crystalline 1 |
| | | (pink) | | | | | |
| | | (OR at 8 d) | | | | | |
| | TFE | dark, UM | N | pink blades | Y | F10 | crystalline 1 |
| | | (pink at 8 d) | | | | | |
| | | dark, UM | | white blades | Y | F11 | crystalline 1 |
| | | (off-white at | | | | | |
| | | 8 d) | | | | | |
| | | dark, UM | | white fibers, | Y | F12 | amorphous |
| | | (white at 8 d) | | needles | | | |

^{a}MeOH = methanol, MEK = methyl ethyl ketone, TFE = 2,2,2-trifluoroethanol.
^{b}B = birefringence, E = extinction; samples observed under microscope with crossed polarized
light; DR = dark rings, d = days, yw = yellow, OR = orange, clear = colorless, UM = unknown
morphology, Y = yes, N = no. Singles = well contained particles suitable for structure
determination submission. All wells exhibited dark rings upon final observation. Visual
observations for color.

| | | | | | | | |
|---|---|---|---|---|---|---|---|
| H₃PO₄ | acetone | DR, few | Y | yw, UM | N | G1 | crystalline 2 |
| | | platy | | | | | |
| | | particles | | | | | |
| | | (yw) | | | | | |
| | | DR (yw) | N | | | G2 | crystalline 2 |
| | | dark solids | N | | | G3 | crystalline |
| | | of UM (yw) | | | | | 1 + peaks |
| | MeOH | DR (yw at | N | off-white, UM | partial | G4 | crystalline 1 |
| | | 8 d) | | | | | |
| | | DR (white | N | white blades, | Y | G5 | low |
| | | at 8 d) | | UM | | | crystalline 1 |
| | | DR, rosette | Y | white, UM | N | G6 | crystalline 1 |
| | | clusters of | | needles | Y | | |
| | | fine needles | | | | | |
| | | (white at 8 d) | | | | | |
| | MEK | DR, oil (yw) | N | red, UM | N | G7 | crystalline 3 |
| | | (OR at 8 d) | N | | partial | G8 | crystalline 4 |
| | | dark solids | N | red oil, UM | N | G9 | amorphous |
| | | of UM | | | | | with peaks |
| | | (pink) | | | | | |
| | | (red at 8 d) | | | | | |
| | TFE^{c} | dark solids | N | off-white, UM | N | G10 | crystalline 1 |
| | | of UM (off- | | needles | Y | | |
| | | white at 8 d) | | white, UM | N | G11 | crystalline 1 |
| | | | | needles | Y | | |
| | | dark solids | | white, UM | N | G12 | crystalline 1 |
| | | of UM | | needles | Y | | |
| | | (white at 8 d) | | | | | |
| none | acetone | DR, dark | N | yw glass | N | H1 | amorphous |
| | | chunks of | | UM | Y | | |
| | | UM (yw) | | yw glass | N | H2 | amorphous |
| | | | | UM, one fiber | Y | | |
| | MeOH | DR (clear at | N | clear fibers, UM | Y | H4 | amorphous |
| | | 8 d) | | clear glass | N | H5 | amorphous |
| | | | | UM | Y | | |
| | MEK | DR, platy | Y | OR blades, | Y | H7 | amorphous |
| | | particles, | | irregular plates | | | |
| | | specks (red) | | | | | |
| | | (yw at 8 d) | | | | | |
| | | DR, oil (yw) | N | OR oil | N | H8 | amorphous |
| | | | | needles, UM | Y | | |
| | | DR, oil (yw) | N | OR oil | N | H9 | amorphous |
| | | | | UM | Y | | |
| | TFE | DR (clear at | N | clear glass | N | H10 | amorphous |
| | | 8 d) | | UM | Y | | |
| | | | | clear glass | N | H11 | amorphous |

^{a}MeOH = methanol, MEK = methyl ethyl ketone, TFE = 2,2,2-trifluoroethanol.
^{b}B = birefringence, E = extinction; samples observed under microscope with crossed polarized
light; DR = dark rings, d = days, yw = yellow, OR = orange, clear = colorless, UM = unknown
morphology, Y = yes, N = no. Singles = well contained particles suitable for structure
determination submission. All wells exhibited dark rings upon final observation. Visual
observations for color.
^{c}White precipitate produced upon acid addition.

Recrystallization of Salts in Wellplates

Wellplate 3

Recrystallization of wellplate 3 was conducted using solvent/antisolvent evaporation. The solids in wells were dissolved in methanol. Acetonitrile, ethyl acetate, 1-propanol, and toluene were used as the antisolvents. The wells with sufficient amounts of non-glassy solids were analyzed by XRPD and the results are summarized in Table 20A and Table 18A above.

TABLE 20A
Recrystallization of Wellplate 3
to all wells methanol was added; solvent:antisolvent 1:1

| | | Anti- | | | | XRPD |
|---|---|---|---|---|---|---|
| Acid | Solvent^{a} | solvent^{b} | Observations | B^{c} | Well No. | Results |
| acetic | MeOH | ACN | dark brown ring, | N | A1 | — |
| | | | broken glass | | | |
| | | | dark brown ring, | N | A2 | — |
| | | | glass | | | |
| | | | morphology | Y | A3 | crystalline 1 |
| | | | unknown | | | |
| | | EtOAc | a few needles | Y | A4 | — |
| | | | glassy solid | N | A5 | — |
| | | | morphology | N | A6 | crystalline 1 |
| | | | unknown | | | |
| | | 1-PrOH | glassy solid | N | A7 | — |
| | | | glassy solid | N | A8 | — |
| | | | glassy solid | N | A9 | — |
| | | toluene | glassy solid | N | A10 | — |
| | | | Morphology | N | A11 | — |
| | | | unknown, a few | | | |
| | | | birefringent particles | | | |
| | | | glassy solid | N | A12 | — |
| adipic | MeOH | ACN | morphology | N | B1 | crystalline 1 |
| | | | unknown | | | |
| | | | morphology | N | B2 | crystalline 1 |
| | | | unknown | | | |
| | | | morphology | N | B3 | crystalline 1 |
| | | | unknown | | | |
| | | EtOAc | dark brown circle | N | B4 | — |
| | | | morphology | Part. Y | B5 | crystalline 1 |
| | | | unknown | | | |
| | | | morphology | Part. Y | B6 | crystalline 1 |
| | | | unknown | | | minus peaks |
| | | 1-PrOH | glassy solid with a | N | B7 | — |
| | | | few birefringent | | | |
| | | | particles | | | |
| | | | glassy solid with a | N | B8 | — |
| | | | few birefringent | | | |
| | | | particles | | | |
| | | | glassy solid | N | B9 | — |
| | | toluene | glassy solid | N | B10 | — |
| | | | Glassy solid | N | B11 | — |
| | | | Morphology | Y | | — |
| | | | unknown | | | |
| | | | glassy solid with a | N | B12 | — |
| | | | few birefringent | | | |
| | | | particles | | | |
| citric | MeOH | ACN | light brown, | N | C1 | crystalline 1 |
| | | | morphology unknown | | | |
| | | | light brown, | N | C2 | — |
| | | | morphology unknown | | | |
| | | | brown, morphology | part. Y | C3 | crystalline 1 |
| | | | unknown | | | |
| | | EtOAc | light brown, | N | C4 | crystalline 1 |
| | | | morphology unknown | | | |
| | | | yellow plates | Y | C5 | crystalline 1 |
| | | | orange, morphology | N | C6 | crystalline 1 |
| | | | unknown | | | |
| | | 1-PrOH | dark brown solid | N | C7 | — |
| | | | brown, morphology | N | C8 | — |
| | | | unknown | | | |
| | | | dark brown solid | N | C9 | — |
| | | toluene | light brown, glass | N | C10 | — |
| | | | light brown, glass | N | C11 | — |
| | | | light brown, glass | N | C12 | — |
| gentisic | MeOH | ACN | dark brown, glass | N | D1 | — |
| | | | dark brown, glass | N | D2 | — |
| | | | dark brown, glass | N | D3 | — |
| | | EtOAc | dark brown, glass | N | D4 | — |
| | | | yellow solid | N | D5 | crystalline 1 |
| | | | light brown, stacked | Y | D6 | crystalline 2 |
| | | | plates | | | |
| | | 1-PrOH | clear, glass | N | D7 | — |
| | | | clear brown, glass | N | D8 | — |
| | | | clear brown, glass | N | D9 | — |
| | | toluene | clear brown, glass | N | D10 | — |
| | | | clear brown, glass | N | D11 | — |
| | | | clear brown, glass | N | D12 | — |
| glutaric | MeOH | ACN | dark brown, morphology | Part. Y | E1 | crystalline 1 |
| | | | unknown | | | |
| | | | dark brown, morphology | Part. Y | E2 | crystalline 1 |
| | | | unknown | | | |
| | | | dark brown, morphology | Part. Y | E3 | low |
| | | | unknown | | | crystalline 1 |
| | | EtOAc | dark brown, morphology | Part. Y | E4 | crystalline 1 |
| | | | unknown | | | |
| | | | orange, morphology | N | E5 | crystalline 1 |
| | | | unknown | | | |
| | | | orange, morphology | Part. Y | E6 | crystalline 1 |
| | | | unknown | | | |
| | | 1-PrOH | clear brown, glass | N | E7 | — |
| | | | clear brown, glass | N | E8 | — |
| | | | clear brown, glass | N | E9 | — |
| | | toluene | dark brown, glass | N | E10 | — |
| | | | dark brown, glass | N | E11 | — |
| | | | dark brown, glass | N | E12 | — |
| glycolic | MeOH | ACN | brown, morphology | N | F1 | crystalline 1 |
| | | | unknown | | | |
| | | | brown, morphology | N | F2 | low |
| | | | unknown | | | crystalline 1 |
| | | | brown, morphology | N | F3 | low |
| | | | unknown | | | crystalline 1 |
| | | EtOAc | brown, morphology | N | F4 | — |
| | | | unknown | | | |
| | | | orange, morphology | N | F5 | — |
| | | | unknown | | | |
| | | | orange, morphology | N | F6 | — |
| | | | unknown | | | |
| | | 1-PrOH | dark brown, morphology | N | F7 | — |
| | | | unknown | | | |
| | | | small amount of dark | N | F8 | — |
| | | | brown, morphology | | | |
| | | | unknown | | | |
| | | | small amount of dark | N | F9 | — |
| | | | brown, morphology | | | |
| | | | unknown | | | |
| | | toluene | glass and some | N | F10 | — |
| | | | birefringent particles | | | |
| | | | brown, glass | N | F11 | — |
| | | | brown, glass | N | F12 | — |
| L-malic | MeOH | ACN | brown, morphology | Part. Y | G1 | crystalline 1 |
| | | | unknown | | | |
| | | | brown, morphology | Part. Y | G2 | — |
| | | | unknown | | | |
| | | | brown, morphology | Part. Y | G3 | crystalline 1 |
| | | | unknown | | | |
| | | EtOAc | brown solid | N | G4 | — |
| | | | brown solid | N | G5 | — |
| | | | brown solid | N | G6 | — |
| | | 1-PrOH | brown glass | N | G7 | — |
| | | | clear glass | N | G8 | — |
| | | | brown glass | N | G9 | — |
| | | toluene | clear brown glass | N | G10 | — |
| | | | brown, morphology | Y | G11 | amorphous |
| | | | unknown | | | with peaks |
| | | | clear brown glass | N | G12 | — |
| none | MeOH | ACN | clear brown glass | N | H1 | — |
| | | | clear brown glass | N | H2 | — |
| | | EtOAc | clear brown glass | N | H4 | — |
| | | | clear brown glass | N | H5 | — |
| | | 1-PrOH | clear glass | N | H7 | — |
| | | | clear glass | N | H8 | — |
| | | toluene | dark brown glass | N | H10 | — |
| | | | dark brown glass | N | H11 | — |

^{a}MeOH = methanol.
^{b}ACN = acetonitrile, EtOAc = ethyl acetate, 1-PrOH = 1-propanol.
^{c}B = birefringence, samples observed under microscope with crossed polarized light; Y = yes, N = no, Part. = partial.

Wellplate 4

Recrystallization of wellplate 3 was conducted using solvent/antisolvent evaporation. The solids in wells were dissolved in methanol. Acetonitrile, ethyl acetate, 1-propanol, and toluene were used as the antisolvents. The wells with sufficient amounts of non-glassy solids were analyzed by XRPD and the results are summarized in Table 21A and Table 18A above.

TABLE 21A
Recrystallization of Wellplate 4
to all wells methanol was added; solvent:antisolvent 1:1

| | | Anti- | | | Well | XRPD |
|---|---|---|---|---|---|---|
| Acid | Solvent^{a} | solvent^{b} | Observations | B/E^{c} | No. | Results |
| HBr | MeOH | ACN | orange, morphology unknown | partial | A1 | crystalline 2 |
| | | | yellow fibers | Y | A2 | low |
| | | | | | | crystalline 2 |
| | | | yellow needles | Y | A3 | crystalline 2 |
| | | EtOAc | off-white, morphology | N | A4 | amorphous |
| | | | unknown | | | with peaks |
| | | | fibers, morphology unknown | Y | | |
| | | | off-white, morphology | N | A5 | crystalline 1 |
| | | | unknown | | | |
| | | | off-white, morphology | partial | A6 | crystalline 1 |
| | | | unknown | | | |
| | | IPA | colorless fibers | Y | A7 | amorphous |
| | | | caramel-colored, morphology | N | A8 | crystalline 1 |
| | | | unknown | | | |
| | | | caramel-colored, morphology | Y | A9 | crystalline 2 |
| | | | unknown | | | |
| | | toluene | yellow, morphology unknown | N | A10 | crystalline 1 |
| | | | yellow, morphology unknown | N | A11 | crystalline 1 |
| | | | yellow, morphology unknown | N | A12 | crystalline 1 |
| lactic | MeOH | ACN | yellow glass | N | B1 | amorphous |
| | | | morphology unknown | Y | | |
| | | | yellow glass | N | B2 | amorphous |
| | | | morphology unknown | Y | | |
| | | | yellow irregular plates and | Y | B3 | amorphous |
| | | | morphology unknown | | | |
| | | EtOAc | colorless glass | N | B4 | amorphous |
| | | | one fiber | Y | | |
| | | | colorless glass | N | B5 | amorphous |
| | | | morphology unknown | Y | | |
| | | | colorless fibers | Y | B6 | amorphous |
| | | IPA | off-white, morphology | partial | B7 | amorphous |
| | | | unknown | | | |
| | | | off-white, morphology | N | B8 | amorphous |
| | | | unknown | | | |
| | | | off-white, morphology | partial | B9 | amorphous |
| | | | unknown | | | |
| | | toluene | colorless glass | N | B10 | amorphous |
| | | | one fiber | Y | | |
| | | | colorless oil | N | B11 | amorphous |
| | | | morphology unknown | Y | | with peaks |
| | | | white, morphology unknown | N | B12 | crystalline 1 |
| maleic | MeOH | ACN | orange, morphology | N | C1 | crystalline 1 + |
| | | | unknown | | | one peak |
| | | | caramel-colored, | N | C2 | crystalline 1 |
| | | | morphology unknown | | | |
| | | | caramel-colored, | N | C3 | crystalline 1 + |
| | | | morphology unknown | | | one peak |
| | | EtOAc | yellow, morphology | N | C4 | crystalline 1 + |
| | | | unknown | | | one peak |
| | | | off-white, morphology | N | C5 | crystalline 1 |
| | | | unknown | | | |
| | | | pink, morphology unknown | partial | C6 | crystalline 1 + |
| | | | | | | one peak |
| | | IPA | caramel-colored glass | N | C7 | amorphous |
| | | | blades | Y | | |
| | | | caramel-colored glass | N | C8 | amorphous |
| | | | blades | Y | | |
| | | | caramel-colored glass | N | C9 | amorphous |
| | | | morphology unknown | Y | | |
| | | toluene | pink, morphology unknown | N | C10 | crystalline 1 + |
| | | | | | | one peak |
| | | | off-white, morphology | N | C11 | crystalline 1 + |
| | | | unknown | | | one peak |
| | | | white, morphology | N | C12 | crystalline 1 + |
| | | | unknown | | | one peak |
| methane- | MeOH | ACN | yellow, glass | N | D1 | amorphous |
| sulfonic | | | fibers | Y | | |
| | | | yellow glass | N | D2 | amorphous |
| | | | morphology unknown | Y | | |
| | | | yellow glass | N | D3 | amorphous |
| | | | fibers | N | | |
| | | EtOAc | yellow glass | N | D4 | amorphous |
| | | | yellow glass | N | D5 | amorphous |
| | | | fibers | Y | | |
| | | | yellow glass | N | D6 | amorphous |
| | | | fibers | Y | | |
| | | IPA | colorless glass | N | D7 | amorphous |
| | | | morphology unknown | Y | | |
| | | | yellow oil | N | D8 | amorphous |
| | | | yellow oil | N | D9 | amorphous |
| | | | morphology unknown | Y | | |
| | | toluene | orange glass | N | D10 | amorphous |
| | | | red glass and morphology | N | D11 | amorphous |
| | | | unknown | | | with peaks |
| | | | orange glass | N | D12 | amorphous |
| | | | | | | with peaks |
| succinic | MeOH | ACN | caramel-colored, | Y | E1 | crystalline 1 |
| | | | morphology unknown | | | |
| | | | caramel-colored, | partial | E2 | crystalline 1 |
| | | | morphology unknown | | | |
| | | | caramel-colored, | partial | E3 | crystalline 1 |
| | | | morphology unknown | | | |
| | | EtOAc | off-white, morphology | N | E4 | crystalline 1 |
| | | | unknown | | | |
| | | | off-white, morphology | N | E5 | crystalline 1 |
| | | | unknown | | | |
| | | | blades | Y | | |
| | | | pink, morphology unknown | N | E6 | crystalline 1 |
| | | IPA | brown glass | N | E7 | amorphous |
| | | | fibers and blades | Y | | |
| | | | brown glass | N | E8 | amorphous |
| | | | morphology unknown | Y | | |
| | | | brown glass | N | E9 | amorphous |
| | | | morphology unknown | Y | | |
| | | toluene | pink blades and rectangular | Y | E10 | crystalline 2 |
| | | | plates | | | |
| | | | colorless blades and | Y | E11 | crystalline 2 |
| | | | rectangular plates | | | minus peaks |
| | | | colorless irregular plates | Y | E12 | crystalline 2 |
| sulfuric | MeOH | ACN | caramel-colored, | Y | F1 | crystalline 1 |
| | | | morphology unknown | | | |
| | | | off-white, morphology | N | F2 | crystalline 1 |
| | | | unknown | | | |
| | | | caramel-colored, | Y | F3 | crystalline 1 |
| | | | morphology unknown | | | |
| | | EtOAc | off-white, morphology | Y | F4 | crystalline 1 |
| | | | unknown | | | minus peaks |
| | | | colorless, morphology | Y | F5 | crystalline 1 |
| | | | unknown | | | minus peaks |
| | | | colorless, morphology | Y | F6 | crystalline 2 |
| | | | unknown | | | minus peaks |
| | | IPA | brown, morphology | N | F7 | crystalline 1 |
| | | | unknown | | | |
| | | | brown, morphology | N | F8 | crystalline 1 |
| | | | unknown | | | minus peaks |
| | | | brown, morphology | N | F9 | crystalline 1 |
| | | | unknown | | | |
| | | toluene | off-white, morphology | partial | F10 | crystalline 1 |
| | | | unknown | | | |
| | | | white, morphology unknown | N | F11 | crystalline 1 |
| | | | white, morphology unknown | N | F12 | crystalline 1 |
| phosphoric | MeOH | ACN | orange, morphology | N | G1 | crystalline 2 |
| | | | unknown | | | |
| | | | orange, morphology | N | G2 | crystalline 1 |
| | | | unknown | | | |
| | | | orange, morphology | N | G3 | crystalline 1 |
| | | | unknown | | | |
| | | EtOAc | off-white, morphology | N | G4 | crystalline 1 |
| | | | unknown | | | |
| | | | off-white, morphology | N | G5 | crystalline 1 |
| | | | unknown | | | |
| | | | off-white, morphology | N | G6 | crystalline 1 + |
| | | | unknown | | | peaks |
| | | | blades | Y | | |
| | | IPA | brown, morphology | N | G7 | crystalline 3 |
| | | | unknown | | | |
| | | | caramel-colored, | N | G8 | crystalline 4 |
| | | | morphology unknown | | | |
| | | | pink, morphology unknown | N | G9 | amorphous |
| | | | | | | with peaks |
| | | toluene | off-white, morphology | N | G10 | crystalline 1 |
| | | | unknown | | | |
| | | | white, morphology unknown | N | G11 | crystalline 1 |
| | | | white, morphology unknown | N | G12 | crystalline 1 |
| none | MeOH | ACN | yellow glass | N | H1 | amorphous |
| | | | morphology unknown | Y | | |
| | | | yellow glass | N | H2 | amorphous |
| | | | morphology unknown | Y | | |
| | | EtOAc | colorless, morphology | N | H4 | amorphous |
| | | | unknown | | | |
| | | | fibers | Y | | |
| | | | colorless fibers | Y | H5 | amorphous |
| | | IPA | yellow fibers and | Y | H7 | amorphous |
| | | | morphology unknown | | | |
| | | | yellow glass | N | H8 | amorphous |
| | | | morphology unknown | Y | | |
| | | | yellow oil | N | H9 | amorphous |
| | | | morphology unknown | Y | | |
| | | toluene | yellow glass | N | H10 | amorphous |
| | | | morphology unknown | Y | | |
| | | | colorless oil and | N | H11 | amorphous |
| | | | morphology unknown | | | |

^{a}MeOH = methanol.
^{b}ACN = acetonitrile, EtOAc = ethyl acetate, IPA = isopropanol.
^{c}B = birefringence, E = extinction; samples observed under microscope with crossed polarized light; Y = yes, N = no.

Summary of Crystalline Salts from Wellplates: Salt MicroScreen™

The following new crystalline salts were discovered from wellplate crystallization experiments:

acetate,

adipate,

citrate,

gentisate,

glutarate,

glycolate,

hydrobromide,

lactate,

L-malate,

maleate,

phosphate,

succinate,

sulfate.

The crystalline salts are summarized in Table 18A above. The preparation and crystallization experiments are discussed below.

Acetate Salt

A new crystalline XRPD pattern (crystalline 1) was observed in the experiments with acetic acid in acetone and methanol (FIG. 21). Material exhibiting this XRPD pattern was also produced in the microplate recrystallization experiments using methanol:acetonitrile 1:1 and methanol: ethyl acetate 1:1 (see summary table).

The acetate salt (crystalline 1) was initially prepared on approximately 50-mg scale from methanol solution (evaporation to dryness, Table 22A). The salt structure was confirmed by proton NMR (FIG. 22, Table 23A). Approximate solubility data for the acetate salt are given in Table 61A.

The acetate salt (crystalline 1) was crystallized with approximately 70% yield by fast evaporation from methanol (Table 24A). The material was characterized using thermal techniques (FIG. 23, Table 25A). A two-step weight loss of approximately 16% was observed in TG at higher temperatures and was likely due to salt decomposition with the loss of the acetic acid. An endotherm at approximately 190° C. with a shoulder at 194° C. in DSC corresponded to the weight loss in TG. Thus, the shoulder at 194° C. probably indicated the melt of the free base. Therefore, the acetate salt decomposed on heating to higher temperatures (approximately 100-150° C.).

The aqueous solubility of the acetate salt was approximately 14 mg/mL (Table 64A).

TABLE 22A
Salt Preparation Attempts from Compound 2

| | Solvent | | | XRPD |
|---|---|---|---|---|
| Acid^{a} | System | Conditions^{b} | Description^{c} | Result^{d} |
| acetic | MeOH | FE | translucent glassy film, not | crystalline 1 |
| | | | birefringent; white, | |
| | | | morphology unknown, | |
| | | | birefringent | |
| | acetone | FE | brownish glassy solid, not | — |
| | | | birefringent | |
| | | SE | brownish glassy solid, not | — |
| | | | birefringent | |
| adipic | MeOH | FE | white needles, birefringent; | crystalline 1 |
| | | | white, morphology unknown, | |
| | | | not birefringent | |
| | acetone:MeOH | FE | yellow glassy solid, not | — |
| | 95:5 | | birefringent | |
| | | SE | brownish glassy solid, not | — |
| | | | birefringent | |
| citric | MeOH | FE | white flakes, partially | crystalline 1 |
| | | | birefringent; clear oily film, | |
| | | | not birefringent | |
| | acetone:MeOH | FE | clear glassy solid, not | — |
| | 96:4 | | birefringent | |
| | | SE | off-white spherulites of tiny | crystalline 2 |
| | | | needles | |
| gentisic | MeOH | RT slurry, 4d^{e} | clear solution | — |
| | | CP w/ ether, RT | off-white wispy chunks | IS |
| | | 3d^{f} | (visual) | |
| | MeOH:EtOAc | FE | clear oily film, not | crystalline 1 |
| | 1:1 | | birefringent; white, | |
| | | | morphology unknown, | |
| | | | birefringent | |
| glutaric | MeOH:EtOAc | FE | white dendridic fibers and | crystalline 1 |
| | 1:1 | | morphology unknown, | |
| | | | birefringent | |
| glycolic | MeOH:ACN | FE | white, morphology unknown, | crystalline 1 |
| | 1:1 | | partially birefringent | |

^{a}Acid/API molar ratio is 1:1 unless specified otherwise
^{b}CP = crash precipitation, FE = fast evaporation, SE = slow evaporation, RT = ambient
temperature, d = days; reported times are approximate
^{c}Samples observed under microscope with crossed polarized light
^{d}IS = insufficient solids for analysis
^{e}Precipitate generated upon acid addition
^{f}Opaque liquid generated upon antisolvent addition
^{g}1:1 equivalents Acid/API

| | Solvent | | | XRPD |
|---|---|---|---|---|
| Acid^{a} | System | Conditions^{b} | Description^{c} | Result^{d} |
| HBr | acetone | FE | off-white needles, blades, and | crystalline 3 |
| | | | morphology unknown, | |
| | | | birefringent | |
| | MEK | FE | clear fibers, birefringent; | — |
| | | | purple sticky film, not | |
| | | | birefringent | |
| | | | clear, morphology unknown, | — |
| | | | birefringent; purple sticky | |
| | | | film, not birefringent | |
| | TFE | spontaneous | white, morphology unknown, | crystalline 1 |
| | | precipitation | not birefringent | |
| lactic | MeOH:toluene | FE | clear glassy film, not | amorphous |
| | 1:1 | | birefringent; colorless fibers, | |
| | | | birefringent | |
| maleic | MeOH | FE | white, morphology unknown, | crystalline 1 + |
| | | | birefringent | peaks |
| | acetone:MeOH | FE | white, morphology unknown, | crystalline 1 + |
| | 96:4 | | birefringent and yellowish | peaks |
| | | | film, not birefringent | |
| L-malic | MeOH | RT slurry, 4d^{f} | clear solution | — |
| | | CP w/ ether, RT | dark, wispy solids, not | amorphous |
| | | 3d^{e} | birefringent | |
| | | FE | white, morphology unknown, | crystalline 1 |
| | | | birefringent | |
| phosphoric | MeOH | RT stir 3d^{f} | dark wispy solids, irregular | crystalline 6 |
| | | | particles, birefringent | |
| | TFE/MeOH | RT stir 3d^{f} | dark wispy solids, irregular | low crystalline 7 |
| | | | particles, birefringent | |
| | acetone | FE | white flakes, birefringent | amorphous |
| | MeOH | FE | white, morphology unknown, | crystalline 5 |
| | | | partially birefringent | |

^{a}Acid/API molar ratio is 1:1 unless specified otherwise
^{b}CP = crash precipitation, FE = fast evaporation, SE = slow evaporation, RT = ambient
temperature, d = days; reported times are approximate
^{c}Samples observed under microscope with crossed polarized light
^{d}IS = insufficient solids for analysis
^{e}Opaque liquid generated upon antisolvent addition
^{f}Precipitate generated upon acid addition
^{g}1:1 equivalents Acid/API

| | Solvent | | | XRPD |
|---|---|---|---|---|
| Acid^{a} | System | Conditions^{b} | Description^{c} | Result^{d} |
| phosphoric | MEK | FE | clear fibers, birefringent; | — |
| | | | light brown sticky film, not | |
| | | | birefringent | |
| | | | purple sticky film, not | — |
| | | | birefringent | |
| succinic | MeOH | FE | white, morphology unknown, | crystalline 1 |
| | | | birefringent | |
| | TFE:MeOH | FE | clear, glassy, not birefringent | — |
| | 5:1 | | | |
| | TFE:MeOH | FE | white, morphology unknown, | crystalline 3 |
| | 10:1 | | birefringent | |
| | | SE | off-white, morphology | crystalline 1 |
| | | | unknown, birefringent | |
| | toluene:MeOH | FE | white, morphology unknown, | crystalline 1 |
| | 1:1 | | partially birefringent | |
| sulfuric | MeOH:EtOAc | FE | off-white needles, | crystalline 6 |
| | 1:1 | | birefringent | |
| | acetone | API/Acid (2/1); FE | white, glassy, not birefringent | amorphous |
| | MeOH | API/Acid (2/1); FE | white, small needles, | crystalline 1 |
| | | | birefringent | |
| | acetone | API/Acid (2/1); | off-white, clump of irregular | crystalline 7 |
| | | slurry | shaped particles, birefringent | |
| | acetone | API/Acid (1/1); FE | white, irregular shape, | crystalline 5 |
| | | | birefringent | |
| | MeOH | API/Acid (1/1); FE | white, fragments, birefringent | crystalline 6 |
| | MeOH | API/Acid (1/1); SE | white, fragments, birefringent | crystalline 6 |
| | acetone/MeOH | RT stir 1d/SE | wisps, irregular particles, | crystalline 1 |
| | | (RT stir 4d total)^{e} | blades, birefringent | (small amount |
| | | | | of sample) |
| | TFE/MeOH | RT stir 3d^{e} | dark fine wisps, not | low crystalline 8 |
| | | | birefringent | |

^{a}Acid/API molar ratio is 1:1 unless specified otherwise
^{b}CP = crash precipitation, FE = fast evaporation, SE = slow evaporation, RT = ambient
temperature, d = days; reported times are approximate
^{c}Samples observed under microscope with crossed polarized light
^{d}IS = insufficient solids for analysis
^{e}Precipitate generated upon acid addition
^{f}1:1 equivalents Acid/API

TABLE 23A
Characterization of Acetate Salt

| Technique | Analysis/Result |
|---|---|
| XRPD | crystalline 1 |
| ¹H NMR | consistent w/structure |

TABLE 24A
Salt Preparation Scale-up Experiments using compound 2

| | Solvent/Solvent | | | Yield | XRPD |
|---|---|---|---|---|---|
| Acid | System | Method^{a} | Description | (%) | Result^{d} |
| acetic | MeOH | SC | clear solution | — | — |
| | MeOH | FE | off-white solid, | 70.2 | crystalline 1 |
| | | | morphology unknown, | | |
| | | | birefringent | | |
| | acetonitrile:MeOH | FE | yellow, dendridic | 74.4 | crystalline 1 |
| | 1:1 | | formations, | | |
| | | | birefringent | | |
| adipic | MeOH | SC | clear solution | — | — |
| | MeOH | FE | off-white solid, | 72.4 | crystalline 1 |
| | | | morphology unknown, | | |
| | | | birefringent | | |
| | acetonitrile:MeOH | FE | light yellow, | 58.1 | crystalline 1 |
| | 1:1 | | spherulites of blades, | | |
| | | | birefringent | | |
| citric | acetone:MeOH | SC | off-white, spherulites | 109.6^{b} | crystalline 2 |
| | 98:2 | | of needles, | | |
| | | | birefringent | | |
| glycolic | acetonitrile:MeOH | SC | white, blades, | 80.5 | crystalline 1 |
| | 1:1 | | birefringent | | |
| HBr | acetonitrile:MeOH | SC | clear solution | — | — |
| | 1:1 | | | | |
| | acetonitrile:MeOH | SC, then | yellowish solid, | 63.7 | crystalline 1 |
| | 1:1 | FE | morphology unknown, | | |
| | | | partially birefringent | | |
| | | | yellow solid, | 47.6 | crystalline 1 |
| | | | morphology unknown, | | |
| | | | not birefringent | | |
| phosphoric | MeOH | precipitation | white solid | 89.4 | crystalline 2 |
| | | at | | | |
| | | 55° C. | | | |
| | MeOH | FE | white solid, | 82 | crystalline 8, |
| | | | morphology unknown, | | (crystalline 5 |
| | | | not birefringent | | is crystalline |
| | | | | | 8 + peaks) |
| | MeOH | FE | white, morphology | 88.2 | crystalline 8 |
| | | | unknown, birefringent | | |
| | | | and off-white solid, | | |
| | | | rosettes from irregular | | |
| | | | crystals, birefringent | | |

^{a}FE = fast evaporation, SC = slow cool
^{b}possible dihydrate, acetone solvate, or mixed hydrate/solvate obtained

TABLE 25A
Characterization of Acetate Salt

| Technique | Analysis/Result |
|---|---|
| XRPD | crystalline 1 |
| DSC^{a} | endo 190, 194 (shoulder) |
| TGA^{b} | 9.88 @ 15-160 |
| | 6.37 @160-195 |

^{a}endo = endotherm, temperatures (C.°) reported are transition maxima. Temperatures are rounded to the nearest degree.
^{b}weight loss (%) at a certain temperature; weight changes (%) are rounded to 2 decimal places; temperatures are rounded to the nearest degree

Adipate

A new crystalline XRPD pattern and a similar low crystalline pattern (crystalline 1 and low crystalline 1) were observed in the experiments with adipic acid in acetone. Material exhibiting the XRPD pattern of crystalline 1 without some peaks was produced from methanol (FIGS. 24a to d).

Material exhibiting the XRPD pattern of crystalline 1 also resulted from the microplate recrystallization experiment using methanol:acetonitrile 1:1 and methanol: ethyl acetate 1:1 (see summary table).

The adipate salt (crystalline 1) was prepared on approximately 50-mg scale by fast evaporation in methanol (to dryness, Table 22A above). The salt structure was confirmed by proton NMR (FIG. 25, Table 26A). Approximate solubility data for the adipate salt are given in Table 62A.

The adipate salt (crystalline 1) was crystallized by fast evaporation in methanol (approx. 72% yield) and acetonitrile:methanol 1:1 (approx. 58% yield) (Table 24A above). The sample prepared from methanol was analyzed by thermal techniques (FIG. 26, Table 27A). The sample exhibited a gradual weight loss of approximately 5.0% from 20 to 155° C. in TG. A smaller broad endotherm (likely desolvation/dehydration) at approximately 91° C. in DSC was followed by a broad intense endotherm at approximately 145° C. The DSC data likely indicated melt/decomposition occurred simultaneously.

The aqueous solubility of the adipate salt was approximately 10 mg/mL (Table 64A).

TABLE 26A
Characterization of Adipate Salt

| Technique | Analysis/Result |
|---|---|
| XRPD | crystalline 1 |
| ¹H NMR | consistent w/structure |

TABLE 27A
Characterization of Adipate Salt

| Technique | Analysis/Result |
|---|---|
| XRPD | crystalline 1 |
| DSC^{a} | endo 91(small), 145 |
| TGA^{b} | 5.00 @ 20-155 |

a and b as above

Citrate

A new crystalline XRPD pattern (crystalline 1) was observed in the experiment with citric acid in acetone. A similar low crystalline XPRD pattern (low crystalline 1) was observed in the experiments utilizing acetone, methanol, and methyl ethyl ketone as solvents (FIG. 27a to d).

Material exhibiting the XRPD pattern of crystalline 1 also resulted from a microplate recrystallization experiment using methanol:acetonitrile 1:1 and methanol: ethyl acetate 1:1 (see summary table).

Two crystalline forms of the citrate salt were prepared from scale-up experiments (Table 22A). Material exhibiting the XRPD pattern of crystalline 1 resulted from a fast evaporation experiment in methanol. A new material with an XRPD pattern designated as crystalline 2 was produced in a slow evaporation experiment in acetone:methanol 96:4 (Table 22A). The salt structure was confirmed by proton NMR for both samples (FIG. 29, FIG. 30, Table 28A, Table 29A). Based on NMR, impurities were present in the crystalline 2 material.

The citrate salt (crystalline 2) was scaled up by crystallization in acetone:methanol 98:2 (slow cool, Table 24A). Approximately 110% yield was calculated, however, an insignificant weight loss (0.3%) was observed after the material had been dried in vacuum for three days. Based on proton NMR, approximately 0.5 moles of acetone were found per one mole of the compound (FIG. 35).

The citrate salt was characterized by thermal techniques (FIG. 31, Table 30A). A weight loss of approximately 1% between 25 and 115° C. in TG was probably due to desolvation. A broad endotherm was observed in DSC at approximately 82° C., likely due to loss of solvent. The DSC exhibited a sharper intensive endotherm at approximately 148° C. Based on weight loss in TG, the endotherm likely resulted from simultaneous melt/decomposition.

The aqueous solubility of the citrate salt was approximately 12 mg/mL (Table 64A).

TABLE 28A
Characterization of Citrate Salt, crystalline 1

| Technique | Analysis/Result |
|---|---|
| XRPD | crystalline 1 |
| ¹H NMR | consistent w/structure |

TABLE 29A
Characterization of Citrate Salt, crystalline 2

| Technique | Analysis/Result |
|---|---|
| XRPD | crystalline 2 |
| ¹H NMR | impurities present |

TABLE 30A
Characterization of Citrate Salt, crystalline 2

| Technique | Analysis/Result |
|---|---|
| XRPD | crystalline 2 |
| ¹H NMR | consistent w/structure |
| DSC^{a} | endo 82 (small), 148 |
| TGA^{b} | 1.01 @ 25-115 |

^{a}endo = endotherm, temperatures (C.°) reported are transition maxima. Temperatures are rounded to the nearest degree.
^{b}weight loss (%) at a certain temperature; weight changes (%) are rounded to 2 decimal places; temperatures are rounded to the nearest degree

Gentisate

No crystalline materials were generated in the experiments with gentisic acid in the original wellplate salt preparation (Table 17A).

Two crystalline materials exhibiting XRPD patterns designated as crystalline 1 and crystalline 2 resulted from wellplate recrystallization experiments in methanol: ethyl acetate 1:1 (FIGS. 32a, 32b and 32c, Table 20A). Based on proton NMR, the crystalline 2 material was the gentisate salt that contained approximately 0.7 moles of ethyl acetate (FIG. 34, Table 32A).

The crystalline 1 material was obtained in a scale-up attempt by fast evaporation in methanol: ethyl acetate 1:1 (evaporation to dryness,). Based on ¹H NMR, the material was a likely mixture of the free base and the gentisate salt (FIG. 33, Table 31A).

The aqueous solubility of the gentisate salt was lower than 1 mg/mL (Table 63A)

TABLE 31A
Characterization of Gentisate Salt, crystalline 1

| Technique | Analysis/Result |
|---|---|
| XRPD | crystalline 1 |
| ¹H NMR | salt + free base |

TABLE 32A
Characterization of Gentisate Salt, crystalline 2

| Technique | Analysis/Result |
|---|---|
| XRPD | crystalline 2 |
| ¹H NMR | 0.7 mole of EtOAc per 1 mole of |
| | compound |

Glutarate

No crystalline materials were generated in the experiments with glutaric acid in the original wellplate salt preparation (Table 17A).

Material exhibiting an XRPD pattern designated as crystalline 1 was generated in the microplate recrystallization experiments using methanol:acetonitrile 1:1 and methanol: ethyl acetate 1:1 (FIGS. 35a, 35b and 35c, Table 20A).

The glutarate salt (crystalline 1) was crystallized by fast evaporation in methanol: ethyl acetate 1:1 (evaporation to dryness, Table 22A). The salt structure was confirmed by ¹H NMR (FIG. 36, Table 33A).

The aqueous solubility of the glutarate salt was approximately 3 mg/mL (Table 63A).

TABLE 33A
Characterization of Glutarate Salt

| Technique | Analysis/Result |
|---|---|
| XRPD | crystalline 1 |
| ¹H NMR | consistent w/structure |

Glycolate

No crystalline materials were generated in the experiments with glycolic acid in the original wellplate salt preparation (Table 17A).

Material exhibiting an XRPD pattern designated as crystalline 1 resulted from the microplate recrystallization experiment in methanol:acetonitrile 1:1 (FIGS. 37a, 37b and 37c, Table 20A).

The glycolate salt (crystalline 1) was produced on approx. 50-mg scale by fast evaporation using methanol:acetonitrile 1:1 (Table 22A). The salt structure was confirmed by ¹H NMR (FIG. 38, Table 34A, residual acetonitrile present).

The glycolate salt was prepared with approx. 80% yield by slow cooling in acetonitrile:methanol 1:1 (Table 24A). The material was analyzed using thermal techniques (FIG. 39, Table 35A). The baseline in DSC at lower temperatures indicated possible loss of residual solvent. A weight loss of approximately 8.5% in TG was accompanied by a sharp endotherm at approximately 147° C., probably due to the melt and concurrent decomposition. DSC and TG thermograms exhibited further decomposition above 150° C. (endotherms at 192 and 204° C.).

The aqueous solubility of the glycolate salt was approximately 27 mg/mL (Table 64A).

TABLE 34A
Characterization of Glycolate Salt

| Technique | Analysis/Result |
|---|---|
| XRPD | crystalline 1 |
| ¹H NMR | consistent w/structure, |
| | residual acetonitrile |

TABLE 35A
Characterization of Glycolate Salt

| Technique | Analysis/Result |
|---|---|
| XRPD | crystalline 1 |
| DSC | endo 147 (87 J/g), 192, 204 |
| TGA | 8.52 @ 20-155 |

Hydrobromide

The crystalline XRPD patterns of the hydrobromide salt found in the screen are presented in FIGS. 40a to 40e.

Two new crystalline XRPD patterns were observed in the wellplate preparation experiments with hydrobromic acid in trifluoroethanol (crystalline 1) and in acetone and methyl ethyl ketone (crystalline 2) (Table 19A).

Material exhibiting the XRPD pattern of crystalline 1 was also produced in wellplate recrystallization experiments using methanol: ethyl acetate, methanol: isopropanol, and methanol:toluene 1:1 solvent systems (Table 21A).

Material exhibiting the XRPD pattern of crystalline 2 was obtained in wellplate recrystallization experiments using methanol: acetonitrile and methanol:isopropanol 1:1 (Table 21A). Presence of impurities was noted in proton NMR (FIG. 42, Table 37A). A low crystalline pattern 2 was detected by XRPD in a recrystallization experiment in methanol:acetonitrile 1:1.

Two crystalline forms of the HBr salt were prepared from the scale-up experiments (Table 22A). Material exhibiting the XRPD pattern of crystalline 1 resulted from a fast evaporation experiment in 2,2,2-trifluoroethanol (TFE) and contained residual trifluoroethanol, based on ¹H NMR (FIG. 41, Table 36A). Material exhibiting a new XRPD pattern designated as crystalline 3 was produced by fast evaporation in acetone. It contained impurities as shown by proton NMR (FIG. 43, Table 38A).

The hydrobromide salt was crystallized from acetonitrile:methanol 1:1 with approx. 64% yield and characterized by thermal techniques (Table 24A, FIG. 44, Table 39A). Crystalline 1 material was produced from two preparation experiments. A weight loss of approximately 0.72% was observed in TG between 19 and 205° C. The DSC indicated initial loss of residual solvent (broad endotherm at approx. 48° C.). The endotherm at approximately 234° C. was likely due to the melt.

The aqueous solubility of the hydrobromide salt was approximately 16 mg/mL (Table 64A).

TABLE 36A
Characterization of Hydrobromide Salt, Crystalline 1

| Technique | Analysis/Result |
|---|---|
| XRPD | crystalline 1 |
| ¹H NMR | consistent w/structure, |
| | residual trifluoroethanol |

TABLE 37A
Characterization of Hydrobromide Salt, Crystalline 2

| Technique | Analysis/Result |
|---|---|
| XRPD | crystalline 2 |
| ¹H NMR | impurities present |

TABLE 38A
Characterization of Hydrobromide Salt, Crystalline 3

| Technique | Analysis/Result |
|---|---|
| XRPD | crystalline 3 |
| ¹H NMR | impurities present |

TABLE 39A
Characterization of Hydrobromide Salt, Crystalline 1

| Technique | Analysis/Result |
|---|---|
| XRPD | crystalline 1 |
| DSC^{a} | endo 48 (small), 198 (small), 234 (77 J/g) |
| TGA^{b} | 0.72 @ 19-205 |

^{a}endo = endotherm, temperatures (C.°) reported are transition maxima. Temperatures are rounded to the nearest degree.
^{b}weight loss (%) at a certain temperature; weight changes (%) are rounded to 2 decimal places; temperatures are rounded to the nearest degree

Lactate

No crystalline materials were generated in the experiments with lactic acid in the original wellplate salt preparation (Table 19A).

Material exhibiting an XRPD pattern designated as crystalline 1 resulted from the microplate recrystallization experiment in methanol:toluene 1:1 (FIG. 45, Table 21A). A mixture of the free base and a small amount of lactic acid with impurities was detected by proton NMR (very small amount of material, FIG. 46, Table 40A).

A scale-up attempt by fast evaporation using the same solvent system was unsuccessful and resulted in amorphous material (Table 22A).

TABLE 40A
Characterization of Lactate Salt

| Technique | Analysis/Result |
|---|---|
| XRPD | crystalline 1 |
| ¹H NMR | free base + small amount of lactic acid |
| | (very small concentration) |

L-Malate

A new crystalline XRPD pattern (crystalline 1) was observed in the original wellplate salt preparation with L-malic acid in methanol (FIGS. 47a and 47b, Table 17A). Material exhibiting the XRPD pattern of crystalline 1 was also produced in a wellplate recrystallization experiment in methanol:acetonitrile 1:1 (Table 20A).

The L-malate salt was also prepared on approx. 50-mg scale by fast evaporation in methanol (evaporation to dryness, Table 22A). The salt structure was confirmed by proton NMR (FIG. 48, Table 41A).

The aqueous solubility of the L-malate salt was approximately 4 mg/mL (Table 63A).

TABLE 41A
Characterization of L-Malate Salt

| Technique | Analysis/Result |
|---|---|
| XRPD | crystalline 1 |
| ¹H NMR | consistent w/structure |

Maleate

Two new crystalline XRPD patterns were observed in the experiments with maleic acid in acetone and methanol (crystalline 1 and crystalline 1 plus one peak). Both results were obtained from both solvents. A low crystalline material with the XRPD pattern similar to crystalline 1 (low crystalline 1) resulted from trifluoroethanol (FIGS. 49a to 49d, Table 19A).

Two crystalline materials exhibiting the XRPD patterns of crystalline 1 and crystalline 1 plus peak were produced in the wellplate recrystallization experiments in methanol: acetonitrile and methanol: ethyl acetate 1:1 solvent systems (FIG. 49, Table 21A). Material exhibiting the XRPD pattern of crystalline 1 plus peak was also produced in methanol:toluene 1:1.

The maleate salt (crystalline 1 plus peaks) was prepared on approximately 50-mg scale by fast evaporation in methanol and acetone:methanol 96:4 (Table 22A). The salt structure was confirmed by proton NMR (FIG. 50, Table 42A).

The aqueous solubility of the maleate salt was approximately 3 mg/mL (Table 63A).

TABLE 42A
Characterization of Maleate Salt

| Technique | Analysis/Result |
|---|---|
| XRPD | maleate |
| | (crystalline 1 + peaks) |
| ¹H NMR | consistent w/structure |

Phosphate

Four new crystalline XRPD patterns were found in the wellplate experiments with phosphoric acid (FIGS. 51a to 51i and FIG. 52, Table 19A). Material exhibiting an XRPD pattern designated as crystalline 1 was produced from methanol and trifluoroethanol. Material exhibiting an XRPD pattern designated as crystalline 1 plus peaks was produced from acetone. Material with a low crystalline 1 pattern resulted from an experiment in methanol.

Material exhibiting an XRPD pattern designated as crystalline 2 resulted from experiments in acetone.

Two crystalline materials exhibiting XRPD patterns designated as crystalline 3 and crystalline 4 were produced in experiments in methyl ethyl ketone.

All the four new crystalline materials were reproduced in wellplate recrystallization experiments by addition of antisolvents such as acetonitrile, ethyl acetate, toluene, and isopropanol to methanol solutions (Table 21A). Based on proton NMR, materials of crystalline 2, crystalline 3, and crystalline 4 had impurities (FIG. 53, FIG. 54, FIG. 55 and Table 44A, Table 45A, Table 46A).

The phosphate salt exhibiting a new XRPD pattern of crystalline 5 (also called crystal modification X) was produced in a scale-up experiment by fast evaporation to dryness in methanol (Table 22A). The salt structure was confirmed by proton NMR (FIG. 56, Table 43A). Two new XRPD patterns for the phosphate salt—crystalline 6 and low crystalline 7—resulted from the scale-up slurry experiments (Table 22A).

Attempts to prepare additional quantities of crystalline materials 1-4 were not successful. Amorphous material resulted from fast evaporation to dryness in acetone.

The phosphate salt (crystalline 2) was crystallized with approx. 89% yield by precipitation from methanol at approx. 55° C. (Table 24A).

The phosphate salt exhibiting a new XRPD pattern designated as crystalline 8 was prepared with approx. 82% yield by fast evaporation from methanol (Table 24A). Crystalline 8 is probably a more thermodynamically stable form of the phosphate salt. After comparison of the XRPD data, crystalline pattern 5 appeared to be very similar to crystalline pattern 8 with some peaks (FIG. 52).

The phosphate salt, crystalline 8, was reproduced in the second scale-up experiment using the same crystallization conditions (Table 24A). The material was analyzed using proton NMR and thermal techniques (FIG. 57, FIG. 58, Table 47A). The TG data showed an insignificant weight loss of approximately 0.24% from 18 to 200° C. A single endotherm in DSC at approximately 233° C. probably corresponded to the melt and initial decomposition.

The aqueous solubility of the phosphate salt was approximately 2-3 mg/mL (Table 64A).

TABLE 43A
Characterization of Phosphate Salt,
Crystalline 5 (Crystalline 8 + peaks)

| Technique | Analysis/Result |
|---|---|
| XRPD | crystalline 5 |
| ¹H NMR | consistent w/structure |

TABLE 44A
Characterization of Phosphate Salt, Crystalline 2

| Technique | Analysis/Result |
|---|---|
| XRPD | crystalline 2 |
| ¹H NMR | impurities present |

TABLE 45A
Characterization of Phosphate Salt, Crystalline 3

| Technique | Analysis/Result |
|---|---|
| XRPD | crystalline 3 |
| ¹H NMR | impurities present |

TABLE 46A
Characterization of Phosphate Salt, Crystalline 4

| Technique | Analysis/Result |
|---|---|
| XRPD | crystalline 4 |
| ¹H NMR | impurities present |

TABLE 47A
Characterization of Phosphate Salt, Crystalline 8

| Technique | Analysis/Result |
|---|---|
| XRPD | crystalline 8 |
| ¹H NMR | consistent w/structure |
| DSC^{a} | endo 233 (134 J/g) |
| TGA^{b} | 0.24 @ 18-200 |

^{a}endo = endotherm, temperatures (C.°) reported are transition maxima. Temperatures are rounded to the nearest degree.
^{b}weight loss (%) at a certain temperature; weight changes (%) are rounded to 2 decimal places; temperatures are rounded to the nearest degree

Succinate

Material exhibiting an XRPD pattern designated as crystalline 1 was observed in the experiments with succinic acid in acetone, methanol, and trifluoroethanol (FIG. 60, Table 19A). Experiments utilizing acetone and trifluoroethanol also produced low crystalline 1 material.

Material exhibiting the XRPD pattern of crystalline 1 was then produced in recrystallization experiments using methanol: acetonitrile and methanol: ethyl acetate 1:1 (Table 21A).

Two new crystalline materials exhibiting XRPD patterns designated as crystalline 2 and crystalline 2 minus peaks were generated in recrystallization experiments in methanol:toluene 1:1 (Table 21A). Based on ¹H NMR, impurities were present in the succinate salt of crystalline 2 (FIG. 61, Table 49A).

Two crystalline forms of the succinate salt were prepared from the scale-up experiments (Table 22A). Material exhibiting the XRPD pattern of crystalline 1 resulted from the following experiments: fast evaporation in methanol, fast evaporation in toluene:methanol 1:1, and slow evaporation in methanol: TFE 1:10. The structure of the succinate salt produced from methanol was confirmed by ¹H NMR (FIG. 60, Table 49A).

A new material with an XRPD pattern designated as crystalline 3 was produced from a fast evaporation experiment in methanol: TFE 1:10. Based on proton NMR, the succinate salt of crystalline 3 had residual amounts of trifluoroethanol (FIG. 62, Table 50A).

The aqueous solubility of the succinate salt was approximately 7-8 mg/mL (Table 63A).

TABLE 48A
Characterization of Succinate Salt, Crystalline 1

| Technique | Analysis/Result |
|---|---|
| XRPD | crystalline 1 |
| ¹H NMR | consistent w/structure |

TABLE 49A
Characterization of Succinate Salt, Crystalline 2

| Technique | Analysis/Result |
|---|---|
| XRPD | crystalline 2 |
| ¹H NMR | impurities present |

TABLE 50A
Characterization of Succinate Salt, Crystalline 3

| Technique | Analysis/Result |
|---|---|
| XRPD | crystalline 3 |
| ¹H NMR | 0.38 mole of TFE per 1 mole of compound (residual TFE) |

Sulfate

Four new crystalline XRPD patterns were observed in the wellplate experiments with sulfuric acid (FIGS. 63a to 63l, Table 19A, Table 21A):
crystalline 1 was produced in experiments in acetone, methyl ethyl ketone, and trifluoroethanol. It was also observed in crystallization experiments using methanol solutions with acetonitrile, isopropanol, and toluene as antisolvents. Low crystalline 1 material resulted from experiments utilizing methanol and methyl ethyl ketone as solvents. Material exhibiting an XRPD pattern designated as crystalline 1 minus peaks was produced in experiments in methanol: ethyl acetate and methanol:isopropanol 1:1;
crystalline 2 was produced in an experiment in methanol; crystalline 2 minus peaks was produced in a recrystallization experiment using methanol: ethyl acetate 1:1;
crystalline 3 was produced in an experiment in acetone;
crystalline 4 was produced in an experiment in methanol.

Five crystalline forms of the sulfate salt were prepared from the scale-up experiments (Table 22A). Material exhibiting the XRPD pattern of crystalline 1 resulted from a fast evaporation experiment in methanol. Two equivalents of the free base were utilized in the salt preparation. The structure of the sulfate salt was confirmed by proton NMR (FIG. 64).

The sulfate salt (crystalline 1) was characterized using thermal techniques (FIG. 65). Two weight losses were observed in TG: an immediate weight loss of approximately 1.7% from 25 to 50° C. followed by a weight loss of approximately 1.5% from 50 to 150° C. The DSC thermogram exhibited two endotherms at 115 and 215° C. The first endotherm was broader than what is typically attributed to the melt and probably resulted from a simultaneous melt and dehydration. The second endotherm overlapping with an exotherm at approximately 223° C. probably corresponded to decomposition.

Materials with crystalline patterns 2-4 observed earlier in the wellplate preparations were not reproduced. Material of crystalline 2 minus peaks was determined to be the hydrosulfate salt by proton NMR (one equivalent of sulfuric acid used FIG. 66, Table 52A). Impurities were present in the material.

Materials exhibiting new XRPD patterns designated as crystalline 5, 6, 7, and low crystalline 8 were prepared from the scale-up experiments as summarized in FIGS. 63i to 63l and Table 22A. The following salts were analyzed by ¹H NMR:
crystalline 5, hydrosulfate (one equivalent of free base used, FIG. 67, Table 53A);
crystalline 6, sulfate (one equivalent of free base used, FIG. 68, Table 54A);
crystalline 7, sulfate (two equivalents of free base used, FIG. 69, Table 55A).

The aqueous solubility of the sulfate salt was lower than 1 mg/mL, and the hydrosulfate salt approximately 1 mg/mL (Table 63A).

TABLE 51A
Characterization of Sulfate Salt, Crystalline 1

| Technique | Analysis/Result |
|---|---|
| XRPD | Form A (crystalline 1) |
| ¹H NMR | sulfate (2:1 API:acid^{c}) |
| DSC^{a} | endo 115 (broad), 215, exo 223 |
| TGA^{b} | 1.68 @ 25-50 |
| | 1.54 @ 50-150 |

^{a}endo = endotherm, exo = exotherm, temperatures (C.°) reported are transition maxima. Temperatures are rounded to the nearest degree.
^{b}weight loss (%) at a certain temperature; weight changes (%) are rounded to 2 decimal places; temperatures are rounded to the nearest degree
^{c}actual ratio used to make the salt

TABLE 52A
Characterization of Hydrosulfate Salt, Crystalline 2 minus peaks

| Technique | Analysis/Result |
|---|---|
| XRPD | crystalline 2 minus peaks |
| ¹H NMR | hydrosulfate, impurities present |

TABLE 53A
Characterization of Hydrosulfate Salt, Crystalline 5

| Technique | Analysis/Result |
|---|---|
| XRPD | crystalline 5 |
| ¹H NMR | hydrosulfate (1:1 API:acid^{a}) |

^{a}actual ratio used to make the salt

TABLE 54A
Characterization of Sulfate Salt, Crystalline 6

| Technique | Analysis/Result |
|---|---|
| XRPD | crystalline 6 |
| ¹H NMR | sulfate (1:1 API:acid^{a}) |

^{a}actual ratio used to make the salt

TABLE 55A
Characterization of Sulfate Salt, Crystalline 7

| Technique | Analysis/Result |
|---|---|
| XRPD | crystalline 7 |
| ¹H NMR | sulfate (2:1 APL:acid^{a}) |

^{a}actual ratio used to make the salt

Solubility of the Salts

(1R)-10-Camphorsulfonate Salt

Approximate solubilities of (1R)-10-camphorsulfonate (camsylate) salt were determined in solvents listed in Table 56A. The (1R)-10-camphorsulfonate salt showed low solubilities in methanol and 2,2,2-trifluoroethanol (approx. 3 mg/mL) and was practically insoluble in other organic solvents and water.

Fumarate Salt

Approximate solubilities of the fumarate salt were determined in solvents listed in Table 57A. The fumarate salt was poorly soluble in water (approx. 1.4 mg/mL) and insoluble in organic solvents.

Malonate Salt

Approximate solubilities of the malonate salt were determined in solvents listed in Table 58A. The malonate salt showed low solubilities in methanol, water, acetone, and 2,2,2-trifluoroethanol and no solubility in other organic solvents.

L-Tartrate Salt

Approximate solubilities of the L-tartrate salt were determined in solvents listed in Table 59A. The L-tartrate salt showed low solubilities in methanol (approx. 8 mg/mL), acetone and water (approx. 1 mg/mL) and no solubility in other organic solvents.

Tosylate Salt

Approximate solubilities of the tosylate salt were determined in solvents listed in Table 60A.

Other Salts

Aqueous solubilities of the crystalline salts from the wellplates or scale-up preparations were estimated (Table 63A).

TABLE 56A
Approximate solubilities of (1R)-10-Camphorsulfonate salt

| Solvent | Solubility (mg/mL)^{a} |
|---|---|
| acetone | <2 |
| acetonitrile | <2 |
| 1,4-dioxane | <2 |
| ethanol | <2 |
| ethyl acetate | <2 |
| iso-propanol | <2 |
| methanol | 3 |
| methyl ethyl ketone | <2 |
| tetrahydrofuran (THF) | <2 |
| toluene | <2 |
| 2,2,2-trifluoroethanol | 3 |
| water | <2 |

^{a}Solubilities are calculated based on the total solvent used to give a solution; actual solubilities may be greater because of the volume of the solvent portions utilized or a slow rate of dissolution. Solubilities are reported to the nearest mg/mL.

TABLE 57A
Approximate Solubilities of Fumarate salt

| Solvent | Solubility (mg/mL)^{a} |
|---|---|
| acetone | <1 |
| acetonitrile | <1 |
| 1,4-dioxane | <1 |
| ethanol | <1 |
| ethyl acetate | <1 |
| iso-propanol | <1 |
| methanol | <1 |
| methyl ethyl ketone | <1 |
| tetrahydrofuran (THF) | <1 |
| toluene | <1 |
| 2,2,2-trifluoroethanol | <1 |
| water | 1.3^{b} |

^{a}Solubilities are calculated based on the total solvent used to give a solution; actual solubilities may be greater because of the volume of the solvent portions utilized or a slow rate of dissolution. Solubilities are reported to the nearest mg/mL.
^{b}A more precise measurement of solubility was required for this solvent.

TABLE 58A
Approximate Solubilities of Malonate Salt

| Solvent | Solubility (mg/mL)^{a} |
|---|---|
| acetone | 1 |
| acetonitrile | <1 |
| 1,4-dioxane | <1 |
| ethanol | <1 |
| ethyl acetate | <1 |
| iso-propanol | <1 |
| methanol | 3 |
| methyl ethyl ketone | <1 |
| tetrahydrofuran (THF) | <1 |
| toluene | <1 |
| 2,2,2-trifluoroethanol | 1 |
| water | 3 |

^{a}Solubilities are calculated based on the total solvent used to give a solution; actual solubilities may be greater because of the volume of the solvent portions utilized or a slow rate of dissolution. Solubilities are reported to the nearest mg/mL.

TABLE 59A
Approximate Solubilities of L-Tartrate Salt

| Solvent | Solubility (mg/mL)^{a} |
|---|---|
| acetone | 1 |
| acetonitrile | <1 |
| 1,4-dioxane | <1 |
| ethanol | <1 |
| ethyl acetate | <1 |
| iso-propanol | <1 |
| methanol | 8 |
| methyl ethyl ketone | <1 |
| tetrahydrofuran (THF) | <1 |
| toluene | <1 |
| 2,2,2-trifluoroethanol | <1 |
| water | 1 |

^{a}Solubilities are calculated based on the total solvent used to give a solution; actual solubilities may be greater because of the volume of the solvent portions utilized or a slow rate of dissolution. Solubilities are reported to the nearest mg/mL.

TABLE 60A
Approximate Solubilities of Tosylate salt

| Solvent | Solubility (mg/mL)^{a} |
|---|---|
| acetone | 1^{b} |
| acetonitrile | <1 |
| 1,4-dioxane | 1^{c} |
| ethanol | 5 |
| ethyl acetate | <1 |
| iso-propanol | <1 |
| methanol | 19 |
| methyl ethyl ketone | 1^{b} |
| tetrahydrofuran (THF) | <1 |
| toluene | <1 |
| 2,2,2-trifluoroethanol | 4 |
| water | 6 |

^{a}Solubilities are calculated based on the total solvent used to give a solution; actual solubilities may be greater because of the volume of the solvent portions utilized or a slow rate of dissolution. Solubilities are reported to the nearest mg/mL.
^{b}Dissolved after approximately 2 days.
^{c}Dissolved after approximately 0.5 h.

TABLE 61A
Approximate Solubilities of Acetate salt

| Solvent | Solubility (mg/mL)^{a} |
|---|---|
| acetone | 2 |
| ethyl acetate | <1 |
| iso-propanol | 1 |
| methyl ethyl ketone | <1 |

^{a}Solubilities are calculated based on the total solvent used to give a solution; actual solubilities may be greater because of the volume of the solvent portions utilized or a slow rate of dissolution. Solubilities are reported to the nearest mg/mL.

TABLE 62A
Approximate Solubilities of Adipate salt

| Solvent | Solubility (mg/mL)^{a} |
|---|---|
| acetone | 3 |
| ethyl acetate | <1 |
| iso-propanol | 1 |
| methyl ethyl ketone | 1 |

^{a}Solubilities are calculated based on the total solvent used to give a solution; actual solubilities may be greater because of the volume of the solvent portions utilized or a slow rate of dissolution. Solubilities are reported to the nearest mg/mL.

TABLE 63A
Approximate Aqueous Solubilities of Compound 2 Salts
(crude materials)

| Salt | Solubility (mg/mL)^{a} |
|---|---|
| acetate | 18 |
| adipate | 10 |
| citrate-crystalline 1 | 2 |
| citrate-crystalline 2 | 7 |
| gentisate | <1 |
| glutarate | 3 |
| glycolate | 10 |
| hydrobromide-crystalline 1 | >32 |
| hydrobromide-crystalline 3 | >34 |
| L-malate | 4 |
| maleate | 3 |
| succinate-crystalline 1 | 8 |
| succinate-crystalline 3 | 7 |
| phosphate- | 9 |
| crystalline 5 ≡ crystalline 8 + peaks | |
| sulfate-crystalline 1 | <1 |
| sulfate-crystalline 6 | <1 |
| hydrosulfate-crystalline 5 | 1 |

^{a}Solubilities are calculated based on the total solvent used to give a solution; actual solubilities may be greater because of the volume of the solvent portions utilized or a slow rate of dissolution. Solubilities are reported to the nearest mg/mL.

TABLE 64A
Approximate Aqueous Solubilities of Compound 2 Salts (scale-up
crystallizations)

| Salt | Solubility (mg/mL)^{a} |
|---|---|
| acetate | 14.3 |
| adipate | 9.5 |
| citrate-crystalline 2 | 11.5 |
| glycolate | 26.5 |
| hydrobromide-crystalline 1 | 16^{b} |
| phosphate-crystalline 2 | 1.8 |
| phosphate-crystalline 8 | 3.4 |

^{a}Solubilities are calculated based on the total solvent used to give a solution; actual solubilities may be greater because of the volume of the solvent portions utilized or a slow rate of dissolution.
^{b}Mean value of 22.5 mg/mL (2449-53-01) and 10.4 mg/mL (2449-84-01).

The most preferred methods of preparing the various polymorphic forms are given below. Each process description defines a further aspect of the present invention.

After each process, the resulting material was analyzed by XRPD and in some instances other analytical methods and designated as the titled material.

### A. Preparation of L-Tartrate Salt Form A

20.1 mg of L-Tartrate salt was left to slurry in 20 mL of acetonitrile for 7 days under ambient conditions.

### 25B. Preparation of L-Tartrate Salt Form B

24.0 mg of L-Tartrate salt was left to slurry in 20 mL of ethyl acetate for 7 days under ambient conditions.

### C. Preparation of Malonate Salt

24.5 mg malonate salt was left to slurry in 20 mL of methyl ethyl ketone for 7 days under ambient conditions.

### D. Preparation of Tosylate Salt Form A

A filtered solution of 21.2 mg of tosylate salt in 1.1 mL of methanol was allowed to fast evaporate under ambient conditions.

### E. Preparation of Tosylate Salt Form B

21.6 mg of tosylate salt was left to slurry in 20 mL of acetonitrile for 7 days under ambient conditions.

### F. Preparation of Tosylate Salt Form C

44.5 mg of tosylate salt was left to slurry in 2 mL of iso-propanol for 4 days under ambient conditions.

### G. Preparation of Tosylate Salt Form E

(a) 49.1 mg of tosylate salt was dissolved in 10 mL of 2,2,2-trifluoroethanol with sonication. 3 of 10 mL of 2,2,2-trifluoroethanol were added with sonication, the rest without. Solution was filtered then allowed to fast evaporate under ambient conditions in a hood.

(b) A filtered solution of 21.6 mg of tosylate salt in 5.0 mL of 2,2,2-trifluoroethanol was allowed to fast evaporate under ambient conditions.

### H. Preparation of Tosylate Salt Form F

20.3 mg of tosylate salt was left to slurry in 20 mL of ethyl acetate for 7 days under ambient conditions.

### I. Preparation of Tosylate Salt Form G

A filtered solution of tosylate salt in 4 mL of water was allowed to fast evaporate under ambient conditions.

### J. Preparation of Tosylate Salt Form H

51.8 mg of tosylate salt was left to slurry in 2 mL of tetrahydrofuran (THF) for 4 days under ambient conditions.

### K. Preparation of (1R)-10-Camphorsulfonate Salt

21.1 mg of camsylate salt was left to slurry in 10 mL of acetone under ambient conditions.

### L. Preparation of Fumarate Salt

22.8 mg of fumarate salt was left to slurry in 20 mL of acetone for 7 days under ambient conditions.

### M. Preparation of Acetate Salt Form 1

5 mL of methanol was dispensed into 50.0 mg of compound 2 with sonication. 10 μL of glacial acetic acid was dispensed into the solution with stirring. The solution was then allowed to fast evaporate to dryness under ambient conditions.

### N. Preparation of Adipate Salt Form 1

Approximately 200 mg of compound 2 was dissolved in 5.5 mL of methanol with stirring on a hot plate. Temperature in the solution was measured at 55° C. 98.9 mg of adipic acid were dissolved in 0.3 mL of methanol at 55° C. The clear acid solution was added to the compound 2 solution with stirring. The solution was allowed to fast evaporate to dryness under ambient conditions in a hood.

### O. Preparation of Glutaric Salt Form 1

51.1 mg of compound 2 was dissolved in 3.5 mL of methanol with sonication. 23.1 mg of glutaric acid were dissolved in 0.5 mL of methanol and added to the free base solution. 4 mL of ethyl acetate was added to the solution. The solution was allowed to fast evaporate to dryness under ambient conditions in a hood.

### P. Preparation of Glycolic Salt Form 1

202.8 mg of compound 2 was dissolved in 6 mL of methanol with stirring on a hot plate. Temperature in the solution was measured at 50° C. 52.0 mg of glycolic acid were dissolved in 0.1 mL of methanol at 50° C. The clear acid solution was added to the free base solution. 6.1 mL of acetonitrile was added to the solution. The solution was allowed to slow cool under ambient conditions.

### Q. Preparation of L-malic Salt Form 1

51.5 mg of compound 2 was dissolved in 4 mL of methanol with sonication. 23.8 mg of L-malic acid were dissolved in 0.1 mL of methanol and added to the free base solution. The solution was allowed to fast evaporate to dryness under ambient conditions in a hood.

### R. Preparation of Citric Salt Crystalline Form 1

Preparation of the citric salt crystalline form 1 was carried out in a 96-well polypropylene plate using the following procedure. A solution was prepared by dissolving compound 2 in acetone at approximately 10 mg/mL, adding 0.1 mL of the solution in a well. Dilute citric acid solution was added (in methanol, 0.1M) to the well at slightly more than half a molar equivalent with respect to the active pharmaceutical ingredient (API). The plate was covered with a selfadhesive aluminum foil cover and allowed to mix at approximately 25 RPM on an ambient temperature orbital shaker for 11 days. Some evaporation occurred during mixing. The plate was left uncovered to complete evaporation under ambient conditions. The plate was then used in a recrystallization experiment. 25 μL of methanol was added to the well and the plate was loaded on an ambient-temperature orbital shaker at approximately 150 RPM for 1 hour. 75 μL of acetonitrile were added to the well C03. Finally, the plate was placed in a hood and allowed to evaporate until dry under ambient conditions.

### S. Preparation of Citric Salt Crystalline Form 2

Approximately 200 mg of compound 2 was dissolved in 8 mL of acetone with stirring on a hot plate. Temperature in the solution was measured at 50° C. 141.9 mg of citric acid monohydrate were dissolved in 0.2 mL of methanol on a hot plate with stirring. The citric acid solution was added to the free base solution with stirring. Temperature in the solution was measured at 50° C. The solution was allowed to slow cool under ambient conditions.

### T. Preparation of Gentisic Salt Crystalline Form 1

50.8 mg of compound 2 was dissolved in 3.5 mL of methanol with sonication. 26.9 mg of gentisic acid were dissolved in 0.5 mL of methanol and added to the free base solution. 4 mL of ethyl acetate was added to the solution. The solution was allowed to fast evaporate to dryness in a hood under ambient conditions.

### U. Preparation of Gentisic Salt Crystalline Form 2

Preparation of the gentisic salt crystalline form 2 was carried out in a 96-well polypropylene plate using the following procedure. A solution was prepared by dissolving compound 2 in methanol at approximately 10 mg/mL, adding 0.1 mL of the solution in a well. Dilute gentisic acid solution was added (in methanol, 0.1M) to the well at slightly more than one molar equivalent with respect to the API. The plate was covered with a self-adhesive aluminum foil cover and allowed to mix at approximately 25 RPM on an ambient-temperature orbital shaker for 11 days. Some evaporation occurred during mixing. The plate was observed after 3 days by optical microscopy and returned to the shaker. The plate was left uncovered to complete evaporation under ambient conditions. The plate was then used in a recrystallization experiment. 75 μL of methanol was added to the well and the plate was loaded on an ambient-temperature orbital shaker at approximately 150 RPM for 1 hour. 75 μL of ethyl acetate were added to the well D06. Finally, the plate was placed in a hood and allowed to evaporate until dry under ambient conditions. The resulting material was analyzed by XRPD and designated as gentisate salt crystalline form 2.

### V. Preparation of Maleic Salt Crystalline Pattern 1

Preparation of the maleic salt crystalline pattern 1 was carried out in a 96-well polypropylene plate using the following procedure. A solution was prepared by dissolving compound 2 in methanol at approximately 10 mg/mL, adding 0.1 mL of the solution in a well. Dilute maleic acid solution was added (in methanol, 0.1M) to the well at slightly more than half a molar equivalent with respect to the API. The plate was covered with a self-adhesive aluminum foil cover and allowed to mix at approximately 25 RPM on an ambient-temperature orbital shaker for 8 days. Some evaporation occurred during mixing. The plate was observed after 3 days by optical microscopy and returned to the shaker. The plate was left uncovered to complete evaporation under ambient conditions. The plate was then used in a recrystallization experiment. 75 μL of methanol was added to the well and the plate was loaded on an ambient-temperature orbital shaker at approximately 150 RPM for 30 minutes. 75 μL of ethyl acetate were added to the well C05. Finally, the plate was fast evaporated until dry under ambient conditions.

### W. Preparation of Maleic Salt Crystalline 1 Plus Peaks

50.3 mg of compound 2, batch AB060109/1 was dissolved in 4 mL of methanol with sonication. 19.6 mg of maleic acid were dissolved in 0.2 mL of methanol and added to the free base solution. The solution was fast evaporated until dryness under ambient conditions in a hood.

### X. Preparation of Hydrobromide Salt Crystalline Form 1

Preparation of the hydrobromide salt crystalline form 1 was carried out in a 96-well polypropylene plate using the following procedure. A solution was prepared by dissolving compound 2 in 2,2,2-trifluoroethanol at approximately 10 mg/mL, adding 0.1 mL of the solution in a well. Dilute HBr acid solution was added (in methanol, 0.1M) to the well at slightly more than one molar equivalent with respect to the API. The plate was covered with a self-adhesive aluminum foil cover and allowed to mix at approximately 25 RPM on an ambient temperature orbital shaker for 8 days. Some evaporation occurred during mixing. The plate was observed after 3 days by optical microscopy and returned to the shaker. The plate was left uncovered to complete evaporation under ambient conditions. The plate was then used in a recrystallization experiment. 75 μL of methanol was added to the well and the plate was loaded on an ambient-temperature orbital shaker at approximately 150 RPM for 30 minutes. 75 μl, of toluene were added to the well A12. Finally, the plate was fast evaporated until dry under ambient conditions.

### Y. Preparation of Hydrobromide Salt Crystalline Form 2

Preparation of the hydrobromide salt crystalline form 2 was carried out in a 96-well polypropylene plate using the following procedure. A solution was prepared by dissolving compound 2 in acetone at approximately 10 mg/mL, adding 0.1 mL of the solution in a well. Dilute HBr acid solution was added (in methanol, 0.1M) to the well at slightly more than one molar equivalent with respect to the API. The plate was covered with a self-adhesive aluminum foil cover and allowed to mix at approximately 25 RPM on an ambient-temperature orbital shaker for 0.8 days. Some evaporation occurred during mixing. The plate was left uncovered to complete evaporation under ambient conditions. The plate was then used in a recrystallization experiment. 75 μL of methanol was added to the well and the plate was loaded on an ambient-temperature orbital shaker at approximately 150 RPM for 30 minutes. 75 μL of acetonitrile were added to the well A01. Finally, the plate was fast evaporated until dry under ambient conditions.

### Z. Preparation of Hydrobromide Salt Crystalline Form 3

50.2 mg of compound 2 was dissolved in 6 mL of acetone with sonication. 18.7 μL of HBr acid were dispensed into the free base solution with sieving. The solution was fast evaporated until dryness under ambient conditions.

### AA. Preparation of Succinate Salt Crystalline Form 1

Preparation of the succinate salt crystalline form 1 was carried out in a 96-well polypropylene plate using the following procedure. A solution was prepared by dissolving compound 2 in methanol at approximately 10 mg/mL, adding 0.1 mL of the solution in a well. Dilute succinic acid solution was added (in methanol, 0.1M) to the well E06 at slightly more than half a molar equivalent with respect to the API. The plate was covered with a self-adhesive aluminum foil cover and allowed to mix at approximately 25 RPM on an ambient-temperature orbital shaker for 8 days. Some evaporation occurred during mixing. The plate was left uncovered to complete evaporation under ambient conditions.

### BB. Preparation of Succinate Salt Crystalline Form 2

Preparation of the succinate salt crystalline form 2 was carried out in a 96-well polypropylene plate using the following procedure. A solution was prepared by dissolving compound 2 in 2,2,2-trifluoroethanol at approximately 10 mg/mL, adding 0.1 mL of the solution in well E12. Dilute succinic acid solution was added (in methanol, 0.1M) to the well at slightly more than half a molar equivalent with respect to the API. The plate was covered with a self-adhesive aluminum foil cover and allowed to mix at approximately 25 RPM on an ambient-temperature orbital shaker for 8 days. Some evaporation occurred during mixing. The plate was left uncovered to complete evaporation under ambient conditions. The plate was then used in a recrystallization experiment. 75 μL of methanol was added to the well and the plate was loaded on an ambient-temperature orbital shaker at approximately 150 RPM for 30 minutes. 75 μL of toluene were added to the well. Finally, the plate was fast evaporated until dry under ambient conditions.

### CC. Preparation of Succinate Salt Crystalline Form 3

102.4 mg of compound 2, batch AB060109/1 was dissolved in 8 mL of 2,2,2-trifluoroethanol. 41.3 mg of succinic acid was dissolved in 0.8 mL of methanol and added to the free base solution. 4.4 mL of the solution were taken out for another sample. The remaining solution was fast evaporated until dryness under ambient conditions in a hood.

### DD. Preparation of Phosphoric Salt Crystalline Form 1

Preparation of the phosphoric salt crystalline form 1 was carried out in a 96-well polypropylene plate using the following procedure. A solution was prepared by dissolving compound 2 in 2,2,2-trifluoroethanol at approximately 10 mg/mL, adding 0.1 mL of the solution in well G12. Dilute phosphoric acid solution was added (in methanol, 0.1M) to the well at slightly more than a third of a molar equivalent with respect to the API. The plate was covered with a self-adhesive aluminum foil cover and allowed to mix at approximately 25 RPM on an ambient-temperature orbital shaker for 8 days. Some evaporation occurred during mixing. The plate was left uncovered to complete evaporation under ambient conditions.

### EE. Preparation of Phosphoric Salt Crystalline Form 2

Preparation of the phosphoric salt crystalline form 2 was carried out in a 96-well polypropylene plate using the following procedure. A solution was prepared by dissolving compound 2 in acetone at approximately 10 mg/mL, adding 0.1 mL of the solution in well G02. Dilute phosphoric acid solution was added (in methanol, 0.1M) to the well at slightly more than a third of a molar equivalent with respect to the API. The plate was covered with a self-adhesive aluminum foil cover and allowed to mix at approximately 25 RPM on an ambient-temperature orbital shaker for 8 days. Some evaporation occurred during mixing. The plate was left uncovered to complete evaporation under ambient conditions.

### FF. Preparation of Phosphoric Salt Crystalline Form 3

Preparation of the phosphoric salt crystalline form 3 was carried out in a 96-well polypropylene plate using the following procedure. A solution was prepared by dissolving compound 2 in methyl ethyl ketone at approximately. 10 mg/mL, adding 0.1 mL of the solution in well G07. Dilute phosphoric acid solution was added (in methanol, 0.1M) to the well at slightly more than a third of a molar equivalent with respect to the API. The plate was covered with a self-adhesive aluminum foil cover and allowed to mix at approximately 25 RPM on an ambient-temperature orbital shaker for 8 days. Some evaporation occurred during mixing. The plate was left uncovered to complete evaporation under ambient conditions. The plate was then used in a recrystallization experiment. 75 μL of methanol was added to the well and the plate was loaded on an ambient-temperature orbital shaker at approximately 150 RPM for 30 minutes. 75 μL of isopropanol were added to the well. Finally, the plate was fast evaporated until dry under ambient conditions.

### GG. Preparation of Phosphoric Salt Crystalline Form 4

Preparation of the phosphoric salt crystalline form 4 was carried out in a 96-well polypropylene plate using the following procedure. A solution was prepared by dissolving compound 2 in methyl ethyl ketone at approximately 10 mg/mL, adding 0.1 mL of the solution in well G08. Dilute phosphoric acid solution was added (in methanol, 0.1M) to the well at slightly more than a third of a molar equivalent with respect to the API. The plate was covered with a self-adhesive aluminum foil cover and allowed to mix at approximately 25 RPM on an ambient-temperature orbital shaker for 8 days. Some evaporation occurred during mixing. The plate was left uncovered to complete evaporation under ambient conditions. The plate was then used in a recrystallization experiment. 75 μL of methanol was added to the well and the plate was loaded on an ambient-temperature orbital shaker at approximately 150 RPM for 30 minutes. 75 μL of isopropanol were added to the well. Finally, the plate was fast evaporated until dry under ambient conditions.

### HH. Preparation of Phosphoric Salt Crystalline Form 5

49.7 mg of Compound 2 was dissolved in 5 mL of methanol with sonication. Dispensed 11.5 μL of phosphoric acid into the free base solution with stirring. The solution was allowed to fast evaporate until dryness under ambient conditions.

### II. Preparation of Phosphoric Salt Crystalline Form 6

1 mL of Compound 2 was dissolved in 1 mL of methanol. The solution was stirred on a RT plate at 60 RPM. 73 μL of phosphoric acid was added. The experiment was performed in a dark fume hood.

### JJ. Preparation of Phosphoric Salt Crystalline Form 7

10 mg of Compound 2 was dissolved in 5 mL of methanol and 1 mL of 2,2,2-trifluoroethanol. The solution was stirred on a RT plate at 60 RPM. 73 μL of phosphoric acid was added. The experiment was performed in a dark fume hood. A white precipitate (solids) was instantly generated upon acid addition.

### KK. Preparation of Phosphoric Salt Crystalline Form 8

103 mg of Compound 2 was dissolved in 10 mL of methanol with sonication. 22.6 μL of 85% phosphoric acid were added to the free base solution with stirring. The solution was allowed to fast evaporate until dryness under ambient conditions in a hood.

### LL. Preparation of Sulfuric Salt Crystalline Form 1

64 mg of Compound 2 was dissolved in 2 mL of methanol. 98 mg of sulfuric acid was dissolved in 1 mL of methanol and added to the free base solution. The solution was shaken then allowed to fast evaporate until dryness under ambient conditions.

### MM. Preparation of Sulfuric Salt Crystalline Form 2

Preparation of the sulfuric salt crystalline form 2 was carried out in a 96-well polypropylene plate using the following procedure. A solution was prepared by dissolving Compound 2 in methanol at approximately 10 mg/mL, adding 0.1 mL of the solution in well F06. Dilute sulfuric acid solution was added (in methanol, 0.1M) to the well at slightly more than half the molar equivalent with respect to the API. The plate was covered with a self-adhesive aluminum foil cover and allowed to mix at approximately 25 RPM on an ambient-temperature orbital shaker for 8 days. Some evaporation occurred during mixing. The plate was observed after 3 days by optical microscopy and returned to the shaker. The plate was left uncovered to complete evaporation under ambient conditions.

### NN. Preparation of Sulfuric Salt Crystalline Form 3

Preparation of the sulfuric salt crystalline form 3 was carried out in a 96-well polypropylene plate using the following procedure. A solution was prepared by dissolving Compound 2 in acetone at approximately 10 mg/mL, adding 0.1 mL of the solution in well F06. Dilute sulfuric acid solution was added (in methanol, 0.1M) to the well, at slightly more than half the molar equivalent with respect to the API. The plate was covered with a self-adhesive aluminum foil cover and allowed to mix at approximately 25 RPM on an ambient-temperature orbital shaker for 0.8 days. Some evaporation occurred during mixing. The plate was observed after 3 days by optical microscopy and returned to the shaker. The plate was left uncovered to complete evaporation under ambient conditions.

### OO. Preparation of Sulfuric Salt Crystalline Form 4

Preparation of the sulfuric salt crystalline form 4 was carried out in a 96-well polypropylene plate using the following procedure. A solution was prepared by dissolving Compound 2 in methanol at approximately 10 mg/mL, adding 0.1 mL of the solution in well F05. Dilute sulfuric acid solution was added (in methanol, 0.1M) to the well at slightly more than half the molar equivalent with respect to the API. The plate was covered with a self-adhesive aluminum foil cover and allowed to mix at approximately 25 RPM on an ambient-temperature orbital shaker for 8 days. Some evaporation occurred during mixing. The plate was observed after 3 days by optical microscopy and returned to the shaker. The plate was left uncovered to complete evaporation under ambient conditions.

### PP. Preparation of Sulfuric Salt Crystalline Form 5

64 mg of Compound 2 was dissolved in 5 mL of acetone. 99 mg of sulfuric acid was dissolved in 1 mL of acetone and added to the free base solution. The solution was shaken and sonicated, then allowed to fast evaporate until dryness under ambient conditions.

### QQ. Preparation of Sulfuric Salt Crystalline Form 6

49.9 mg of Compound 2 was dissolved in 4 mL of methanol with sonication. 9.4 μL of sulfuric acid were added to the free base solution. 4 mL of ethyl acetate were added to the free base solution. The solution was allowed to fast evaporate until dryness under ambient conditions.

### RR. Preparation of Sulfuric Salt Crystalline Form 7

62 mg of Compound 2 was dissolved in 5 mL of acetone. 99 mg of sulfuric acid was dissolved in 1 mL of acetone and added to the free base solution. The solution was shaken and sonicated, then allowed to fast evaporate until dryness under ambient conditions.

41 mg of the material were weighed into a vial. 2 mL of acetone were added. The mixture was shaken and sonicated then slurried at ambient temperature.

### SS. Preparation of Sulfuric Salt Crystalline Form 8

1 mL of Compound 2 was dissolved in 1 mL of 2,2,2-trifluoroethanol. The solution was stirred on a RT plate at 60 RPM. 73 μL of sulfuric acid was added. After a few minutes, the stir rate was briefly increased to 200 RPM, then reduced back to 60 RPM. The experiment was performed in a dark fume hood.

### TT. Preparation of Compound 2 Free Base Form A

30.9 mg of compound 2 was dissolved in 1 mL of acetonitrile with sonication. The solution was left to slurry for 7 days under ambient conditions.

It will be appreciated that the invention may be modified within the scope of the appended claims.

1.-175. (canceled)

176. Crystalline Form 1 (R)-5-(2-Aminoethyl)-1-(6,8-difluorochroman-3-yl)-1,3-dihydroimidazole-2-thione glycolate.

177. A pharmaceutical formulation comprising (R)-5-(2-Aminoethyl)-1-(6,8-difluorochroman-3-yl)-1,3-dihydroimidazole-2-thione glycolate and at least one pharmaceutically acceptable carrier or excipient, wherein the (R)-5-(2-Aminoethyl)-1-(6,8-difluorochroman-3-yl)-1,3-dihydroimidazole-2-thione glycolate comprises crystalline Form 1 (R)-5-(2-Aminoethyl)-1-(6,8-difluorochroman-3-yl)-1,3-dihydroimidazole-2-thione glycolate.

178. The pharmaceutical formulation of claim 177, wherein said (R)-5-(2-Aminoethyl)-1-(6,8-difluorochroman-3-yl)-1,3-dihydroimidazole-2-thione glycolate is present in a therapeutically effective amount.

179. A method of treating a condition in a subject in need thereof, comprising administering (R)-5-(2-Aminoethyl)-1-(6,8-difluorochroman-3-yl)-1,3-dihydroimidazole-2-thione glycolate comprising crystalline Form 1(R)-5-(2-Aminoethyl)-1-(6,8-difluorochroman-3-yl)-1,3-dihydroimidazole-2-thione glycolate to said subject.

180. The method according to claim 179, wherein said condition is a cardiovascular disorder.

181. The method according to claim 179, wherein said method further comprises peripherally-selective inhibition of DβH.
